(19) United States
(12) Patent Application Publication
(10) Pub. No.: US 2023/0329100 A1
(43) Pub. Date: Oct. 12, 2023

(54) LIGHT EMITTING ELEMENT AND AMINE COMPOUND FOR THE SAME

(71) Applicant: Samsung Display Co., Ltd., Yongin-si (KR)

(72) Inventors: SANGHYUN HAN, Yongin-si (KR); YOUNGJIN PARK, Yongin-si (KR); HANKYU PAK, Yongin-si (KR); HEECHOON AHN, Yongin-si (KR); HYUNAH UM, Yongin-si (KR); EUNJAE JEONG, Yongin-si (KR); SEOWON CHO, Yongin-si (KR); SOHEE JO, Yongin-si (KR)

(21) Appl. No.: 18/167,038

(22) Filed: Feb. 9, 2023

(30) Foreign Application Priority Data
Apr. 12, 2022 (KR) 10-2022-0045211

(51) Int. Cl. H10K 85/60; C07D 263/57; C09K 11/06; C07D 277/66; C07D 471/04; C07D 413/14; C07D 417/14

(52) U.S. Cl. CPC H10K 85/657; C07D 263/57; C09K 11/06; H10K 85/636; C07D 277/66; C07D 471/04; C07D 413/14; C07D 417/14; H10K 85/6572; H10K 50/11

(57) ABSTRACT

A light emitting element that includes a first electrode, a second electrode, a functional layer between the first electrode and the second electrode, and a capping layer on the second electrode and including an amine compound represented by Formula 1 is provided.

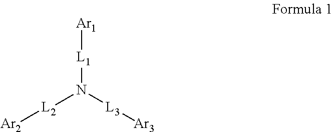

The light emitting element may exhibit high efficiency and high luminance characteristics.

### CROSS-REFERENCE TO RELATED APPLICATION

This application claims priority to and the benefit of Korean Patent Application No. 10-2022-0045211, filed on Apr. 12, 2022, the entire content of which is hereby incorporated by reference.

### BACKGROUND

Aspects of one or more embodiments of the present disclosure herein relate to an amine compound and a light emitting element including the same, and for example, to a light emitting element including a novel amine compound in a capping layer.

As image display devices, organic electroluminescence display devices and/or the like have recently been actively developed. The organic electroluminescence display devices and/or the like are display devices including self-luminescent light emitting elements in which holes and electrons injected from a first electrode and a second electrode recombine in an emission layer, and thus a luminescent material in the emission layer emits light to accomplish display (e.g., to display an image).

For application of light emitting elements to display devices, there is a demand or desire for light emitting elements having a low driving voltage, a high luminous efficiency, and a long life, and development of materials, for light emitting elements, capable of stably or suitably attaining such characteristics is being continuously required (sought).

In order to obtain highly efficient light emitting elements, capping layer materials for increasing light extraction efficiency of light generated from the light emitting elements are under development.

### SUMMARY

An aspect of one or more embodiments of the present disclosure is directed toward a light emitting element exhibiting excellent (high or suitable) luminous efficiency and high luminance characteristics.

An aspect of one or more embodiments of the present disclosure is directed toward an amine compound as a material for a light emitting element, which is designed to increase light efficiency.

Additional aspects will be set forth in part in the description which follows and, in part, will be apparent from the description, or may be learned by practice of the presented embodiments of the disclosure.

An embodiment of the present disclosure provides an amine compound represented by Formula 1.

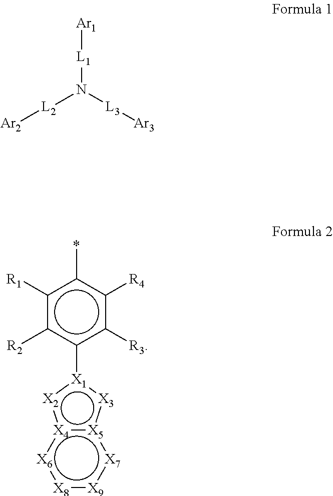

In Formula 1, *-L₁-Ar₁, *-L₂-Ar₂, and *-L₃-Ar₃ may each independently be represented by Formula 2. In Formula 2, R₁ to R₄ may each independently be a hydroxy group or a hydrogen atom, or bonded to an adjacent group to form a single bond; X₁ to X₉ may each independently be CH, C, N, O, or S; and at least one of *-L₁-Ar₁, *-L₂-Ar₂, or *-L₃-Ar₃ may include a hydroxyl group.

In an embodiment, Formula 2 may be represented by any one selected from among I-1 to I-3, and an embodiment in which*-L₁-Ar₁, *-L₂-Ar₂, and *-L₃-Ar₃ in Formula 1 are all I-1 may be excluded.

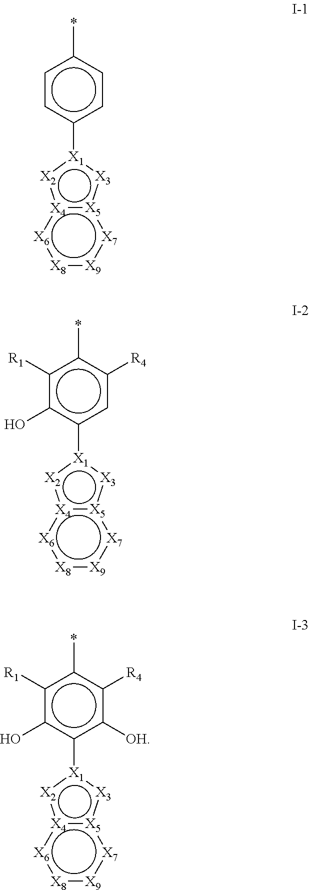

In I-2 and I-3, R₁ and R₄ may each independently be a hydrogen atom, or bonded to an adjacent group to form a single bond; and in I-1 to I-3, X₁ to X₉ may each independently be the same as defined in Formula 2.

In an embodiment, Formula 2 may be represented by any one selected from among I-4 to I-8.

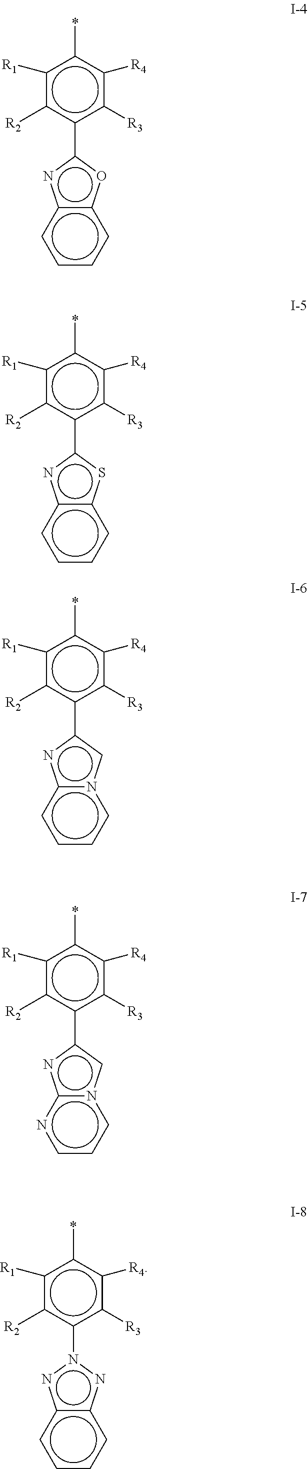

In I-4 to I-8, R₁ to R₄ may each independently be the same as defined in Formula 2.

In an embodiment, in Formula 2, at least one selected from among X₁ to X₃ may be N.

In an embodiment, Formula 1 may be represented by Formula 1-1.

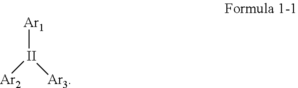

In Formula 1-1, II is represented by II-A or II-B.

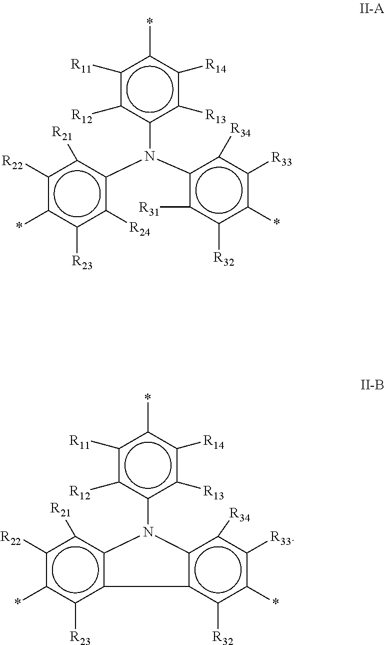

In II-A, at least one selected from among R₁₁ to R₁₄, R₂₁ to R₂₄, and R₃₁ to R₃₄ may be a hydroxy and the others (the R₁₁ to R₁₄, R₂₁ to R₂₄, and R₃₁ to R₃₄ that are not hydroxy) may be hydrogen atoms; and in II-B, at least one selected from among R₁₁ to R₁₄, R₂₁ to R₂₃, and R₃₂ to R₃₄ may be a hydroxy and the others (the R₁₁ to R₁₄, R₂₁ to R₂₃, and R₃₂ to R₃₄ that are not a hydroxy) may be hydrogen atoms.

In an embodiment, in Formula 1-1, II may be represented by any one selected from among II-1 to II-16.

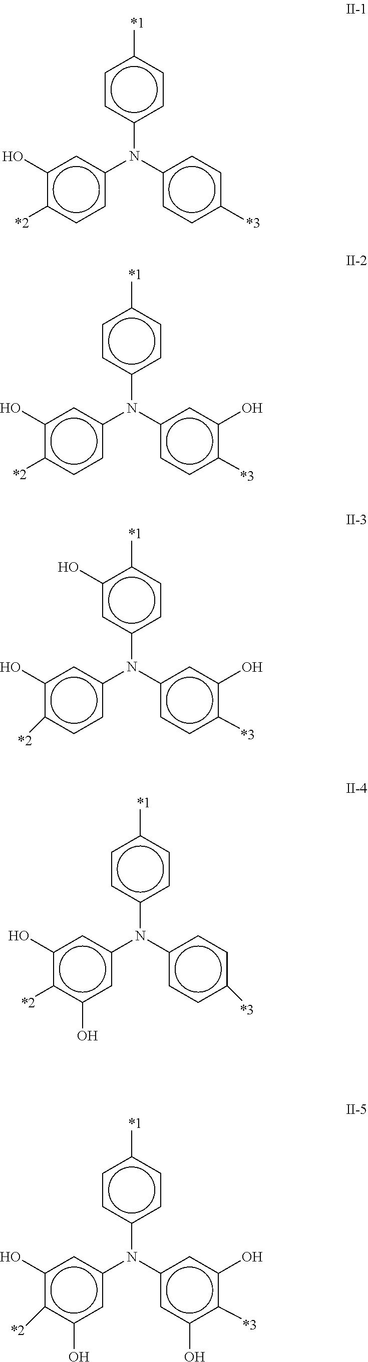

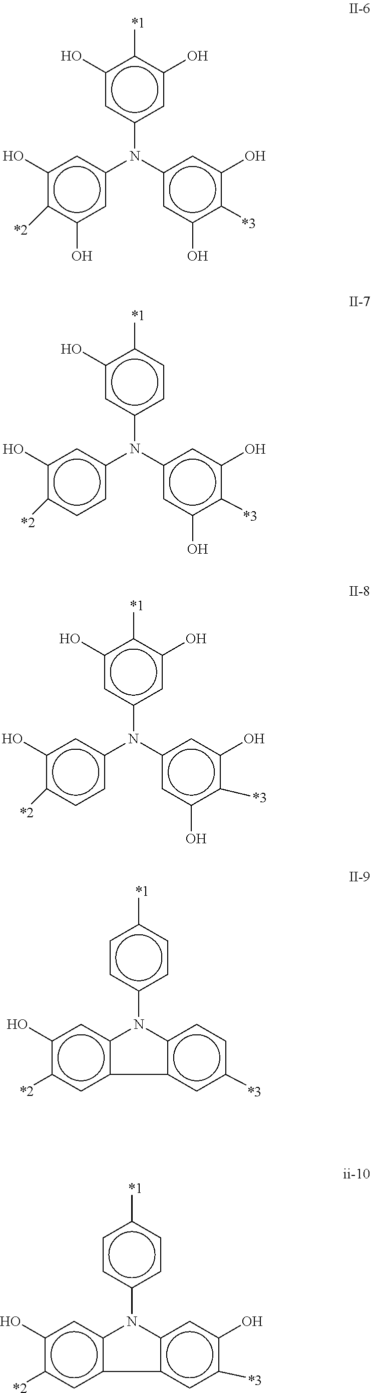

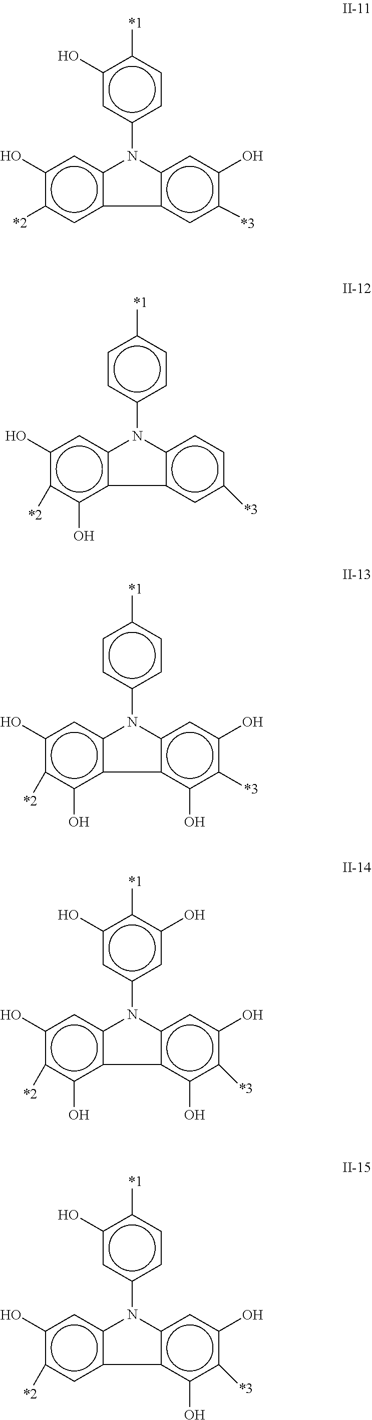

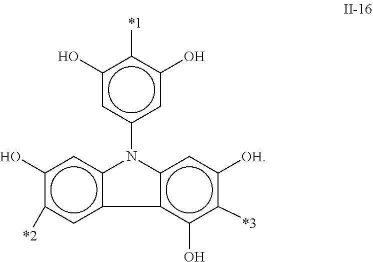

In II-1 to II-16, *1 is a portion to which Ar₁ is bonded, *2 is a portion to which Ar₂ is bonded, and *3 is a portion to which Ar₃ is bonded.

In an embodiment, in Formula 1-1, Ar₁ to Ar₃ may each independently be represented by any one selected from among III-1 to III-5.

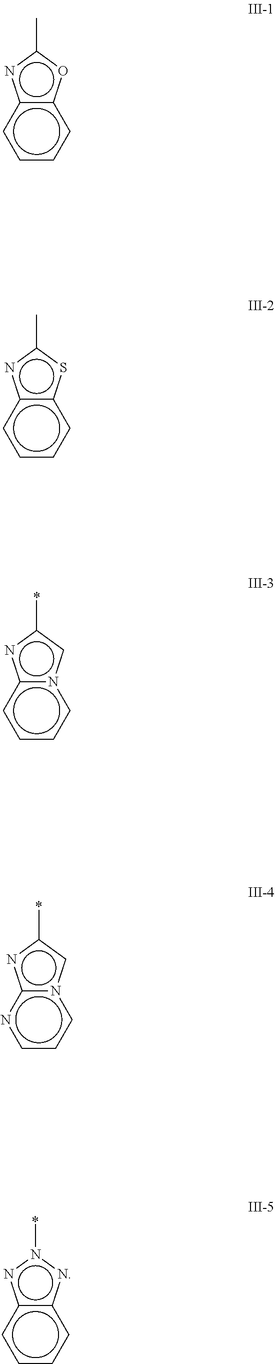

In an embodiment of the present disclosure, provided is an amine compound represented by Formula 1-1.

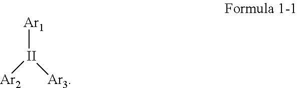

In Formula 1-1, II is represented by II-A or II-B, and Ar₁ to Ar₃ may each independently be a substituted or unsubstituted benzoazole derivative.

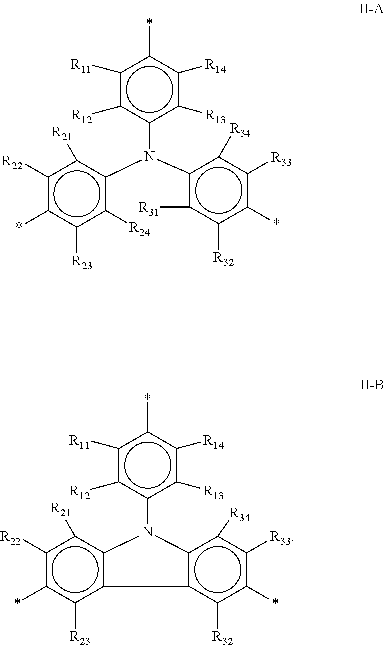

In II-A, at least one selected from among R₁₁ to R₁₄, R₂₁ to R₂₄, and R₃₁ to R₃₄ may be hydroxy and the others (the R₁₁ to R₁₄, R₂₁ to R₂₄, and R₃₁ to R₃₄ that are not hydroxy) may be hydrogen atoms; and in II-B, at least one selected from among R₁₁ to R₁₄, R₂₁ to R₂₃, and R₃₂ to R₃₄ may be hydroxy and the others (the R₁₁ to R₁₄, R₂₁ to R₂₃, and R₃₂ to R₃₄ that are not hydroxy) may be hydrogen atoms.

In an embodiment, in Formula 1-1, II may be represented by any one selected from among II-1 to II-16, and Ar₁ to Ar₃ may each independently be represented by any one selected from among III-1 to III-5.

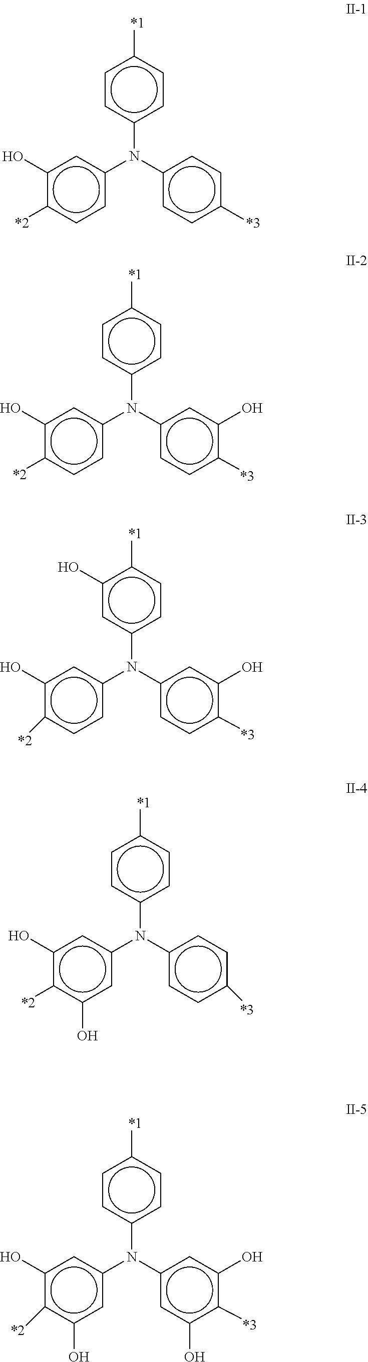

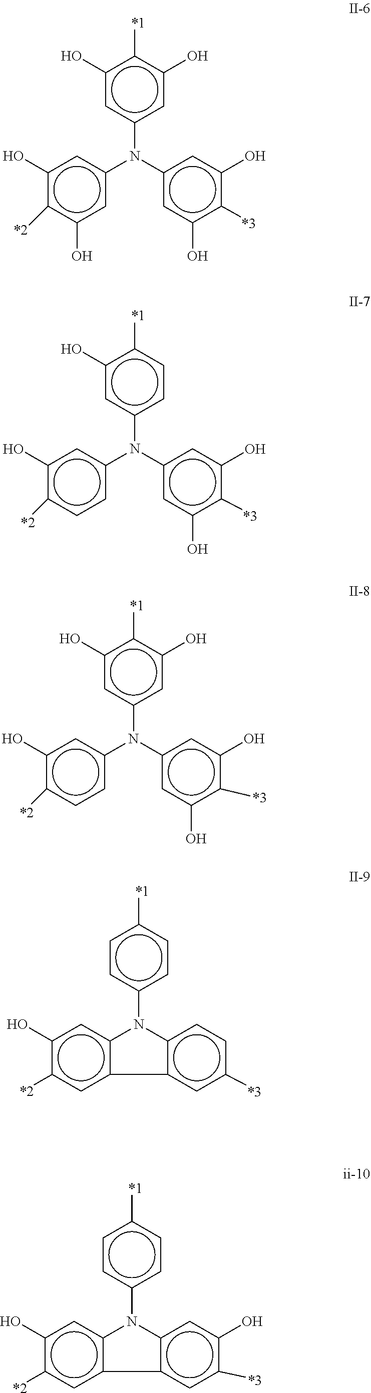

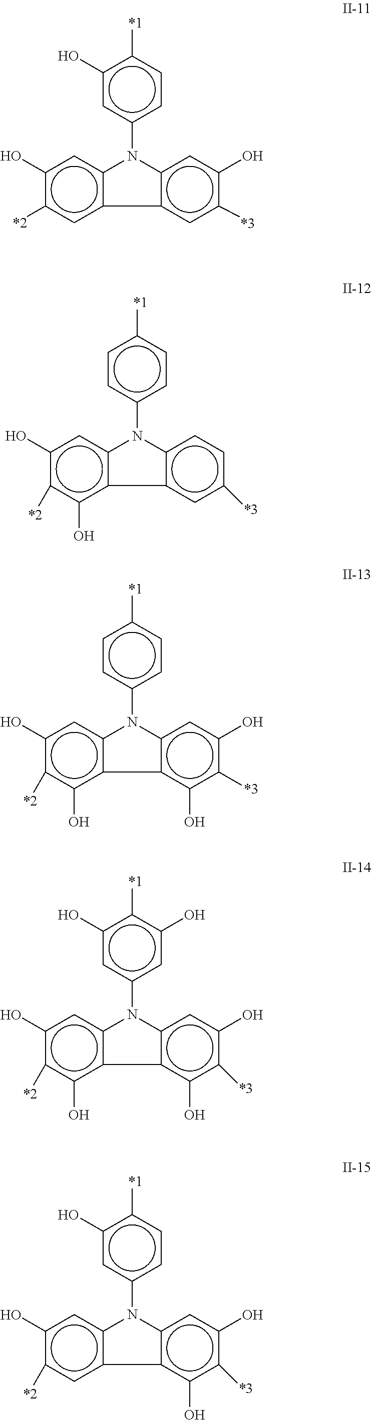

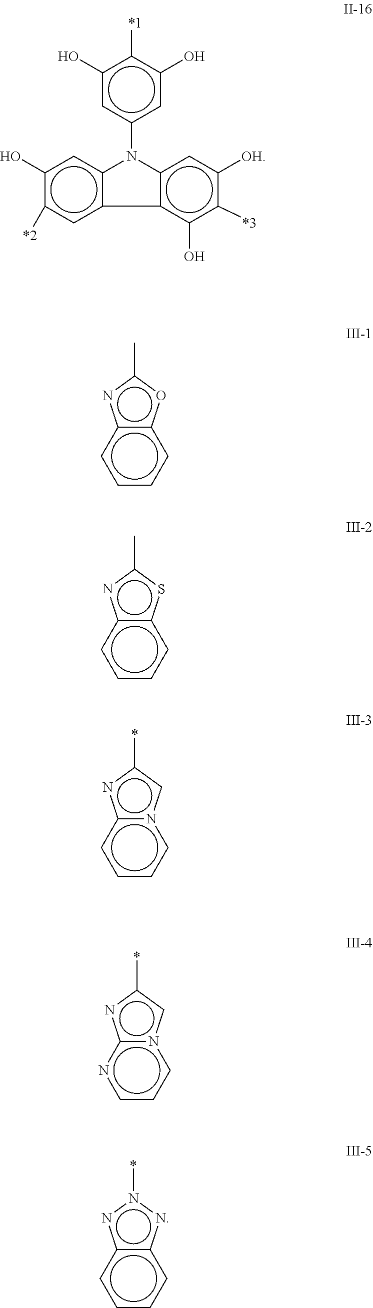

In II-1 to II-16, *1 is a portion to which Ar1 is bonded, *2 is a portion to which Ar2 is bonded, and *3 is a portion to which Ar3 is bonded.

In an embodiment of the present disclosure, a light emitting element includes: a first electrode; a second electrode on the first electrode; a functional layer between the first electrode and the second electrode; and a capping layer on the second electrode and including an amine compound represented by Formula 1.

In an embodiment, the functional layer may include an emission layer, a hole transport region disposed between the first electrode and the emission layer, and an electron transport region disposed between the emission layer and the second electrode.

In an embodiment, the emission layer may include a compound represented by Formula E-1.

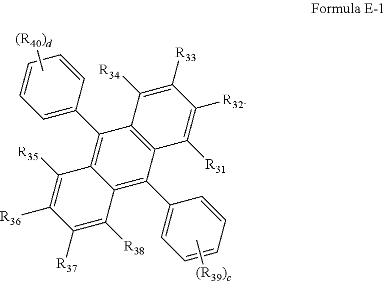

In Formula E-1, c and d may each independently be an integer from 0 to 5, and R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display device according to an embodiment;

FIG. 2 is a cross-sectional view of a display device according to an embodiment;

FIG. 3 is a cross-sectional view schematically showing a light emitting element according to an embodiment;

FIG. 4 is a cross-sectional view schematically showing a light emitting element according to an embodiment;

FIG. 5 is a cross-sectional view schematically showing a light emitting element according to an embodiment;

FIG. 6 is a cross-sectional view of a display device according to an embodiment;

FIG. 7 is a cross-sectional view of a display device according to an embodiment;

FIG. 8 is a cross-sectional view showing a display device according to an embodiment; and

FIG. 9 is a cross-sectional view showing a display device according to an embodiment.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

In describing the drawings, like reference numerals are utilized for like elements. In the drawings, the sizes of elements may be exaggerated for clarity. It will be understood that, although the terms “first”, “second”, etc. may be utilized herein to describe one or more suitable elements, these elements should not be limited by these terms. These terms are only utilized to distinguish one element from another. For example, a first element could be termed a second element, and, similarly, a second element could be termed a first element, without departing from the scope of example embodiments of the present disclosure. The terms of a singular form may include plural forms unless the context clearly indicates otherwise. As used herein, the singular forms “a”, “an” and “the” are intended to include the plural forms as well, unless the context clearly indicates otherwise.

In the present disclosure, it should be understood that the terms “comprise”, include”, or “have” are intended to specify the presence of stated features, integers, steps, operations, elements, components, or combinations thereof in the disclosure, but do not preclude the presence or addition of one or more other features, integers, steps, operations, elements, components, or combinations thereof.

In the present disclosure, it should be understood that when an element such as a layer, a film, a region, or a substrate is referred to as being “on” or “above” another element, it may be “directly on” the other element or intervening elements may also be present. In contrast, it should be understood that when an element such as a layer, a film, a region, or a substrate is referred to as being “beneath” or “under” another element, it may be “directly under” the other element or intervening elements may also be present. In some embodiments, in the present disclosure, it should be understood that when an element is referred to as being “on”, it may be as being “above” or “under” the other element.

In the present disclosure, the term “substituted or unsubstituted” may indicate that one is substituted or unsubstituted with at least one substituent selected from the group including (e.g., consisting of) a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the (example) substituents above may be substituted or unsubstituted. For example, a biphenyl group may be interpreted as an aryl group or as a phenyl group substituted with a phenyl group.

In the present disclosure, the term “linked to an adjacent group to form a ring” may indicate that one is linked to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being linked to each other may be connected to another ring to form a spiro structure.

In the present disclosure, the term “an adjacent group” may refer to a substituent substituted for an atom which is directly connected to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”. In some embodiments, two methyl groups in 4,5-dimethylphenanthrene may be interpreted as mutually “adjacent groups”.

In the present disclosure, examples of a halogen atom may include a fluorine atom, a chlorine atom, a bromine atom, and/or an iodine atom.

In the present disclosure, an alkyl group may be a linear, branched or cyclic type or kind. The number of carbon atoms in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but are not limited thereto.

In the present disclosure, an alkenyl group refers to a hydrocarbon group including at least one carbon double bond in the middle (i.e., not on the end/terminus) or end of an alkyl group having 2 or more carbon atoms. The alkenyl group may be linear or branched. The number of carbon atoms is not limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, etc., but are not limited thereto.

In the present disclosure, an alkynyl group refers to a hydrocarbon group including at least one carbon triple bond in the middle (i.e., not on the end/terminus) or end of an alkyl group having 2 or more carbon atoms. The alkynyl group may be linear or branched. The number of carbon atoms is not limited, but may be 2 to 30, 2 to 20, or 2 to 10. Examples of the alkynyl group may include an ethynyl group, a propynyl group, etc., but are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but are not limited thereto.

In the present disclosure, a fluorenyl group may be substituted, and two substituents may be bonded to each other to form a spiro structure. An example that the fluorenyl group is substituted is as follows. However, the embodiment of the present disclosure is not limited thereto.

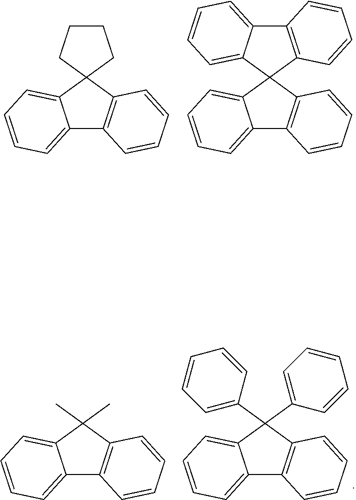

In the present disclosure, a heterocyclic group refers to any functional group or substituent derived from a ring containing at least one of B, O, N, P, Si, or S as a hetero atom. The heterocyclic group includes an aliphatic heterocyclic group and/or an aromatic heterocyclic group. The aromatic heterocyclic group may be a heteroaryl group. The aliphatic heterocycle and the aromatic heterocycle may be (may each be) monocyclic or polycyclic.

In the present disclosure, the heterocyclic group may contain at least one of B, O, N, P, Si or S as a hetero atom. When the heterocyclic group contains two or more hetero atoms, the two or more hetero atoms may be the same as or different from each other. In the present disclosure, the heterocyclic group may be a monocyclic heterocyclic group or a polycyclic heterocyclic group, and may refer to one including a heteroaryl group. The number of ring-forming carbon atoms in the heterocyclic group may be 2 to 30, 2 to 20, or 2 to 10.

In the present disclosure, the aliphatic heterocyclic group may contain at least one of B, O, N, P, Si or S as a hetero atom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but are not limited to thereto

In the present disclosure, a heteroaryl group may include at least one of B, O, N, P, Si, or S as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but are not limited thereto.

In the present disclosure, the above description of the aryl group may be applied to an arylene group, except that the arylene group is a divalent group. The above description of the heteroaryl group may be applied to a heteroarylene group, except that the heteroarylene group is a divalent group.

In the present disclosure, a boron group may refer to a group in which a boron atom is bonded to an alkyl group or aryl group as defined above. The boron group includes an alkyl boron group and/or an aryl boron group. Examples of the boron group include a dimethyl boron group, a diethyl boron group, a t-butylmethyl boron group, a diphenyl boron group, a phenyl boron group, etc., but are not limited thereto.

In the present disclosure, a silyl group includes an alkyl silyl group and/or an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but are not limited thereto.

In the present disclosure, the number of carbon atoms in a carbonyl group is not limited, but may be 1 to 40, 1 to 30, or 1 to 20. For example, the carbonyl group may have the following structure, but is not limited thereto.

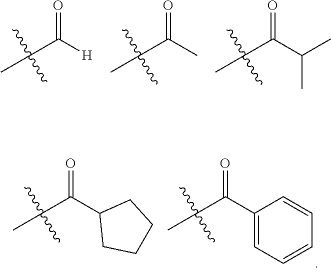

In the present disclosure, the number of carbon atoms in a sulfinyl group and a sulfonyl group is not limited, but may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkyl thio group and/or an aryl thio group. The thio group may indicate the one that a sulfur atom is bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., but are not limited to thereto.

In the present disclosure, an oxy group may indicate a group in which an oxygen atom is bonded to an alkyl group or aryl group as defined above. The oxy group may include an alkoxy group and/or an aryl oxy group. The alkoxy group may be linear, branched or cyclic. The number of carbon atoms in the alkoxy group is not limited, but may be, for example, 1 to 20, or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but are not limited thereto.

In the present disclosure, the number of carbon atoms in an amine group is not limited, but may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, etc., but are not limited thereto.

In the present disclosure, examples of the alkyl group may include an alkylthio group, an alkyl sulfoxy group, an alkylaryl group, an alkylamino group, an alkyl boron group, an alkyl silyl group, and/or an alkyl amine group.

In the present disclosure, examples of the aryl group may include an aryloxy group, an arylthio group, an aryl sulfoxy group, an arylamino group, an aryl boron group, an aryl silyl group, and/or an aryl amine group.

In the present disclosure, a direct linkage may refer to a single bond.

In some embodiments, in the present disclosure

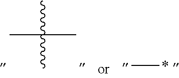

refers to a site to be connected.

Hereinafter, embodiments of the present disclosure will be described with reference to the accompanying drawings.

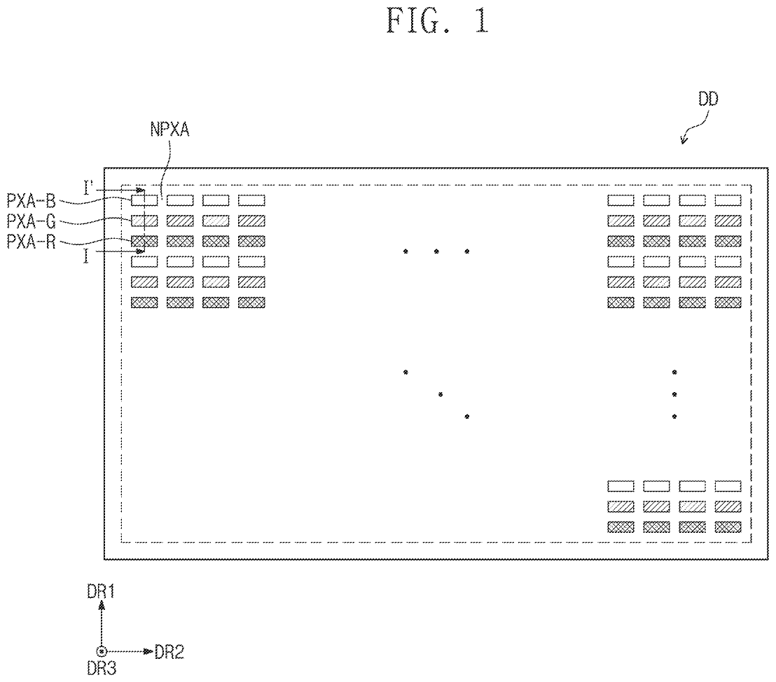

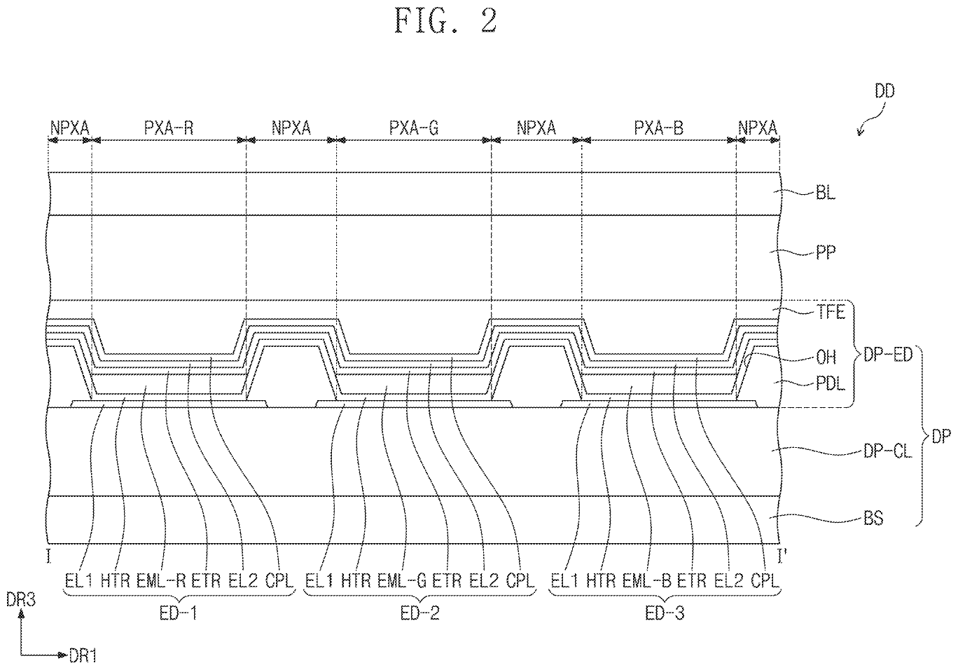

FIG. 1 is a plan view showing an embodiment of a display device DD. FIG. 2 is a cross-sectional view of a display device DD of an embodiment. FIG. 2 is a cross-sectional view showing a portion corresponding to line I-I′ of FIG. 1.

The display device DD may include a display panel DP and an optical layer PP disposed on the display panel DP. The display panel DP includes light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarizing layer or a color filter layer. In some embodiments, the optical layer PP may not be provided in the display device DD of an embodiment.

A base substrate BL may be on the optical layer PP. The base substrate BL may be a member providing a base surface on which the optical layer PP is disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided in an embodiment.

The display device DD according to an embodiment may further include a filling layer. The filling layer may be disposed between a display element layer DP-ED and the base substrate BL. The filling layer may be an organic material layer. The filling layer may include at least one selected from among an acrylic resin, a silicone-based resin, and an epoxy-based resin.

The display panel DP may include a base layer BS, a circuit layer DP-CL provided on the base layer BS, and a display element layer DP-ED. The display element layer DP-ED may include pixel defining films PDL, a plurality of light emitting elements ED-1, ED-2, and ED-3 disposed between (e.g., defined by) the pixel defining films PDL, and an encapsulation layer TFE on the plurality of light emitting elements ED-1, ED-2, and ED-3.

The base layer BS may be a member providing a base surface in which the display element layer DP-ED is disposed. The base layer BS may be a glass substrate, a metal substrate, a plastic substrate, etc. However, the embodiment of the present disclosure is not limited thereto, and the base layer BS may be an inorganic layer, an organic layer, or a composite material layer.

In an embodiment, the circuit layer DP-CL may be on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the plurality of light emitting elements ED-1, ED-2 and ED-3 of the display element layer DP-ED.

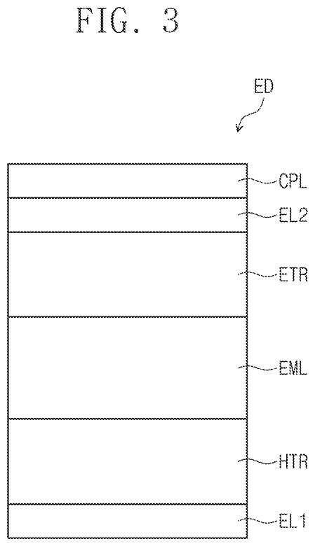

The light emitting elements ED-1, ED-2, and ED-3 may each have a structure of a light emitting element ED of an embodiment of FIGS. 3 to 5, which will be described in more detail. The light emitting elements ED-1, ED-2, and ED-3 may each include a first electrode EL1, a hole transport region HTR, emission layers EML-R, EML-G, and EML-B, an electron transport region ETR, a second electrode EL2, and a capping layer CPL.

FIG. 2 shows an embodiment in which the emission layers EML-R, EML-G, and EML-B of the light emitting elements ED-1, ED-2, and ED-3 are disposed in openings OH defined in the pixel defining films PDL, and the hole transport region HTR, the electron transport region ETR, and the second electrode EL2, and the capping layer CPL are provided as a common layer throughout the light emitting elements ED-1, ED-2, and ED-3. However, the embodiment of the present disclosure is not limited thereto, and in an embodiment, the hole transport region HTR and the electron transport region ETR may be provided to be patterned inside the openings OH defined in the pixel defining films PDL. For example, in an embodiment, the hole transport region HTR, the emission layers EML-R, EML-G, and EML-B, and the electron transport region ETR, etc., of the light emitting elements ED-1, ED-2, and ED-3 may be patterned and provided through an inkjet printing method.

An encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a laminated layer of a plurality of layers. The encapsulation layer TFE includes at least one insulating layer. The encapsulation layer TFE according to an embodiment may include at least one inorganic film (hereinafter, an encapsulation inorganic film). In some embodiments, the encapsulation layer TFE according to an embodiment may include at least one organic film (hereinafter, an encapsulation organic film) and at least one encapsulation inorganic film.

The encapsulation inorganic film protects (or reduces exposure to moisture/oxygen) the display element layer DP-ED from moisture/oxygen, and the encapsulation organic film protects (or reduces exposure to foreign substances) the display element layer DP-ED from foreign substances such as dust particles. The encapsulation inorganic film may include silicon nitride, silicon oxy nitride, silicon oxide, titanium oxide, aluminum oxide, etc., but is not limited thereto. The encapsulation organic layer may include an acrylic compound, an epoxy-based compound, etc. The encapsulation organic layer may include a photopolymerizable organic material, but should not be particularly limited to this material.

The encapsulation layer TFE may be disposed on the capping layer CPL, and may be disposed to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region emitting light generated from each of the light emitting elements ED-1, ED-2, and ED-3. The light emitting regions PXA-R, PXA-G, and PXA-B may be spaced apart from (separated from) each other when viewed on a plane (e.g., in a plan view).

The light emitting regions PXA-R, PXA-G, and PXA-B may each be a region separated by the pixel defining films PDL. The non-light emitting regions NPXA may be regions between neighboring light emitting regions PXA-R, PXA-G, and PXA-B, and may correspond to the pixel defining films PDL. In some embodiments, in the present disclosure, the light emitting regions PXA-R, PXA-G, and PXA-B may each correspond to a pixel. The pixel defining films PDL may separate the light emitting elements ED-1, ED-2 and ED-3. The emission layers EML-R, EML-G, and EML-B of the light emitting elements ED-1, ED-2 and ED-3 may be disposed and separated in openings OH defined by the pixel defining films PDL.

The light emitting regions PXA-R, PXA-G, and PXA-B may be divided into a plurality of groups according to the color of light generated from the light emitting elements ED-1, ED-2, and ED-3. In the display device DD of an embodiment shown in FIGS. 1 and 2, three light emitting regions PXA-R, PXA-G, and PXA-B which emit red light, green light, and blue light, are presented as an example. For example, the display device DD of an embodiment may include a red light emitting region PXA-R, a green light emitting region PXA-G, and a blue light emitting region PXA-B, which are distinct from one another.

In the display device DD according to an embodiment, the plurality of light emitting elements ED-1, ED-2, and ED-3 may emit light having different wavelength ranges. For example, in an embodiment, the display device DD may include a first light emitting element ED-1 emitting red light, a second light emitting element ED-2 emitting green light, and a third light emitting element ED-3 emitting blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2 and ED-3 may emit light in substantially the same wavelength range or emit light in at least one different wavelength range. For example, the first to third light emitting elements ED-1, ED-2, and ED-3 all may emit blue light.

The light emitting regions PXA-R, PXA-G, and PXA-B in the display device DD according to an embodiment may be arranged in the form of a stripe. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may each be arranged with each other along the second direction axis DR2. In some embodiments, the red light emitting region PXA-R, the green light emitting region PXA-G, and the blue light emitting region PXA-B may be alternately arranged in turn along a first direction axis DR1. (DR3 is a third direction which is normal or perpendicular to the plane defined by the first direction DR1 and the second direction DR2).

FIGS. 1 and 2 show that the light emitting regions PXA-R, PXA-G, and PXA-B are all similar in size, but the embodiment of the present disclosure is not limited thereto, and the light emitting regions PXA-R, PXA-G and PXA-B may be different in size from each other according to wavelength range of emitted light. In some embodiments, the areas of the light emitting regions PXA-R, PXA-G, and PXA-B may refer to areas when viewed on a plane defined by the first direction axis DR1 and the second direction axis DR2 (e.g., when viewed in a plan view).

In some embodiments, the arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are in one or more suitable combinations according to display quality characteristics required for the display device DD. For example, the light emitting regions PXA-R, PXA-G, and PXA-B may be arranged in the form of a pentile (PENTILE®) (for example, an RGBG matrix, an RGBG structure, or RGBG matrix structure) or a diamond (Diamond Pixel™) (e.g., a display (e.g., an OLED display) containing red, blue, and green (RGB) light emitting regions arranged in the shape of diamonds. PENTILE® is a duly registered trademark of Samsung Display Co., Ltd. Diamond Pixel™ is a trademark of Samsung Display Co., Ltd.

In some embodiments, areas of each of the light emitting regions PXA-R, PXA-G, and PXA-B may be different in size from one another. For example, in an embodiment, the green light emitting region PXA-G may be smaller than the blue light emitting region PXA-B in size, but the embodiment of the present disclosure is not limited thereto.

Hereinafter, FIGS. 3 to 5 are cross-sectional views schematically showing a light emitting element according to an embodiment. The light emitting element ED according to an embodiment may include a first electrode EL1, a second electrode EL2 facing the first electrode EL1, and at least one functional layer between the first electrode EL1 and the second electrode EL2, and a capping layer CPL on the second electrode EL2. The light emitting element ED according to an embodiment may include an amine compound of an embodiment, which will be described in more detail, in the capping layer CPL.

The light emitting element ED may include a hole transport region HTR, an emission layer EML, and an electron transport region ETR, which are sequentially stacked (in the stated order), as at least one functional layer. Referring to FIG. 3, the light emitting element ED according to an embodiment may include a first electrode EL1, a hole transport region HTR, an emission layer EML, an electron transport region ETR, a second electrode EL2, and the capping layer CPL.

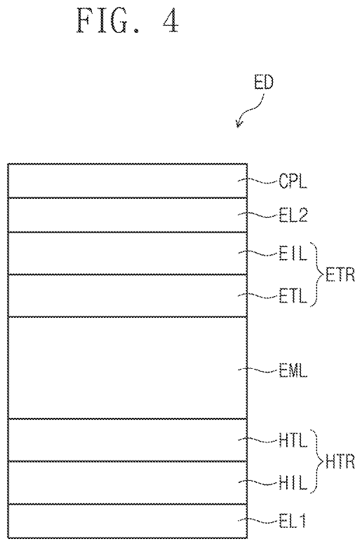

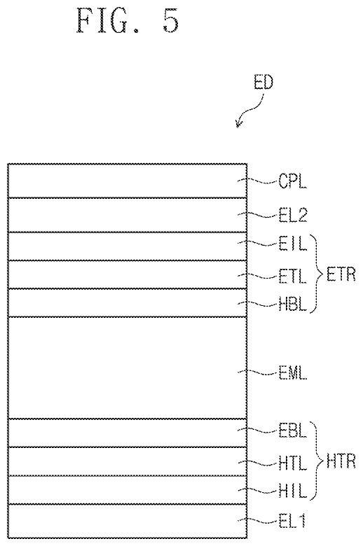

FIG. 4 shows, compared with FIG. 3, a cross-sectional view of a light emitting element ED of an embodiment in which the hole transport region HTR includes a hole injection layer HIL and a hole transport layer HTL, and the electron transport region ETR includes an electron injection layer EIL and an electron transport layer ETL. In some embodiments, FIG. 5 shows, compared with FIG. 3, a cross-sectional view of a light emitting element ED of an embodiment in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL.

In the light emitting element ED according to an embodiment, the first electrode EL1 has conductivity (e.g., is a conductor). The first electrode EL1 may be formed of a metal material, a metal alloy or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode may include at least one selected from among Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, or one or more oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, one or more compounds thereof, or one or more mixtures thereof (e.g., a mixture of Ag and Mg). In some embodiments, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), etc. For example, the first electrode EL1 may have a three-layer structure of ITO/Ag/ITO, but is not limited thereto. For example, the first electrode EL1 may include the above-described metal materials, a combination of two or more metal materials selected from the above-described metal materials, or one or more oxides of the above-described metal materials. The first electrode EL1 may have a thickness of about 700 Å to about 10000 Å. For example, the first electrode EL1 may have a thickness of 1000 Å to about 3000 Å.

The hole transport region HTR is provided on the first electrode EL1. The hole transport region HTR may have a single layer formed of a single material, a single layer formed of a plurality of different materials, or a multilayer structure having a plurality of layers formed of a plurality of different materials.

The hole transport region HTR may include at least one selected from among a hole injection layer HIL, a hole transport layer HTL, and an electron blocking layer EBL. In some embodiments, the hole transport region HTR may include a plurality of hole transport layers that are stacked.

In some embodiments, alternatively, the hole transport region HTR may have a single-layer structure formed of the hole injection layer HIL or the hole transport layer HTL, or a single-layer structure formed of a hole injection material or a hole transport material. In an embodiment, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the present disclosure is not limited thereto.

The hole transport region HTR may have, for example, a thickness of about 50 Å to about 15000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

In the light emitting element ED according to an embodiment, the hole transport region HTR may include a compound represented by Formula H-1.

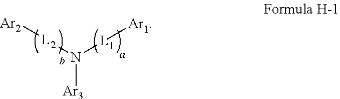

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer from 0 to 10. In some embodiments, when a or b is an integer of 2 or greater, a plurality of L₁s and L₂s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

A compound represented by Formula H-1 may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound including a substituted or unsubstituted carbazole group in at least one of Ar₁ or Ar₂ or a substituted or unsubstituted fluorene-based group in at least one of Ar₁ or Ar₂.

The compound represented by Formula H-1 may be represented by any one selected from among compounds from Compound Group H. However, the compounds listed in Compound Group H are presented merely as examples, and the compound represented by Formula H-1 is not limited to the those listed in Compound Group H. Compound Group H

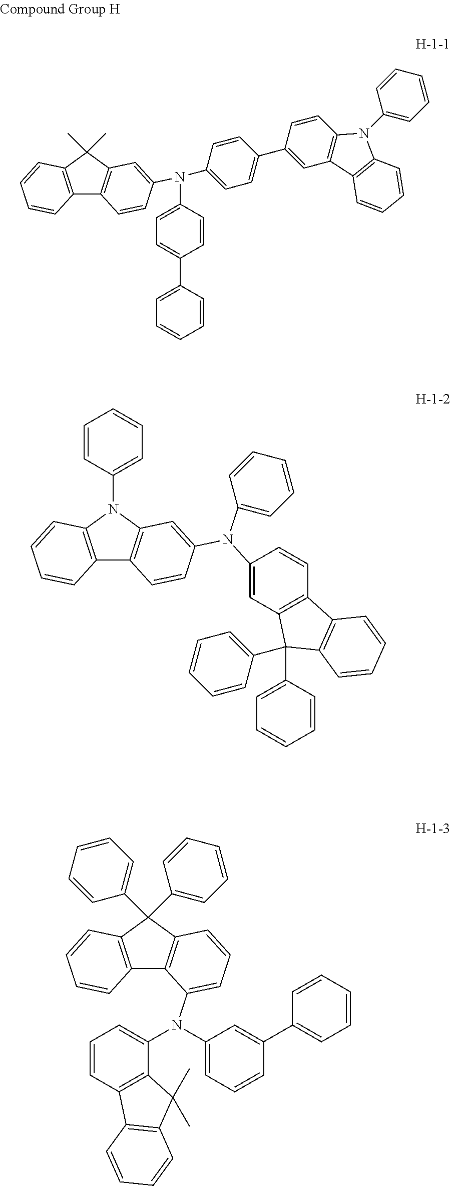 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

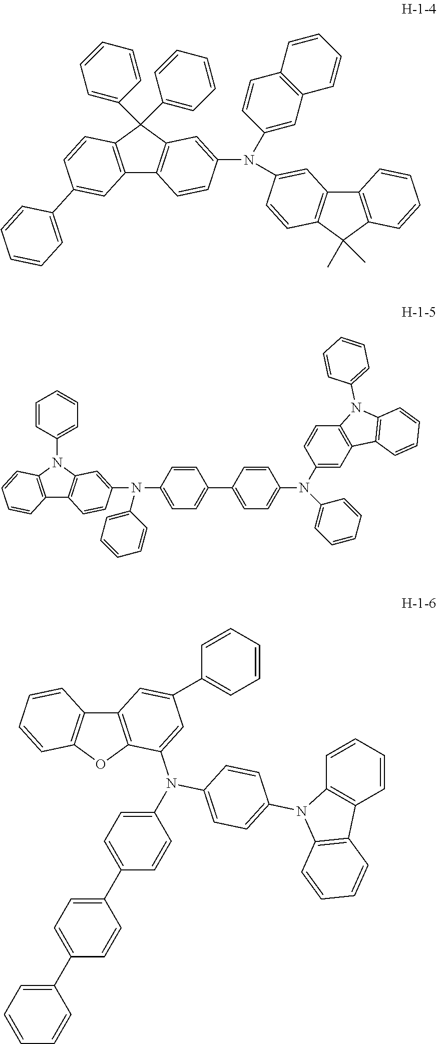 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

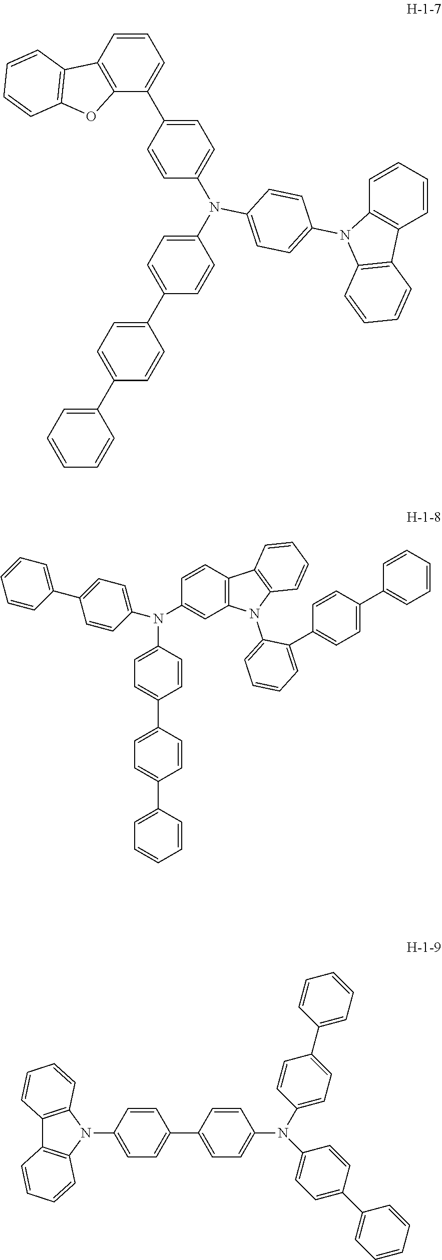 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

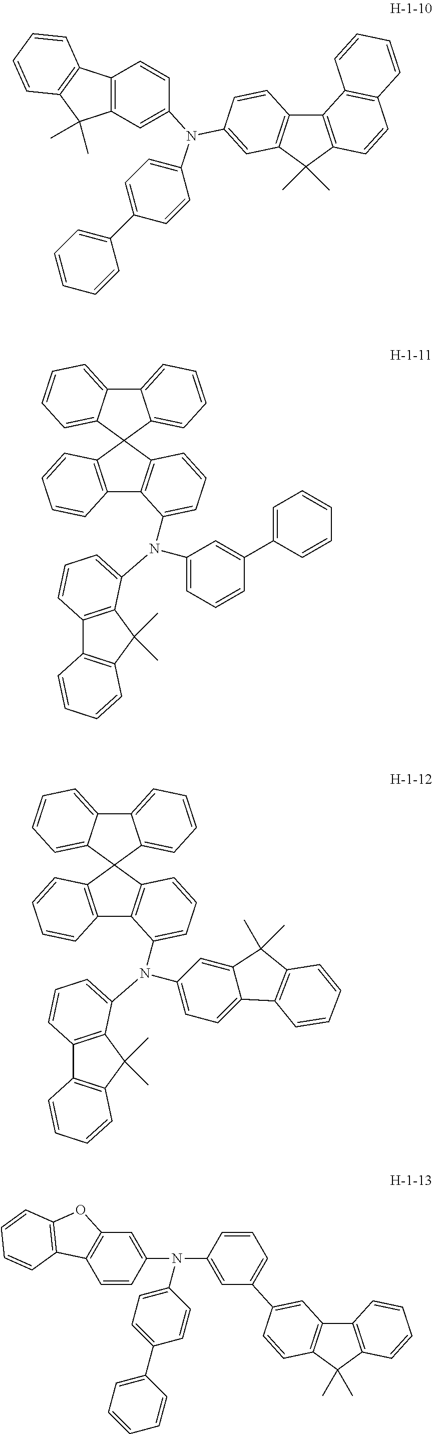 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

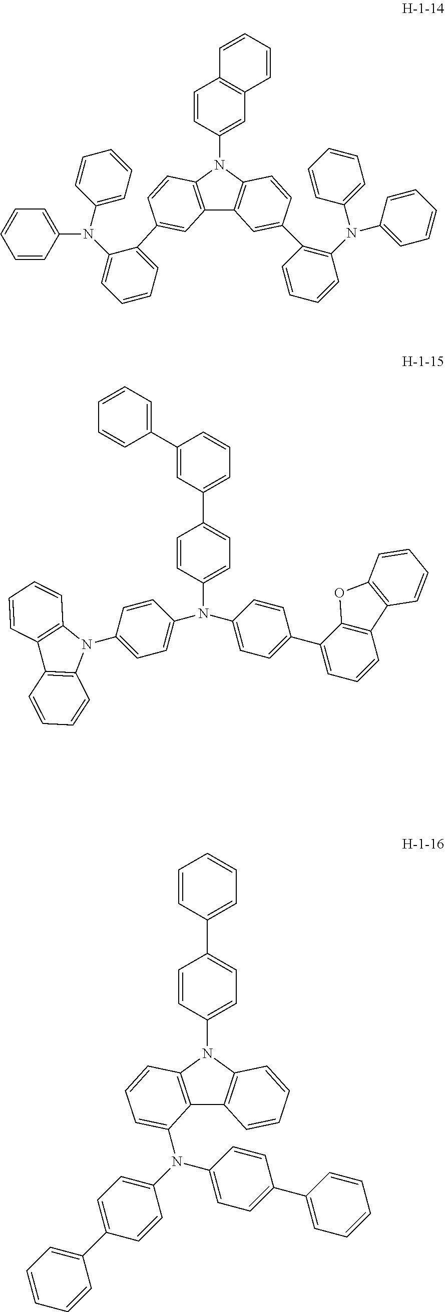 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

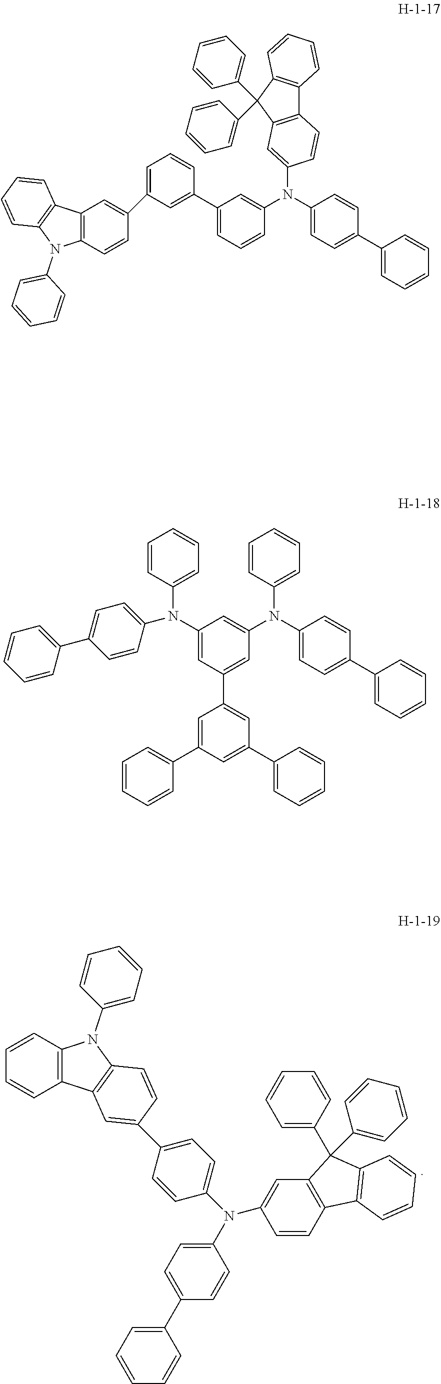 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N,N′-diphenyl-N,N′-bis-[4-(phenyl-m-tolyl-amino)-phenyl]-biphenyl-4,4′-diamine (DNTPD), 4,4′,4″-[tris(3-methylphenyl)phenylamino]triphenylamine] (m-MTDATA), 4,4′,4′-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB or NPD), triphenylamine-containing polyetherketone (TPAPEK), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (HATCN), etc.

The hole transport region HTR may further include carbazole-based derivatives such as N-phenyl carbazole and/or polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-I-yl)-N,N′-diphenyl-benzidine (NPB), 4,4′-Cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may further include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region described above in at least one selected from among the hole injection layer HIL, the hole transport layer HTL, and the electron blocking layer EBL.

The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the above-described ranges, satisfactory (suitable) hole transport properties may be obtained without a substantial increase in driving voltage.

The hole transport region HTR may further include, in addition to the above-described materials, a charge generation material to increase conductivity. The charge generation material may be substantially uniformly or non-uniformly dispersed in the hole transport region HTR. The charge generation material may be, for example, a p-dopant. The p-dopant may include at least one of halogenated metal compounds, quinone derivatives, metal oxides, or cyano group-containing compounds, but is not limited thereto. For example, the p-dopant may include one or more halogenated metal compounds such as CuI and RbI, quinone derivatives such as tetracyanoquinodimethane (TCNQ) and/or 2,3,5,6-tetrafluoro-7,7,8,8-tetracyanoquinodimethane (F4-TCNQ), metal oxides such as tungsten oxides and/or molybdenum oxides, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but is not limited thereto.

As described above, the hole transport region HTR may further include at least one of a buffer layer or an electron blocking layer EBL in addition to the hole injection layer HIL and the hole transport layer HTL. The buffer layer may compensate for a resonance distance according to wavelengths of light emitted from an emission layer EML, and may thus increase light emitting efficiency. Materials which may be included in the hole transport region HTR may be utilized as materials included in the buffer layer. The electron blocking layer EBL is a layer that serves to prevent or reduce electrons from being injected from the electron transport region ETR to the hole transport region HTR.

The emission layer EML is provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting element ED of an embodiment, the emission layer EML may emit blue light. The light emitting element ED according to an embodiment may include the amine compound of an embodiment, which is described above in the hole transport region HTR, and may thus exhibit high efficiency and long lifespan characteristics in the blue light emitting region. However, the embodiment of the present disclosure is not limited thereto.

In the light emitting element ED of an embodiment, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light emitting element ED of the embodiment shown in FIGS. 3 to 5, the emission layer EML may include a host and a dopant, and the emission layer EML may include a compound represented by Formula E-1. The compound represented by Formula E-1 may be utilized as a fluorescent host material.

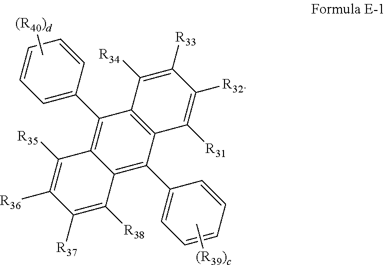

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In some embodiments, R₃₁ to R₄₀ may be linked to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

Formula E-1 may be represented by any one selected from among compounds E1 to E19.

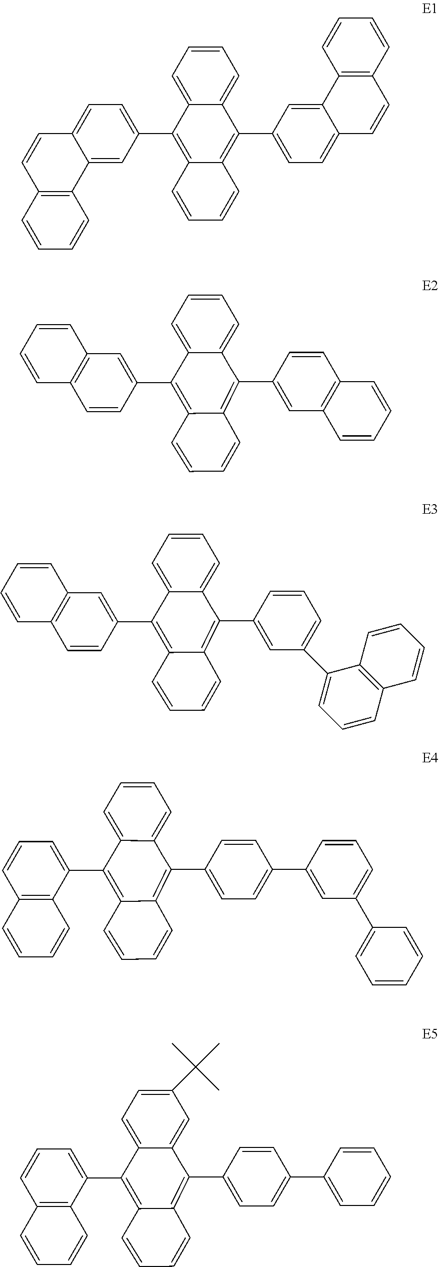

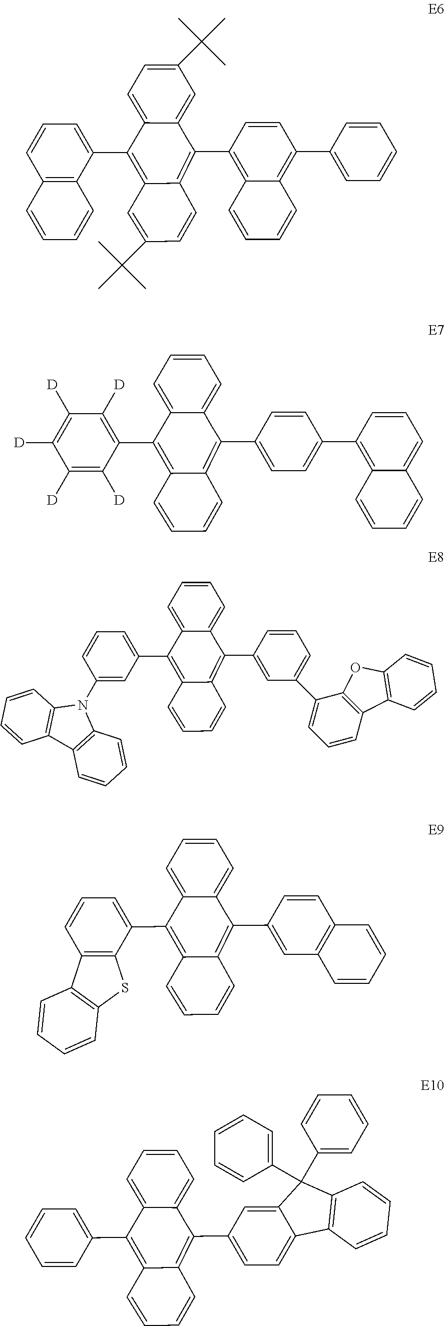

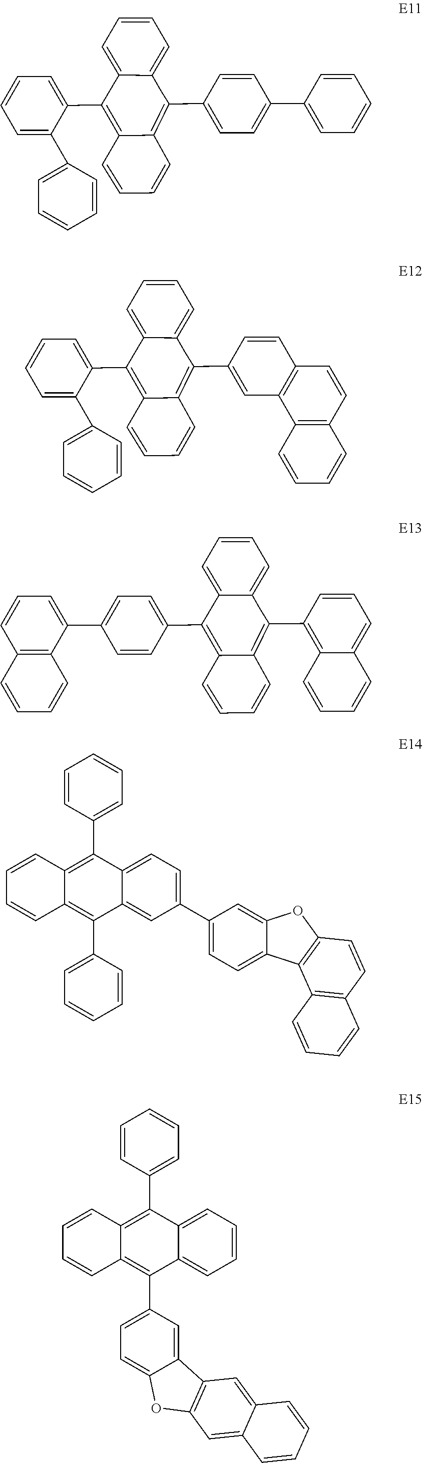

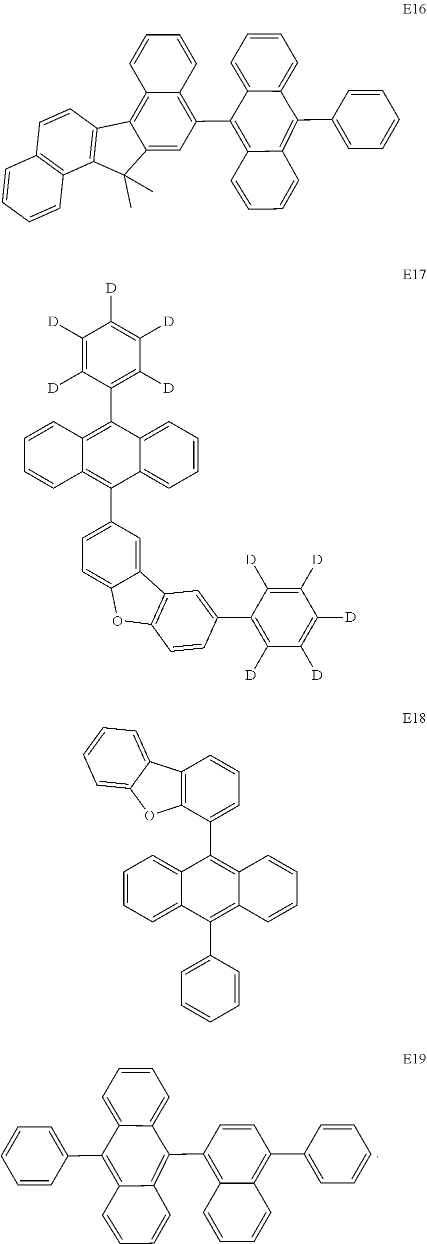

In an embodiment, the emission layer EML may include a compound represented by Formula E-2a or Formula E-2b. The compound represented by Formula E-2a or Formula E-2b may be utilized as a phosphorescent host material.

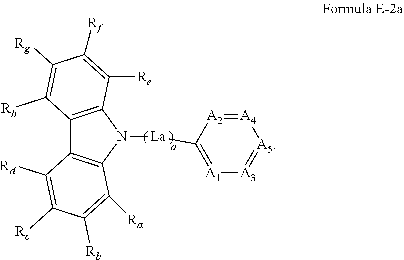

In Formula E-2a, a may be an integer from 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of Las may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may be N or Crᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or linked to an adjacent group to form a ring. Rₐ to Rᵢ may be linked to an adjacent group to form a hydrocarbon ring or a heterocycle containing N, O, S, etc. as a ring-forming atom.

In some embodiments, in Formula E-2a, two or three selected from among A₁ to A₅ may be N, and the rest (those that are not N) may be Crᵢ.

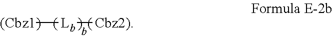

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer from 0 to 10, and when b is an integer of 2 or greater, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds from Compound Group E-2. However, the compounds listed in Compound Group E-2 are presented merely as examples, and the compound represented by Formula E-2a or Formula E-2b is not limited to those listed in Compound Group E-2.

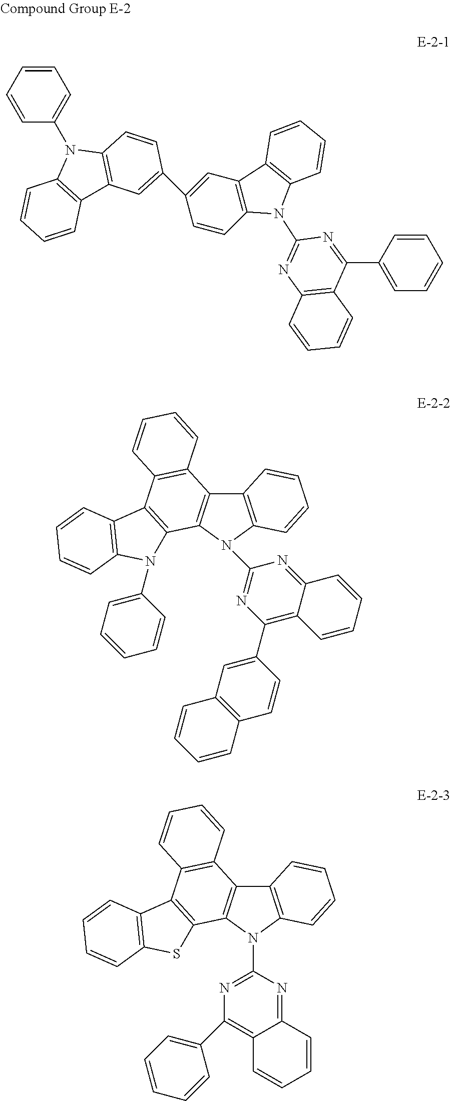

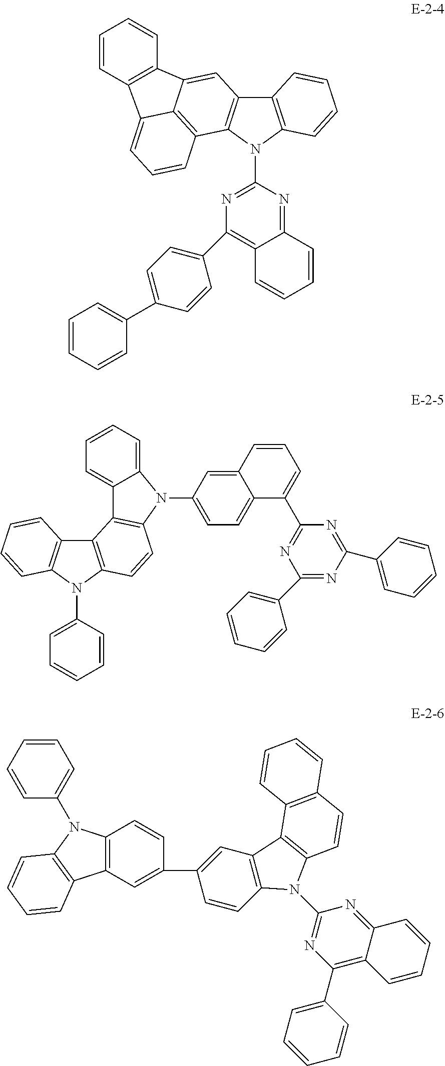 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

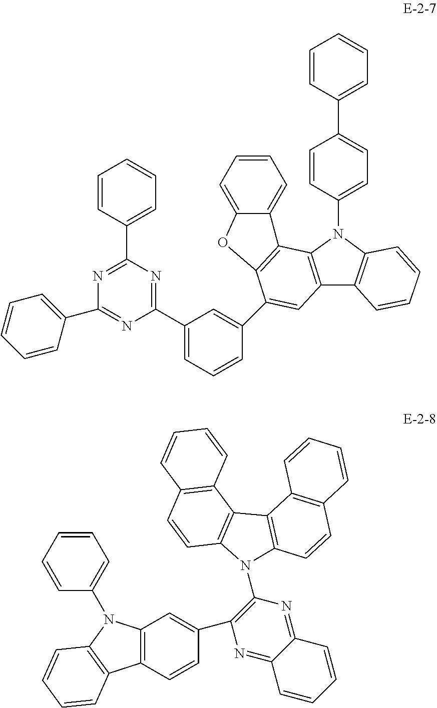 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

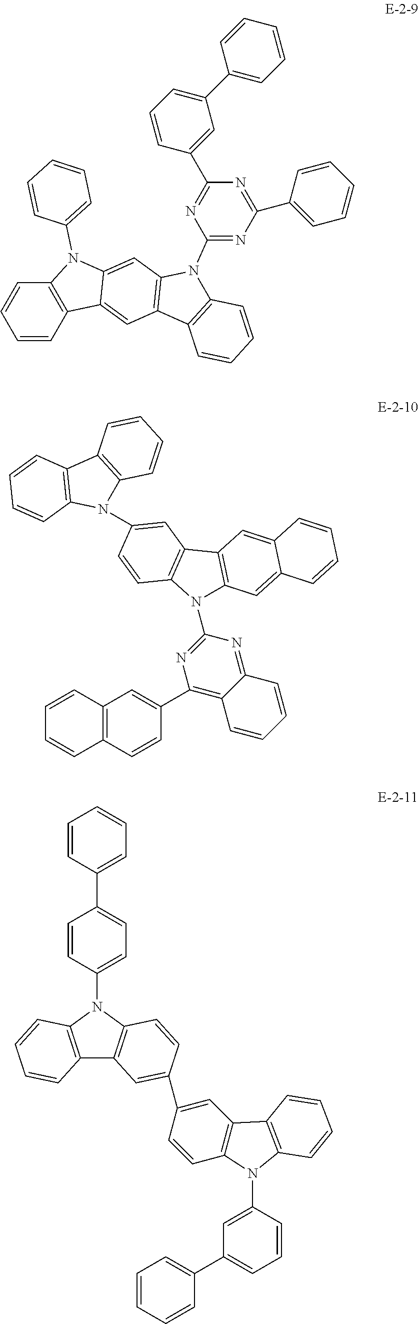 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

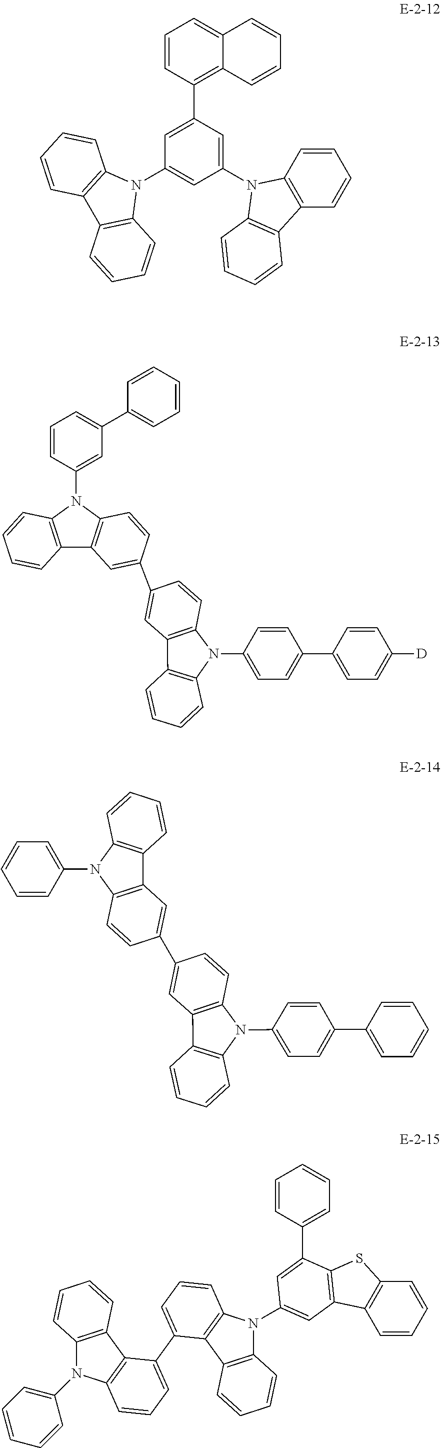 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

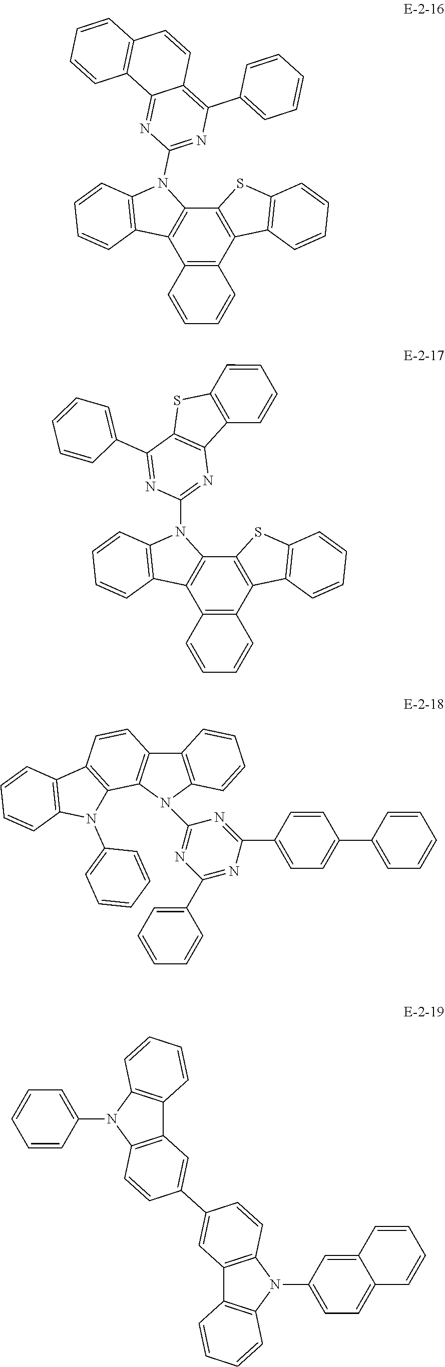

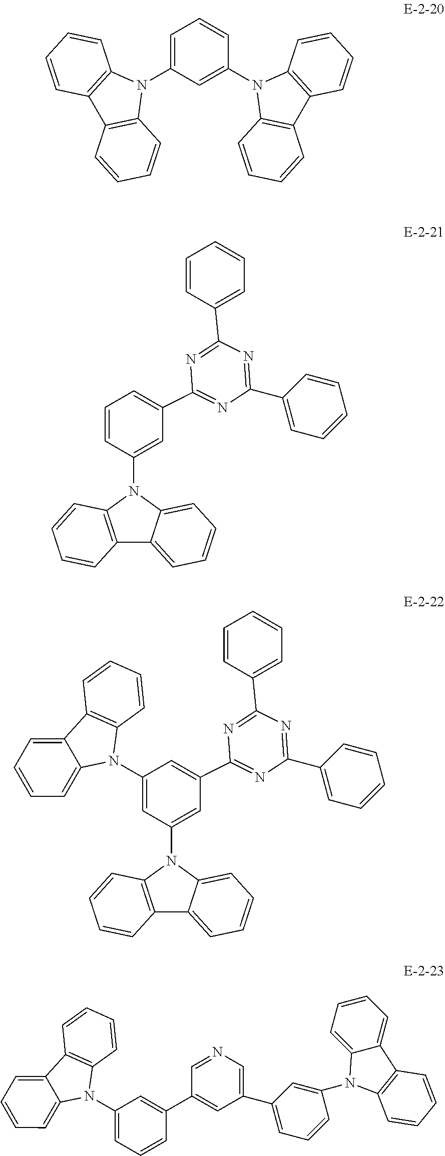 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

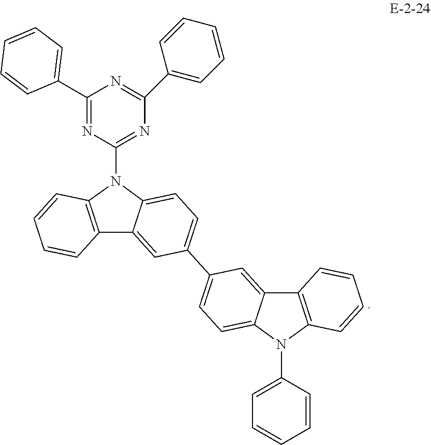 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material generally utilized/generally available in the art as a host material. For example, the emission layer EML may include, as a host material, at least one selected from among bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazolyl-9-yl)benzene (mCP), 2,8-bis(diphenylphosphoryl)dibenzofuran (PPF), 4,4′,4″-tris(carbazol-9-yl)-triphenylamine (TCTA), and 1,3,5-tris(1-phenyl-1H-benzo[d]imidazol-2-yl)benzene (TPBi). However, the embodiment of the present disclosure is not limited thereto, and for example, tris(8-hydroxyquinolino)aluminum (Alq₃), 9,10-di(naphthalene-2-yl)anthracene (ADN), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may be utilized as a host material.

The emission layer EML may include a compound represented by Formula M-a or Formula M-b. The compound represented by Formula M-a or Formula M-b may be utilized as a phosphorescent dopant material. In some embodiments, the compound represented by Formula M-a or Formula M-b may be utilized as an assistant dopant material.

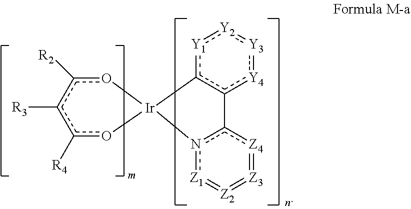

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be represented by any one selected from among compounds M-a1 to M-a25. However, the compounds M-a1 to M-a25 are presented merely as examples, and the compound represented by Formula M-a is not limited to those represented by the compounds M-a1 to M-a25.

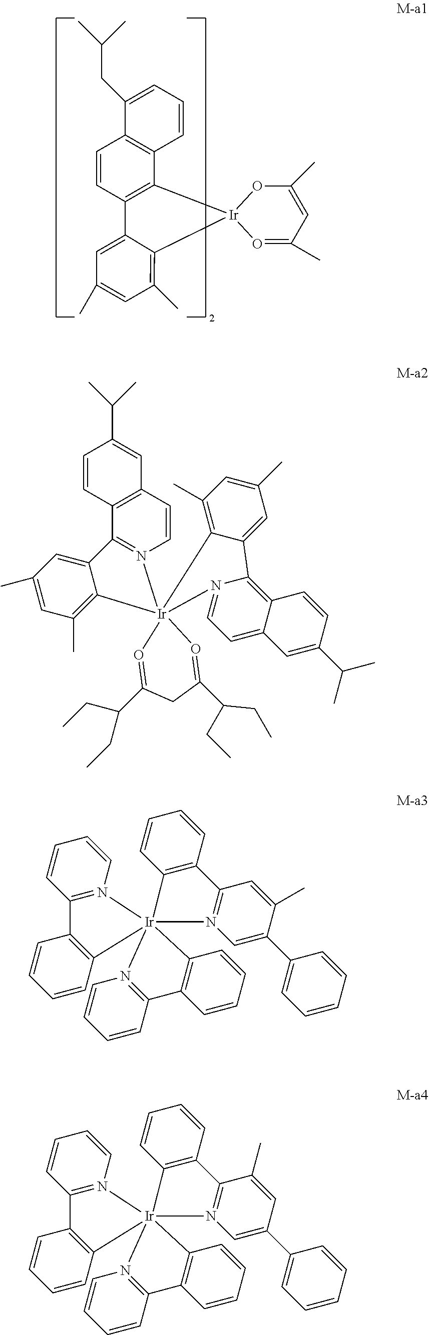

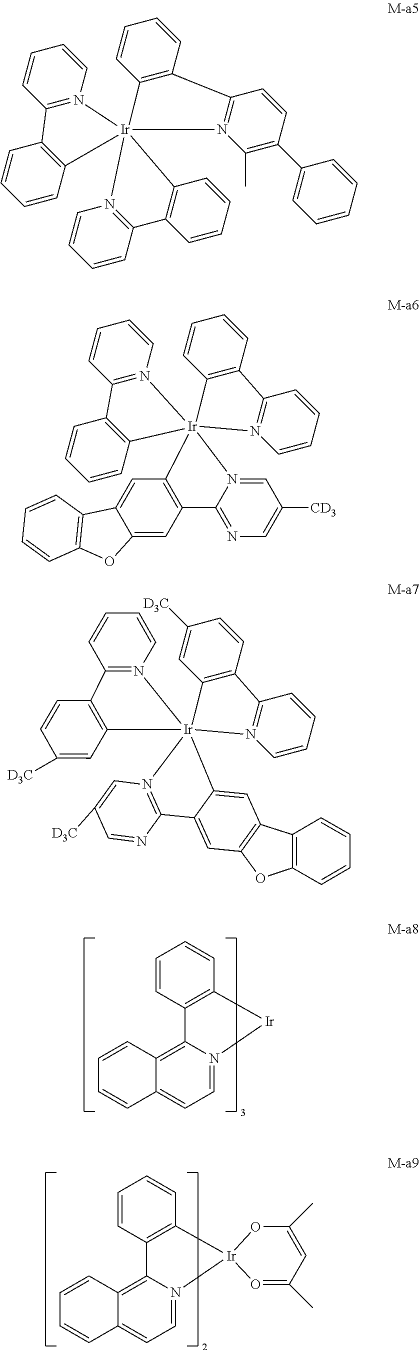

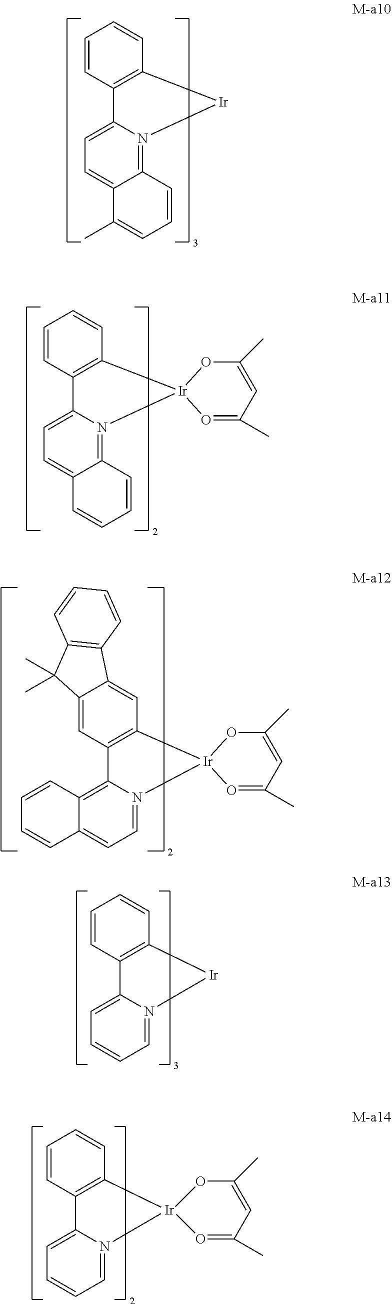

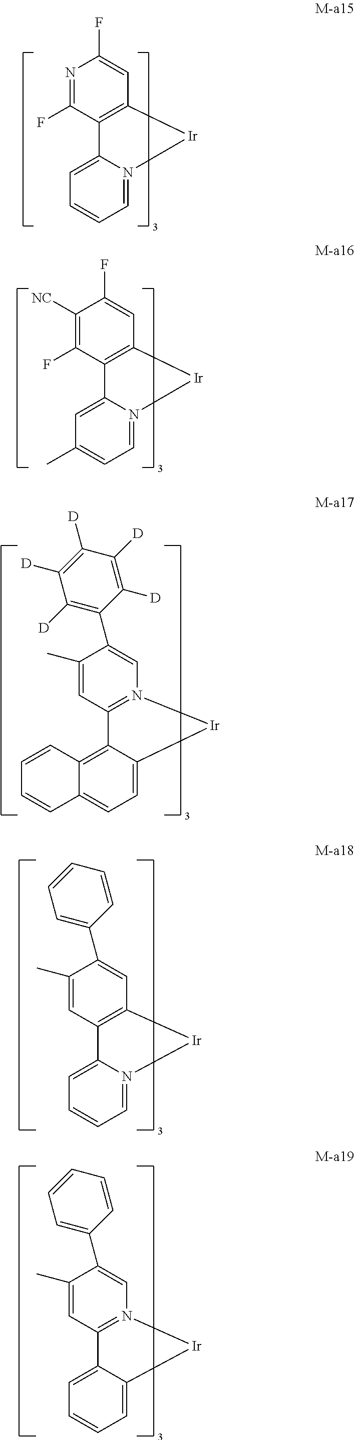

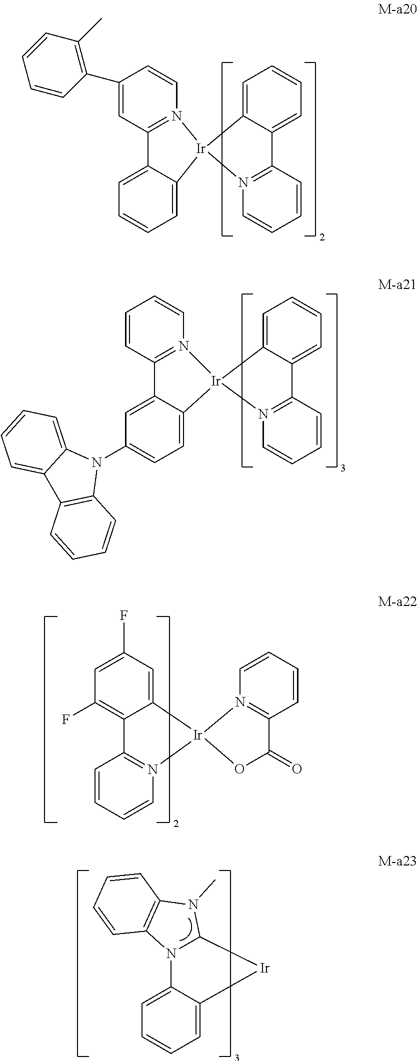

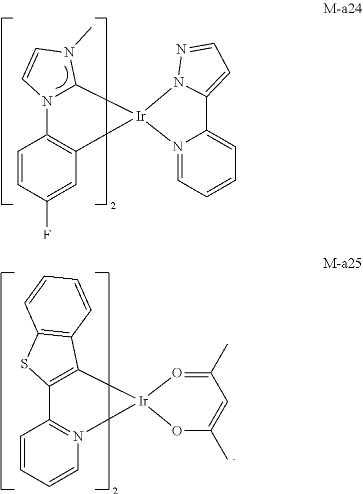

The compounds M-a1 and M-a2 may be utilized as a red dopant material, and the compounds M-a3 to M-a7 may be utilized as a green dopant material.

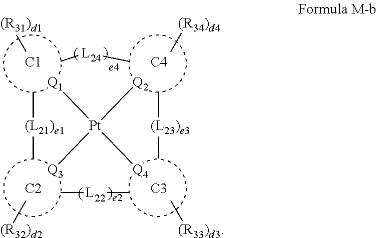

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

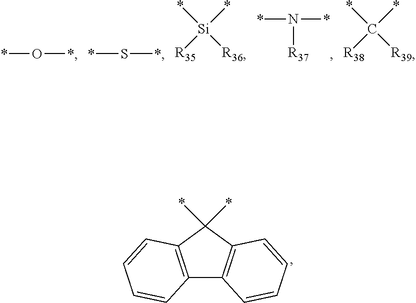

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and d1 to d4 may each independently be an integer from 0 to 4.

The compound represented by Formula M-b may be utilized as a blue phosphorescent dopant or a green phosphorescent dopant. In some embodiments, the compound represented by Formula M-b may be further included as an assistant dopant in the emission layer EML

The compound represented by Formula M-b may be represented by any one selected from among compounds below. However, the compounds below are presented merely as examples, and the compound represented by Formula M-b is not limited to those represented by the compounds below.

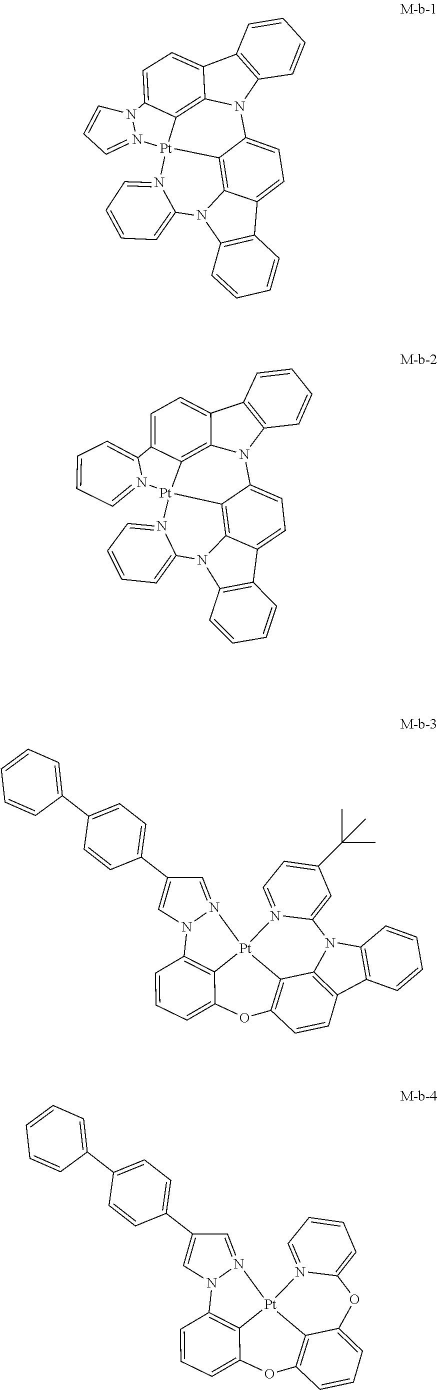

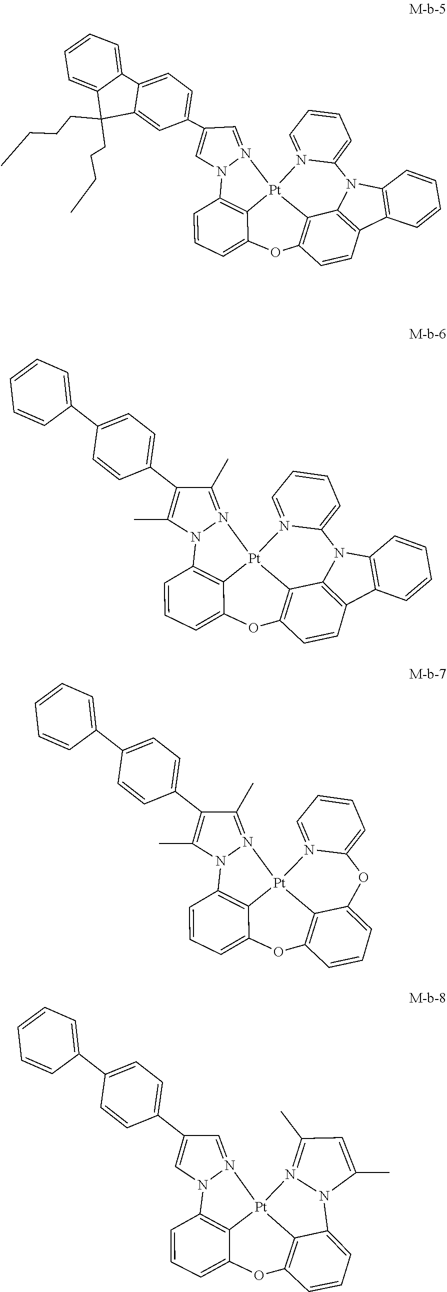

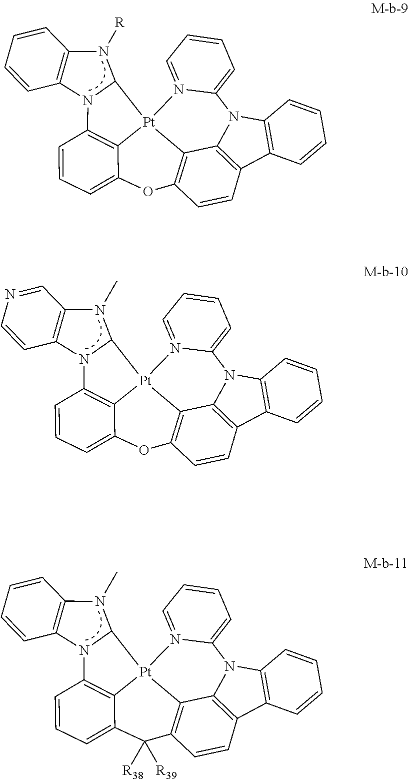

In the compounds above, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by any one selected from among Formulas F-a to F-c. The compounds represented by Formulas F-a to F-c may be utilized as a fluorescent dopant material.

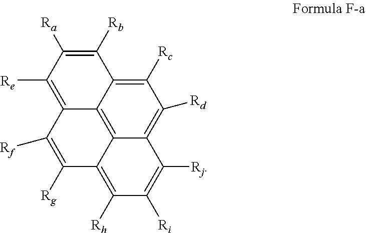

In Formula F-a, two selected from among Rₐ to Rⱼ may each independently be substituted with *-NAr₁Ar₂. The others among Rₐ to Rⱼ which are not substituted with *-NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *-NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

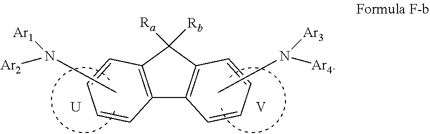

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, and/or linked to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, one ring forms a fused ring in a portion indicated by U or V, and when the number of U or V is 0, it refers to no ring indicated by U or V being present. For example, when the number of U is 0 and the number of V is 1, or when the number of U is 1 and the number of V is 0, a fused ring having a fluorene core of Formula F-b may be a cyclic compound having four rings. In some embodiments, when both (e.g., simultaneously) U and V are 0, the fused ring of Formula F-b may be a cyclic compound having three rings. In some embodiments, when both (e.g., simultaneously) U and V are 1, the fused ring having a fluorene core of Formula F-b may be a cyclic compound having five rings.

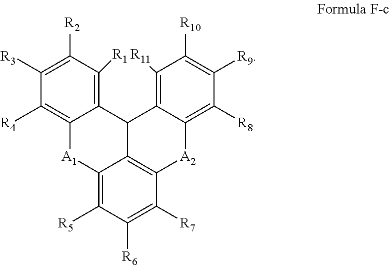

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In Formula F-c, A₁ and A₂ may each independently be bonded to substituents of neighboring rings to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

The emission layer EML may include, as a generally utilized/generally available dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi)), perylene and/or derivatives thereof (e.g., 2,5,8,11-tetra-t-butylperylene (TBP)), pyrene and derivatives thereof (e.g., 1,1-dipyrene, 1,4-dipyrenylbenzene, 1,4-bis(N,N-diphenylamino)pyrene), etc.

In an embodiment, when a plurality of emission layers EML are included, at least one emission layer EML may include a generally utilized/generally available phosphorescent dopant material. For example, as a phosphorescent dopant, a metal complex including iridium (Ir), platinum (Pt), osmium (Os), gold (Au), titanium (Ti), zirconium (Zr), hafnium (Hf), europium (Eu), and terbium (Tb), or thulium (Tm) may be utilized. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), platinum octaethyl porphyrin (PtOEP), etc. may be utilized as a phosphorescent dopant. However, the embodiment of the present disclosure is not limited thereto.

In some embodiments, the emission layer EML may include a hole transporting host and an electron transporting host. In some embodiments, the emission layer EML may include an auxiliary dopant and a light emitting dopant. In some embodiments, the auxiliary dopant may include a phosphorescent dopant material or a thermally activated delayed fluorescent dopant material. For example, in an embodiment, the emission layer EML may include a hole transporting host, an electron transporting host, an auxiliary dopant, and a light emitting dopant.

In some embodiments, in the emission layer EML, the hole transporting host and the electron transporting host may form an exciplex. In this embodiment, the triplet energy of the exciplex formed by the hole transporting host and the electron transporting host may correspond to T1, which is a gap between LUMO energy level of the electron transporting host and HOMO energy level of the hole transporting host.

In an embodiment, the triplet energy level T1 of the exciplex formed by the hole transporting host and the electron transporting host may be about 2.4 eV to about 3.0 eV. In some embodiments, the triplet energy of the exciplex may have a value smaller than the energy gap of each host material. Accordingly, the exciplex may have a triplet energy of 3.0 eV or less, which is an energy gap between the hole transporting host and the electron transporting host.

In some embodiments, at least one emission layer EML may include a quantum dot material. The core of a quantum dot may be selected from among a Group II-VI compound, a Group III-VI compound, a Group I-III-VI compound, a Group III-V compound, a Group III-II-V compound, a Group IV-VI compound, a Group IV element, a Group IV compound, and one or more combinations thereof.

The Group II-VI compound may be selected from the group including (e.g., consisting of) a binary compound selected from the group including (e.g., consisting of) CdSe, CdTe, CdS, ZnS, ZnSe, ZnTe, ZnO, HgS, HgSe, HgTe, MgSe, MgS, and a mixture thereof, a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and one or more compounds or mixtures thereof, and a quaternary compound selected from the group including (e.g., consisting of) HgZnTeS, CdZnSeS, CdZnSeTe, CdZnSTe, CdHgSeS, CdHgSeTe, CdHgSTe, HgZnSeS, HgZnSeTe, HgZnSTe, and one or more compounds or mixtures thereof.

The Group III-VI compound may include a binary compound such as In₂S₃ and In₂Se₃, a ternary compound such as InGaS₃ and InGaSe₃, or one or more combinations thereof.

The Group I-III-VI compound may include a ternary compound selected from the group including (e.g., consisting of) AgInS, AgInS₂, CuInS, CulnS₂, AgGaS₂, CuGaS₂ CuGaO₂, AgGaO₂, AgAlO₂, or one or more compounds or mixtures thereof, or a quaternary compound such as AgInGaS₂ and CuInGaS₂ (the quaternary compound may be used alone or in combination with any of the foregoing compounds or mixtures, and the quaternary compound may also be combined with other quaternary compounds).

The Group III-V compound may be selected from the group consisting of a binary compound selected from the group including (e.g., consisting of) GaN, GaP, GaAs, GaSb, AlN, AlP, AlAs, AlSb, InN, InP, InAs, InSb, and one or more compounds or mixtures thereof, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more compounds or mixtures thereof, and a quaternary compound selected from the group including (e.g., consisting of) GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and one or more compounds or mixtures thereof. In some embodiments, the Group III-V compound may further include a Group II metal. For example, InZnP, etc. may be selected as a Group III-II-V compound.

The Group IV-VI compound may be selected from the group including (e.g., consisting of) a binary compound selected from the group including (e.g., consisting of) SnS, SnSe, SnTe, PbS, PbSe, PbTe, and a mixture thereof, a ternary compound selected from the group including (e.g., consisting of) SnSeS, SnSeTe, SnSTe, PbSeS, PbSeTe, PbSTe, SnPbS, SnPbSe, SnPbTe, and one or more compounds or mixtures thereof, and a quaternary compound selected from the group including (e.g., consisting of) SnPbSSe, SnPbSeTe, SnPbSTe, and one or more compounds or mixtures thereof. The Group IV element may be selected from the group including (e.g., consisting of) Si, Ge, and one or more elements of mixtures thereof. The Group IV compound may be a binary compound selected from the group including (e.g., consisting of) SiC, SiGe, and one or more compounds or mixtures thereof.

In this embodiment, the binary compound, the ternary compound, or the quaternary compound may be present in a particle form having a substantially uniform concentration distribution, or may be present in substantially the same particle form having a partially different concentration distribution. In some embodiments, a core/shell structure in which one quantum dot surrounds another quantum dot may be present. The core/shell structure may have a concentration gradient in which the concentration of an element present in the shell decreases towards the core.

In some embodiments, a quantum dot may have the core/shell structure including a core having nano-crystals, and a shell around (e.g., surrounding) the core, which are described above. The shell SL of the quantum dots QD may serve as a protective layer for maintaining semiconductor characteristics by preventing or reducing the core CR from being chemically modified (adversely chemically modified), and/or serve as a charging layer for imparting electrophoretic characteristics to the quantum dots QD. The shell may be a single layer or multiple layers. Examples of the shell of the quantum dot may be a metal or non-metal oxide, a semiconductor compound, or one or more combinations thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, or a ternary compound such as MgAl₂O₄, CoFe₂O₄, NiFe₂O₄, and CoMn₂O₄, but the embodiment of the present disclosure is not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

A quantum dot may have a full width of half maximum (FWHM) of a light emitting wavelength spectrum of about 45 nm or less, about 40 nm or less, or about 30 nm or less, and color purity or color reproducibility may be enhanced in the above ranges. In some embodiments, light emitted through such the quantum dots are emitted in all directions, so that wide viewing angle characteristics may be improved.

In some embodiments, shapes of the quantum dots are not limited to a shape generally utilized/generally available in the art. For example, the quantum dots may have a shape such as a substantially spherical shape, a pyramidal shape, a multi-arm shape, or a cubic nanoparticle, a nanotube, a nanowire, a nanofiber, a nanoplatelet particle, etc.

The quantum dot may control (select) the colors of emitted light according to the particle size thereof, and thus the quantum dot may have one or more suitable light emission colors such as blue, red, green, etc.

In the light emitting element ED of an embodiment shown in FIGS. 3 to 5, an electron transport region ETR is provided on the emission layer EML. The electron transport region ETR may include at least one selected from among a hole blocking layer HBL, an electron transport layer ETL, and an electron injection layer EIL, but the embodiment of the present disclosure is not limited thereto.

The electron transport region ETR may have a single layer formed of a single material, a single layer formed of a plurality of different materials, or a multilayer structure having a plurality of layers formed of a plurality of different materials.

For example, the electron transport region ETR may have a single layer structure of an electron injection layer EIL or an electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, or an electron transport layer ETL/buffer layer/electron injection layer EIL are stacked in order (in the stated order) from the emission layer EML, but is not limited thereto. The electron transport region ETR may have a thickness of, for example, about 1000 Å to about 1500 Å.

The electron transport region ETR may be formed utilizing one or more suitable methods such as a vacuum deposition method, a spin coating method, a cast method, a Langmuir-Blodgett (LB) method, an inkjet printing method, a laser printing method, a laser induced thermal imaging (LITI) method, etc.

The electron transport region ETR may include a compound represented by Formula ET-1.

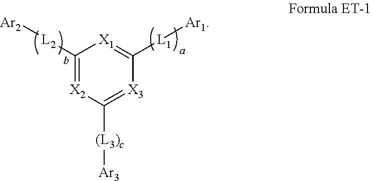

In Formula ET-1, at least one selected from among X₁ to X₃ is N and the rest (those that are not N) are Crₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq2), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or one or more compounds or mixtures thereof.

The electron transport region ETR may include at least one selected from among compounds ET1 to ET36.

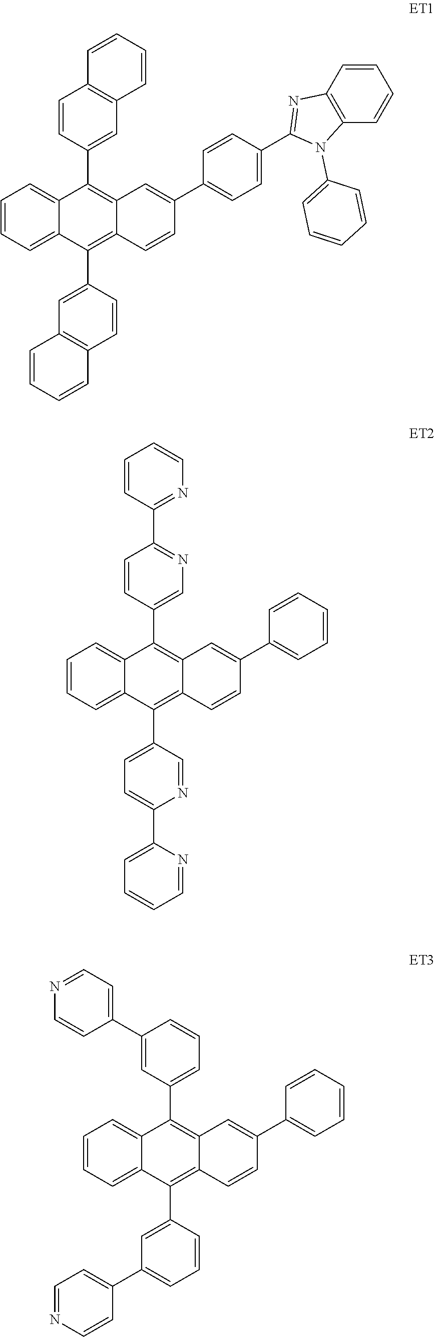 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

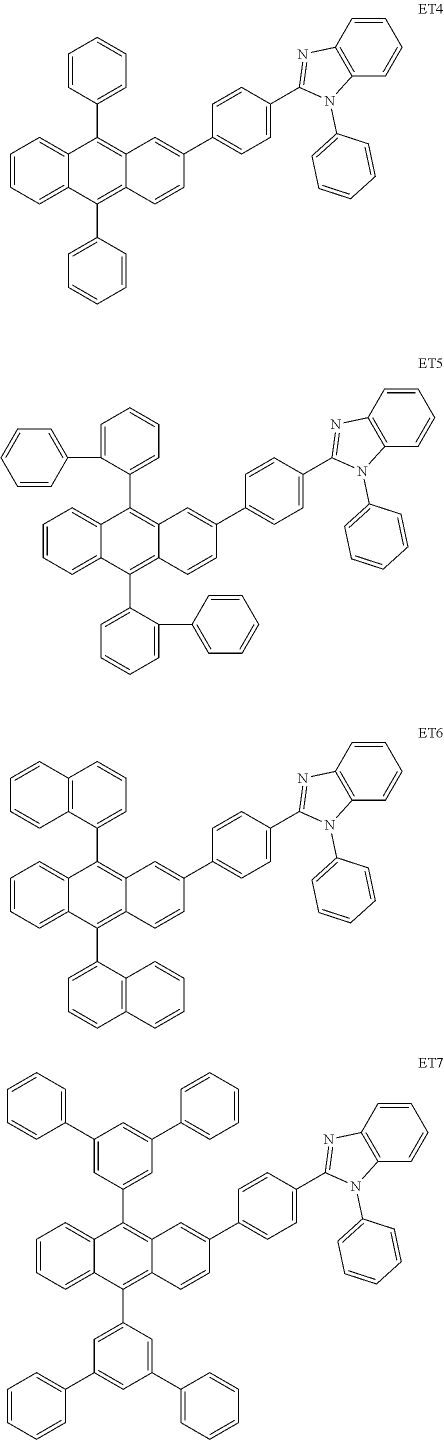 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

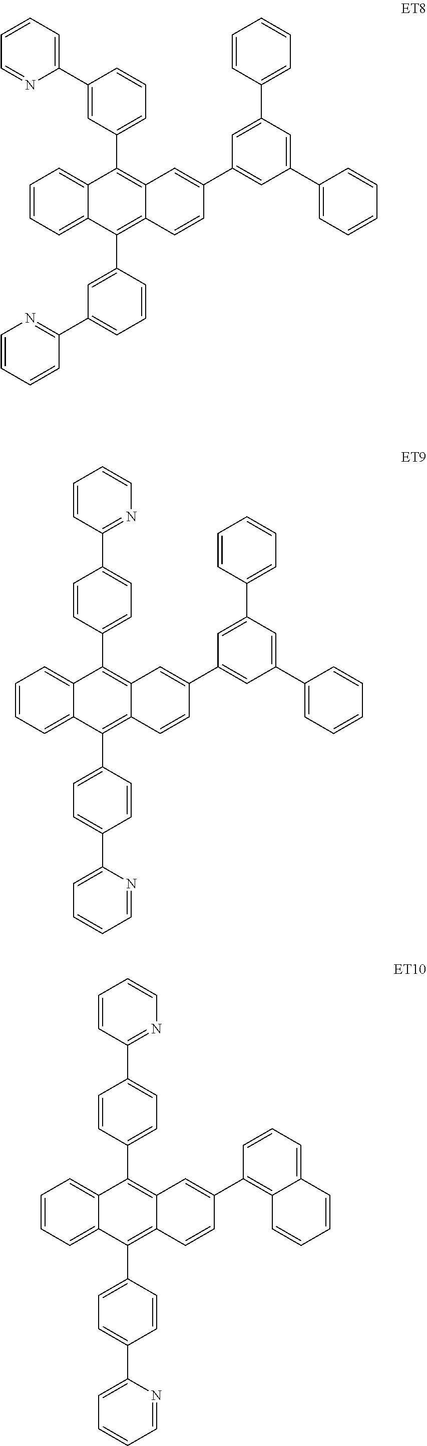 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

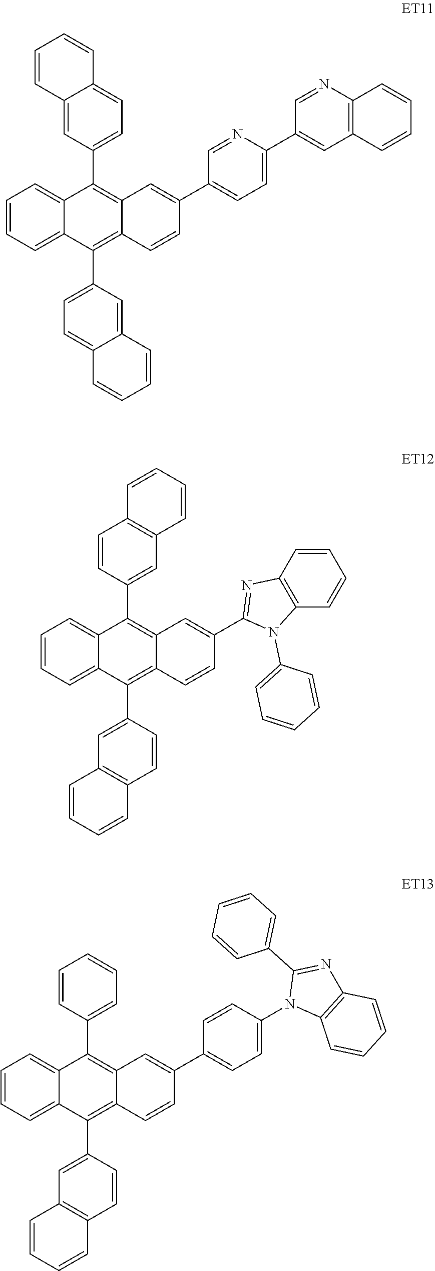 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

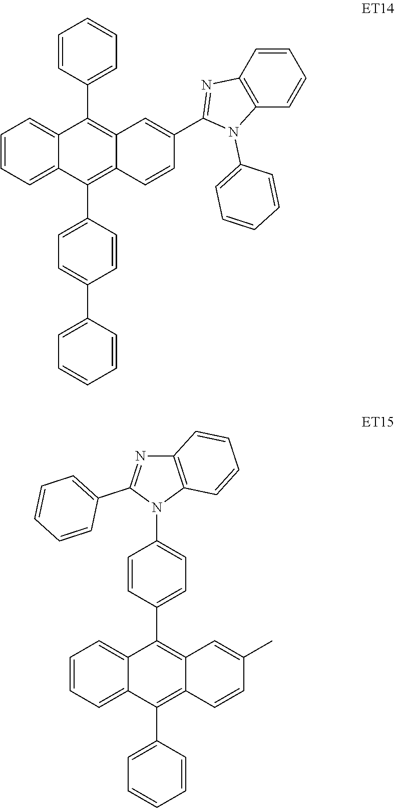 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

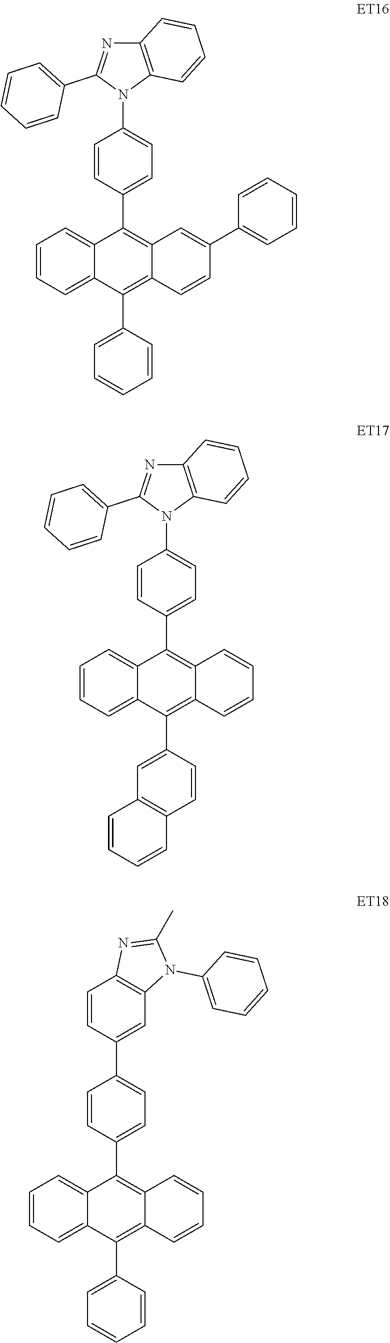 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

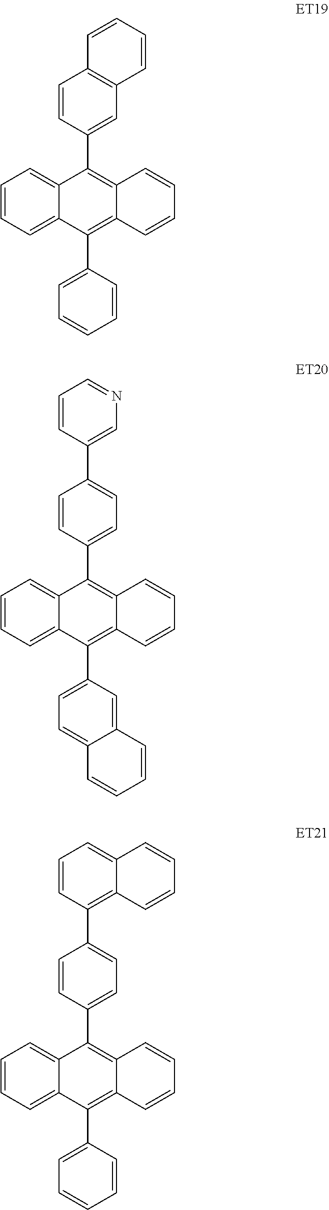

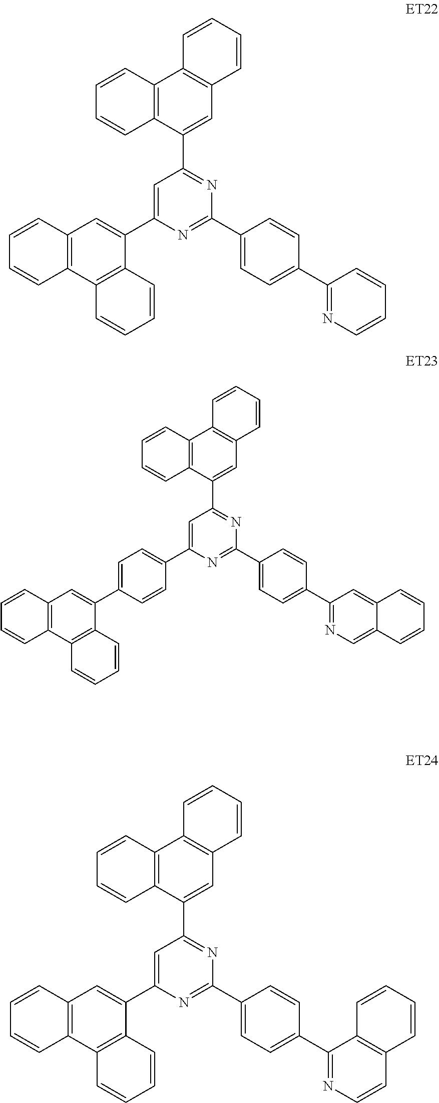 [SMILES: c1ccc(-c2ccc(-c3nc(-c4cc5ccccc5c5ccccc45)cc(-c4cc5ccccc5c5ccccc45)n3)cc2)nc1 ; c1ccc2cc(-c3ccc(-c4nc(-c5ccc(-c6cc7ccccc7c7ccccc67)cc5)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1]

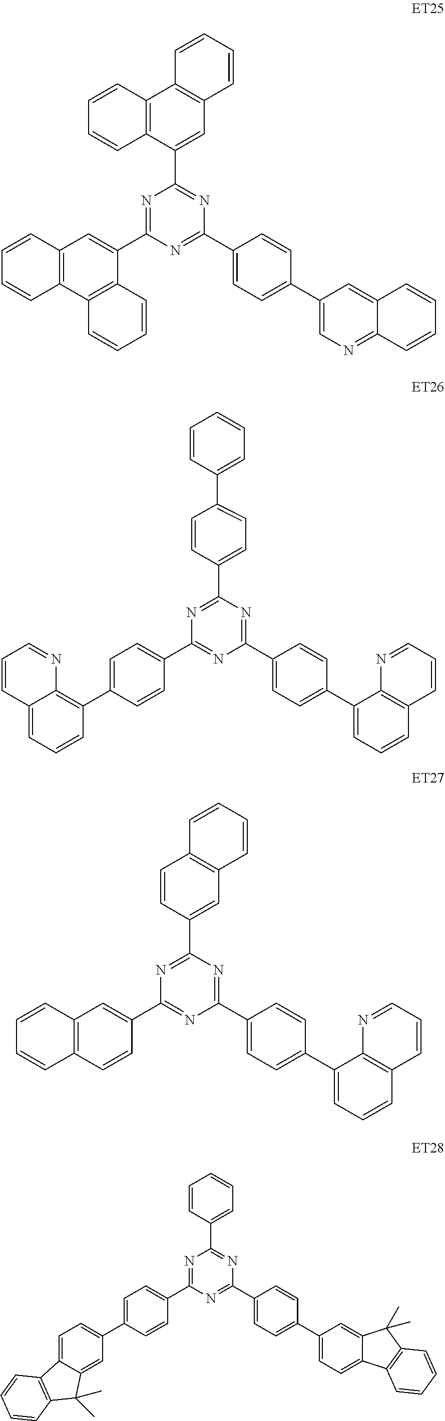 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

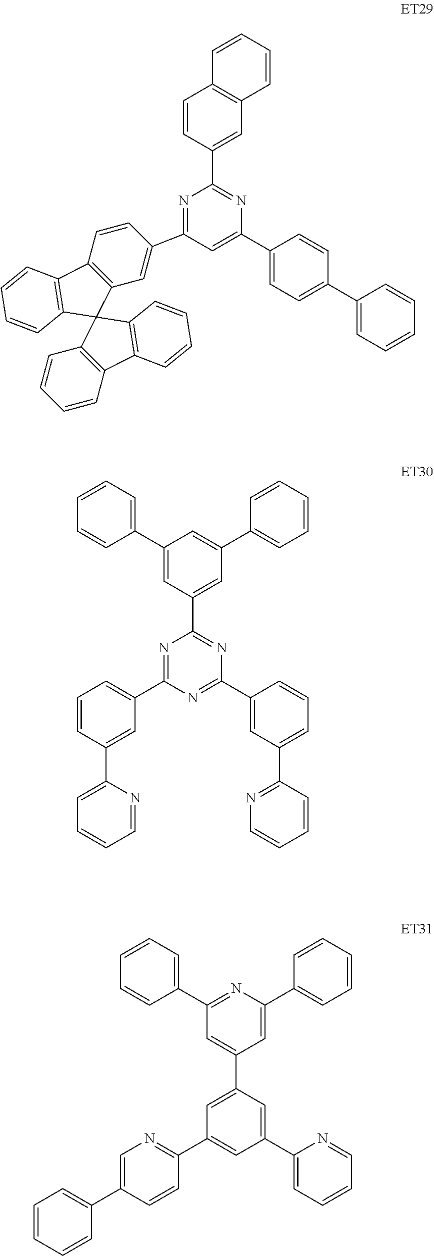 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

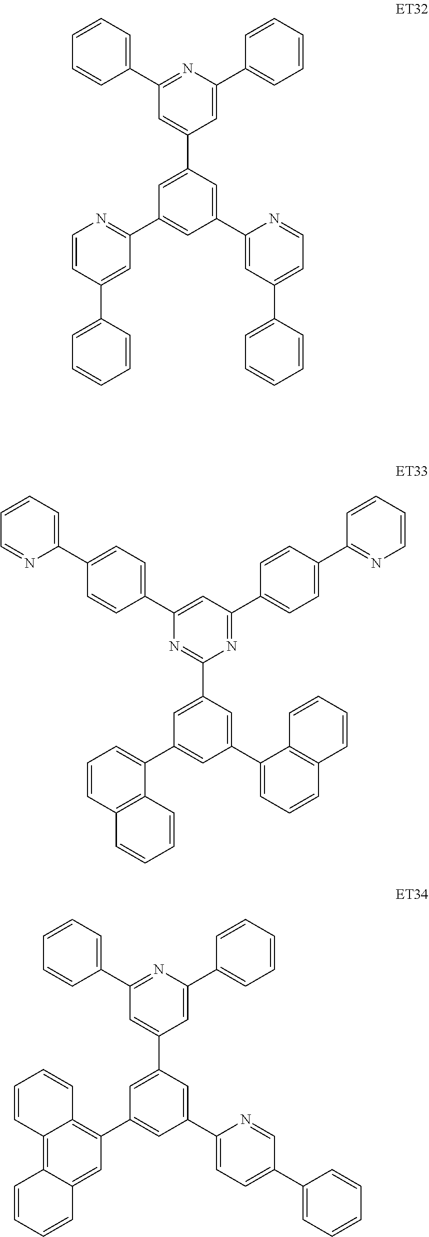 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

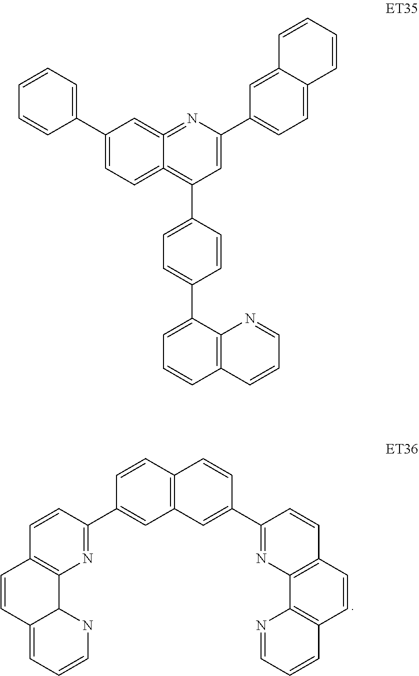

In some embodiments, the electron transport region ETR may include halogenated metals such as LiF, NaCl, CsF, RbCl, RbI, CuI, and/or KI, lanthanide metals such as Yb, co-deposition materials of a halogenated metal and/or a lanthanide metal. For example, the electron transport region ETR may include KI:Yb, RbI:Yb, LiF:Yb, etc. as a co-deposition material. In some embodiments, for the electron transport region ETR, a metal oxide such as Li₂O and BaO, or 8-hydroxyl-lithium quinolate (Liq), etc. may be utilized, but the embodiment of the present disclosure is limited thereto. The electron transport region ETR may also be formed of a mixture material of an electron transport material and an insulating organo-metal salt. The organo-metal salt may be a material having an energy band gap of about 4 eV or greater. For example, the organo-metal salt may include, for example, metal acetates, metal benzoates, metal acetoacetates, metal acetylacetonates, or metal stearates.

The electron transport region ETR may further include, for example, at least one selected from among 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the materials described above, but the embodiment of the present disclosure is not limited thereto.

The electron transport region ETR may include the compounds of the electron transport region described above in at least one selected from among the electron injection layer EIL, the electron transport layer ETL, and the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL satisfies the above-described range, satisfactory (suitable) electron transport properties may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described ranges, satisfactory (suitable) electron injection properties may be obtained without a substantial increase in driving voltage.

The second electrode EL2 is provided on the electron transport region ETR. The second electrode EL2 may be a common electrode. The second electrode EL2 may be a cathode or an anode but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode may include at least one selected from among Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, or one or more oxides thereof.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, compounds thereof, or mixtures thereof (e.g., AgMg, AgYb, or MgYb). In some embodiments, the second electrode EL2 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), etc. For example, the second electrode EL2 may include the above-described metal materials, a combination of two or more metal materials selected from the above-described metal materials, or one or more oxides of the above-described metal materials.

The second electrode EL2 may be connected with an auxiliary electrode. When the second electrode EL2 is connected with the auxiliary electrode, the resistance of the second electrode EL2 may decrease.

A capping layer CPL may be on the second electrode EL2 of the light emitting element ED of an embodiment. The capping layer CPL may include a multilayer or a single layer.

In an embodiment, the capping layer CPL may include an amine compound represented by Formula 1.

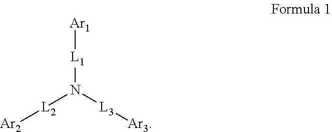

In Formula 1, *-L₁-Ar₁, *-L₂-Ar₂, and *-L₃-Ar₃ may each independently be represented by Formula 2. In Formula 1, *-L₁-Ar₁, *-L₂-Ar₂, and *-L₃-Ar₃ may all be the same or at least one may be different from the others.

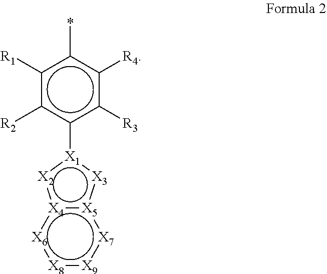

In Formula 2, R₁ to R₄ may each independently be a hydroxy group or a hydrogen atom, or bonded to an adjacent group to form a single bond. The forming of a single bond as R₁ to R₄ are bonded to an adjacent group may be forming a ring as a substituent of R₁ of a group represented by Formula 2 and a substituent of R₄ of an adjacent group represented by Formula 2 are bonded together. When R₁ to R₄ are bonded to an adjacent group to form a single bond, the formed ring may include a nitrogen atom (N) of an amine compound as a ring-forming atom.

In Formula 2, X₁ to X₉ may each independently be CH, C, N, O, or S. At least one selected from among X₁ to X₉ may be N, and for example, at least one selected from among X₁ to X₃ in Formula 2 may be N.

For example, the amine compound according to an embodiment may include a benzoazole derivative. For example, the amine compound according to an embodiment may be a tertiary amine compound including three benzoazole derivatives.

In Formula 1, at least one of *-L₁-Ar₁, *-L₂-Ar₂, or *-L₃-Ar₃ may include a hydroxy group. For example, the amine compound according to an embodiment, which is represented by Formula 1, may include at least one hydroxy group as a substituent.

Formula 2 may be represented by any one selected from among 1-1 to 1-3. However, in the embodiment in which *-L₁-Ar₁, *-L₂-Ar₂, and *-L₃-Ar₃ of Formula 1 may each independently be represented by any one selected from among I-1 to I-3, an embodiment in which *-L₁-Ar₁, *-L₂-Ar₂, and *-L₃-Ar₃ are all represented by I-1 is excluded.

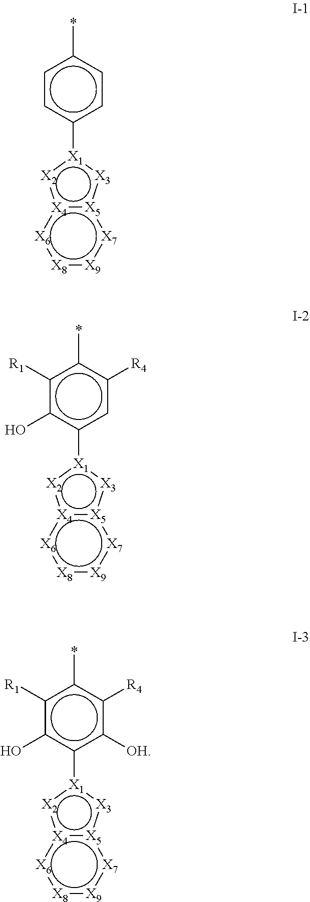

In I-2 and I-3, R₁ and R₄ may each independently be a hydrogen atom, or bonded to an adjacent group to form a single bond. In some embodiments, in I-1 to I-3, the same descriptions as in Formula 2 may be applied to X₁ to X₉.

In some embodiments, Formula 2 may be represented by any one selected from among I-4 to I-8.

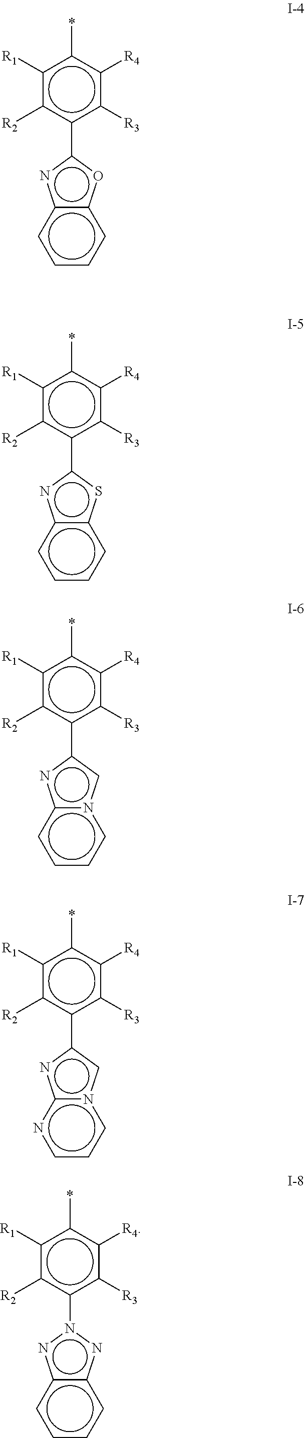

In Formulas I-4 and I-8, the same descriptions as in Formula 2 may be applied to R₁ and R₄. For example, in the amine compound according to an embodiment, which is represented by Formula 1, *-L₁-Ar₁, *-L₂-Ar₂, and *-L₃-Ar₃ may each independently be represented by any one selected from among I-4 to I-8. In this embodiment, at least one of *-L₁-Ar₁, *-L₂-Ar₂, or *-L₃-Ar₃ represented by any one selected from among I-4 to I-8 may include a hydroxy group.

In some embodiments, the polycyclic compound according to an embodiment may be represented by Formula 1-1. Formula 1-1 may be displayed by indicating portion

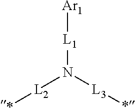

as “II” in Formula 1 described above.

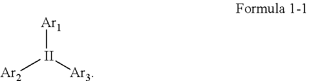

In Formula 1-1, II may be represented by II-A or II-B.

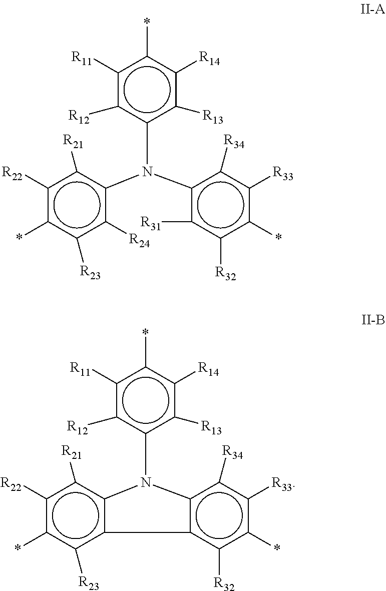

In II-A, at least one selected from among R₁₁ to R₁₄, R₂₁ to R₂₄, and R₃₁ to R₃₄ may be hydroxy and the others (the R₁₁ to R₁₄, R₂₁ to R₂₄, and R₃₁ to R₃₄ that are not a hydroxy) may be hydrogen atoms. In some embodiments, in II-B, at least one selected from among R₁₁ to R₁₄, R₂₁ to R₂₃, and R₃₂ to R₃₄ may be hydroxy and the others (the R₁₁ to R₁₄, R₂₁ to R₂₃, and R₃₂ to R₃₄ that are not hydroxy) may be hydrogen atoms.

In Formula 1-1, II may be represented by any one selected from among II-1 to II-16. In II-1 to II-16, *1 is a portion to which Ar₁ is bonded in Formula 1-1, *2 is a portion to which Ar₂ is bonded in Formula 1-1, and *3 is a portion to which Ar₃ is bonded in Formula 1-1.

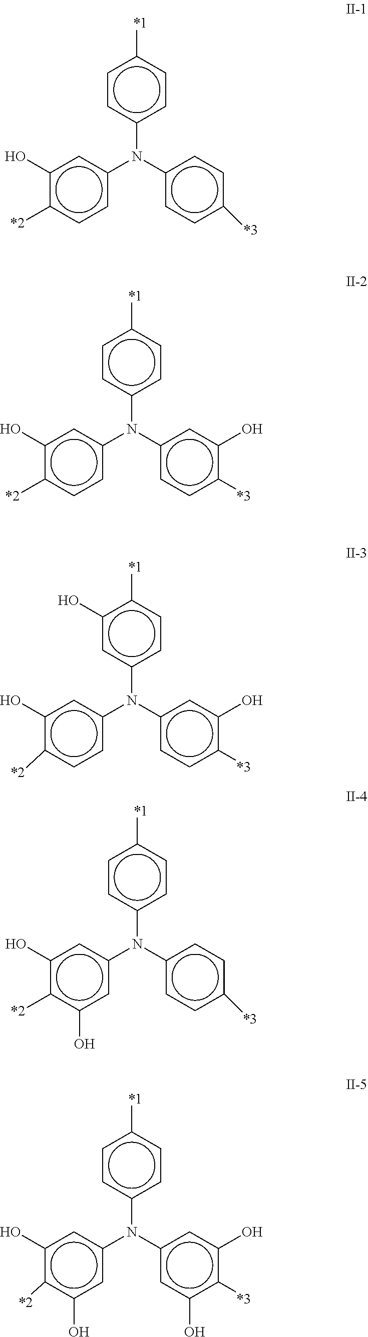

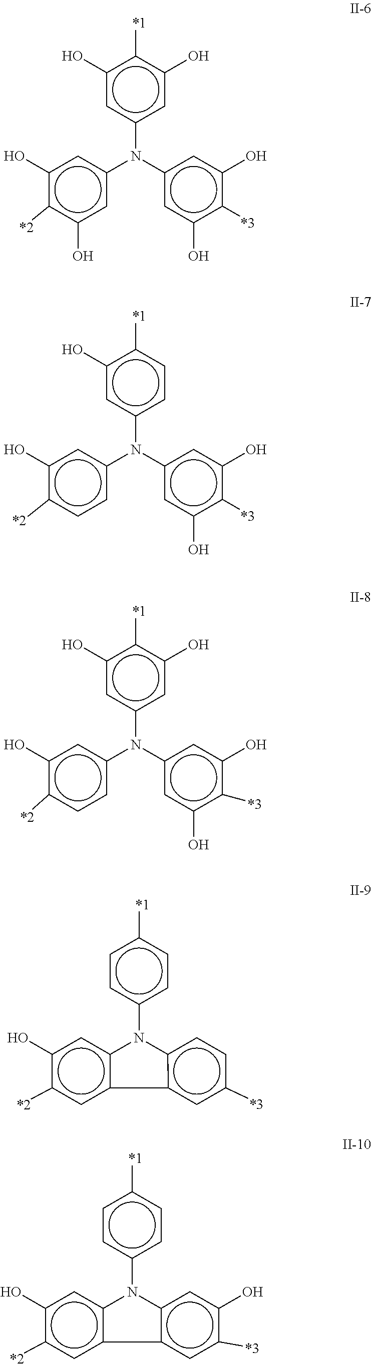

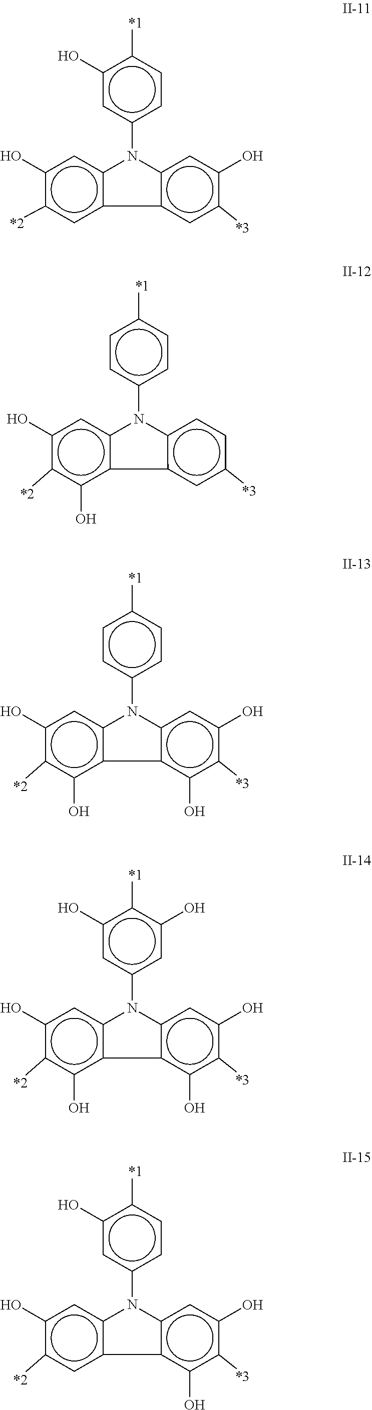

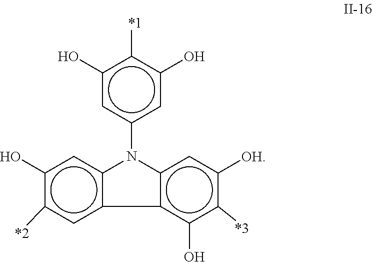

In Formula 1-1, Ar₁ to Ar₃ may each independently be a substituted or unsubstituted benzoazole derivative. In an embodiment, Ar₁ to Ar₃ may each independently be represented by any one selected from among III-1 to III-5. Ar₁ to Ar₃ may all be the same, or at least one selected from among Ar₁ to Ar₃ may be different from the others.

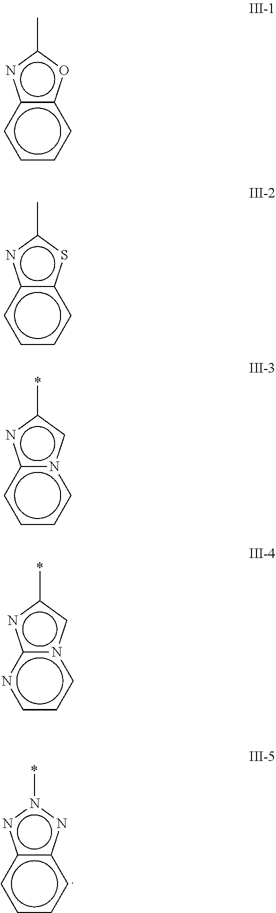

The amine compound according to an embodiment may include a benzoazole derivative and may include at least one hydroxy group as a substituent. In the amine compound according to an embodiment, hydrogen bonding between molecules takes place including hydroxy (i.e., the hydroxy is involved in hydrogen bonding), and the bonding of the amine compound molecules may thus increase planarity. Accordingly, the capping layer CPL including the amine compounds according to an embodiment, which are arranged in high planarity may exhibit excellent or suitable light extraction. Therefore, the light emitting element ED according to an embodiment, that includes the amine compound according to an embodiment in the capping layer CPL may exhibit high luminance and increased luminous efficiency characteristics.

The amine compound according to an embodiment, which is represented by Formula 1 may be represented by any one selected from among compounds. The capping layer CPL of the light emitting element ED according to an embodiment may include at least one selected from among the amine compounds shown in the following table.

TABLE 1

| Compound | | |
|---|---|---|
| classification | II | [Ar₂/Ar₁/Ar₃] |
| 1 | II-1 | [III-1/III-1/III-1] |
| 2 | II-1 | [III-1/III-1/III-2] |
| 3 | II-1 | [III-1/III-1/III-3] |
| 4 | II-1 | [III-1/III-1/III-4] |
| 5 | II-1 | [III-1/III-1/III-5] |
| 6 | II-1 | [III-1/III-2/III-1] |
| 7 | II-1 | [III-1/III-2/III-2] |
| 8 | II-1 | [III-1/III-2/III-3] |
| 9 | II-1 | [III-1/III-2/III-4] |
| 10 | II-1 | [III-1/III-2/III-5] |
| 11 | II-1 | [III-1/III-3/III-1] |
| 12 | II-1 | [III-1/III-3/III-2] |
| 13 | II-1 | [III-1/III-3/III-3] |
| 14 | II-1 | [III-1/III-3/III-4] |
| 15 | II-1 | [III-1/III-3/III-5] |
| 16 | II-1 | [III-1/III-4/III-1] |
| 17 | II-1 | [III-1/III-4/III-2] |
| 18 | II-1 | [III-1/III-4/III-3] |
| 19 | II-1 | [III-1/III-4/III-4] |
| 20 | II-1 | [III-1/III-4/III-5] |
| 21 | II-1 | [III-1/III-5/III-1] |
| 22 | II-1 | [III-1/III-5/III-2] |
| 23 | II-1 | [III-1/III-5/III-3] |
| 24 | II-1 | [III-1/III-5/III-4] |
| 25 | II-1 | [III-1/III-5/III-5] |
| 26 | II-1 | [III-2/III-1/III-1] |
| 27 | II-1 | [III-2/III-1/III-2] |
| 28 | II-1 | [III-2/III-1/III-3] |
| 29 | II-1 | [III-2/III-1/III-4] |
| 30 | II-1 | [III-2/III-1/III-5] |
| 31 | II-1 | [III-2/III-2/III-1] |
| 32 | II-1 | [III-2/III-2/III-2] |
| 33 | II-1 | [III-2/III-2/III-3] |
| 34 | II-1 | [III-2/III-2/III-4] |
| 35 | II-1 | [III-2/III-2/III-5] |
| 36 | II-1 | [III-2/III-3/III-1] |
| 37 | II-1 | [III-2/III-3/III-2] |
| 38 | II-1 | [III-2/III-3/III-3] |
| 39 | II-1 | [III-2/III-3/III-4] |
| 40 | II-1 | [III-2/III-3/III-5] |
| 41 | II-1 | [III-2/III-4/III-1] |
| 42 | II-1 | [III-2/III-4/III-2] |
| 43 | II-1 | [III-2/III-4/III-3] |
| 44 | II-1 | [III-2/III-4/III-4] |
| 45 | II-1 | [III-2/III-4/III-5] |
| 46 | II-1 | [III-2/III-5/III-1] |
| 47 | II-1 | [III-2/III-5/III-2] |
| 48 | II-1 | [III-2/III-5/III-3] |
| 49 | II-1 | [III-2/III-5/III-4] |
| 50 | II-1 | [III-2/III-5/III-5] |
| 51 | II-1 | [III-3/III-1/III-1] |
| 52 | II-1 | [III-3/III-1/III-2] |
| 53 | II-1 | [III-3/III-1/III-3] |
| 54 | II-1 | [III-3/III-1/III-4] |
| 55 | II-1 | [III-3/III-1/III-5] |
| 56 | II-1 | [III-3/III-2/III-1] |
| 57 | II-1 | [III-3/III-2/III-2] |
| 58 | II-1 | [III-3/III-2/III-3] |
| 59 | II-1 | [III-3/III-2/III-4] |
| 60 | II-1 | [III-3/III-2/III-5] |
| 61 | II-1 | [III-3/III-3/III-1] |
| 62 | II-1 | [III-3/III-3/III-2] |
| 63 | II-1 | [III-3/III-3/III-3] |
| 64 | II-1 | [III-3/III-3/III-4] |
| 65 | II-1 | [III-3/III-3/III-5] |
| 66 | II-1 | [III-3/III-4/III-1] |
| 67 | II-1 | [III-3/III-4/III-2] |
| 68 | II-1 | [III-3/III-4/III-3] |
| 69 | II-1 | [III-3/III-4/III-4] |
| 70 | II-1 | [III-3/III-4/III-5] |
| 71 | II-1 | [III-3/III-5/III-1] |
| 72 | II-1 | [III-3/III-5/III-2] |
| 73 | II-1 | [III-3/III-5/III-3] |
| 74 | II-1 | [III-3/III-5/III-4] |
| 75 | II-1 | [III-3/III-5/III-5] |
| 76 | II-1 | [III-4/III-1/III-1] |
| 77 | II-1 | [III-4/III-1/III-2] |
| 78 | II-1 | [III-4/III-1/III-3] |
| 79 | II-1 | [III-4/III-1/III-4] |
| 80 | II-1 | [III-4/III-1/III-5] |
| 81 | II-1 | [III-4/III-2/III-1] |
| 82 | II-1 | [III-4/III-2/III-2] |
| 83 | II-1 | [III-4/III-2/III-3] |
| 84 | II-1 | [III-4/III-2/III-4] |
| 85 | II-1 | [III-4/III-2/III-5] |
| 86 | II-1 | [III-4/III-3/III-1] |
| 87 | II-1 | [III-4/III-3/III-2] |
| 88 | II-1 | [III-4/III-3/III-3] |
| 89 | II-1 | [III-4/III-3/III-4] |
| 90 | II-1 | [III-4/III-3/III-5] |
| 91 | II-1 | [III-4/III-4/III-1] |
| 92 | II-1 | [III-4/III-4/III-2] |
| 93 | II-1 | [III-4/III-4/III-3] |
| 94 | II-1 | [III-4/III-4/III-4] |
| 95 | II-1 | [III-4/III-4/III-5] |
| 96 | II-1 | [III-4/III-5/III-1] |
| 97 | II-1 | [III-4/III-5/III-2] |
| 98 | II-1 | [III-4/III-5/III-3] |
| 99 | II-1 | [III-4/III-5/III-4] |
| 100 | II-1 | [III-4/III-5/III-5] |
| 101 | II-1 | [III-5/III-1/III-1] |
| 102 | II-1 | [III-5/III-1/III-2] |
| 103 | II-1 | [III-5/III-1/III-3] |
| 104 | II-1 | [III-5/III-1/III-4] |
| 105 | II-1 | [III-5/III-1/III-5] |
| 106 | II-1 | [III-5/III-2/III-1] |
| 107 | II-1 | [III-5/III-2/III-2] |
| 108 | II-1 | [III-5/III-2/III-3] |
| 109 | II-1 | [III-5/III-2/III-4] |
| 110 | II-1 | [III-5/III-2/III-5] |
| 111 | II-1 | [III-5/III-3/III-1] |
| 112 | II-1 | [III-5/III-3/III-2] |
| 113 | II-1 | [III-5/III-3/III-3] |
| 114 | II-1 | [III-5/III-3/III-4] |
| 115 | II-1 | [III-5/III-3/III-5] |
| 116 | II-1 | [III-5/III-4/III-1] |
| 117 | II-1 | [III-5/III-4/III-2] |
| 118 | II-1 | [III-5/III-4/III-3] |
| 119 | II-1 | [III-5/III-4/III-4] |
| 120 | II-1 | [III-5/III-4/III-5] |
| 121 | II-1 | [III-5/III-5/III-1] |
| 122 | II-1 | [III-5/III-5/III-2] |
| 123 | II-1 | [III-5/III-5/III-3] |
| 124 | II-1 | [III-5/III-5/III-4] |
| 125 | II-1 | [III-5/III-5/III-5] |
| 126 | II-2 | [III-1/III-1/III-1] |
| 127 | II-2 | [III-1/III-1/III-2] |
| 128 | II-2 | [III-1/III-1/III-3] |
| 129 | II-2 | [III-1/III-1/III-4] |
| 130 | II-2 | [III-1/III-1/III-5] |
| 131 | II-2 | [III-1/III-2/III-1] |
| 132 | II-2 | [III-1/III-2/III-2] |
| 133 | II-2 | [III-1/III-2/III-3] |
| 134 | II-2 | [III-1/III-2/III-4] |
| 135 | II-2 | [III-1/III-2/III-5] |
| 136 | II-2 | [III-1/III-3/III-1] |
| 137 | II-2 | [III-1/III-3/III-2] |
| 138 | II-2 | [III-1/III-3/III-3] |
| 139 | II-2 | [III-1/III-3/III-4] |
| 140 | II-2 | [III-1/III-3/III-5] |
| 141 | II-2 | [III-1/III-4/III-1] |
| 142 | II-2 | [III-1/III-4/III-2] |
| 143 | II-2 | [III-1/III-4/III-3] |
| 144 | II-2 | [III-1/III-4/III-4] |
| 145 | II-2 | [III-1/III-4/III-5] |
| 146 | II-2 | [III-1/III-5/III-1] |
| 147 | II-2 | [III-1/III-5/III-2] |
| 148 | II-2 | [III-1/III-5/III-3] |
| 149 | II-2 | [III-1/III-5/III-4] |
| 150 | II-2 | [III-1/III-5/III-5] |
| 151 | II-2 | [III-2/III-1/III-2] |
| 152 | II-2 | [III-2/III-1/III-3] |
| 153 | II-2 | [III-2/III-1/III-4] |
| 154 | II-2 | [III-2/III-1/III-5] |
| 155 | II-2 | [III-2/III-2/III-2] |
| 156 | II-2 | [III-2/III-2/III-3] |
| 157 | II-2 | [III-2/III-2/III-4] |
| 158 | II-2 | [III-2/III-2/III-5] |
| 159 | II-2 | [III-2/III-3/III-2] |
| 160 | II-2 | [III-2/III-3/III-3] |
| 161 | II-2 | [III-2/III-3/III-4] |
| 162 | II-2 | [III-2/III-3/III-5] |
| 163 | II-2 | [III-2/III-4/III-2] |
| 164 | II-2 | [III-2/III-4/III-3] |
| 165 | II-2 | [III-2/III-4/III-4] |
| 166 | II-2 | [III-2/III-4/III-5] |
| 167 | II-2 | [III-2/III-5/III-2] |
| 168 | II-2 | [III-2/III-5/III-3] |
| 169 | II-2 | [III-2/III-5/III-4] |
| 170 | II-2 | [III-2/III-5/III-5] |
| 171 | II-2 | [III-3/III-1/III-3] |
| 172 | II-2 | [III-3/III-1/III-4] |
| 173 | II-2 | [III-3/III-1/III-5] |
| 174 | II-2 | [III-3/III-2/III-3] |
| 175 | II-2 | [III-3/III-2/III-4] |
| 176 | II-2 | [III-3/III-2/III-5] |
| 177 | II-2 | [III-3/III-3/III-3] |
| 178 | II-2 | [III-3/III-3/III-4] |
| 179 | II-2 | [III-3/III-3/III-5] |
| 180 | II-2 | [III-3/III-4/III-3] |
| 181 | II-2 | [III-3/III-4/III-4] |
| 182 | II-2 | [III-3/III-4/III-5] |
| 183 | II-2 | [III-3/III-5/III-3] |
| 184 | II-2 | [III-3/III-5/III-4] |
| 185 | II-2 | [III-3/III-5/III-5] |
| 186 | II-2 | [III-4/III-1/III-4] |
| 187 | II-2 | [III-4/III-1/III-5] |
| 188 | II-2 | [III-4/III-2/III-4] |
| 189 | II-2 | [III-4/III-2/III-5] |
| 190 | II-2 | [III-4/III-3/III-4] |
| 191 | II-2 | [III-4/III-3/III-5] |
| 192 | II-2 | [III-4/III-4/III-4] |
| 193 | II-2 | [III-4/III-4/III-5] |
| 194 | II-2 | [III-4/III-5/III-4] |
| 195 | II-2 | [III-4/III-5/III-5] |
| 196 | II-2 | [III-5/III-1/III-5] |
| 197 | II-2 | [III-5/III-2/III-5] |
| 198 | II-2 | [III-5/III-1/III-1] |
| 199 | II-2 | [III-5/III-2/III-2] |
| 200 | II-2 | [III-5/III-3/III-2] |
| 201 | II-3 | [III-1/III-1/III-1] |
| 202 | II-3 | [III-1/III-1/III-2] |
| 203 | II-3 | [III-1/III-1/III-3] |
| 204 | II-3 | [III-1/III-1/III-4] |
| 205 | II-3 | [III-1/III-1/III-5] |
| 206 | II-3 | [III-1/III-2/III-1] |
| 207 | II-3 | [III-1/III-2/III-2] |
| 208 | II-3 | [III-1/III-2/III-3] |
| 209 | II-3 | [III-1/III-2/III-4] |
| 210 | II-3 | [III-1/III-2/III-5] |
| 211 | II-3 | [III-1/III-3/III-1] |
| 212 | II-3 | [III-1/III-3/III-2] |
| 213 | II-3 | [III-1/III-3/III-3] |
| 214 | II-3 | [III-1/III-3/III-4] |
| 215 | II-3 | [III-1/III-3/III-5] |
| 216 | II-3 | [III-1/III-4/III-1] |
| 217 | II-3 | [III-1/III-4/III-2] |
| 218 | II-3 | [III-1/III-4/III-3] |
| 219 | II-3 | [III-1/III-4/III-4] |
| 220 | II-3 | [III-1/III-4/III-5] |
| 221 | II-3 | [III-1/III-5/III-1] |
| 222 | II-3 | [III-1/III-5/III-2] |
| 223 | II-3 | [III-1/III-5/III-3] |
| 224 | II-3 | [III-1/III-5/III-4] |
| 225 | II-3 | [III-1/III-5/III-5] |
| 226 | II-3 | [III-2/III-1/III-1] |
| 227 | II-3 | [III-2/III-1/III-2] |
| 228 | II-3 | [III-2/III-1/III-3] |
| 229 | II-3 | [III-2/III-1/III-4] |
| 230 | II-3 | [III-2/III-1/III-5] |
| 231 | II-3 | [III-2/III-2/III-1] |
| 232 | II-3 | [III-2/III-2/III-2] |
| 233 | II-3 | [III-2/III-2/III-3] |
| 234 | II-3 | [III-2/III-2/III-4] |
| 235 | II-3 | [III-2/III-2/III-5] |
| 236 | II-3 | [III-2/III-3/III-1] |
| 237 | II-3 | [III-2/III-3/III-2] |
| 238 | II-3 | [III-2/III-3/III-3] |
| 239 | II-3 | [III-2/III-3/III-4] |
| 240 | II-3 | [III-2/III-3/III-5] |
| 241 | II-3 | [III-2/III-4/III-1] |
| 242 | II-3 | [III-2/III-4/III-2] |
| 243 | II-3 | [III-2/III-4/III-3] |
| 244 | II-3 | [III-2/III-4/III-4] |
| 245 | II-3 | [III-2/III-4/III-5] |
| 246 | II-3 | [III-2/III-5/III-1] |
| 247 | II-3 | [III-2/III-5/III-2] |
| 248 | II-3 | [III-2/III-5/III-3] |
| 249 | II-3 | [III-2/III-5/III-4] |
| 250 | II-3 | [III-2/III-5/III-5] |
| 251 | II-3 | [III-3/III-1/III-1] |
| 252 | II-3 | [III-3/III-1/III-2] |
| 253 | II-3 | [III-3/III-1/III-3] |
| 254 | II-3 | [III-3/III-1/III-4] |
| 255 | II-3 | [III-3/III-1/III-5] |
| 256 | II-3 | [III-3/III-2/III-1] |
| 257 | II-3 | [III-3/III-2/III-2] |
| 258 | II-3 | [III-3/III-2/III-3] |
| 259 | II-3 | [III-3/III-2/III-4] |
| 260 | II-3 | [III-3/III-2/III-5] |
| 261 | II-3 | [III-3/III-3/III-1] |
| 262 | II-3 | [III-3/III-3/III-2] |
| 263 | II-3 | [III-3/III-3/III-3] |
| 264 | II-3 | [III-3/III-3/III-4] |
| 265 | II-3 | [III-3/III-3/III-5] |
| 266 | II-3 | [III-3/III-4/III-1] |
| 267 | II-3 | [III-3/III-4/III-2] |
| 268 | II-3 | [III-3/III-4/III-3] |
| 269 | II-3 | [III-3/III-4/III-4] |
| 270 | II-3 | [III-3/III-4/III-5] |
| 271 | II-3 | [III-3/III-5/III-1] |
| 272 | II-3 | [III-3/III-5/III-2] |
| 273 | II-3 | [III-3/III-5/III-3] |
| 274 | II-3 | [III-3/III-5/III-4] |
| 275 | II-3 | [III-3/III-5/III-5] |
| 276 | II-3 | [III-4/III-1/III-1] |
| 277 | II-3 | [III-4/III-1/III-2] |
| 278 | II-3 | [III-4/III-1/III-3] |
| 279 | II-3 | [III-4/III-1/III-4] |
| 280 | II-3 | [III-4/III-1/III-5] |
| 201 | II-3 | [III-4/III-2/III-1] |
| 282 | II-3 | [III-4/III-2/III-2] |
| 283 | II-3 | [III-4/III-2/III-3] |
| 284 | II-3 | [III-4/III-2/III-4] |
| 285 | II-3 | [III-4/III-2/III-5] |
| 286 | II-3 | [III-4/III-3/III-1] |
| 287 | II-3 | [III-4/III-3/III-2] |
| 288 | II-3 | [III-4/III-3/III-3] |
| 289 | II-3 | [III-4/III-3/III-4] |
| 290 | II-3 | [III-4/III-3/III-5] |
| 291 | II-3 | [III-4/III-4/III-1] |
| 292 | II-3 | [III-4/III-4/III-2] |
| 293 | II-3 | [III-4/III-4/III-3] |
| 294 | II-3 | [III-4/III-4/III-4] |
| 295 | II-3 | [III-4/III-4/III-5] |
| 296 | II-3 | [III-4/III-5/III-1] |
| 297 | II-3 | [III-4/III-5/III-2] |
| 298 | II-3 | [III-4/III-5/III-3] |
| 299 | II-3 | [III-4/III-5/III-4] |
| 300 | II-3 | [III-4/III-5/III-5] |
| 301 | II-3 | [III-5/III-1/III-1] |
| 302 | II-3 | [III-5/III-1/III-2] |
| 303 | II-3 | [III-5/III-1/III-3] |
| 304 | II-3 | [III-5/III-1/III-4] |
| 305 | II-3 | [III-5/III-1/III-5] |
| 306 | II-3 | [III-5/III-2/III-1] |
| 307 | II-3 | [III-5/III-2/III-2] |
| 308 | II-3 | [III-5/III-2/III-3] |
| 309 | II-3 | [III-5/III-2/III-4] |
| 310 | II-3 | [III-5/III-2/III-5] |
| 311 | II-3 | [III-5/III-3/III-1] |
| 312 | II-3 | [III-5/III-3/III-2] |
| 313 | II-3 | [III-5/III-3/III-3] |
| 314 | II-3 | [III-5/III-3/III-4] |
| 315 | II-3 | [III-5/III-3/III-5] |
| 316 | II-3 | [III-5/III-4/III-1] |
| 317 | II-3 | [III-5/III-4/III-2] |
| 318 | II-3 | [III-5/III-4/III-3] |
| 319 | II-3 | [III-5/III-4/III-4] |
| 320 | II-3 | [III-5/III-4/III-5] |
| 321 | II-3 | [III-5/III-5/III-1] |
| 322 | II-3 | [III-5/III-5/III-2] |
| 323 | II-3 | [III-5/III-5/III-3] |
| 324 | II-3 | [III-5/III-5/III-4] |
| 325 | II-3 | [III-5/III-5/III-5] |
| 326 | II-4 | [III-1/III-1/III-1] |
| 327 | II-4 | [III-1/III-1/III-2] |
| 328 | II-4 | [III-1/III-1/III-3] |
| 329 | II-4 | [III-1/III-1/III-4] |
| 330 | II-4 | [III-1/III-1/III-5] |
| 331 | II-4 | [III-1/III-2/III-1] |
| 332 | II-4 | [III-1/III-2/III-2] |
| 333 | II-4 | [III-1/III-2/III-3] |
| 334 | II-4 | [III-1/III-2/III-4] |
| 335 | II-4 | [III-1/III-2/III-5] |
| 336 | II-4 | [III-1/III-3/III-1] |
| 337 | II-4 | [III-1/III-3/III-2] |
| 338 | II-4 | [III-1/III-3/III-3] |
| 339 | II-4 | [III-1/III-3/III-4] |
| 340 | II-4 | [III-1/III-3/III-5] |
| 341 | II-4 | [III-1/III-4/III-1] |
| 342 | II-4 | [III-1/III-4/III-2] |
| 343 | II-4 | [III-1/III-4/III-3] |
| 344 | II-4 | [III-1/III-4/III-4] |
| 345 | II-4 | [III-1/III-4/III-5] |
| 346 | II-4 | [III-1/III-5/III-1] |
| 347 | II-4 | [III-1/III-5/III-2] |
| 348 | II-4 | [III-1/III-5/III-3] |
| 349 | II-4 | [III-1/III-5/III-4] |
| 350 | II-4 | [III-1/III-5/III-5] |
| 351 | II-4 | [III-2/III-1/III-1] |
| 352 | II-4 | [III-2/III-1/III-2] |
| 353 | II-4 | [III-2/III-1/III-3] |
| 354 | II-4 | [III-2/III-1/III-4] |
| 355 | II-4 | [III-2/III-1/III-5] |
| 356 | II-4 | [III-2/III-2/III-1] |
| 357 | II-4 | [III-2/III-2/III-2] |
| 358 | II-4 | [III-2/III-2/III-3] |
| 359 | II-4 | [III-2/III-2/III-4] |
| 360 | II-4 | [III-2/III-2/III-5] |
| 361 | II-4 | [III-2/III-3/III-1] |
| 362 | II-4 | [III-2/III-3/III-2] |
| 363 | II-4 | [III-2/III-3/III-3] |
| 364 | II-4 | [III-2/III-3/III-4] |
| 365 | II-4 | [III-2/III-3/III-5] |
| 366 | II-4 | [III-2/III-4/III-1] |
| 367 | II-4 | [III-2/III-4/III-2] |
| 368 | II-4 | [III-2/III-4/III-3] |
| 369 | II-4 | [III-2/III-4/III-4] |
| 370 | II-4 | [III-2/III-4/III-5] |
| 371 | II-4 | [III-2/III-5/III-1] |
| 373 | II-4 | [III-2/III-5/III-2] |
| 373 | II-4 | [III-2/III-5/III-3] |
| 374 | II-4 | [III-2/III-5/III-4] |
| 375 | II-4 | [III-2/III-5/III-5] |
| 376 | II-4 | [III-3/III-1/III-1] |
| 377 | II-4 | [III-3/III-1/III-2] |
| 378 | II-4 | [III-3/III-1/III-3] |
| 379 | II-4 | [III-3/III-1/III-4] |
| 380 | II-4 | [III-3/III-1/III-5] |
| 381 | II-4 | [III-3/III-2/III-1] |
| 382 | II-4 | [III-3/III-2/III-2] |
| 383 | II-4 | [III-3/III-2/III-3] |
| 384 | II-4 | [III-3/III-2/III-4] |
| 385 | II-4 | [III-3/III-2/III-5] |
| 386 | II-4 | [III-3/III-3/III-1] |
| 387 | II-4 | [III-3/III-3/III-2] |
| 388 | II-4 | [III-3/III-3/III-3] |
| 389 | II-4 | [III-3/III-3/III-4] |
| 390 | II-4 | [III-3/III-3/III-5] |
| 391 | II-4 | [III-3/III-4/III-1] |
| 392 | II-4 | [III-3/III-4/III-2] |
| 393 | II-4 | [III-3/III-4/III-3] |
| 394 | II-4 | [III-3/III-4/III-4] |
| 395 | II-4 | [III-3/III-4/III-5] |
| 396 | II-4 | [III-3/III-5/III-1] |
| 397 | II-4 | [III-3/III-5/III-2] |
| 398 | II-4 | [III-3/III-5/III-3] |
| 399 | II-4 | [III-3/III-5/III-4] |
| 400 | II-4 | [III-3/III-5/III-5] |
| 401 | II-4 | [III-4/III-1/III-1] |
| 402 | II-4 | [III-4/III-1/III-2] |
| 403 | II-4 | [III-4/III-1/III-3] |
| 404 | II-4 | [III-4/III-1/III-4] |
| 405 | II-4 | [III-4/III-1/III-5] |
| 406 | II-4 | [III-4/III-2/III-1] |
| 407 | II-4 | [III-4/III-2/III-2] |
| 408 | II-4 | [III-4/III-2/III-3] |
| 409 | II-4 | [III-4/III-2/III-4] |
| 410 | II-4 | [III-4/III-2/III-5] |
| 411 | II-4 | [III-4/III-3/III-1] |
| 412 | II-4 | [III-4/III-3/III-2] |
| 413 | II-4 | [III-4/III-3/III-3] |
| 414 | II-4 | [III-4/III-3/III-4] |
| 415 | II-4 | [III-4/III-3/III-5] |
| 416 | II-4 | [III-4/III-4/III-1] |
| 417 | II-4 | [III-4/III-4/III-2] |
| 418 | II-4 | [III-4/III-4/III-3] |
| 419 | II-4 | [III-4/III-4/III-4] |
| 420 | II-4 | [III-4/III-4/III-5] |
| 421 | II-4 | [III-4/III-5/III-1] |
| 422 | II-4 | [III-4/III-5/III-2] |
| 423 | II-4 | [III-4/III-5/III-3] |
| 424 | II-4 | [III-4/III-5/III-4] |
| 425 | II-4 | [III-4/III-5/III-5] |
| 426 | II-4 | [III-5/III-1/III-1] |
| 427 | II-4 | [III-5/III-1/III-2] |
| 428 | II-4 | [III-5/III-1/III-3] |
| 429 | II-4 | [III-5/III-1/III-4] |
| 430 | II-4 | [III-5/III-1/III-5] |
| 431 | II-4 | [III-5/III-2/III-1] |
| 432 | II-4 | [III-5/III-2/III-2] |
| 433 | II-4 | [III-5/III-2/III-3] |
| 434 | II-4 | [III-5/III-2/III-4] |
| 435 | II-4 | [III-5/III-2/III-5] |
| 436 | II-4 | [III-5/III-3/III-1] |
| 437 | II-4 | [III-5/III-3/III-2] |
| 438 | II-4 | [III-5/III-3/III-3] |
| 439 | II-4 | [III-5/III-3/III-4] |
| 440 | II-4 | [III-5/III-3/III-5] |
| 441 | II-4 | [III-5/III-4/III-1] |
| 442 | II-4 | [III-5/III-4/III-2] |
| 443 | II-4 | [III-5/III-4/III-3] |
| 444 | II-4 | [III-5/III-4/III-4] |
| 445 | II-4 | [III-5/III-4/III-5] |
| 446 | II-4 | [III-5/III-5/III-1] |
| 447 | II-4 | [III-5/III-5/III-2] |
| 448 | II-4 | [III-5/III-5/III-3] |
| 449 | II-4 | [III-5/III-5/III-4] |
| 450 | II-4 | [III-5/III-5/III-5] |
| 451 | II-5 | [III-1/III-1/III-1] |
| 452 | II-5 | [III-1/III-1/III-2] |
| 453 | II-5 | [III-1/III-1/III-3] |
| 454 | II-5 | [III-1/III-1/III-4] |
| 455 | II-5 | [III-1/III-1/III-5] |
| 456 | II-5 | [III-1/III-2/III-1] |
| 457 | II-5 | [III-1/III-2/III-2] |
| 458 | II-5 | [III-1/III-2/III-3] |
| 459 | II-5 | [III-1/III-2/III-4] |
| 460 | II-5 | [III-1/III-2/III-5] |
| 461 | II-5 | [III-1/III-3/III-1] |
| 462 | II-5 | [III-1/III-3/III-2] |
| 463 | II-5 | [III-1/III-3/III-3] |
| 464 | II-5 | [III-1/III-3/III-4] |
| 465 | II-5 | [III-1/III-3/III-5] |
| 466 | II-5 | [III-1/III-4/III-1] |
| 467 | II-5 | [III-1/III-4/III-2] |
| 468 | II-5 | [III-1/III-4/III-3] |
| 469 | II-5 | [III-1/III-4/III-4] |
| 470 | II-5 | [III-1/III-4/III-5] |
| 471 | II-5 | [III-1/III-5/III-1] |
| 472 | II-5 | [III-1/III-5/III-2] |
| 473 | II-5 | [III-1/III-5/III-3] |
| 474 | II-5 | [III-1/III-5/III-4] |
| 475 | II-5 | [III-1/III-5/III-5] |
| 476 | II-5 | [III-2/III-1/III-2] |
| 477 | II-5 | [III-2/III-1/III-3] |
| 478 | II-5 | [III-2/III-1/III-4] |
| 479 | II-5 | [III-2/III-1/III-5] |
| 480 | II-5 | [III-2/III-2/III-2] |
| 481 | II-5 | [III-2/III-2/III-3] |
| 482 | II-5 | [III-2/III-2/III-4] |
| 483 | II-5 | [III-2/III-2/III-5] |
| 484 | II-5 | [III-2/III-3/III-2] |
| 485 | II-5 | [III-2/III-3/III-3] |
| 486 | II-5 | [III-2/III-3/III-4] |
| 487 | II-5 | [III-2/III-3/III-5] |
| 488 | II-5 | [III-2/III-4/III-2] |
| 489 | II-5 | [III-2/III-4/III-3] |
| 490 | II-5 | [III-2/III-4/III-4] |
| 491 | II-5 | [III-2/III-4/III-5] |
| 492 | II-5 | [III-2/III-5/III-2] |
| 493 | II-5 | [III-2/III-5/III-3] |
| 494 | II-5 | [III-2/III-5/III-4] |
| 495 | II-5 | [III-2/III-5/III-5] |
| 496 | II-5 | [III-3/III-1/III-3] |
| 497 | II-5 | [III-3/III-1/III-4] |
| 498 | II-5 | [III-3/III-1/III-5] |
| 499 | II-5 | [III-3/III-2/III-3] |
| 500 | II-5 | [III-3/III-2/III-4] |
| 501 | II-5 | [III-3/III-2/III-5] |
| 502 | II-5 | [III-3/III-3/III-3] |
| 503 | II-5 | [III-3/III-3/III-4] |
| 504 | II-5 | [III-3/III-3/III-5] |
| 505 | II-5 | [III-3/III-4/III-3] |
| 506 | II-5 | [III-3/III-4/III-4] |
| 507 | II-5 | [III-3/III-4/III-5] |
| 508 | II-5 | [III-3/III-5/III-3] |
| 509 | II-5 | [III-3/III-5/III-4] |
| 510 | II-5 | [III-3/III-5/III-5] |
| 511 | II-5 | [III-4/III-1/III-4] |
| 512 | II-5 | [III-4/III-1/III-5] |
| 513 | II-5 | [III-4/III-2/III-4] |
| 514 | II-5 | [III-4/III-2/III-5] |
| 515 | II-5 | [III-4/III-3/III-4] |
| 516 | II-5 | [III-4/III-3/III-5] |
| 517 | II-5 | [III-4/III-4/III-4] |
| 518 | II-5 | [III-4/III-4/III-5] |
| 519 | II-5 | [III-4/III-5/III-4] |
| 520 | II-5 | [III-4/III-5/III-5] |
| 521 | II-5 | [III-5/III-1/III-5] |
| 522 | II-5 | [III-5/III-2/III-5] |
| 523 | II-5 | [III-5/III-3/III-5] |
| 524 | II-5 | [III-5/III-4/III-5] |
| 525 | II-5 | [III-5/III-5/III-5] |
| 526 | II-6 | [III-1/III-1/III-1] |
| 527 | II-6 | [III-1/III-1/III-2] |
| 528 | II-6 | [III-1/III-1/III-3] |
| 529 | II-6 | [III-1/III-1/III-4] |
| 530 | II-6 | [III-1/III-1/III-5] |
| 531 | II-6 | [III-1/III-2/III-1] |
| 532 | II-6 | [III-1/III-2/III-2] |
| 533 | II-6 | [III-1/III-2/III-3] |
| 534 | II-6 | [III-1/III-2/III-4] |
| 535 | II-6 | [III-1/III-2/III-5] |
| 536 | II-6 | [III-1/III-3/III-1] |
| 537 | II-6 | [III-1/III-3/III-2] |
| 538 | II-6 | [III-1/III-3/III-3] |
| 539 | II-6 | [III-1/III-3/III-4] |
| 540 | II-6 | [III-1/III-3/III-5] |
| 541 | II-6 | [III-1/III-4/III-1] |
| 542 | II-6 | [III-1/III-4/III-2] |
| 543 | II-6 | [III-1/III-4/III-3] |
| 544 | II-6 | [III-1/III-4/III-4] |
| 545 | II-6 | [III-1/III-4/III-5] |
| 546 | II-6 | [III-1/III-5/III-1] |
| 547 | II-6 | [III-1/III-5/III-2] |
| 548 | II-6 | [III-1/III-5/III-3] |
| 549 | II-6 | [III-1/III-5/III-4] |
| 550 | II-6 | [III-1/III-5/III-5] |
| 551 | II-6 | [III-2/III-1/III-2] |
| 552 | II-6 | [III-2/III-1/III-3] |
| 553 | II-6 | [III-2/III-1/III-4] |
| 554 | II-6 | [III-2/III-1/III-5] |
| 555 | II-6 | [III-2/III-2/III-2] |
| 556 | II-6 | [III-2/III-2/III-3] |
| 557 | II-6 | [III-2/III-2/III-4] |
| 558 | II-6 | [III-2/III-2/III-5] |
| 559 | II-6 | [III-2/III-3/III-3] |
| 560 | II-6 | [III-2/III-3/III-4] |
| 561 | II-6 | [III-2/III-3/III-5] |
| 562 | II-6 | [III-2/III-4/III-2] |
| 563 | II-6 | [III-2/III-4/III-3] |
| 564 | II-6 | [III-2/III-4/III-4] |
| 565 | II-6 | [III-2/III-4/III-5] |
| 566 | II-6 | [III-2/III-5/III-1] |
| 567 | II-6 | [III-2/III-5/III-2] |
| 568 | II-6 | [III-2/III-5/III-3] |
| 569 | II-6 | [III-2/III-5/III-4] |
| 570 | II-6 | [III-2/III-5/III-5] |
| 571 | II-6 | [III-3/III-1/III-3] |
| 572 | II-6 | [III-3/III-1/III-4] |
| 573 | II-6 | [III-3/III-1/III-5] |
| 574 | II-6 | [III-3/III-2/III-3] |
| 575 | II-6 | [III-3/III-2/III-4] |
| 576 | II-6 | [III-3/III-2/III-5] |
| 577 | II-6 | [III-3/III-3/III-3] |
| 578 | II-6 | [III-3/III-3/III-4] |
| 579 | II-6 | [III-3/III-3/III-5] |
| 580 | II-6 | [III-3/III-4/III-3] |
| 581 | II-6 | [III-3/III-4/III-4] |
| 582 | II-6 | [III-3/III-4/III-5] |
| 583 | II-6 | [III-3/III-5/III-3] |
| 584 | II-6 | [III-3/III-5/III-4] |
| 585 | II-6 | [III-3/III-5/III-5] |
| 586 | II-6 | [III-4/III-1/III-4] |
| 587 | II-6 | [III-4/III-1/III-5] |
| 588 | II-6 | [III-4/III-2/III-4] |
| 589 | II-6 | [III-4/III-2/III-5] |
| 590 | II-6 | [III-4/III-3/III-4] |
| 591 | II-6 | [III-4/III-3/III-5] |
| 592 | II-6 | [III-4/III-4/III-4] |
| 593 | II-6 | [III-4/III-4/III-5] |
| 594 | II-6 | [III-4/III-5/III-4] |
| 595 | II-6 | [III-4/III-5/III-5] |
| 596 | II-6 | [III-5/III-1/III-5] |
| 597 | II-6 | [III-5/III-2/III-5] |
| 598 | II-6 | [III-5/III-3/III-5] |
| 599 | II-6 | [III-5/III-4/III-5] |
| 600 | II-6 | [III-5/III-5/III-5] |
| 601 | II-7 | [III-1/III-1/III-1] |
| 602 | II-7 | [III-1/III-1/III-2] |
| 603 | II-7 | [III-1/III-1/III-3] |
| 604 | II-7 | [III-1/III-1/III-4] |
| 605 | II-7 | [III-1/III-1/III-5] |
| 606 | II-7 | [III-1/III-2/III-1] |
| 607 | II-7 | [III-1/III-2/III-2] |
| 608 | II-7 | [III-1/III-2/III-3] |
| 609 | II-7 | [III-1/III-2/III-4] |
| 610 | II-7 | [III-1/III-2/III-5] |
| 611 | II-7 | [III-1/III-3/III-1] |
| 612 | II-7 | [III-1/III-3/III-2] |
| 613 | II-7 | [III-1/III-3/III-3] |
| 614 | II-7 | [III-1/III-3/III-4] |
| 615 | II-7 | [III-1/III-3/III-5] |
| 616 | II-7 | [III-1/III-4/III-1] |
| 617 | II-7 | [III-1/III-4/III-2] |
| 618 | II-7 | [III-1/III-4/III-3] |
| 619 | II-7 | [III-1/III-4/III-4] |
| 620 | II-7 | [III-1/III-4/III-5] |
| 621 | II-7 | [III-1/III-5/III-1] |
| 622 | II-7 | [III-1/III-5/III-2] |
| 623 | II-7 | [III-1/III-5/III-3] |
| 624 | II-7 | [III-1/III-5/III-4] |
| 625 | II-7 | [III-1/III-5/III-5] |
| 626 | II-7 | [III-2/III-1/III-1] |
| 627 | II-7 | [III-2/III-1/III-2] |
| 628 | II-7 | [III-2/III-1/III-3] |
| 629 | II-7 | [III-2/III-1/III-4] |
| 630 | II-7 | [III-2/III-1/III-5] |
| 631 | II-7 | [III-2/III-2/III-1] |
| 632 | II-7 | [III-2/III-2/III-2] |
| 633 | II-7 | [III-2/III-2/III-3] |
| 634 | II-7 | [III-2/III-2/III-4] |
| 635 | II-7 | [III-2/III-2/III-5] |
| 636 | II-7 | [III-2/III-3/III-1] |
| 637 | II-7 | [III-2/III-3/III-2] |
| 638 | II-7 | [III-2/III-3/III-3] |
| 639 | II-7 | [III-2/III-3/III-4] |
| 640 | II-7 | [III-2/III-3/III-5] |
| 641 | II-7 | [III-2/III-4/III-1] |
| 642 | II-7 | [III-2/III-4/III-2] |
| 643 | II-7 | [III-2/III-4/III-3] |
| 644 | II-7 | [III-2/III-4/III-4] |
| 645 | II-7 | [III-2/III-4/III-5] |
| 646 | II-7 | [III-2/III-5/III-1] |
| 647 | II-7 | [III-2/III-5/III-2] |
| 648 | II-7 | [III-2/III-5/III-3] |
| 649 | II-7 | [III-2/III-5/III-4] |
| 650 | II-7 | [III-2/III-5/III-5] |
| 651 | II-7 | [III-3/III-1/III-1] |
| 652 | II-7 | [III-3/III-1/III-2] |
| 653 | II-7 | [III-3/III-1/III-3] |
| 654 | II-7 | [III-3/III-1/III-4] |
| 655 | II-7 | [III-3/III-1/III-5] |
| 656 | II-7 | [III-3/III-2/III-1] |
| 657 | II-7 | [III-3/III-2/III-2] |
| 658 | II-7 | [III-3/III-2/III-3] |
| 659 | II-7 | [III-3/III-2/III-4] |
| 660 | II-7 | [III-3/III-2/III-5] |
| 661 | II-7 | [III-3/III-3/III-1] |
| 662 | II-7 | [III-3/III-3/III-2] |
| 663 | II-7 | [III-3/III-3/III-3] |
| 664 | II-7 | [III-3/III-3/III-4] |
| 665 | II-7 | [III-3/III-3/III-5] |
| 666 | II-7 | [III-3/III-4/III-1] |
| 667 | II-7 | [III-3/III-4/III-2] |
| 668 | II-7 | [III-3/III-4/III-3] |
| 669 | II-7 | [III-3/III-4/III-4] |
| 670 | II-7 | [III-3/III-4/III-5] |
| 671 | II-7 | [III-3/III-5/III-1] |
| 672 | II-7 | [III-3/III-5/III-2] |
| 673 | II-7 | [III-3/III-5/III-3] |
| 674 | II-7 | [III-3/III-5/III-4] |
| 675 | II-7 | [III-3/III-5/III-5] |
| 676 | II-7 | [III-4/III-1/III-1] |
| 677 | II-7 | [III-4/III-1/III-2] |
| 678 | II-7 | [III-4/III-1/III-3] |
| 679 | II-7 | [III-4/III-1/III-4] |
| 680 | II-7 | [III-4/III-1/III-5] |
| 681 | II-7 | [III-4/III-2/III-1] |
| 682 | II-7 | [III-4/III-2/III-2] |
| 683 | II-7 | [III-4/III-2/III-3] |
| 684 | II-7 | [III-4/III-2/III-4] |
| 685 | II-7 | [III-4/III-2/III-5] |
| 686 | II-7 | [III-4/III-3/III-1] |
| 687 | II-7 | [III-4/III-3/III-2] |
| 688 | II-7 | [III-4/III-3/III-3] |
| 689 | II-7 | [III-4/III-3/III-4] |
| 690 | II-7 | [III-4/III-3/III-5] |
| 691 | II-7 | [III-4/III-4/III-1] |
| 692 | II-7 | [III-4/III-4/III-2] |
| 693 | II-7 | [III-4/III-4/III-3] |
| 694 | II-7 | [III-4/III-4/III-4] |
| 695 | II-7 | [III-4/III-4/III-5] |
| 696 | II-7 | [III-4/III-5/III-1] |
| 697 | II-7 | [III-4/III-5/III-2] |
| 698 | II-7 | [III-4/III-5/III-3] |
| 699 | II-7 | [III-4/III-5/III-4] |
| 700 | II-7 | [III-4/III-5/III-5] |
| 701 | II-7 | [III-5/III-1/III-1] |
| 702 | II-7 | [III-5/III-1/III-2] |
| 703 | II-7 | [III-5/III-1/III-3] |
| 704 | II-7 | [III-5/III-1/III-4] |
| 705 | II-7 | [III-5/III-1/III-5] |
| 706 | II-7 | [III-5/III-2/III-1] |
| 707 | II-7 | [III-5/III-2/III-2] |
| 708 | II-7 | [III-5/III-2/III-3] |
| 709 | II-7 | [III-5/III-2/III-4] |
| 710 | II-7 | [III-5/III-2/III-5] |
| 711 | II-7 | [III-5/III-3/III-1] |
| 712 | II-7 | [III-5/III-3/III-2] |
| 713 | II-7 | [III-5/III-3/III-3] |
| 714 | II-7 | [III-5/III-3/III-4] |
| 715 | II-7 | [III-5/III-3/III-5] |
| 716 | II-7 | [III-5/III-4/III-1] |
| 717 | II-7 | [III-5/III-4/III-2] |
| 718 | II-7 | [III-5/III-4/III-3] |
| 719 | II-7 | [III-5/III-4/III-4] |
| 720 | II-7 | [III-5/III-4/III-5] |
| 721 | II-7 | [III-5/III-5/III-1] |
| 722 | II-7 | [III-5/III-5/III-2] |
| 723 | II-7 | [III-5/III-5/III-3] |
| 724 | II-7 | [III-5/III-5/III-4] |
| 725 | II-7 | [III-5/III-5/III-5] |
| 726 | II-8 | [III-1/III-1/III-1] |
| 727 | II-8 | [III-1/III-1/III-2] |
| 728 | II-8 | [III-1/III-1/III-3] |
| 729 | II-8 | [III-1/III-1/III-4] |
| 730 | II-8 | [III-1/III-1/III-5] |
| 731 | II-8 | [III-1/III-2/III-1] |
| 732 | II-8 | [III-1/III-2/III-2] |
| 733 | II-8 | [III-1/III-2/III-3] |
| 734 | II-8 | [III-1/III-2/III-4] |
| 735 | II-8 | [III-1/III-2/III-5] |
| 736 | II-8 | [III-1/III-3/III-1] |
| 737 | II-8 | [III-1/III-3/III-2] |
| 738 | II-8 | [III-1/III-3/III-3] |
| 739 | II-8 | [III-1/III-3/III-4] |
| 740 | II-8 | [III-1/III-3/III-5] |
| 741 | II-8 | [III-1/III-4/III-1] |
| 742 | II-8 | [III-1/III-4/III-2] |
| 743 | II-8 | [III-1/III-4/III-3] |
| 744 | II-8 | [III-1/III-4/III-4] |
| 745 | II-8 | [III-1/III-4/III-5] |
| 746 | II-8 | [III-1/III-5/III-1] |
| 747 | II-8 | [III-1/III-5/III-2] |
| 748 | II-8 | [III-1/III-5/III-3] |
| 749 | II-8 | [III-1/III-5/III-4] |
| 750 | II-8 | [III-1/III-5/III-5] |
| 751 | II-8 | [III-2/III-1/III-1] |
| 752 | II-8 | [III-2/III-1/III-2] |
| 753 | II-8 | [III-2/III-1/III-3] |
| 754 | II-8 | [III-2/III-1/III-4] |
| 755 | II-8 | [III-2/III-1/III-5] |
| 756 | II-8 | [III-2/III-2/III-1] |
| 757 | II-8 | [III-2/III-2/III-2] |
| 758 | II-8 | [III-2/III-2/III-3] |
| 759 | II-8 | [III-2/III-2/III-4] |
| 760 | II-8 | [III-2/III-2/III-5] |
| 761 | II-8 | [III-2/III-3/III-1] |
| 762 | II-8 | [III-2/III-3/III-2] |
| 763 | II-8 | [III-2/III-3/III-3] |
| 764 | II-8 | [III-2/III-3/III-4] |
| 765 | II-8 | [III-2/III-3/III-5] |
| 766 | II-8 | [III-2/III-4/III-1] |
| 767 | II-8 | [III-2/III-4/III-2] |
| 768 | II-8 | [III-2/III-4/III-3] |
| 769 | II-8 | [III-2/III-4/III-4] |
| 770 | II-8 | [III-2/III-4/III-5] |
| 771 | II-8 | [III-2/III-5/III-1] |
| 772 | II-8 | [III-2/III-5/III-2] |
| 773 | II-8 | [III-2/III-5/III-3] |
| 774 | II-8 | [III-2/III-5/III-4] |
| 775 | II-8 | [III-2/III-5/III-5] |
| 776 | II-8 | [III-3/III-1/III-1] |
| 777 | II-8 | [III-3/III-1/III-2] |
| 778 | II-8 | [III-3/III-1/III-3] |
| 779 | II-8 | [III-3/III-1/III-4] |
| 780 | II-8 | [III-3/III-1/III-5] |
| 781 | II-8 | [III-3/III-2/III-1] |
| 782 | II-8 | [III-3/III-2/III-2] |
| 783 | II-8 | [III-3/III-2/III-3] |
| 784 | II-8 | [III-3/III-2/III-4] |
| 785 | II-8 | [III-3/III-2/III-5] |
| 786 | II-8 | [III-3/III-3/III-1] |
| 787 | II-8 | [III-3/III-3/III-2] |
| 788 | II-8 | [III-3/III-3/III-3] |
| 789 | II-8 | [III-3/III-3/III-4] |
| 790 | II-8 | [III-3/III-3/III-5] |
| 791 | II-8 | [III-3/III-4/III-1] |
| 792 | II-8 | [III-3/III-4/III-2] |
| 793 | II-8 | [III-3/III-4/III-3] |
| 794 | II-8 | [III-3/III-4/III-4] |
| 795 | II-8 | [III-3/III-4/III-5] |
| 796 | II-8 | [III-3/III-5/III-1] |
| 797 | II-8 | [III-3/III-5/III-2] |
| 798 | II-8 | [III-3/III-5/III-3] |
| 799 | II-8 | [III-3/III-5/III-4] |
| 800 | II-8 | [III-3/III-5/III-5] |
| 801 | II-8 | [III-4/III-1/III-1] |
| 802 | II-8 | [III-4/III-1/III-2] |
| 803 | II-8 | [III-4/III-1/III-3] |
| 804 | II-8 | [III-4/III-1/III-4] |
| 305 | II-8 | [III-4/III-1/III-5] |
| 806 | II-8 | [III-4/III-2/III-1] |
| 807 | II-8 | [III-4/III-2/III-2] |
| 808 | II-8 | [III-4/III-2/III-3] |
| 809 | II-8 | [III-4/III-2/III-4] |
| 810 | II-8 | [III-4/III-2/III-5] |
| 811 | II-8 | [III-4/III-3/III-1] |
| 812 | II-8 | [III-4/III-3/III-2] |
| 813 | II-8 | [III-4/III-3/III-3] |
| 814 | II-8 | [III-4/III-3/III-4] |
| 815 | II-8 | [III-4/III-3/III-5] |
| 816 | II-8 | [III-4/III-4/III-1] |
| 817 | II-8 | [III-4/III-4/III-2] |
| 818 | II-8 | [III-4/III-4/III-3] |
| 819 | II-8 | [III-4/III-4/III-4] |
| 820 | II-8 | [III-4/III-4/III-5] |
| 821 | II-8 | [III-4/III-5/III-1] |
| 822 | II-8 | [III-4/III-5/III-2] |
| 823 | II-8 | [III-4/III-5/III-3] |
| 824 | II-8 | [III-4/III-5/III-4] |
| 825 | II-8 | [III-4/III-5/III-5] |
| 826 | II-8 | [III-5/III-1/III-1] |
| 827 | II-8 | [III-5/III-1/III-2] |
| 828 | II-8 | [III-5/III-1/III-3] |
| 829 | II-8 | [III-5/III-1/III-4] |
| 830 | II-8 | [III-5/III-1/III-5] |
| 831 | II-8 | [III-5/III-2/III-1] |
| 832 | II-8 | [III-5/III-2/III-2] |
| 833 | II-8 | [III-5/III-2/III-3] |
| 834 | II-8 | [III-5/III-2/III-4] |
| 835 | II-8 | [III-5/III-2/III-5] |
| 836 | II-8 | [III-5/III-3/III-1] |
| 837 | II-8 | [III-5/III-3/III-2] |
| 838 | II-8 | [III-5/III-3/III-3] |
| 839 | II-8 | [III-5/III-3/III-4] |
| 840 | II-8 | [III-5/III-3/III-5] |
| 841 | II-8 | [III-5/III-4/III-1] |
| 842 | II-8 | [III-5/III-4/III-2] |
| 843 | II-8 | [III-5/III-4/III-3] |
| 844 | II-8 | [III-5/III-4/III-4] |
| 845 | II-8 | [III-5/III-4/III-5] |
| 846 | II-8 | [III-5/III-5/III-1] |
| 847 | II-8 | [III-5/III-5/III-2] |
| 848 | II-8 | [III-5/III-5/III-3] |
| 849 | II-8 | [III-5/III-5/III-4] |
| 850 | II-8 | [III-5/III-5/III-5] |
| 851 | II-9 | [III-1/III-1/III-1] |
| 852 | II-9 | [III-1/III-1/III-2] |
| 853 | II-9 | [III-1/III-1/III-3] |
| 854 | II-9 | [III-1/III-1/III-4] |
| 855 | II-9 | [III-1/III-1/III-5] |
| 856 | II-9 | [III-1/III-2/III-1] |
| 857 | II-9 | [III-1/III-2/III-2] |
| 858 | II-9 | [III-1/III-2/III-3] |
| 859 | II-9 | [III-1/III-2/III-4] |
| 860 | II-9 | [III-1/III-2/III-5] |
| 861 | II-9 | [III-1/III-3/III-1] |
| 862 | II-9 | [III-1/III-3/III-2] |
| 863 | II-9 | [III-1/III-3/III-3] |
| 864 | II-9 | [III-1/III-3/III-4] |
| 865 | II-9 | [III-1/III-3/III-5] |
| 866 | II-9 | [III-1/III-4/III-1] |
| 867 | II-9 | [III-1/III-4/III-2] |
| 868 | II-9 | [III-1/III-4/III-3] |
| 869 | II-9 | [III-1/III-4/III-4] |
| 870 | II-9 | [III-1/III-4/III-5] |
| 871 | II-9 | [III-1/III-5/III-1] |
| 872 | II-9 | [III-1/III-5/III-2] |
| 873 | II-9 | [III-1/III-5/III-3] |
| 874 | II-9 | [III-1/III-5/III-4] |
| 875 | II-9 | [III-1/III-5/III-5] |
| 876 | II-9 | [III-2/III-1/III-1] |
| 877 | II-9 | [III-2/III-1/III-2] |
| 878 | II-9 | [III-2/III-1/III-3] |
| 879 | II-9 | [III-2/III-1/III-4] |
| 880 | II-9 | [III-2/III-1/III-5] |
| 881 | II-9 | [III-2/III-2/III-1] |
| 882 | II-9 | [III-2/III-2/III-2] |
| 883 | II-9 | [III-2/III-2/III-3] |
| 884 | II-9 | [III-2/III-2/III-4] |
| 885 | II-9 | [III-2/III-2/III-5] |
| 886 | II-9 | [III-2/III-3/III-1] |
| 887 | II-9 | [III-2/III-3/III-2] |
| 888 | II-9 | [III-2/III-3/III-3] |
| 889 | II-9 | [III-2/III-3/III-4] |
| 890 | II-9 | [III-2/III-3/III-5] |
| 891 | II-9 | [III-2/III-4/III-1] |
| 892 | II-9 | [III-2/III-4/III-2] |
| 893 | II-9 | [III-2/III-4/III-3] |
| 894 | II-9 | [III-2/III-4/III-4] |
| 895 | II-9 | [III-2/III-4/III-5] |
| 896 | II-9 | [III-2/III-5/III-1] |
| 897 | II-9 | [III-2/III-5/III-2] |
| 898 | II-9 | [III-2/III-5/III-3] |
| 899 | II-9 | [III-2/III-5/III-4] |
| 900 | II-9 | [III-2/III-5/III-5] |
| 901 | II-9 | [III-3/III-1/III-1] |
| 902 | II-9 | [III-3/III-1/III-2] |
| 903 | II-9 | [III-3/III-1/III-3] |
| 904 | II-9 | [III-3/III-1/III-4] |
| 905 | II-9 | [III-3/III-1/III-5] |
| 906 | II-9 | [III-3/III-2/III-1] |
| 907 | II-9 | [III-3/III-2/III-2] |
| 908 | II-9 | [III-3/III-2/III-3] |
| 909 | II-9 | [III-3/III-2/III-4] |
| 910 | II-9 | [III-3/III-2/III-5] |
| 911 | II-9 | [III-3/III-3/III-1] |
| 912 | II-9 | [III-3/III-3/III-2] |
| 913 | II-9 | [III-3/III-3/III-3] |
| 914 | II-9 | [III-3/III-3/III-4] |
| 915 | II-9 | [III-3/III-3/III-5] |
| 916 | II-9 | [III-3/III-4/III-1] |
| 917 | II-9 | [III-3/III-4/III-2] |
| 918 | II-9 | [III-3/III-4/III-3] |
| 919 | II-9 | [III-3/III-4/III-4] |
| 920 | II-9 | [III-3/III-4/III-5] |
| 921 | II-9 | [III-3/III-5/III-1] |
| 922 | II-9 | [III-3/III-5/III-2] |
| 923 | II-9 | [III-3/III-5/III-3] |
| 924 | II-9 | [III-3/III-5/III-4] |
| 925 | II-9 | [III-3/III-5/III-5] |
| 926 | II-9 | [III-4/III-1/III-1] |
| 927 | II-9 | [III-4/III-1/III-2] |
| 928 | II-9 | [III-4/III-1/III-3] |
| 929 | II-9 | [III-4/III-1/III-4] |
| 930 | II-9 | [III-4/III-1/III-5] |
| 931 | II-9 | [III-4/III-2/III-1] |
| 932 | II-9 | [III-4/III-2/III-2] |
| 933 | II-9 | [III-4/III-2/III-3] |
| 934 | II-9 | [III-4/III-2/III-4] |
| 935 | II-9 | [III-4/III-2/III-5] |
| 936 | II-9 | [III-4/III-3/III-1] |
| 937 | II-9 | [III-4/III-3/III-2] |
| 938 | II-9 | [III-4/III-3/III-3] |
| 939 | II-9 | [III-4/III-3/III-4] |
| 940 | II-9 | [III-4/III-3/III-5] |
| 941 | II-9 | [III-4/III-4/III-1] |
| 942 | II-9 | [III-4/III-4/III-2] |
| 943 | II-9 | [III-4/III-4/III-3] |
| 944 | II-9 | [III-4/III-4/III-4] |
| 945 | II-9 | [III-4/III-4/III-5] |
| 946 | II-9 | [III-4/III-5/III-1] |
| 947 | II-9 | [III-4/III-5/III-2] |
| 948 | II-9 | [III-4/III-5/III-3] |
| 949 | II-9 | [III-4/III-5/III-4] |
| 950 | II-9 | [III-4/III-5/III-5] |
| 951 | II-9 | [III-5/III-1/III-1] |
| 952 | II-9 | [III-5/III-1/III-2] |
| 953 | II-9 | [III-5/III-1/III-3] |
| 954 | II-9 | [III-5/III-1/III-4] |
| 955 | II-9 | [III-5/III-1/III-5] |
| 956 | II-9 | [III-5/III-2/III-1] |
| 957 | II-9 | [III-5/III-2/III-2] |
| 958 | II-9 | [III-5/III-2/III-3] |
| 959 | II-9 | [III-5/III-2/III-4] |
| 960 | II-9 | [III-5/III-2/III-5] |
| 961 | II-9 | [III-5/III-3/III-1] |
| 962 | II-9 | [III-5/III-3/III-2] |
| 963 | II-9 | [III-5/III-3/III-3] |
| 964 | II-9 | [III-5/III-3/III-4] |
| 965 | II-9 | [III-5/III-3/III-5] |
| 966 | II-9 | [III-5/III-4/III-1] |
| 967 | II-9 | [III-5/III-4/III-2] |
| 968 | II-9 | [III-5/III-4/III-3] |
| 969 | II-9 | [III-5/III-4/III-4] |
| 970 | II-9 | [III-5/III-4/III-5] |
| 971 | II-9 | [III-5/III-5/III-1] |
| 972 | II-9 | [III-5/III-5/III-2] |
| 973 | II-9 | [III-5/III-5/III-3] |
| 974 | II-9 | [III-5/III-5/III-4] |
| 975 | II-9 | [III-5/III-5/III-5] |
| 976 | II-10 | [III-1/III-1/III-1] |
| 977 | II-10 | [III-1/III-1/III-2] |
| 978 | II-10 | [III-1/III-1/III-3] |
| 979 | II-10 | [III-1/III-1/III-4] |
| 980 | II-10 | [III-1/III-1/III-5] |
| 981 | II-10 | [III-1/III-2/III-1] |
| 982 | II-10 | [III-1/III-2/III-2] |
| 983 | II-10 | [III-1/III-2/III-3] |
| 984 | II-10 | [III-1/III-2/III-4] |
| 985 | II-10 | [III-1/III-2/III-5] |
| 986 | II-10 | [III-1/III-3/III-1] |
| 987 | II-10 | [III-1/III-3/III-2] |
| 988 | II-10 | [III-1/III-3/III-3] |
| 989 | II-10 | [III-1/III-3/III-4] |
| 990 | II-10 | [III-1/III-3/III-5] |
| 991 | II-10 | [III-1/III-4/III-1] |
| 992 | II-10 | [III-1/III-4/III-2] |
| 993 | II-10 | [III-1/III-4/III-3] |
| 994 | II-10 | [III-1/III-4/III-4] |
| 995 | II-10 | [III-1/III-4/III-5] |
| 996 | II-10 | [III-1/III-5/III-1] |
| 997 | II-10 | [III-1/III-5/III-2] |
| 998 | II-10 | [III-1/III-5/III-3] |
| 999 | II-10 | [III-1/III-5/III-4] |
| 1000 | II-10 | [III-1/III-5/III-5] |
| 1001 | II-10 | [III-2/III-1/III-2] |
| 1002 | II-10 | [III-2/III-1/III-3] |
| 1003 | II-10 | [III-2/III-1/III-4] |
| 1004 | II-10 | [III-2/III-1/III-5] |
| 1005 | II-10 | [III-2/III-2/III-2] |
| 1006 | II-10 | [III-2/III-2/III-3] |
| 1007 | II-10 | [III-2/III-2/III-4] |
| 1008 | II-10 | [III-2/III-2/III-5] |
| 1009 | II-10 | [III-2/III-3/III-2] |
| 1010 | II-10 | [III-2/III-3/III-3] |
| 1011 | II-10 | [III-2/III-3/III-4] |
| 1012 | II-10 | [III-2/III-3/III-5] |
| 1013 | II-10 | [III-2/III-4/III-2] |
| 1014 | II-10 | [III-2/III-4/III-3] |
| 1015 | II-10 | [III-2/III-4/III-4] |
| 1016 | II-10 | [III-2/III-4/III-5] |
| 1017 | II-10 | [III-2/III-5/III-2] |
| 1018 | II-10 | [III-2/III-5/III-3] |
| 1019 | II-10 | [III-2/III-5/III-4] |
| 1020 | II-10 | [III-2/III-5/III-5] |
| 1021 | II-10 | [III-3/III-1/III-3] |
| 1022 | II-10 | [III-3/III-1/III-4] |
| 1023 | II-10 | [III-3/III-1/III-5] |
| 1024 | II-10 | [III-3/III-2/III-3] |
| 1025 | II-10 | [III-3/III-2/III-4] |
| 1026 | II-10 | [III-3/III-2/III-5] |
| 1027 | II-10 | [III-3/III-3/III-3] |
| 1028 | II-10 | [III-3/III-3/III-4] |
| 1029 | II-10 | [III-3/III-3/III-5] |
| 1030 | II-10 | [III-3/III-4/III-3] |
| 1031 | II-10 | [III-3/III-4/III-4] |
| 1032 | II-10 | [III-3/III-4/III-5] |
| 1033 | II-10 | [III-3/III-5/III-3] |
| 1034 | II-10 | [III-3/III-5/III-4] |
| 1035 | II-10 | [III-3/III-5/III-5] |
| 1036 | II-10 | [III-4/III-1/III-4] |
| 1037 | II-10 | [III-4/III-1/III-5] |
| 1038 | II-10 | [III-4/III-2/III-4] |
| 1039 | II-10 | [III-4/III-2/III-5] |
| 1040 | II-10 | [III-4/III-3/III-4] |
| 1041 | II-10 | [III-4/III-3/III-5] |
| 1042 | II-10 | [III-4/III-4/III-4] |
| 1043 | II-10 | [III-4/III-4/III-5] |
| 1044 | II-10 | [III-4/III-5/III-4] |
| 1045 | II-10 | [III-4/III-5/III-5] |
| 1046 | II-10 | [III-5/III-1/III-5] |
| 1047 | II-10 | [III-5/III-2/III-5] |
| 1048 | II-10 | [III-5/III-3/III-5] |
| 1049 | II-10 | [III-5/III-4/III-5] |
| 1050 | II-10 | [III-5/III-5/III-5] |
| 1051 | II-11 | [III-1/III-1/III-1] |
| 1052 | II-11 | [III-1/III-1/III-2] |
| 1053 | II-11 | [III-1/III-1/III-3] |
| 1054 | II-11 | [III-1/III-1/III-4] |
| 1055 | II-11 | [III-1/III-1/III-5] |
| 1056 | II-11 | [III-1/III-2/III-1] |
| 1057 | II-11 | [III-1/III-2/III-2] |
| 1058 | II-11 | [III-1/III-2/III-3] |
| 1059 | II-11 | [III-1/III-2/III-4] |
| 1060 | II-11 | [III-1/III-2/III-5] |
| 1061 | II-11 | [III-1/III-3/III-1] |
| 1062 | II-11 | [III-1/III-3/III-2] |
| 1063 | II-11 | [III-1/III-3/III-3] |
| 1064 | II-11 | [III-1/III-3/III-4] |
| 1065 | II-11 | [III-1/III-3/III-5] |
| 1066 | II-11 | [III-1/III-4/III-1] |
| 1067 | II-11 | [III-1/III-4/III-2] |
| 1069 | II-11 | [III-1/III-4/III-3] |
| 1069 | II-11 | [III-1/III-4/III-4] |
| 1070 | II-11 | [III-1/III-4/III-5] |
| 1071 | II-11 | [III-1/III-5/III-1] |
| 1072 | II-11 | [III-1/III-5/III-2] |
| 1073 | II-11 | [III-1/III-5/III-3] |
| 1074 | II-11 | [III-1/III-5/III-4] |
| 1075 | II-11 | [III-1/III-5/III-5] |
| 1076 | II-11 | [III-2/III-1/III-1] |
| 1077 | II-11 | [III-2/III-1/III-2] |
| 1078 | II-11 | [III-2/III-1/III-3] |
| 1079 | II-11 | [III-2/III-1/III-4] |
| 1080 | II-11 | [III-2/III-1/III-5] |
| 1081 | II-11 | [III-2/III-2/III-1] |
| 1082 | II-11 | [III-2/III-2/III-2] |
| 1083 | II-11 | [III-2/III-2/III-3] |
| 1084 | II-11 | [III-2/III-2/III-4] |
| 1085 | II-11 | [III-2/III-2/III-5] |
| 1086 | II-11 | [III-2/III-3/III-1] |
| 1087 | II-11 | [III-2/III-3/III-2] |
| 1088 | II-11 | [III-2/III-3/III-3] |
| 1089 | II-11 | [III-2/III-3/III-4] |
| 1090 | II-11 | [III-2/III-3/III-5] |
| 1091 | II-11 | [III-2/III-4/III-1] |
| 1092 | II-11 | [III-2/III-4/III-2] |
| 1093 | II-11 | [III-2/III-4/III-3] |
| 1094 | II-11 | [III-2/III-4/III-4] |
| 1095 | II-11 | [III-2/III-4/III-5] |
| 1096 | II-11 | [III-2/III-5/III-1] |
| 1097 | II-11 | [III-2/III-5/III-2] |
| 1098 | II-11 | [III-2/III-5/III-3] |
| 1099 | II-11 | [III-2/III-5/III-4] |
| 1100 | II-11 | [III-2/III-5/III-5] |
| 1101 | II-11 | [III-3/III-1/III-1] |
| 1102 | II-11 | [III-3/III-1/III-2] |
| 1103 | II-11 | [III-3/III-1/III-3] |
| 1104 | II-11 | [III-3/III-1/III-4] |
| 1105 | II-11 | [III-3/III-1/III-5] |
| 1106 | II-11 | [III-3/III-2/III-1] |
| 1107 | II-11 | [III-3/III-2/III-2] |
| 1108 | II-11 | [III-3/III-2/III-3] |
| 1109 | II-11 | [III-3/III-2/III-4] |
| 1110 | II-11 | [III-3/III-2/III-5] |
| 1111 | II-11 | [III-3/III-3/III-1] |
| 1112 | II-11 | [III-3/III-3/III-2] |
| 1113 | II-11 | [III-3/III-3/III-3] |
| 1114 | II-11 | [III-3/III-3/III-4] |
| 1115 | II-11 | [III-3/III-3/III-5] |
| 1116 | II-11 | [III-3/III-4/III-1] |
| 1117 | II-11 | [III-3/III-4/III-2] |
| 1118 | II-11 | [III-3/III-4/III-3] |
| 1119 | II-11 | [III-3/III-4/III-4] |
| 1120 | II-11 | [III-3/III-4/III-5] |
| 1121 | II-11 | [III-3/III-5/III-1] |
| 1122 | II-11 | [III-3/III-5/III-2] |
| 1123 | II-11 | [III-3/III-5/III-3] |
| 1124 | II-11 | [III-3/III-5/III-4] |
| 1125 | II-11 | [III-3/III-5/III-5] |
| 1126 | II-11 | [III-4/III-1/III-1] |
| 1127 | II-11 | [III-4/III-1/III-2] |
| 1128 | II-11 | [III-4/III-1/III-3] |
| 1129 | II-11 | [III-4/III-1/III-4] |
| 1130 | II-11 | [III-4/III-1/III-5] |
| 1131 | II-11 | [III-4/III-2/III-1] |
| 1132 | II-11 | [III-4/III-2/III-2] |
| 1133 | II-11 | [III-4/III-2/III-3] |
| 1134 | II-11 | [III-4/III-2/III-4] |
| 1135 | II-11 | [III-4/III-2/III-5] |
| 1136 | II-11 | [III-4/III-3/III-1] |
| 1137 | II-11 | [III-4/III-3/III-2] |
| 1138 | II-11 | [III-4/III-3/III-3] |
| 1139 | II-11 | [III-4/III-3/III-4] |
| 1140 | II-11 | [III-4/III-3/III-5] |
| 1141 | II-11 | [III-4/III-4/III-1] |
| 1142 | II-11 | [III-4/III-4/III-2] |
| 1143 | II-11 | [III-4/III-4/III-3] |
| 1144 | II-11 | [III-4/III-4/III-4] |
| 1145 | II-11 | [III-4/III-4/III-5] |
| 1146 | II-11 | [III-4/III-5/III-1] |
| 1147 | II-11 | [III-4/III-5/III-2] |
| 1148 | II-11 | [III-4/III-5/III-3] |
| 1149 | II-11 | [III-4/III-5/III-4] |
| 1150 | II-11 | [III-4/III-5/III-5] |
| 1151 | II-11 | [III-5/III-1/III-1] |
| 1152 | II-11 | [III-5/III-1/III-2] |
| 1153 | II-11 | [III-5/III-1/III-3] |
| 1154 | II-11 | [III-5/III-1/III-4] |
| 1155 | II-11 | [III-5/III-1/III-5] |
| 1156 | II-11 | [III-5/III-2/III-1] |
| 1157 | II-11 | [III-5/III-2/III-2] |
| 1158 | II-11 | [III-5/III-2/III-3] |
| 1159 | II-11 | [III-5/III-2/III-4] |
| 1160 | II-11 | [III-5/III-2/III-5] |
| 1161 | II-11 | [III-5/III-3/III-1] |
| 1162 | II-11 | [III-5/III-3/III-2] |
| 1163 | II-11 | [III-5/III-3/III-3] |
| 1164 | II-11 | [III-5/III-3/III-4] |
| 1165 | II-11 | [III-5/III-3/III-5] |
| 1166 | II-11 | [III-5/III-4/III-1] |
| 1167 | II-11 | [III-5/III-4/III-2] |
| 1168 | II-11 | [III-5/III-4/III-3] |
| 1169 | II-11 | [III-5/III-4/III-4] |
| 1170 | II-11 | [III-5/III-4/III-5] |
| 1171 | II-11 | [III-5/III-5/III-1] |
| 1172 | II-11 | [III-5/III-5/III-2] |
| 1173 | II-11 | [III-5/III-5/III-3] |
| 1174 | II-11 | [III-5/III-5/III-4] |
| 1175 | II-11 | [III-5/III-5/III-5] |
| 1176 | II-12 | [III-1/III-1/III-1] |
| 1177 | II-12 | [III-1/III-1/III-2] |
| 1178 | II-12 | [III-1/III-1/III-3] |
| 1179 | II-12 | [III-1/III-1/III-4] |
| 1180 | II-12 | [III-1/III-1/III-5] |
| 1181 | II-12 | [III-1/III-2/III-1] |
| 1182 | II-12 | [III-1/III-2/III-2] |
| 1183 | II-12 | [III-1/III-2/III-3] |
| 1184 | II-12 | [III-1/III-2/III-4] |
| 1185 | II-12 | [III-1/III-2/III-5] |
| 1186 | II-12 | [III-1/III-3/III-1] |
| 1187 | II-12 | [III-1/III-3/III-2] |
| 1188 | II-12 | [III-1/III-3/III-3] |
| 1189 | II-12 | [III-1/III-3/III-4] |
| 1190 | II-12 | [III-1/III-3/III-5] |
| 1191 | II-12 | [III-1/III-4/III-1] |
| 1192 | II-12 | [III-1/III-4/III-2] |
| 1193 | II-12 | [III-1/III-4/III-3] |
| 1194 | II-12 | [III-1/III-4/III-4] |
| 1195 | II-12 | [III-1/III-4/III-5] |
| 1196 | II-12 | [III-1/III-5/III-1] |
| 1197 | II-12 | [III-1/III-5/III-2] |
| 1198 | II-12 | [III-1/III-5/III-3] |
| 1199 | II-12 | [III-1/III-5/III-4] |
| 1200 | II-12 | [III-1/III-5/III-5] |
| 1201 | II-12 | [III-2/III-1/III-1] |
| 1202 | II-12 | [III-2/III-1/III-2] |
| 1203 | II-12 | [III-2/III-1/III-3] |
| 1204 | II-12 | [III-2/III-1/III-4] |
| 1205 | II-12 | [III-2/III-1/III-5] |
| 1206 | II-12 | [III-2/III-2/III-1] |
| 1207 | II-12 | [III-2/III-2/III-2] |
| 1208 | II-12 | [III-2/III-2/III-3] |
| 1209 | II-12 | [III-2/III-2/III-4] |
| 1210 | II-12 | [III-2/III-2/III-5] |
| 1211 | II-12 | [III-2/III-3/III-1] |
| 1212 | II-12 | [III-2/III-3/III-2] |
| 1213 | II-12 | [III-2/III-3/III-3] |
| 1214 | II-12 | [III-2/III-3/III-4] |
| 1215 | II-12 | [III-2/III-3/III-5] |
| 1216 | II-12 | [III-2/III-4/III-1] |
| 1217 | II-12 | [III-2/III-4/III-2] |
| 1218 | II-12 | [III-2/III-4/III-3] |
| 1219 | II-12 | [III-2/III-4/III-4] |
| 1220 | II-12 | [III-2/III-4/III-5] |
| 1221 | II-12 | [III-2/III-5/III-1] |
| 1222 | II-12 | [III-2/III-5/III-2] |
| 1223 | II-12 | [III-2/III-5/III-3] |
| 1224 | II-12 | [III-2/III-5/III-4] |
| 1225 | II-12 | [III-2/III-5/III-5] |
| 1226 | II-12 | [III-3/III-1/III-1] |
| 1227 | II-12 | [III-3/III-1/III-2] |
| 1228 | II-12 | [III-3/III-1/III-3] |
| 1229 | II-12 | [III-3/III-1/III-4] |
| 1230 | II-12 | [III-3/III-1/III-5] |
| 1231 | II-12 | [III-3/III-2/III-1] |
| 1232 | II-12 | [III-3/III-2/III-2] |
| 1233 | II-12 | [III-3/III-2/III-3] |
| 1234 | II-12 | [III-3/III-2/III-4] |
| 1235 | II-12 | [III-3/III-2/III-5] |
| 1236 | II-12 | [III-3/III-3/III-1] |
| 1237 | II-12 | [III-3/III-3/III-2] |
| 1238 | II-12 | [III-3/III-3/III-3] |
| 1239 | II-12 | [III-3/III-3/III-4] |
| 1240 | II-12 | [III-3/III-3/III-5] |
| 1241 | II-12 | [III-3/III-4/III-1] |
| 1242 | II-12 | [III-3/III-4/III-2] |
| 1243 | II-12 | [III-3/III-4/III-3] |
| 1244 | II-12 | [III-3/III-4/III-4] |
| 1245 | II-12 | [III-3/III-4/III-5] |
| 1246 | II-12 | [III-3/III-5/III-1] |
| 1247 | II-12 | [III-3/III-5/III-2] |
| 1248 | II-12 | [III-3/III-5/III-3] |
| 1249 | II-12 | [III-3/III-5/III-4] |
| 1250 | II-12 | [III-3/III-5/III-5] |
| 1251 | II-12 | [III-4/III-1/III-1] |
| 1252 | II-12 | [III-4/III-1/III-2] |
| 1253 | II-12 | [III-4/III-1/III-3] |
| 1254 | II-12 | [III-4/III-1/III-4] |
| 1255 | II-12 | [III-4/III-1/III-5] |
| 1256 | II-12 | [III-4/III-2/III-1] |
| 1257 | II-12 | [III-4/III-2/III-2] |
| 1258 | II-12 | [III-4/III-2/III-3] |
| 1259 | II-12 | [III-4/III-2/III-4] |
| 1260 | II-12 | [III-4/III-2/III-5] |
| 1261 | II-12 | [III-4/III-3/III-1] |
| 1262 | II-12 | [III-4/III-3/III-2] |
| 1263 | II-12 | [III-4/III-3/III-3] |
| 1264 | II-12 | [III-4/III-3/III-4] |
| 1265 | II-12 | [III-4/III-3/III-5] |
| 1266 | II-12 | [III-4/III-4/III-1] |
| 1267 | II-12 | [III-4/III-4/III-2] |
| 1268 | II-12 | [III-4/III-4/III-3] |
| 1269 | II-12 | [III-4/III-4/III-4] |
| 1270 | II-12 | [III-4/III-4/III-5] |
| 1271 | II-12 | [III-4/III-5/III-1] |
| 1272 | II-12 | [III-4/III-5/III-2] |
| 1273 | II-12 | [III-4/III-5/III-3] |
| 1274 | II-12 | [III-4/III-5/III-4] |
| 1275 | II-12 | [III-4/III-5/III-5] |
| 1276 | II-12 | [III-5/III-1/III-1] |
| 1277 | II-12 | [III-5/III-1/III-2] |
| 1278 | II-12 | [III-5/III-1/III-3] |
| 1279 | II-12 | [III-5/III-1/III-4] |
| 1280 | II-12 | [III-5/III-1/III-5] |
| 1281 | II-12 | [III-5/III-2/III-1] |
| 1282 | II-12 | [III-5/III-2/III-2] |
| 1283 | II-12 | [III-5/III-2/III-3] |
| 1284 | II-12 | [III-5/III-2/III-4] |
| 1285 | II-12 | [III-5/III-2/III-5] |
| 1286 | II-12 | [III-5/III-3/III-1] |
| 1287 | II-12 | [III-5/III-3/III-2] |
| 1288 | II-12 | [III-5/III-3/III-3] |
| 1289 | II-12 | [III-5/III-3/III-4] |
| 1290 | II-12 | [III-5/III-3/III-5] |
| 1291 | II-12 | [III-5/III-4/III-1] |
| 1292 | II-12 | [III-5/III-4/III-2] |
| 1293 | II-12 | [III-5/III-4/III-3] |
| 1294 | II-12 | [III-5/III-4/III-4] |
| 1295 | II-12 | [III-5/III-4/III-5] |
| 1296 | II-12 | [III-5/III-5/III-1] |
| 1297 | II-12 | [III-5/III-5/III-2] |
| 1298 | II-12 | [III-5/III-5/III-3] |
| 1299 | II-12 | [III-5/III-5/III-4] |
| 1300 | II-12 | [III-5/III-5/III-5] |
| 1301 | II-13 | [III-1/III-1/III-1] |
| 1302 | II-13 | [III-1/III-1/III-2] |
| 1303 | II-13 | [III-1/III-1/III-3] |
| 1304 | II-13 | [III-1/III-1/III-4] |
| 1305 | II-13 | [III-1/III-1/III-5] |
| 1306 | II-13 | [III-1/III-2/III-1] |
| 1307 | II-13 | [III-1/III-2/III-2] |
| 1308 | II-13 | [III-1/III-2/III-3] |
| 1309 | II-13 | [III-1/III-2/III-4] |
| 1310 | II-13 | [III-1/III-2/III-5] |
| 1311 | II-13 | [III-1/III-3/III-1] |
| 1312 | II-13 | [III-1/III-3/III-2] |
| 1313 | II-13 | [III-1/III-3/III-3] |
| 1314 | II-13 | [III-1/III-3/III-4] |
| 1315 | II-13 | [III-1/III-3/III-5] |
| 1316 | II-13 | [III-1/III-4/III-1] |
| 1317 | II-13 | [III-1/III-4/III-2] |
| 1318 | II-13 | [III-1/III-4/III-3] |
| 1319 | II-13 | [III-1/III-4/III-4] |
| 1320 | II-13 | [III-1/III-4/III-5] |
| 1321 | II-13 | [III-1/III-5/III-1] |
| 1322 | II-13 | [III-1/III-5/III-2] |
| 1323 | II-13 | [III-1/III-5/III-3] |
| 1324 | II-13 | [III-1/III-5/III-4] |
| 1325 | II-13 | [III-1/III-5/III-5] |
| 1326 | II-13 | [III-2/III-1/III-2] |
| 1327 | II-13 | [III-2/III-1/III-3] |
| 1328 | II-13 | [III-2/III-1/III-4] |
| 1329 | II-13 | [III-2/III-1/III-5] |
| 1330 | II-13 | [III-2/III-2/III-2] |
| 1331 | II-13 | [III-2/III-2/III-3] |
| 1332 | II-13 | [III-2/III-2/III-4] |
| 1333 | II-13 | [III-2/III-2/III-5] |
| 1334 | II-13 | [III-2/III-3/III-2] |
| 1335 | II-13 | [III-2/III-3/III-3] |
| 1336 | II-13 | [III-2/III-3/III-4] |
| 1337 | II-13 | [III-2/III-3/III-5] |
| 1338 | II-13 | [III-2/III-4/III-2] |
| 1339 | II-13 | [III-2/III-4/III-3] |
| 1340 | II-13 | [III-2/III-4/III-4] |
| 1341 | II-13 | [III-2/III-4/III-5] |
| 1342 | II-13 | [III-2/III-5/III-2] |
| 1343 | II-13 | [III-2/III-5/III-3] |
| 1344 | II-13 | [III-2/III-5/III-4] |
| 1345 | II-13 | [III-2/III-5/III-5] |
| 1346 | II-13 | [III-3/III-1/III-3] |
| 1347 | II-13 | [III-3/III-1/III-4] |
| 1348 | II-13 | [III-3/III-1/III-5] |
| 1349 | II-13 | [III-3/III-2/III-3] |
| 1350 | II-13 | [III-3/III-2/III-4] |
| 1351 | II-13 | [III-3/III-2/III-5] |
| 1352 | II-13 | [III-3/III-3/III-3] |
| 1353 | II-13 | [III-3/III-3/III-4] |
| 1354 | II-13 | [III-3/III-3/III-5] |
| 1355 | II-13 | [III-3/III-4/III-3] |
| 1356 | II-13 | [III-3/III-4/III-4] |
| 1357 | II-13 | [III-3/III-4/III-5] |
| 1358 | II-13 | [III-3/III-5/III-3] |
| 1359 | II-13 | [III-3/III-5/III-4] |
| 1360 | II-13 | [III-3/III-5/III-5] |
| 1361 | II-13 | [III-4/III-1/III-4] |
| 1362 | II-13 | [III-4/III-1/III-5] |
| 1363 | II-13 | [III-4/III-2/III-4] |
| 1364 | II-13 | [III-4/III-2/III-5] |
| 1365 | II-13 | [III-4/III-3/III-4] |
| 1366 | II-13 | [III-4/III-3/III-5] |
| 1367 | II-13 | [III-4/III-4/III-4] |
| 1368 | II-13 | [III-4/III-4/III-5] |
| 1369 | II-13 | [III-4/III-5/III-4] |
| 1370 | II-13 | [III-4/III-5/III-5] |
| 1371 | II-13 | [III-5/III-1/III-5] |
| 1372 | II-13 | [III-5/III-2/III-5] |
| 1373 | II-13 | [III-5/III-3/III-5] |
| 1374 | II-13 | [III-5/III-4/III-5] |
| 1375 | II-13 | [III-5/III-5/III-5] |
| 1376 | II-14 | [III-1/III-1/III-1] |
| 1377 | II-14 | [III-1/III-1/III-2] |
| 1378 | II-14 | [III-1/III-1/III-3] |
| 1379 | II-14 | [III-1/III-1/III-4] |
| 1380 | II-14 | [III-1/III-1/III-5] |
| 1381 | II-14 | [III-1/III-2/III-1] |
| 1382 | II-14 | [III-1/III-2/III-2] |
| 1383 | II-14 | [III-1/III-2/III-3] |
| 1384 | II-14 | [III-1/III-2/III-4] |
| 1385 | II-14 | [III-1/III-2/III-5] |
| 1386 | II-14 | [III-1/III-3/III-1] |
| 1387 | II-14 | [III-1/III-3/III-2] |
| 1388 | II-14 | [III-1/III-3/III-3] |
| 1389 | II-14 | [III-1/III-3/III-4] |
| 1390 | II-14 | [III-1/III-3/III-5] |
| 1391 | II-14 | [III-1/III-4/III-1] |
| 1392 | II-14 | [III-1/III-4/III-2] |
| 1393 | II-14 | [III-1/III-4/III-3] |
| 1394 | II-14 | [III-1/III-4/III-4] |
| 1395 | II-14 | [III-1/III-4/III-5] |
| 1396 | II-14 | [III-1/III-5/III-1] |
| 1397 | II-14 | [III-1/III-5/III-2] |
| 1398 | II-14 | [III-1/III-5/III-3] |
| 1399 | II-14 | [III-1/III-5/III-4] |
| 1400 | II-14 | [III-1/III-5/III-5] |
| 1401 | II-14 | [III-2/III-1/III-2] |
| 1402 | II-14 | [III-2/III-1/III-3] |
| 1403 | II-14 | [III-2/III-1/III-4] |
| 1404 | II-14 | [III-2/III-1/III-5] |
| 1405 | II-14 | [III-2/III-2/III-2] |
| 1406 | II-14 | [III-2/III-2/III-3] |
| 1407 | II-14 | [III-2/III-2/III-4] |
| 1408 | II-14 | [III-2/III-2/III-5] |
| 1409 | II-14 | [III-2/III-3/III-2] |
| 1410 | II-14 | [III-2/III-3/III-3] |
| 1411 | II-14 | [III-2/III-3/III-4] |
| 1412 | II-14 | [III-2/III-3/III-5] |
| 1413 | II-14 | [III-2/III-4/III-2] |
| 1414 | II-14 | [III-2/III-4/III-3] |
| 1415 | II-14 | [III-2/III-4/III-4] |
| 1416 | II-14 | [III-2/III-4/III-5] |
| 1417 | II-14 | [III-2/III-5/III-2] |
| 1418 | II-14 | [III-2/III-5/III-3] |
| 1419 | II-14 | [III-2/III-5/III-4] |
| 1420 | II-14 | [III-2/III-5/III-5] |
| 1421 | II-14 | [III-3/III-1/III-3] |
| 1422 | II-14 | [III-3/III-1/III-4] |
| 1423 | II-14 | [III-3/III-1/III-5] |
| 1424 | II-14 | [III-3/III-2/III-3] |
| 1425 | II-14 | [III-3/III-2/III-4] |
| 1426 | II-14 | [III-3/III-2/III-5] |
| 1427 | II-14 | [III-3/III-3/III-3] |
| 1428 | II-14 | [III-3/III-3/III-4] |
| 1429 | II-14 | [III-3/III-3/III-5] |
| 1430 | II-14 | [III-3/III-4/III-3] |
| 1431 | II-14 | [III-3/III-4/III-4] |
| 1432 | II-14 | [III-3/III-4/III-5] |
| 1433 | II-14 | [III-3/III-5/III-3] |
| 1434 | II-14 | [III-3/III-5/III-4] |
| 1435 | II-14 | [III-3/III-5/III-5] |
| 1436 | II-14 | [III-4/III-1/III-4] |
| 1437 | II-14 | [III-4/III-1/III-5] |
| 1438 | II-14 | [III-4/III-2/III-4] |
| 1439 | II-14 | [III-4/III-2/III-5] |
| 1440 | II-14 | [III-4/III-3/III-4] |
| 1441 | II-14 | [III-4/III-3/III-5] |
| 1442 | II-14 | [III-4/III-4/III-4] |
| 1443 | II-14 | [III-4/III-4/III-5] |
| 1444 | II-14 | [III-4/III-5/III-4] |
| 1445 | II-14 | [III-4/III-5/III-5] |
| 1446 | II-14 | [III-5/III-1/III-5] |
| 1447 | II-14 | [III-5/III-2/III-5] |
| 1448 | II-14 | [III-5/III-3/III-5] |
| 1449 | II-14 | [III-5/III-4/III-5] |
| 1450 | II-14 | [III-5/III-5/III-5] |
| 1451 | II-15 | [III-1/III-1/III-1] |
| 1452 | II-15 | [III-1/III-1/III-2] |
| 1453 | II-15 | [III-1/III-1/III-3] |
| 1454 | II-15 | [III-1/III-1/III-4] |
| 1455 | II-15 | [III-1/III-1/III-5] |
| 1456 | II-15 | [III-1/III-2/III-1] |
| 1457 | II-15 | [III-1/III-2/III-2] |
| 1458 | II-15 | [III-1/III-2/III-3] |
| 1459 | II-15 | [III-1/III-2/III-4] |
| 1460 | II-15 | [III-1/III-2/III-5] |
| 1461 | II-15 | [III-1/III-3/III-1] |
| 1462 | II-15 | [III-1/III-3/III-2] |
| 1463 | II-15 | [III-1/III-3/III-3] |
| 1464 | II-15 | [III-1/III-3/III-4] |
| 1465 | II-15 | [III-1/III-3/III-5] |
| 1466 | II-15 | [III-1/III-4/III-1] |
| 1467 | II-15 | [III-1/III-4/III-2] |
| 1468 | II-15 | [III-1/III-4/III-3] |
| 1469 | II-15 | [III-1/III-4/III-4] |
| 1470 | II-15 | [III-1/III-4/III-5] |
| 1471 | II-15 | [III-1/III-5/III-1] |
| 1472 | II-15 | [III-1/III-5/III-2] |
| 1473 | II-15 | [III-1/III-5/III-3] |
| 1474 | II-15 | [III-1/III-5/III-4] |
| 1475 | II-15 | [III-1/III-5/III-5] |
| 1476 | II-15 | [III-2/III-1/III-1] |
| 1477 | II-15 | [III-2/III-1/III-2] |
| 1478 | II-15 | [III-2/III-1/III-3] |
| 1479 | II-15 | [III-2/III-1/III-4] |
| 1480 | II-15 | [III-2/III-1/III-5] |
| 1481 | II-15 | [III-2/III-2/III-1] |
| 1482 | II-15 | [III-2/III-2/III-2] |
| 1483 | II-15 | [III-2/III-2/III-3] |
| 1484 | II-15 | [III-2/III-2/III-4] |
| 1485 | II-15 | [III-2/III-2/III-5] |
| 1486 | II-15 | [III-2/III-3/III-1] |
| 1487 | II-15 | [III-2/III-3/III-2] |
| 1488 | II-15 | [III-2/III-3/III-3] |
| 1489 | II-15 | [III-2/III-3/III-4] |
| 1490 | II-15 | [III-2/III-3/III-5] |
| 1491 | II-15 | [III-2/III-4/III-1] |
| 1492 | II-15 | [III-2/III-4/III-2] |
| 1493 | II-15 | [III-2/III-4/III-3] |
| 1494 | II-15 | [III-2/III-4/III-4] |
| 1495 | II-15 | [III-2/III-4/III-5] |
| 1496 | II-15 | [III-2/III-5/III-1] |
| 1497 | II-15 | [III-2/III-5/III-2] |
| 1498 | II-15 | [III-2/III-5/III-3] |
| 1499 | II-15 | [III-2/III-5/III-4] |
| 1500 | II-15 | [III-2/III-5/III-5] |
| 1501 | II-15 | [III-3/III-1/III-1] |
| 1502 | II-15 | [III-3/III-1/III-2] |
| 1503 | II-15 | [III-3/III-1/III-3] |
| 1504 | II-15 | [III-3/III-1/III-4] |
| 1505 | II-15 | [III-3/III-1/III-5] |
| 1506 | II-15 | [III-3/III-2/III-1] |
| 1507 | II-15 | [III-3/III-2/III-2] |
| 1508 | II-15 | [III-3/III-2/III-3] |
| 1509 | II-15 | [III-3/III-2/III-4] |
| 1510 | II-15 | [III-3/III-2/III-5] |
| 1511 | II-15 | [III-3/III-3/III-1] |
| 1512 | II-15 | [III-3/III-3/III-2] |
| 1513 | II-15 | [III-3/III-3/III-3] |
| 1514 | II-15 | [III-3/III-3/III-4] |
| 1515 | II-15 | [III-3/III-3/III-5] |
| 1516 | II-15 | [III-3/III-4/III-1] |
| 1517 | II-15 | [III-3/III-4/III-2] |
| 1518 | II-15 | [III-3/III-4/III-3] |
| 1519 | II-15 | [III-3/III-4/III-4] |
| 1520 | II-15 | [III-3/III-4/III-5] |
| 1521 | II-15 | [III-3/III-5/III-1] |
| 1522 | II-15 | [III-3/III-5/III-2] |
| 1523 | II-15 | [III-3/III-5/III-3] |
| 1524 | II-15 | [III-3/III-5/III-4] |
| 1525 | II-15 | [III-3/III-5/III-5] |
| 1526 | II-15 | [III-4/III-1/III-1] |
| 1527 | II-15 | [III-4/III-1/III-2] |
| 1528 | II-15 | [III-4/III-1/III-3] |
| 1529 | II-15 | [III-4/III-1/III-4] |
| 1530 | II-15 | [III-4/III-1/III-5] |
| 1531 | II-15 | [III-4/III-2/III-1] |
| 1532 | II-15 | [III-4/III-2/III-2] |
| 1533 | II-15 | [III-4/III-2/III-3] |
| 1534 | II-15 | [III-4/III-2/III-4] |
| 1535 | II-15 | [III-4/III-2/III-5] |
| 1536 | II-15 | [III-4/III-3/III-1] |
| 1537 | II-15 | [III-4/III-3/III-2] |
| 1538 | II-15 | [III-4/III-3/III-3] |
| 1539 | II-15 | [III-4/III-3/III-4] |
| 1540 | II-15 | [III-4/III-3/III-5] |
| 1541 | II-15 | [III-4/III-4/III-1] |
| 1542 | II-15 | [III-4/III-4/III-2] |
| 1543 | II-15 | [III-4/III-4/III-3] |
| 1544 | II-15 | [III-4/III-4/III-4] |
| 1545 | II-15 | [III-4/III-4/III-5] |
| 1546 | II-15 | [III-4/III-5/III-1] |
| 1547 | II-15 | [III-4/III-5/III-2] |
| 1548 | II-15 | [III-4/III-5/III-3] |
| 1549 | II-15 | [III-4/III-5/III-4] |
| 1550 | II-15 | [III-4/III-5/III-5] |
| 1551 | II-15 | [III-5/III-1/III-1] |
| 1552 | II-15 | [III-5/III-1/III-2] |
| 1553 | II-15 | [III-5/III-1/III-3] |
| 1554 | II-15 | [III-5/III-1/III-4] |
| 1555 | II-15 | [III-5/III-1/III-5] |
| 1556 | II-15 | [III-5/III-2/III-1] |
| 1557 | II-15 | [III-5/III-2/III-2] |
| 1558 | II-15 | [III-5/III-2/III-3] |
| 1559 | II-15 | [III-5/III-2/III-4} |
| 1560 | II-15 | [III-5/III-2/III-5] |
| 1561 | II-15 | [III-5/III-3/III-1] |
| 1562 | II-15 | [III-5/III-3/III-2] |
| 1563 | II-15 | [III-5/III-3/III-3] |
| 1564 | II-15 | [III-5/III-3/III-4] |
| 1565 | II-15 | [III-5/III-3/III-5] |
| 1566 | II-15 | [III-5/III-4/III-1] |
| 1567 | II-15 | [III-5/III-4/III-2] |
| 1568 | II-15 | [III-5/III-4/III-3] |
| 1569 | II-15 | [III-5/III-4/III-4] |
| 1570 | II-15 | [III-5/III-4/III-5] |
| 1571 | II-15 | [III-5/III-5/III-1] |
| 1572 | II-15 | [III-5/III-5/III-2] |
| 1573 | II-15 | [III-5/III-5/III-3] |
| 1574 | II-15 | [III-5/III-5/III-4] |
| 1575 | II-15 | [III-5/III-5/III-5] |
| 1576 | II-16 | [III-1/III-1/III-1] |
| 1577 | II-16 | [III-1/III-1/III-2] |
| 1578 | II-16 | [III-1/III-1/III-3] |
| 1579 | II-16 | [III-1/III-1/III-4] |
| 1580 | II-16 | [III-1/III-1/III-5] |
| 1581 | II-16 | [III-1/III-2/III-1] |
| 1582 | II-16 | [III-1/III-2/III-2] |
| 1583 | II-16 | [III-1/III-2/III-3] |
| 1584 | II-16 | [III-1/III-2/III-4] |
| 1585 | II-16 | [III-1/III-2/III-5] |
| 1586 | II-16 | [III-1/III-3/III-1] |
| 1587 | II-16 | [III-1/III-3/III-2] |
| 1588 | II-16 | [III-1/III-3/III-3] |
| 1589 | II-16 | [III-1/III-3/III-4] |
| 1590 | II-16 | [III-1/III-3/III-5] |
| 1591 | II-16 | [III-1/III-4/III-1] |
| 1592 | II-16 | [III-1/III-4/III-2] |
| 1593 | II-16 | [III-1/III-4/III-3] |
| 1594 | II-16 | [III-1/III-4/III-4] |
| 1595 | II-16 | [III-1/III-4/III-5] |
| 1596 | II-16 | [III-1/III-5/III-1] |
| 1597 | II-16 | [III-1/III-5/III-2] |
| 1598 | II-16 | [III-1/III-5/III-3] |
| 1599 | II-16 | [III-1/III-5/III-4] |
| 1600 | II-16 | [III-1/III-5/III-5] |
| 1601 | II-16 | [III-2/III-1/III-1] |
| 1602 | II-16 | [III-2/III-1/III-2] |
| 1603 | II-16 | [III-2/III-1/III-3] |
| 1604 | II-16 | [III-2/III-1/III-4] |
| 1605 | II-16 | [III-2/III-1/III-5] |
| 1606 | II-16 | [III-2/III-2/III-1] |
| 1607 | II-16 | [III-2/III-2/III-2] |
| 1608 | II-16 | [III-2/III-2/III-3] |
| 1609 | II-16 | [III-2/III-2/III-4] |
| 1610 | II-16 | [III-2/III-2/III-5] |
| 1611 | II-16 | [III-2/III-3/III-1] |
| 1612 | II-16 | [III-2/III-3/III-2] |
| 1613 | II-16 | [III-2/III-3/III-3] |
| 1614 | II-16 | [III-2/III-3/III-4] |
| 1615 | II-16 | [III-2/III-3/III-5] |
| 1616 | II-16 | [III-2/III-4/III-1] |
| 1617 | II-16 | [III-2/III-4/III-2] |
| 1618 | II-16 | [III-2/III-4/III-3] |
| 1619 | II-16 | [III-2/III-4/III-4] |
| 1620 | II-16 | [III-2/III-4/III-5] |
| 1621 | II-16 | [III-2/III-5/III-1] |
| 1622 | II-16 | [III-2/III-5/III-2] |
| 1623 | II-16 | [III-2/III-5/III-3] |
| 1624 | II-16 | [III-2/III-5/III-4] |
| 1625 | II-16 | [III-2/III-5/III-5] |
| 1626 | II-16 | [III-3/III-1/III-1] |
| 1627 | II-16 | [III-3/III-1/III-2] |
| 1628 | II-16 | [III-3/III-1/III-3] |
| 1629 | II-16 | [III-3/III-1/III-4] |
| 1630 | II-16 | [III-3/III-1/III-5] |
| 1631 | II-16 | [III-3/III-2/III-1] |
| 1632 | II-16 | [III-3/III-2/III-2] |
| 1633 | II-16 | [III-3/III-2/III-3] |
| 1634 | II-16 | [III-3/III-2/III-4] |
| 1635 | II-16 | [III-3/III-2/III-5] |
| 1636 | II-16 | [III-3/III-3/III-1] |
| 1637 | II-16 | [III-3/III-3/III-2] |
| 1638 | II-16 | [III-3/III-3/III-3] |
| 1639 | II-16 | [III-3/III-3/III-4] |
| 1640 | II-16 | [III-3/III-3/III-5] |
| 1641 | II-16 | [III-3/III-4/III-1] |
| 1642 | II-16 | [III-3/III-4/III-2] |
| 1643 | II-16 | [III-3/III-4/III-3] |
| 1644 | II-16 | [III-3/III-4/III-4] |
| 1645 | II-16 | [III-3/III-4/III-5] |
| 1646 | II-16 | [III-3/III-5/III-1] |
| 1647 | II-16 | [III-3/III-5/III-2] |
| 1648 | II-16 | [III-3/III-5/III-3] |
| 1649 | II-16 | [III-3/III-5/III-4] |
| 1650 | II-16 | [III-3/III-5/III-5] |
| 1651 | II-16 | [III-4/III-1/III-1] |
| 1652 | II-16 | [III-4/III-1/III-2] |
| 1653 | II-16 | [III-4/III-1/III-3] |
| 1654 | II-16 | [III-4/III-1/III-4] |
| 1655 | II-16 | [III-4/III-1/III-5] |
| 1656 | II-16 | [III-4/III-2/III-1] |
| 1657 | II-16 | [III-4/III-2/III-2] |
| 1658 | II-16 | [III-4/III-2/III-3] |
| 1659 | II-16 | [III-4/III-2/III-4] |
| 1660 | II-16 | [III-4/III-2/III-5] |
| 1661 | II-16 | [III-4/III-3/III-1] |
| 1662 | II-16 | [III-4/III-3/III-2] |
| 1663 | II-16 | [III-4/III-3/III-3] |
| 1664 | II-16 | [III-4/III-3/III-4] |
| 1665 | II-16 | [III-4/III-3/III-5] |
| 1666 | II-16 | [III-4/III-4/III-1] |
| 1667 | II-16 | [III-4/III-4/III-2] |
| 1668 | II-16 | [III-4/III-4/III-3] |
| 1669 | II-16 | [III-4/III-4/III-4] |
| 1670 | II-16 | [III-4/III-4/III-5] |
| 1671 | II-16 | [III-4/III-5/III-1] |
| 1672 | II-16 | [III-4/III-5/III-2] |
| 1673 | II-16 | [III-4/III-5/III-3] |
| 1674 | II-16 | [III-4/III-5/III-4] |
| 1675 | II-16 | [III-4/III-5/III-5] |
| 1676 | II-16 | [III-5/III-1/III-1] |
| 1677 | II-16 | [III-5/III-1/III-2] |
| 1678 | II-16 | [III-5/III-1/III-3] |
| 1679 | II-16 | [III-5/III-1/III-4] |
| 1680 | II-16 | [III-5/III-1/III-5] |
| 1681 | II-16 | [III-5/III-2/III-1] |
| 1682 | II-16 | [III-5/III-2/III-2] |
| 1683 | II-16 | [III-5/III-2/III-3] |
| 1684 | II-16 | [III-5/III-2/III-4] |
| 1685 | II-16 | [III-5/III-2/III-5] |
| 1686 | II-16 | [III-5/III-3/III-1] |
| 1687 | II-16 | [III-5/III-3/III-2] |
| 1688 | II-16 | [III-5/III-3/III-3] |
| 1689 | II-16 | [III-5/III-3/III-4] |
| 1690 | II-16 | [III-5/III-3/III-5] |
| 1691 | II-16 | [III-5/III-4/III-1] |
| 1692 | II-16 | [III-5/III-4/III-2] |
| 1693 | II-16 | [III-5/III-4/III-3] |
| 1694 | II-16 | [III-5/III-4/III-4] |
| 1695 | II-16 | [III-5/III-4/III-5] |
| 1696 | II-16 | [III-5/III-5/III-1] |
| 1697 | II-16 | [III-5/III-5/III-2] |
| 1698 | II-16 | [III-5/III-5/III-3] |
| 1699 | II-16 | [III-5/III-5/III-4] |
| 1700 | II-16 | [III-5/III-5/III-5] |

In the light emitting element ED according to an embodiment, the capping layer GPL may further include an organic material or an inorganic material, in addition to the amine compound according to an embodiment, described above. For example, when the capping layer GPL further includes an inorganic material, the inorganic material may include an alkali metal compound such as LiF, an alkaline earth metal compound such as MgF₂, SiGN, SiN_{X}, SiO_{y}, etc.

For example, when the capping layer CPL further includes an organic material, the organic material may include α-NPD, NPB, TPD, m-MTDATA, Alq₃ CuPc, N4,N4,N4′,N4′-tetra(biphenyl-4-yl) biphenyl-4,4′-diamine (TPD15), 4,4′,4″-tris(carbazol sol-9-yl)triphenylamine (TCTA), etc., or may include epoxy resins or acrylates such as methacrylates. However, the embodiment of the present disclosure is not limited thereto, and the capping layer CPL may further include compounds P1 to P5.

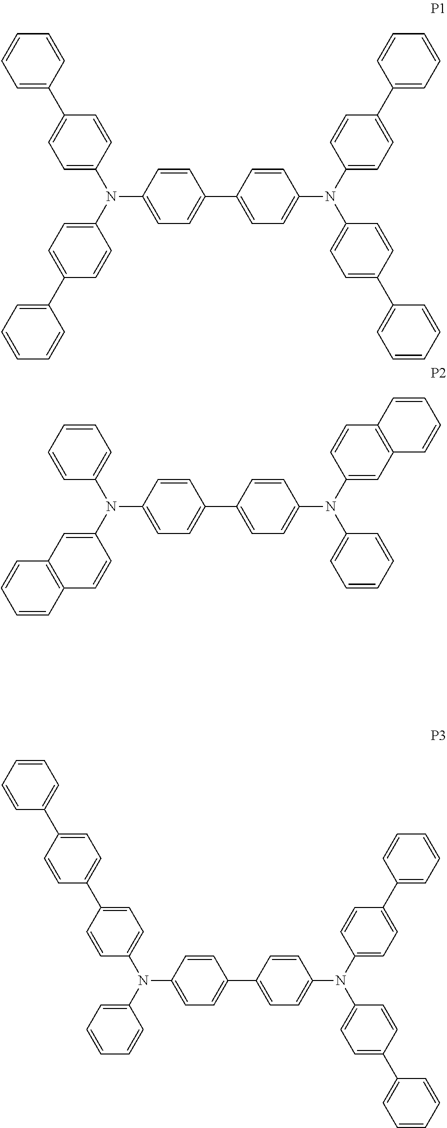

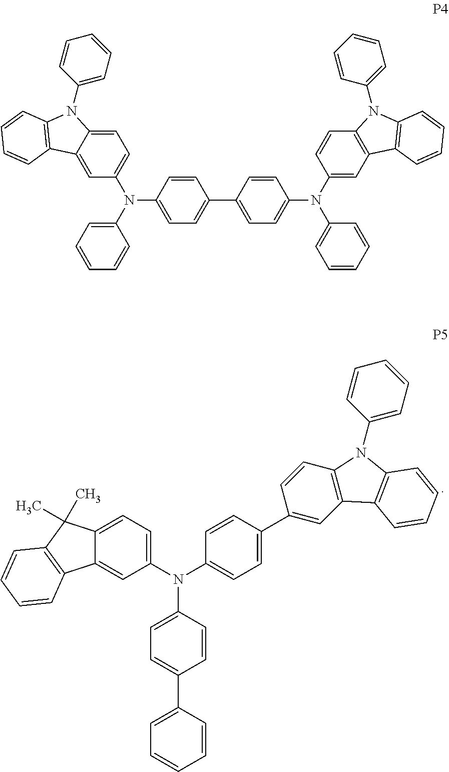 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In the light emitting element ED according to an embodiment, the capping layer CPL may have a refractive index of about 1.6 or greater. For example, the capping layer CPL may have a refractive index of about 1.6 or greater in a wavelength range of about 550 nm to about 660 nm.

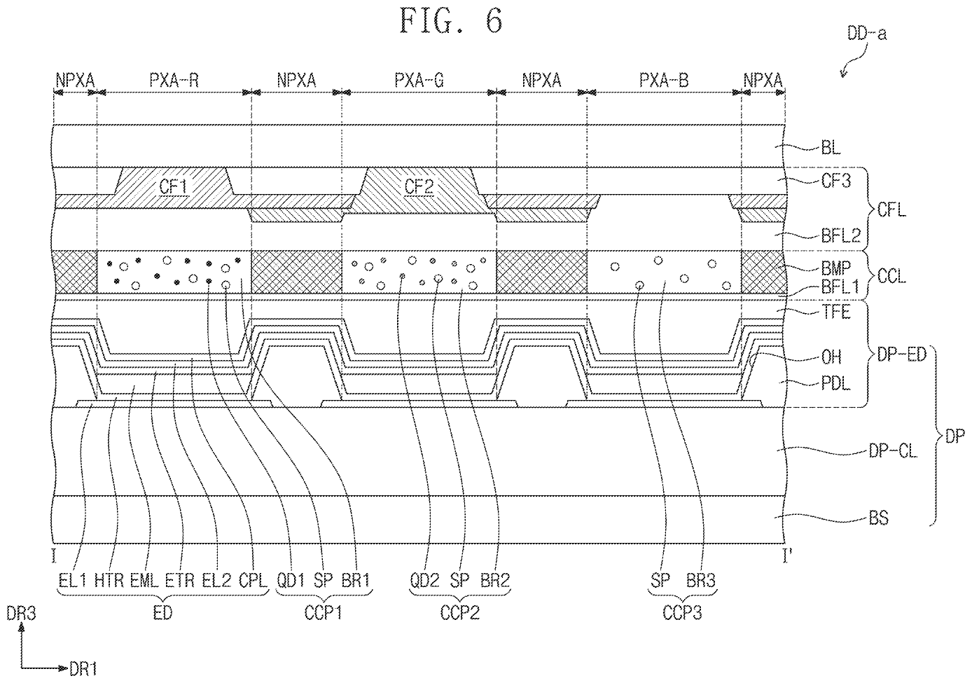

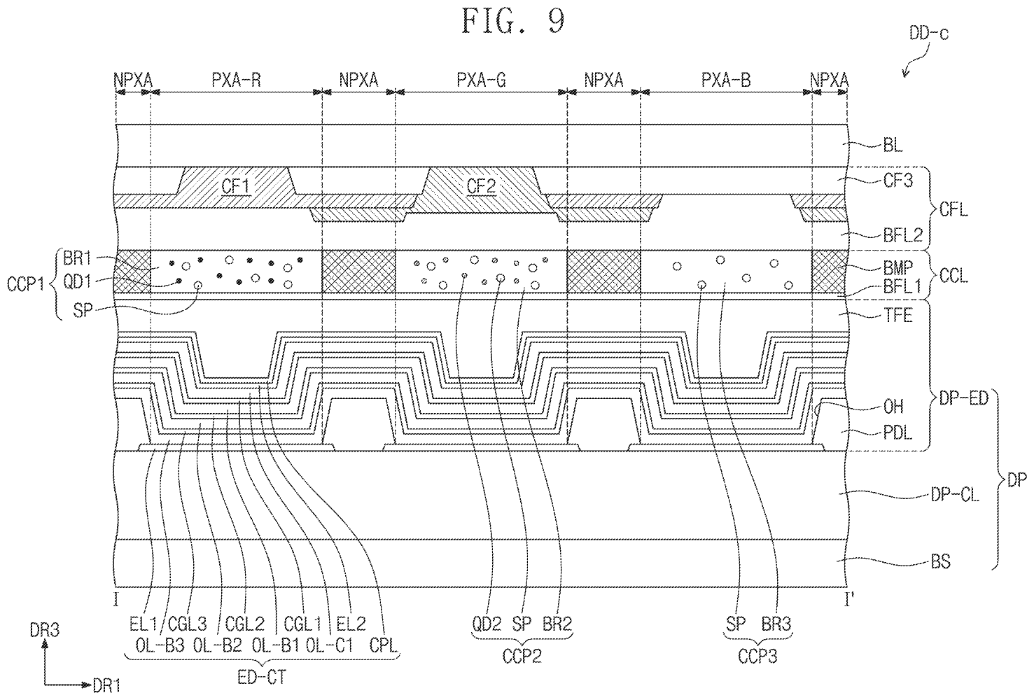

FIGS. 6 to 9 are each a cross-sectional view of a display device according to an embodiment. Hereinafter, in the description of the display device according to an embodiment with reference to FIGS. 6 and 9, content/description overlapping the one described above with reference to FIGS. 1 to 5 may not be described again, and the differences will be primarily described.

Referring to FIG. 6, a display device DD-a according to an embodiment may include a display panel DP having a display element layer DP-ED, a light control layer CCL on the display panel DP, and a color filter layer CFL.

In an embodiment shown in FIG. 6, the display panel DP may include a base layer BS, a circuit layer DP-CL provided on the base layer BS, and a display element layer DP-ED, and the element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, a second electrode EL2 disposed on the electron transport region ETR, and a capping layer CPL on the second electrode EL2. In some embodiments, a structure of the light emitting element ED shown in FIG. 6 may be the same as the structure of the light emitting element of FIGS. 3 to 5 described above.

The capping layer CPL of the light emitting element ED included in a display device DD-a according to an embodiment may include the amine compound of an embodiment, described above.

Referring to FIG. 6, the emission layer EML may be disposed in the openings OH defined in the pixel defining films PDL. For example, the emission layer EML separated by the pixel defining films PDL and provided corresponding to each of light emitting regions PXA-R, PXA-G, and PXA-B may emit light in substantially the same wavelength ranges. In the display device DD-a of an embodiment, the emission layer EML may emit blue light. In an embodiment, the emission layer EML may be provided as a common layer throughout the light emitting regions PXA-R, PXA-G, and PXA-B.

The light control layer CCL may be on the display panel DP. The light control layer CCL may include a light converter. The light converter may be a quantum dot or a phosphor. The light converter may wavelength-convert the provided light and emit the wavelength-converted light. For example, the light control layer CCL may be a layer containing quantum dots or phosphors.

The light control layer CCL may include a plurality of light control units CCP1, CCP2, and CCP3. The light control units CCP1, CCP2, and CCP3 may be spaced apart from (separated from) each other.

Referring to FIG. 6, a division pattern BMP may be disposed between the light control units CCP1, CCP2, and CCP3 spaced apart from (separated from) each other, but the embodiment of the present disclosure is not limited thereto. In FIG. 6, the division pattern BMP is shown to not overlap the light control units CCP1, CCP2, and CCP3. However, edges of the light control units CCP1, CCP2, and CCP3 may overlap at least a portion of the division pattern BMP (along the DR3 direction).

The light control layer CCL may include a first light control unit CCP1 including a first quantum dot QD1 for converting first color light provided from the light emitting element ED into second color light, a second light control unit CCP2 including a second quantum dot QD2 for converting the first color light into third color light, and a third light control unit CCP3 transmitting the first color light.

In an embodiment, the first light control unit CCP1 may provide red light, which is the second color light, and the second light control unit CCP2 may provide green light, which is the third color light. The third light control unit CCP3 may transmit and provide blue light, which is the first color light provided from the light emitting element ED. For example, the first quantum dot QD1 may be a red quantum dot and the second quantum dot QD2 may be a green quantum dot. The same descriptions above may be applied to the quantum dots QD1 and QD2.

In some embodiments, the light control layer CCL may further include scatterers SP. The first light control unit CCP1 may include the first quantum dot QD1 and the scatterers SP, the second light control unit CCP2 may include the second quantum dot QD2 and the scatterers SP, and the third light control unit CCP3 may not include (e.g., may exclude) a quantum dot but may include the scatterers SP.

The scatterers SP may be inorganic particles. For example, the scatterers SP may include at least one selected from among TiO₂, ZnO, Al₂O₃, SiO₂, and hollow silica. The scatterers SP may include any one selected from among TiO₂, ZnO, Al₂O₃, SiO₂, and hollow silica, or may be a mixture of two or more materials selected from among TiO₂, ZnO, Al₂O₃, SiO₂, and hollow silica.

The first light control unit CCP1, the second light control unit CCP2, and the third light control unit CCP3 may include base resins BR1, BR2, and BR3 for dispersing the quantum dots QD1 and QD2 and the scatterers SP. In an embodiment, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3. The base resins BR1, BR2, and BR3 are a medium in which the quantum dots QD1 and QD2 and the scatterers SP are dispersed, and may be formed of one or more suitable resin compositions, which may be generally referred to as a binder. For example, the base resins BR1, BR2, and BR3 may be an acrylic resin, a urethane-based resin, a silicone-based resin, an epoxy-based resin, etc. The base resins BR1, BR2, and BR3 may be a transparent resin. In an embodiment, the first base resin BR1, the second base resin BR2, and the third base resin BR3 may each be the same as or different from each other.

The light control layer CCL may include a barrier layer BFL1. The barrier layer BFL1 may serve to prevent or reduce moisture and/or oxygen (hereinafter referred to as “moisture/oxygen”) from being introduced. The barrier layer BFL1 may be disposed on the light control units CCP1, CCP2, and CCP3 to prevent or reduce the light control units CCP1, CCP2, and CCP3 from being exposed to moisture/oxygen. In some embodiments, the barrier layer BFL1 may cover the light control units CCP1, CCP2, and CCP3. In some embodiments, a barrier layer BFL2 may be provided between the light control units CCP1, CCP2, and CCP3 and the color filter layer CFL.

The barrier layers BFL1 and BFL2 may include (e.g., may each include) at least one inorganic layer. For example, the barrier layers BFL1 and BFL2 may be (e.g., may each be) formed of an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film in which light transmittance is secured, etc. In some embodiments, the barrier layers BFL1 and BFL2 may further include an organic film. The barrier layers BFL1 and BFL2 may be formed of a single layer or a plurality of layers.

In the display device DD of an embodiment, the color filter layer CFL may be on the light control layer CCL. For example, the color filter layer CFL may be directly on the light control layer CCL. In this embodiment, the barrier layer BFL2 may not be provided.

The color filter layer CFL may include filters CF1, CF2, and CF3. For example, the color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. The filters CF1, CF2, and CF3 may each include a polymer photosensitive resin, a pigment and/or a dye. The first filter CF1 may include a red pigment or a red dye, the second filter CF2 may include a green pigment and/or a green dye, and the third filter CF3 may include a blue pigment and/or a blue dye. In some embodiments, the embodiment of the present disclosure is not limited thereto, and the third filter CF3 may not include (e.g., may exclude) any pigment or dye. The third filter CF3 may include a polymer photosensitive resin, but not include any pigment or dye. The third filter CF3 may be transparent. The third filter CF3 may be formed of a transparent photosensitive resin.

In some embodiments, the first filter CF1 and the second filter CF2 may be yellow filters. The first filter CF1 and the second filter CF2 may not be separated and may be provided as a single body. The first to third filters CF1, CF2, and CF3 may be disposed corresponding to the red light emitting region PXA-R, the green light emitting region PXA-G, and the blue light emitting region PXA-B, respectively.

In some embodiments, the color filter layer CFL may include a light blocking unit. The color filter layer CFL may include the light blocking unit disposed to overlap the boundaries of the neighboring filters CF1, CF2, and CF3. The light blocking unit may be a black matrix. The light blocking unit may be formed including an organic light blocking material or an inorganic light blocking material, both (e.g., simultaneously) including a black pigment and/or a black dye. The light blocking unit may separate boundaries between the adjacent filters CF1, CF2, and CF3. In some embodiments, the light blocking unit may be formed of a blue filter.

The base substrate BL may be on the color filter layer CFL. The base substrate BL may be a member providing a base surface on which the color filter layer CFL and the light control layer CCL are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

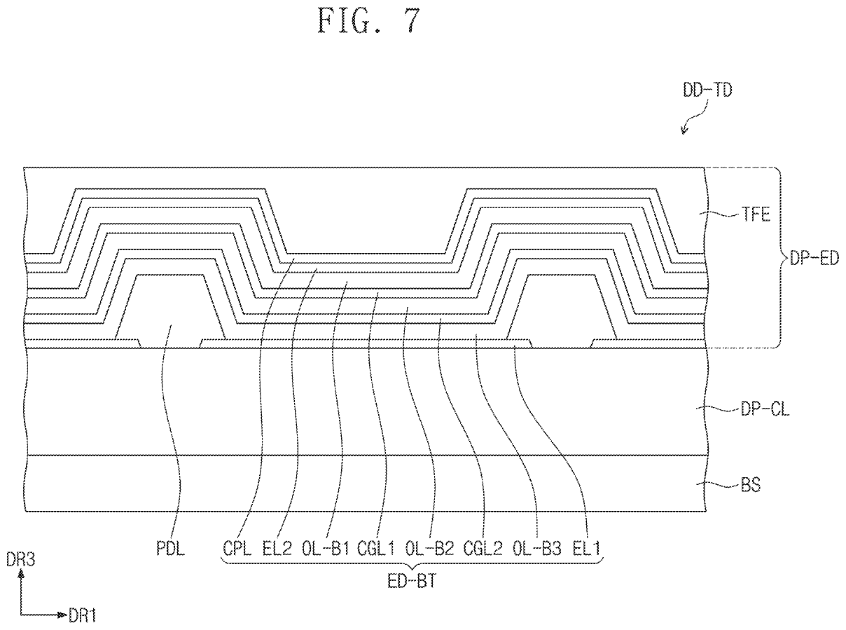

FIG. 7 is a cross-sectional view showing a portion of a display device according to an embodiment. FIG. 7 shows a cross-sectional view of a portion corresponding to the display panel DP of FIG. 6. In a display device DD-TD of an embodiment, a light emitting element ED-BT may include a plurality of light emitting structures OL-B1, OL-B2, and OL-B3. The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. In some embodiments, the light emitting element ED-BT may include the capping layer CPL on the second electrode EL2.

In the display device DD-TD according to an embodiment, the light emitting structures OL-B1, OL-B2, and OL-B3 each may include the emission layer EML (FIG. 6), and a hole transport region HTR and an electron transport region ETR disposed with the emission layer EML (FIG. 6) therebetween. For example, the light emitting element ED-BT included in the display device DD-TD of an embodiment may be a light emitting element having a tandem structure including a plurality of emission layers.

In an embodiment shown in FIG. 7, light emitted from each of the light emitting structures OL-B1, OL-B2, and OL-B3 may all be blue light. However, the embodiment of the present disclosure is not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light (e.g., light beams) in different wavelength ranges may emit white light (e.g., a combined white light).

Charge generation layers CGL1 and CGL2 may be disposed between neighboring light emitting structures OL-B1, OL-B2, and OL-B3. The charge generation layers CGL1 and CGL2 may include a p-type or kind charge generation layer (e.g., P-charge generation layer) and/or an n-type or kind charge generation layer (e.g., N-charge generation layer).

The capping layer CPL included in the display device DD-TD of an embodiment may include the amine compound of an embodiment, described above.

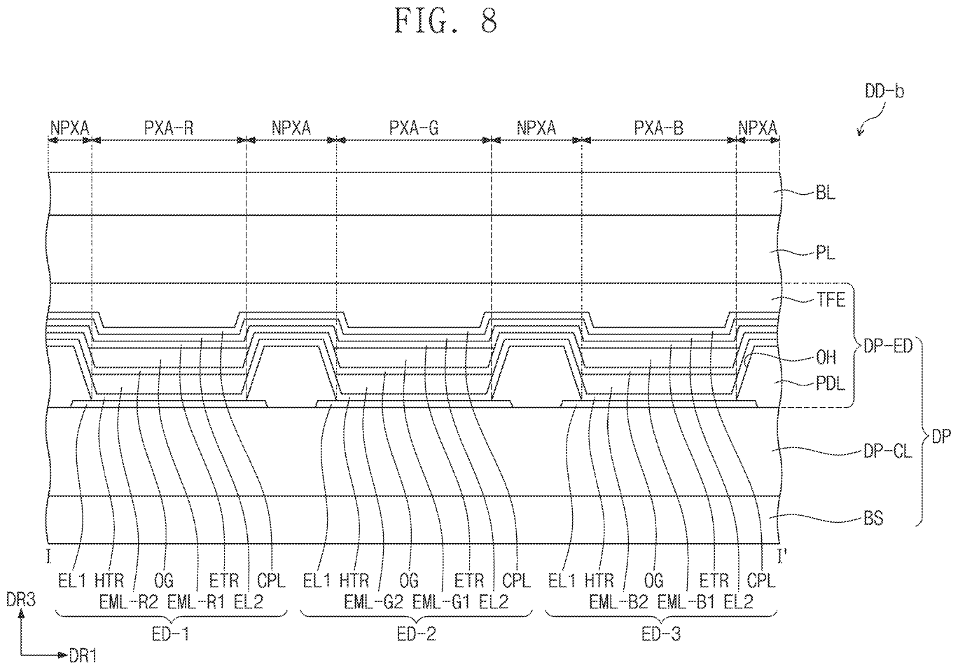

Referring to FIG. 8, a display device DD-b according to an embodiment may include light emitting elements ED-1, ED-2, and ED-3 in which two emission layers are stacked. Compared to the display device DD according to an embodiment shown in FIG. 2, a difference is that in an embodiment shown in FIG. 8, the first to third light emitting elements ED-1, ED-2, and ED-3 each include two emission layers stacked in a thickness direction. In each of the first to third light emitting elements ED-1, ED-2, and ED-3, the two emission layers may emit light in substantially the same wavelength range.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. A light emitting auxiliary portion OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The light emitting auxiliary portion OG may include a single layer or multiple layers. The light emitting auxiliary portion OG may include a charge generation layer. For example, the light emitting auxiliary portion OG may include an electron transport region, a charge generation layer, and a hole transport region that are sequentially stacked (in the stated order). The light emitting auxiliary portion OG may be provided as a common layer throughout the first to third light emitting elements ED-1, ED-2, and ED-3. However, the embodiment of the present disclosure is not limited thereto, and the light emitting auxiliary portion OG may be provided to be patterned inside the openings OH defined in the pixel defining films PDL.

The first red emission layer EML-R1, the first green emission layer EML-G1, and the first blue emission layer EML-B1 may be between the hole transport region HTR and the emission auxiliary portion OG. The second red emission layer EML-R2, the second green emission layer EML-G2, and the second blue emission layer EML-B2 may be between the emission auxiliary portion OG and the electron transport region ETR.

For example, the light emitting element ED-1 may include the first electrode EL1, the hole transport region HTR, the second red emission layer EML-R2, the emission auxiliary portion OG, the first red emission layer EML-R1, the electron transport region ETR, the second electrode EL2, and the capping layer CPL, which are sequentially stacked (in the stated order). The second light emitting element ED-2 may include the first electrode EL1, the hole transport region HTR, the second green emission layer EML-G2, the emission auxiliary portion OG, the first green emission layer EML-G1, the electron transport region ETR, the second electrode EL2, and the capping layer CPL, which are sequentially stacked (in the stated order). The third light emitting element ED-3 may include the first electrode EL1, the hole transport region HTR, the second blue emission layer EML-B2, the emission auxiliary portion OG, the first blue emission layer EML-B1, the electron transport region ETR, the second electrode EL2, and the capping layer CPL, which are sequentially stacked (in the stated order). In an embodiment, the capping layer CPL included in the first light emitting element ED-1 to the third light emitting element ED-3 may include the amine compound according to the embodiment, described above.

In some embodiments, an optical auxiliary layer PL may be on the display element layer DP-ED. The optical auxiliary layer PL may include a polarizing layer. The optical auxiliary layer PL may be on the display panel DP to control reflected light in the display panel DP due to external light. The optical auxiliary layer PL may not be provided in the display device according to an embodiment.

Unlike FIGS. 7 and 8, the display device DD-c of FIG. 9 is shown to include four light emitting structures OL-B1, OL-B2, OL-B3, and OL-C1. The light emitting element ED-BT may include the first electrode EL1 and the second electrode EL2 facing each other, the first to fourth light emitting structures L-B1, OL-B2, OL-B3, and OL-C1 sequentially stacked in a thickness direction between the first electrode EL1 and the second electrode EL2, and the capping layer CPL disposed on the second electrode EL2. Charge generation layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light having different wavelength ranges.

The charge generation layers CGL1, CGL2 and CGL3 disposed between the neighboring light emitting structures OL-B1, OL-B2, OL-B3, and OL-C1 may include a p-type or kind charge generation layer (e.g., P-charge generation layer) and/or an n-type or kind charge generation layer (e.g., N-charge generation layer).

The light emitting element ED-CT included in the display device DD-c according to an embodiment may include the amine compound of an embodiment, described above. For example, the capping layer CPL of the light emitting element ED-CT may include the amine compound according to an embodiment, described above.

A light emitting element according to an embodiment of the present disclosure includes the amine compound of an embodiment, described above, in a capping layer CPL on the second electrode EL2, and may thus exhibit increased luminous efficiency and high luminance characteristics. The light emitting element ED according to an embodiment may further include the amine compound of an embodiment, described above, in at least one functional layer of the hole transport region HTR, the emission layer EML, or the electron transport region ETR, which are disposed between the first electrode EL1 and the second electrode EL2.

The amine compound according to an embodiment, described above, may include a benzoazole derivative and at least one hydroxy group, which is a substituent, and the amine compounds may thus be bonded through a hydrogen bond to increase the bonding strength of the amine compound molecules. Accordingly, planarity of the compounds in a layer including the amine compounds may be increased, and light extraction efficiency of a light emitting element including the amine compounds according to an embodiment may be increased. The light emitting element according to an embodiment includes the amine compound according to an embodiment in a capping layer on the second electrode, which is an upper electrode, and may thus exhibit high luminance and high efficiency characteristics.

Hereinafter, with reference to Examples and Comparative Examples, an amine compound and a light emitting element according to an embodiment of the present disclosure will be described in more detail. In some embodiments, Examples shown below are presented merely for the understanding of the present disclosure, and the scope of the present disclosure is not limited thereto.

### EXAMPLES

### 1. Synthesis of Amine Compounds

First, a process of synthesizing amine compounds according to an embodiment of the present disclosure will be described in more detail by providing a process of synthesizing Example Compounds below as an example. In some embodiments, a process of synthesizing amine compounds, which will be described hereinafter, is provided merely as an example, and thus the process of synthesizing amine compounds according to an embodiment of the present disclosure is not limited to Examples below.

### (1) Synthesis of Compound 1

Amine Compound 1 according to an embodiment may be synthesized by, for example, processes of Reaction Formula 1.

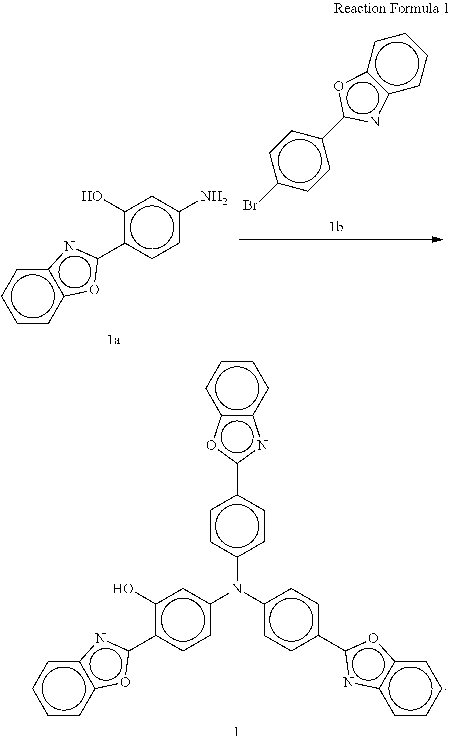

Intermediate Compound 1a (2.26 g, 10 mmol), Intermediate Compound 1b (5.74 g, 20 mmol), Pd₂(dba)₃ (0.34 g), PtBu₃ (0.2 mL), and NaO^{t}Bu (3.6 g) were dissolved in toluene (60 mL) and then stirred at 85° C. for 2 hours. After lowering the temperature of the reaction solution to room temperature, the reaction was quenched with water and the product was then extracted three times with ethyl acetate. The separated organic layer was dried over anhydrous magnesium sulfate and distilled under reduced pressure, and the resulting residue was separated and purified through silica gel column chromatography to obtain Compound 1 (4.7 g, yield: 76%). The obtained compound was confirmed through high resolution (HR) mass measurement. (C₄₇H₃₃N M+1: 612.179)

### (2) Synthesis of Compound 32

Amine Compound 32 according to an embodiment may be synthesized by, for example, processes of Reaction Formula 2.

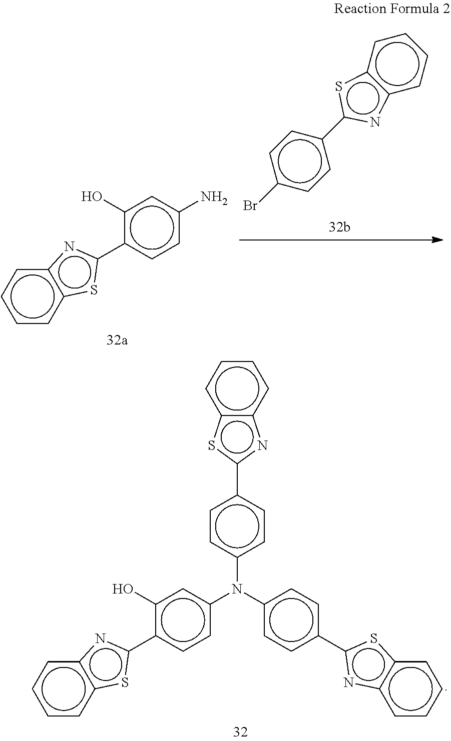

Compound 32 (3.5 g, yield: 78%) was obtained in substantially the same manner as in the synthesis of Compound 1, except that Intermediate Compound 32a was utilized instead of Intermediate Compound 1a and Intermediate Compound 32b was utilized instead of Intermediate Compound 1b. The obtained compound was confirmed through HR mass measurement. (C₃₉H₂₄N₄OS₃ M+1: 661.112)

### (3) Synthesis of Compound 44

Amine Compound 44 according to an embodiment may be synthesized by, for example, processes of Reaction Formula 3.

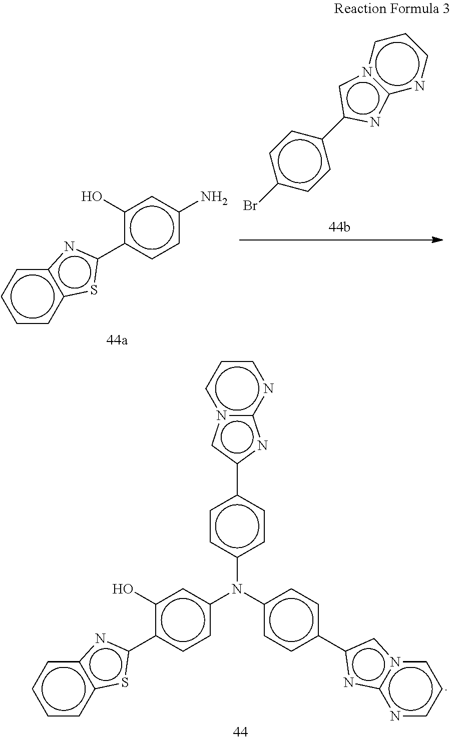

Compound 44 (4.5 g, yield: 72%) was obtained in substantially the same manner as in the synthesis of Compound 1, except that Intermediate Compound 44a was utilized instead of Intermediate Compound 1a and Intermediate Compound 44b was utilized instead of Intermediate Compound 1b. The obtained compound was confirmed through HR mass measurement. (C₃₇H₂₄N₈OS M+1: 629.179)

### (4) Synthesis of Compound 56

Amine Compound 56 according to an embodiment may be synthesized by, for example, processes of Reaction Formula 4.

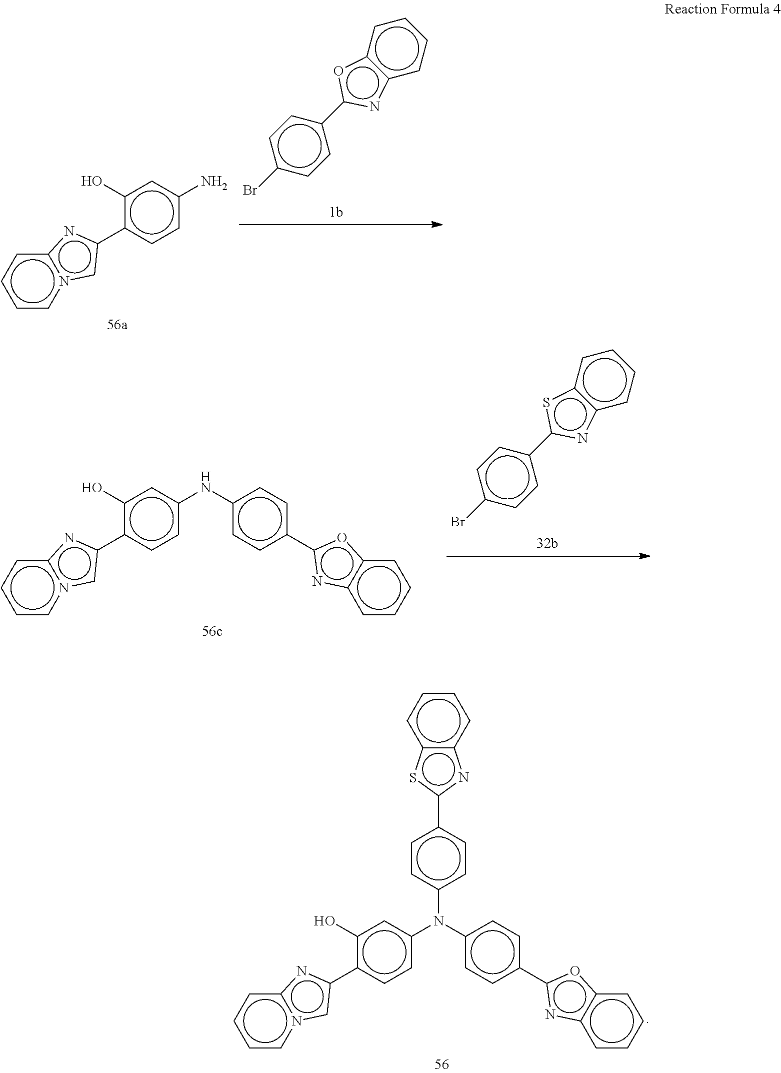

Intermediate Compound 56c was obtained in substantially the same manner as in the synthesis of Compound 1, except that Compound 56a was utilized instead of Intermediate Compound 1a. Compound 56 (4.3 g, yield: 70%) was then obtained in substantially the same manner as in the synthesis of Compound 1, except that Intermediate Compound 56c and Intermediate Compound 32a were utilized. The obtained compound was confirmed through HR mass measurement. (C₃₉H₂₅N₅O₂S M+1: 678.179)

### (5) Synthesis of Compound 57

Amine Compound 57 according to an embodiment may be synthesized by, for example, processes of Reaction Formula 5.

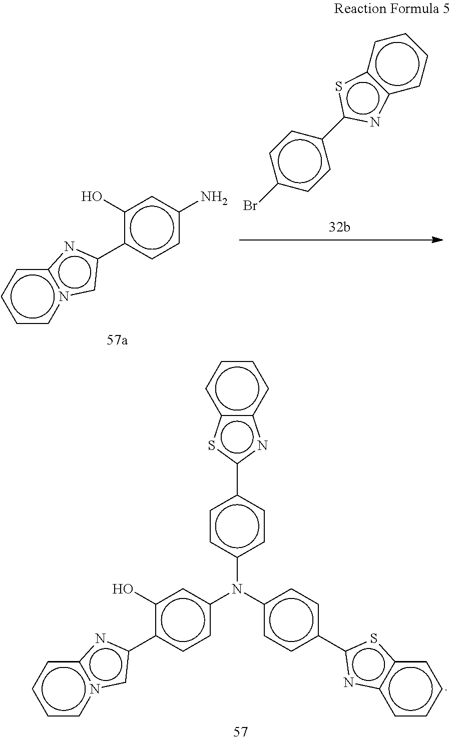

Compound 57 (4.8 g, yield: 75%) was obtained in substantially the same manner as in the synthesis of Compound 1, except that Intermediate Compound 57a was utilized instead of Intermediate Compound 1a and Intermediate Compound 32b was utilized instead of Intermediate Compound 1b. The obtained compound was confirmed through HR mass measurement. (C₃₉H₂₅N₅OS₂ M+1: 644.150)

### (6) Synthesis of Compound 58

Amine Compound 58 according to an embodiment may be synthesized by, for example, processes of Reaction Formula 6.

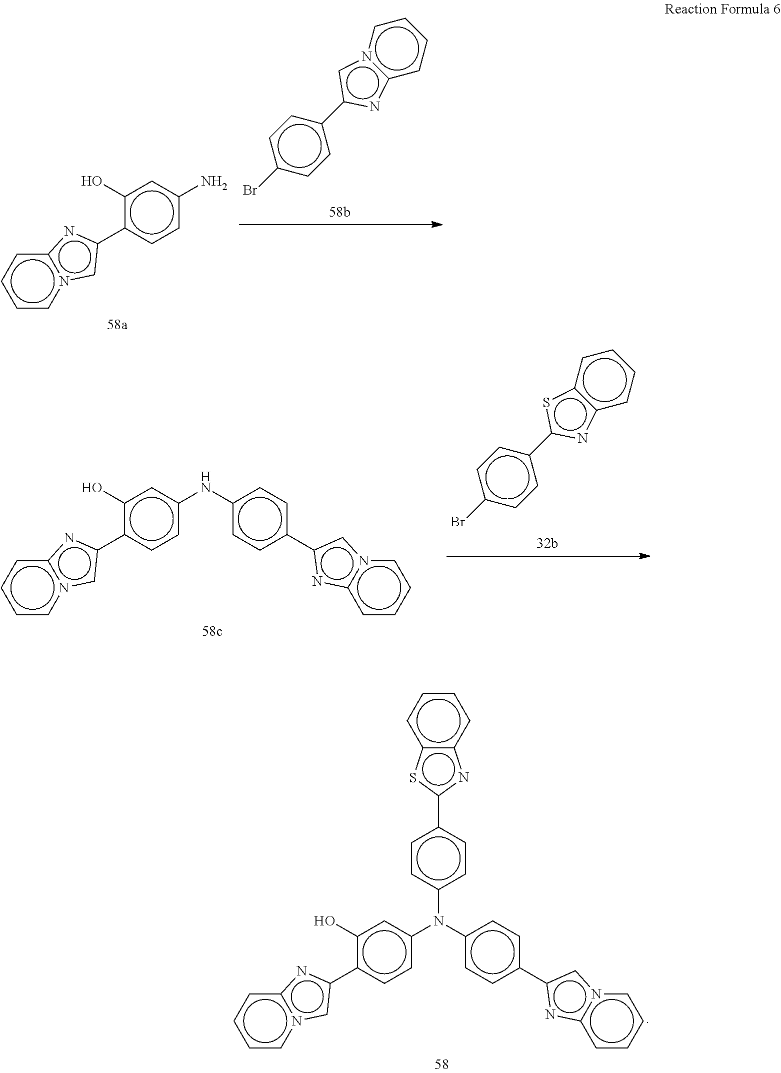

Compound 58c was obtained in substantially the same manner as in the synthesis of Compound 1, except that Intermediate Compound 58a was utilized instead of Intermediate Compound 1a and Intermediate Compound 58b was utilized instead of Intermediate Compound 1b. Compound 58 (4.4 g, yield: 72%) was then obtained in substantially the same manner as in the synthesis of Compound 1, except that Intermediate Compound 32b was utilized instead of Intermediate Compound 58c and Intermediate Compound 1 b. The obtained compound was confirmed through HR mass measurement. (C₃₉H₂₆N₆OS M+1: 627.188)

### (7) Synthesis of Compound 97

Amine Compound 97 according to an embodiment may be synthesized by, for example, processes of Reaction Formula 7.

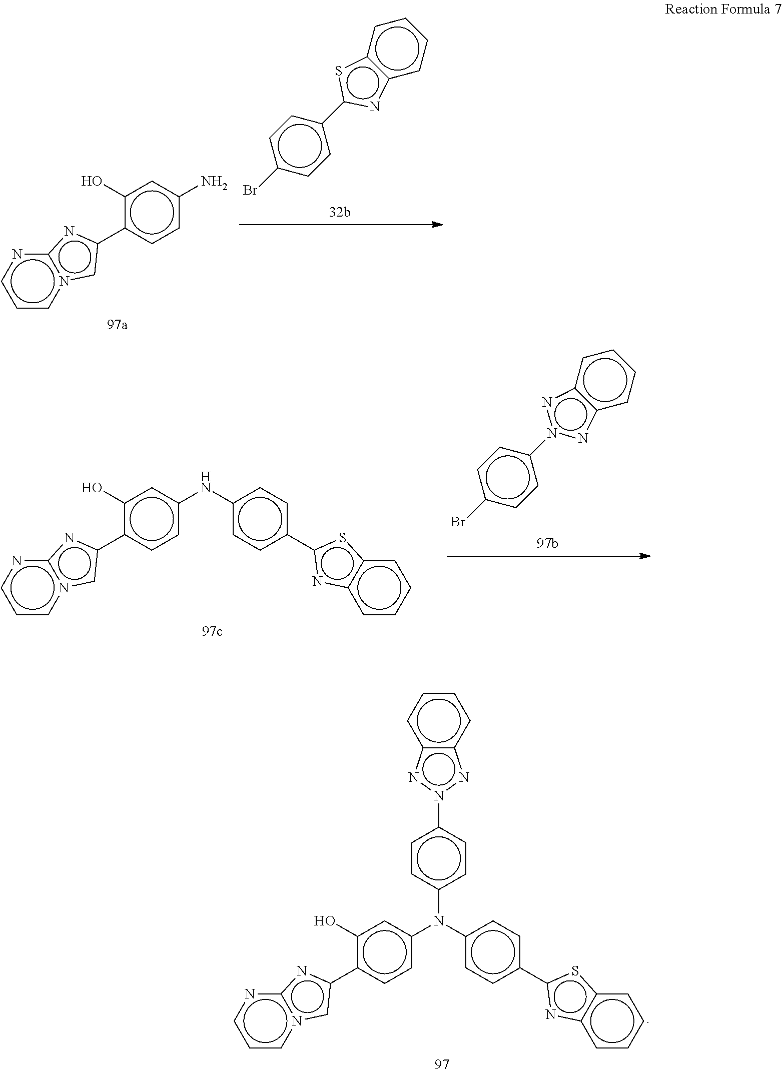

Compound 97b was obtained in substantially the same manner as in the synthesis of Compound 1, except that Intermediate Compound 97a was utilized instead of Intermediate Compound 1a and Intermediate Compound 32b was utilized instead of Intermediate Compound 1b. Compound 97 (4.3 g, yield: 70%) was then obtained in substantially the same manner as in the synthesis of Compound 1, utilizing Intermediate Compound 97b and Intermediate Compound 97c. The obtained compound was confirmed through HR mass measurement. (C₃₇H₂₄N₈OS M+1: 629.179)

### (8) Synthesis of Compound 101

Amine Compound 101 according to an embodiment may be synthesized by, for example, processes of Reaction Formula 8.

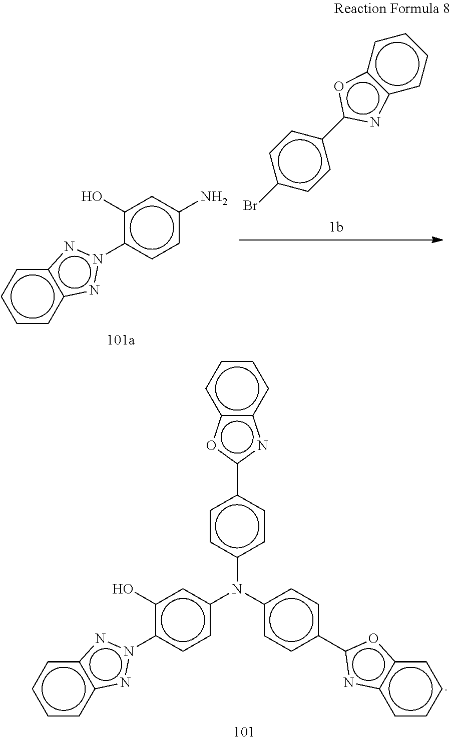

Compound 101 (4.4 g, yield: 72%) was then obtained in substantially the same manner as in the synthesis of Compound 1, except that 101a was utilized instead of Intermediate 1a. The obtained compound was confirmed through HR mass measurement. (C₃₉H₂₆N₆OS M+1: 627.188)

### (9) Synthesis of Compound 115

Amine Compound 115 according to an embodiment may be synthesized by, for example, processes of Reaction Formula 9.

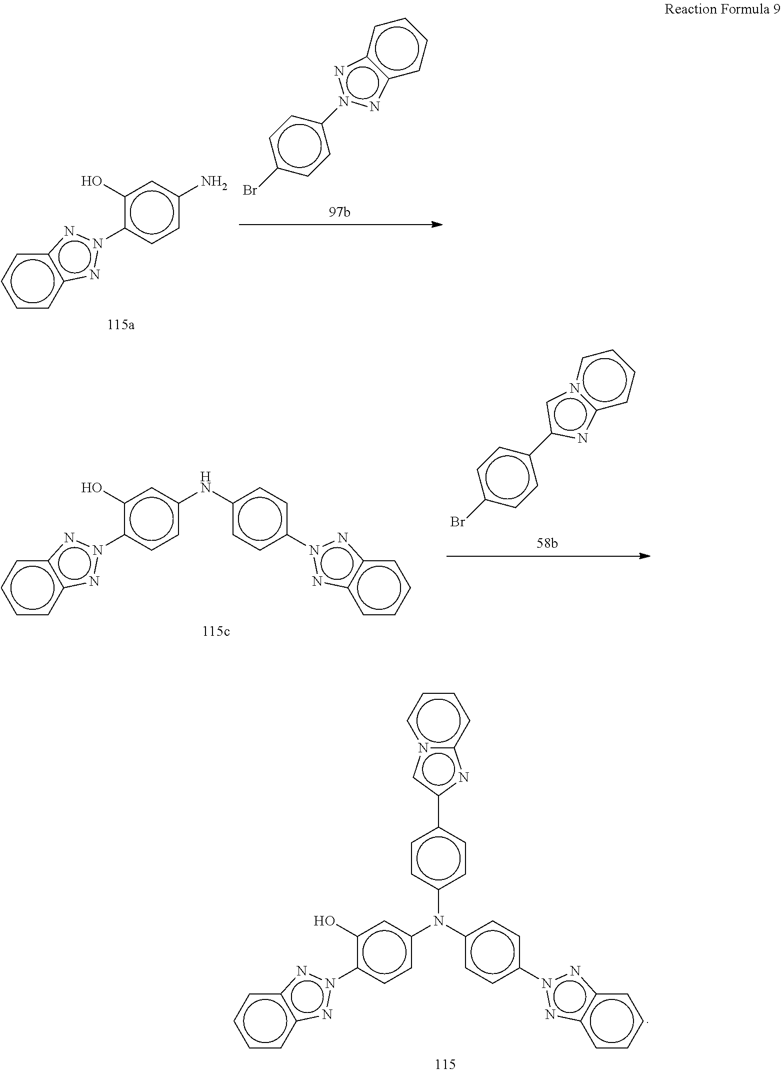

Compound 115b was obtained in substantially the same manner as in the synthesis of Compound 1, except that Intermediate Compound 115a was utilized instead of Intermediate Compound 1a and Intermediate Compound 97c was utilized instead of Intermediate Compound 1b. Compound 115 (4.4 g, yield: 72%) was then obtained in substantially the same manner as in the synthesis of Compound 1, utilizing Intermediate Compound 115b and Intermediate Compound 58b. The obtained compound was confirmed through HR mass measurement. (C₃₇H₂₅N₉O M+1: 612.218)

### (10) Synthesis of Compound 147

Amine Compound 147 according to an embodiment may be synthesized by, for example, processes of Reaction Formula 10.

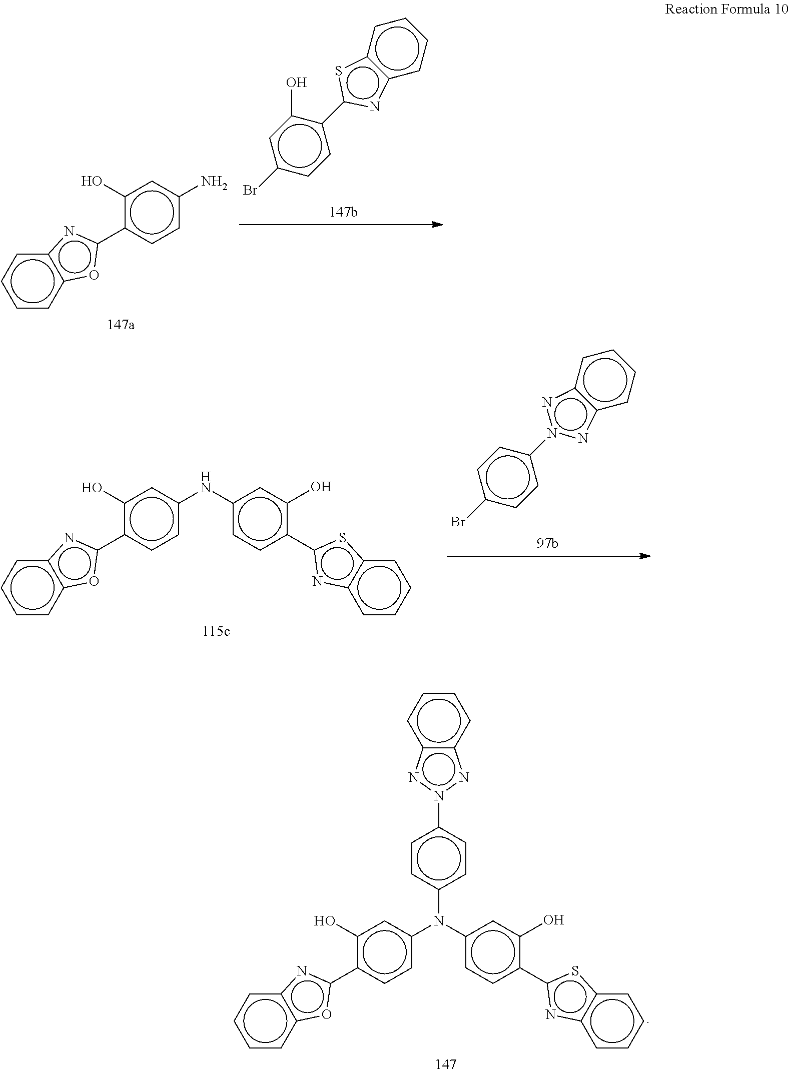

Compound 147c was obtained in substantially the same manner as in the synthesis of Compound 1, except that Intermediate Compound 147a was utilized instead of Intermediate Compound 1a and Intermediate Compound 147b was utilized instead of Intermediate Compound 1b. Compound 147 (4.8 g, yield: 75%) was then obtained in substantially the same manner as in the synthesis of Compound 1, utilizing Intermediate Compound 147c and Intermediate Compound 97c. The obtained compound was confirmed through HR mass measurement. (C₃₈H₂₄N₆O₃S M+1: 645.163)

### (11) Synthesis of Compound 144

Amine Compound 144 according to an embodiment may be synthesized by, for example, processes of Reaction Formula 11.

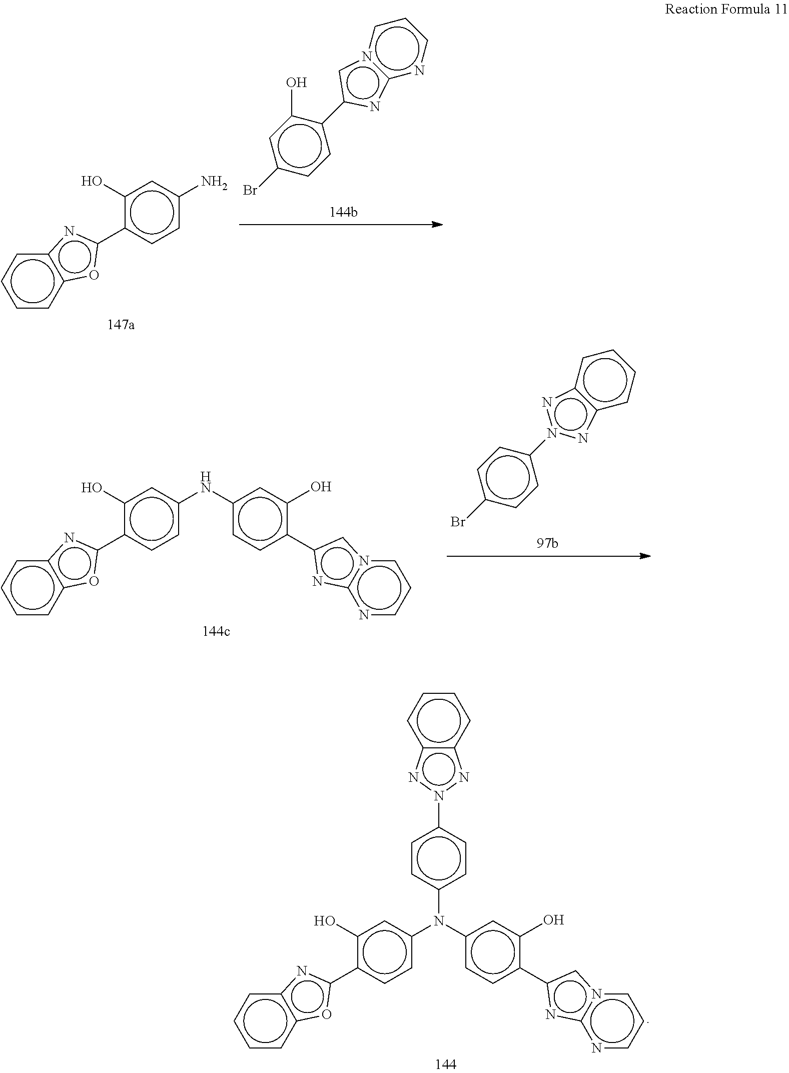

Compound 144c was obtained in substantially the same manner as in the synthesis of Compound 1, except that Intermediate Compound 147a was utilized instead of Intermediate Compound 1a and Intermediate Compound 144b was utilized instead of Intermediate Compound 1b. Compound 144 (4.8 g, yield: 76%) was then obtained in substantially the same manner as in the synthesis of Compound 1, utilizing Intermediate Compound 144c and Intermediate Compound 97c. The obtained compound was confirmed through HR mass measurement. (C₃₇H₂₄N₈O₃ M+1: 629.197)

### (12) Synthesis of Compound 143

Amine Compound 143 according to an embodiment may be synthesized by, for example, processes of Reaction Formula 12.

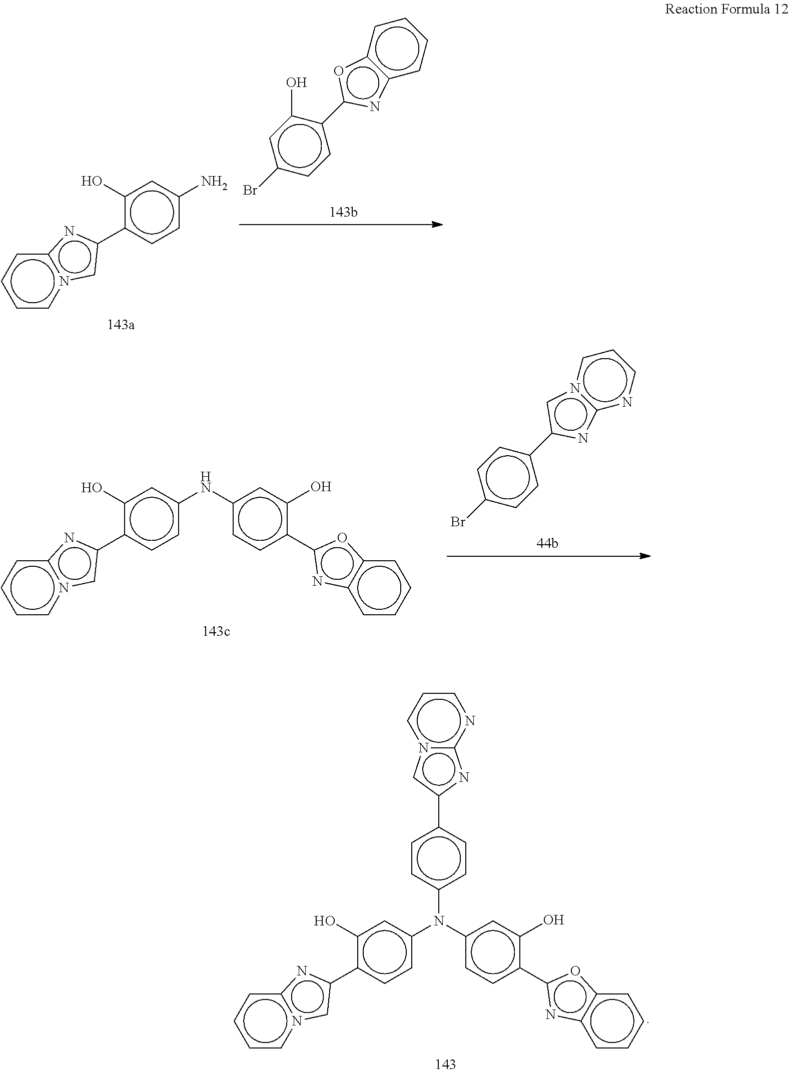

Compound 143c was obtained in substantially the same manner as in the synthesis of Compound 1, except that Intermediate Compound 143a was utilized instead of Intermediate Compound 1a and Intermediate Compound 143b was utilized instead of Intermediate Compound 1b. Compound 143 (4.3 g, yield: 70%) was then obtained in substantially the same manner as in the synthesis of Compound 1, utilizing Intermediate Compound 143c and Intermediate Compound 44b. The obtained compound was confirmed through HR mass measurement. (C₃₈H₂₅N₇O₃ M+1: 628.201)

### (13) Synthesis of Compound 184

Amine Compound 184 according to an embodiment may be synthesized by, for example, processes of Reaction Formula 13.

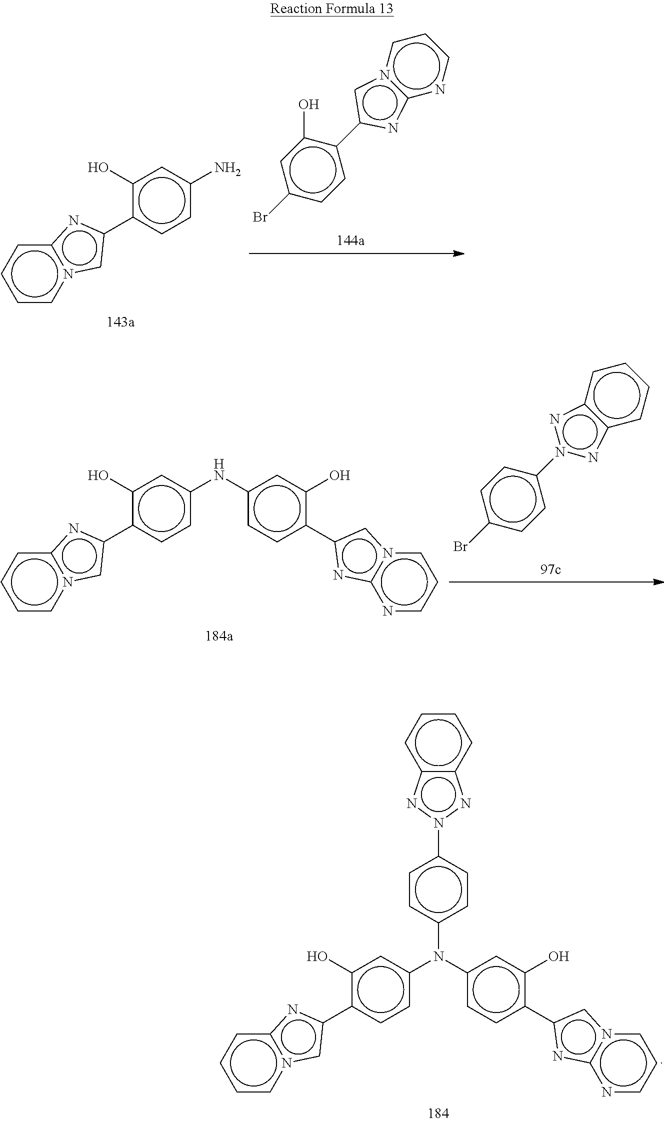

Compound 184a was obtained in substantially the same manner as in the synthesis of Compound 1, except that Intermediate Compound 143a was utilized instead of Intermediate Compound 1a and Intermediate Compound 144a was utilized instead of Intermediate Compound 1b. Compound 184 (4.5 g, yield: 73%) was then obtained in substantially the same manner as in the synthesis of Compound 1, utilizing Intermediate Compound 184a and Intermediate Compound 97c. The obtained compound was confirmed through HR mass measurement. (C₃₇H₂₅N₉O₂ M+1: 628.213)

### (14) Synthesis of Compound 169

Amine Compound 169 according to an embodiment may be synthesized by, for example, processes of Reaction Formula 14.

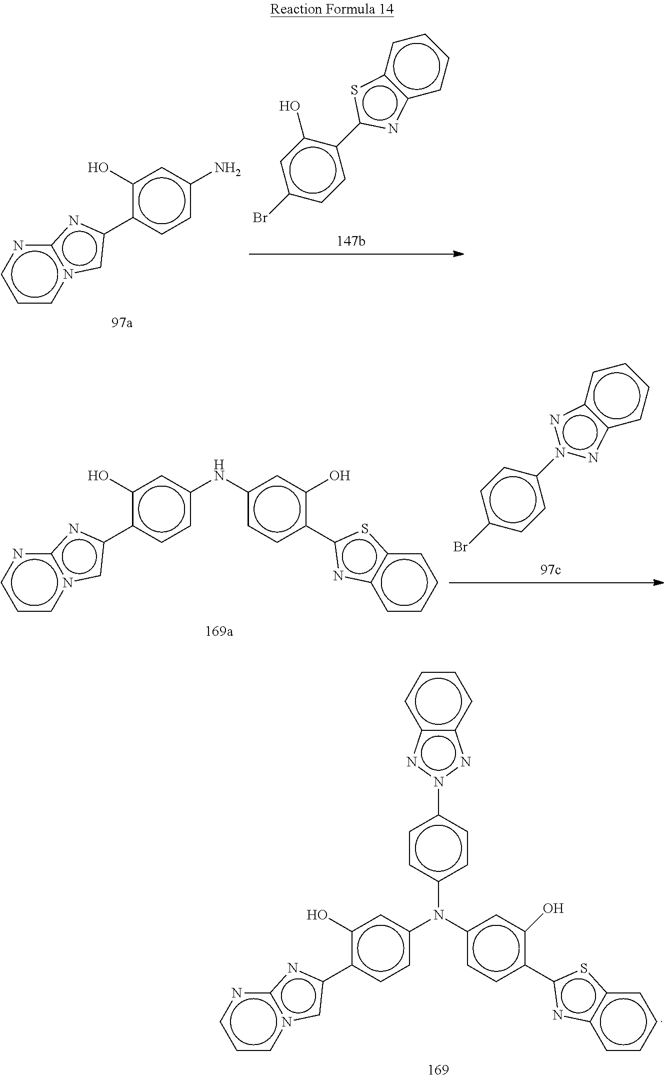

Compound 169a was obtained in substantially the same manner as in the synthesis of Compound 1, except that Intermediate Compound 97a was utilized instead of Intermediate Compound 1a and Intermediate Compound 147b was utilized instead of Intermediate Compound 1b. Compound 169 was then obtained in substantially the same manner as in the synthesis of Compound 1, utilizing Intermediate Compound 169a and Intermediate Compound 97c. The obtained compound was confirmed through HR mass measurement. (C₃₇H₂₄N₈O₂S M+1: 645.17)

### (15) Synthesis of Compound 170

Amine Compound 170 according to an embodiment may be synthesized by, for example, processes of Reaction Formula 15.

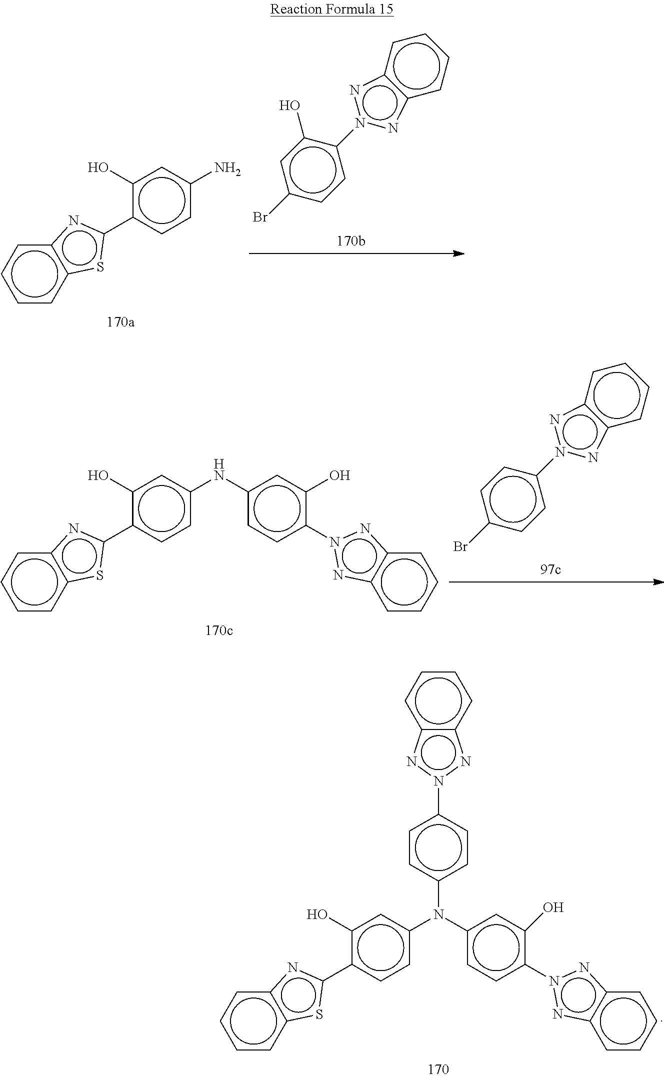

Compound 170c was obtained in substantially the same manner as in the synthesis of Compound 1, except that Intermediate Compound 170a was utilized instead of Intermediate Compound 1a and Intermediate Compound 170b was utilized instead of Intermediate Compound 1b. Compound 170 (4.5 g, yield: 71%) was then obtained in substantially the same manner as in the synthesis of Compound 1, utilizing Intermediate Compound 170c and Intermediate Compound 97c. The obtained compound was confirmed through HR mass measurement. (C₃₇H₂₄N₈O₂S M+1: 645.174)

### (16) Synthesis of Compound 246

Amine Compound 246 according to an embodiment may be synthesized by, for example, processes of Reaction Formula 16.

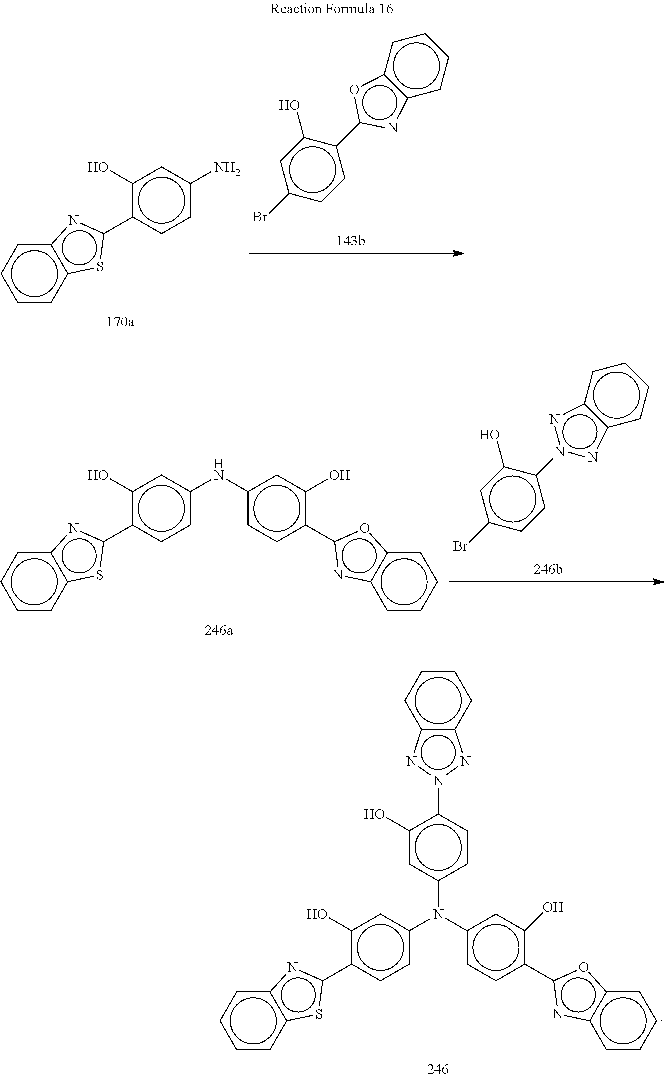

Compound 246a was obtained in substantially the same manner as in the synthesis of Compound 1, except that Intermediate Compound 170a was utilized instead of Intermediate Compound 1a and Intermediate Compound 143b was utilized instead of Intermediate Compound 1b. Compound 246 (5.0 g, yield: 76%) was then obtained in substantially the same manner as in the synthesis of Compound 1, utilizing 20 Intermediate Compound 246a and Intermediate Compound 246c. The obtained compound was confirmed through HR mass measurement. (C₃₈H₂₄N₆O₄S M+1: 661.157)

### (17) Synthesis of Compound 289

Amine Compound 289 according to an embodiment may be synthesized by, for example, processes of Reaction Formula 17.

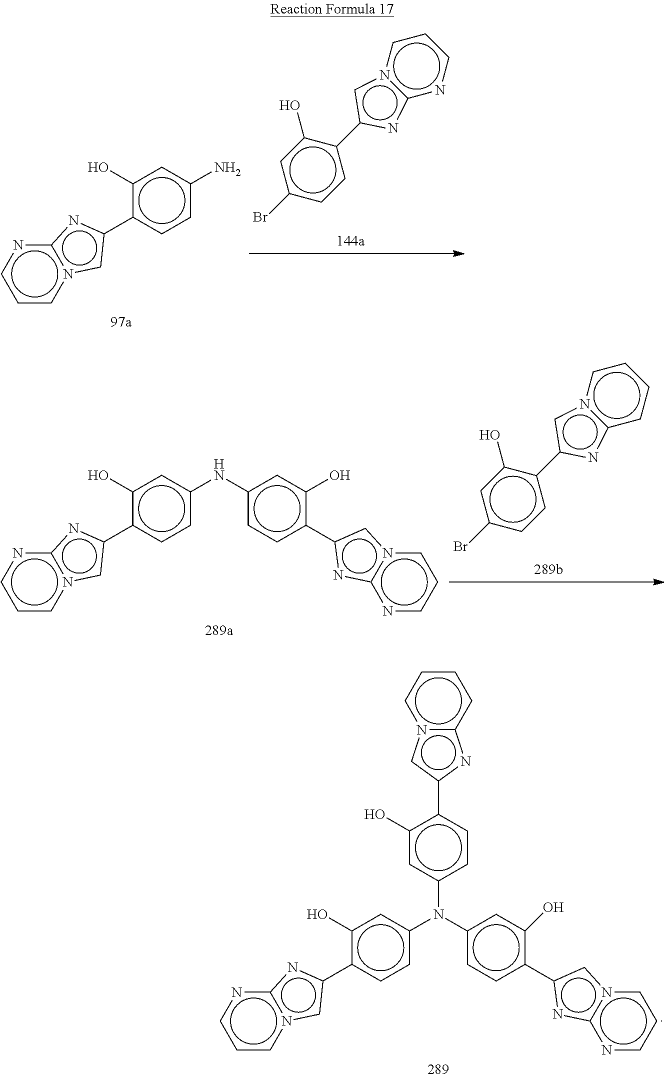

Compound 289a was obtained in substantially the same manner as in the synthesis of Compound 1, except that Intermediate Compound 97a was utilized instead of Intermediate Compound 1a and Intermediate Compound 144a was utilized instead of Intermediate Compound 1b. Compound 289 (4.7 g, yield: 74%) was then obtained in substantially the same manner as in the synthesis of Compound 1, utilizing Intermediate Compound 289a and Intermediate Compound 289b. The obtained compound was confirmed through HR mass measurement. (C₃₇H₂₅N₉O₃ M+1: 644.208)

### (18) Synthesis of Compound 148

Amine Compound 148 according to an embodiment may be synthesized by, for example, processes of Reaction Formula 18.

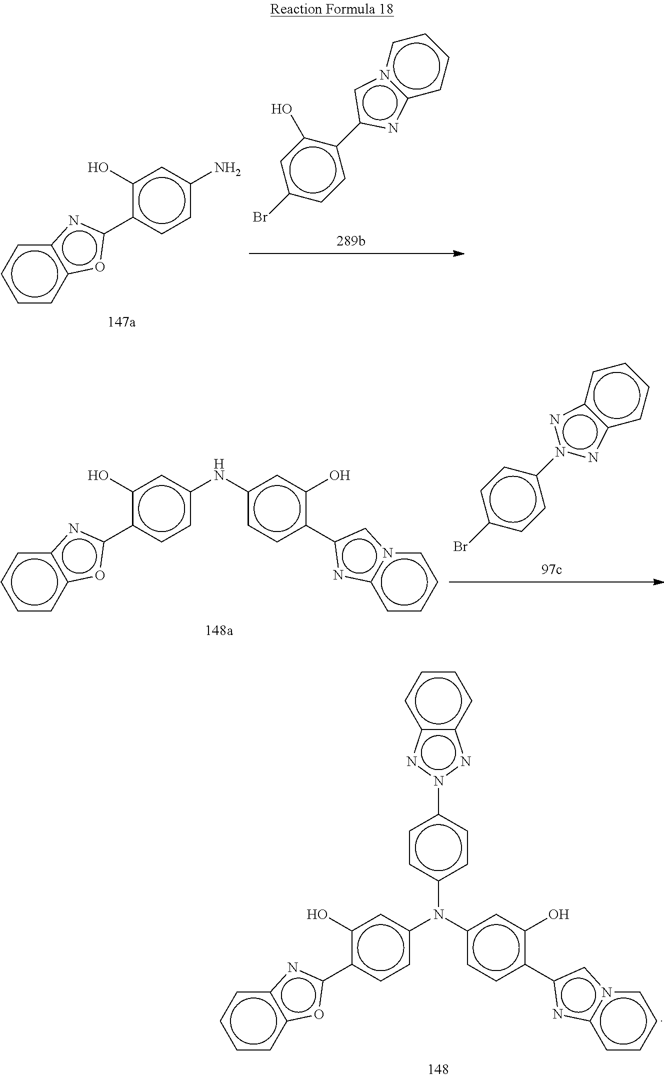

Compound 148a was obtained in substantially the same manner as in the synthesis of Compound 1, except that Intermediate Compound 147a was utilized instead of Intermediate Compound 1a and Intermediate Compound 289b was utilized instead of Intermediate Compound 1b. Compound 148 (4.5 g, yield: 73%) was then obtained in substantially the same manner as in the synthesis of Compound 1, utilizing Intermediate Compound 148a and Intermediate Compound 97c. The obtained compound was confirmed through HR mass measurement. (C₃₈H₂₅N₇O₃ M+1: 628.201)

### (19) Synthesis of Compound 400

Amine Compound 400 according to an embodiment may be synthesized by, for example, processes of Reaction Formula 19.

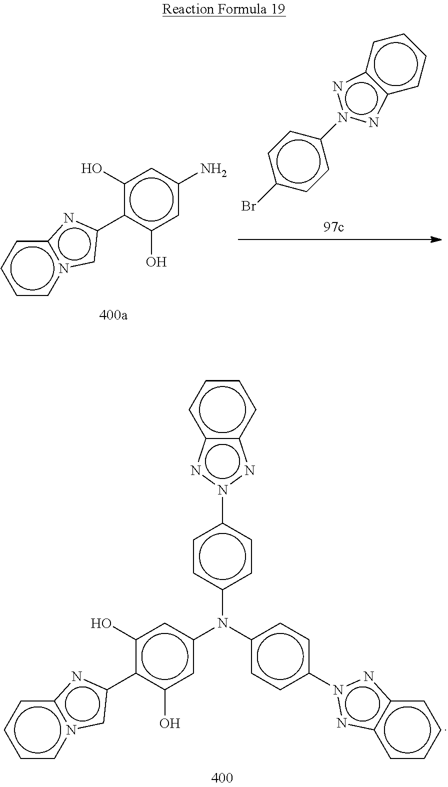

Compound 400 (4.3 g, yield: 70%) was obtained in substantially the same manner as in the synthesis of Compound 1, except that Intermediate Compound 400a was utilized instead of Intermediate Compound 1a and Intermediate Compound 97c was utilized instead of Intermediate Compound 1b. The obtained compound was confirmed through HR mass measurement. (C₃₇H₂₅N₉O₂ M+1: 628.213)

### (20) Synthesis of Compound 510

Amine Compound 510 according to an embodiment may be synthesized by, for example, processes of Reaction Formula 20.

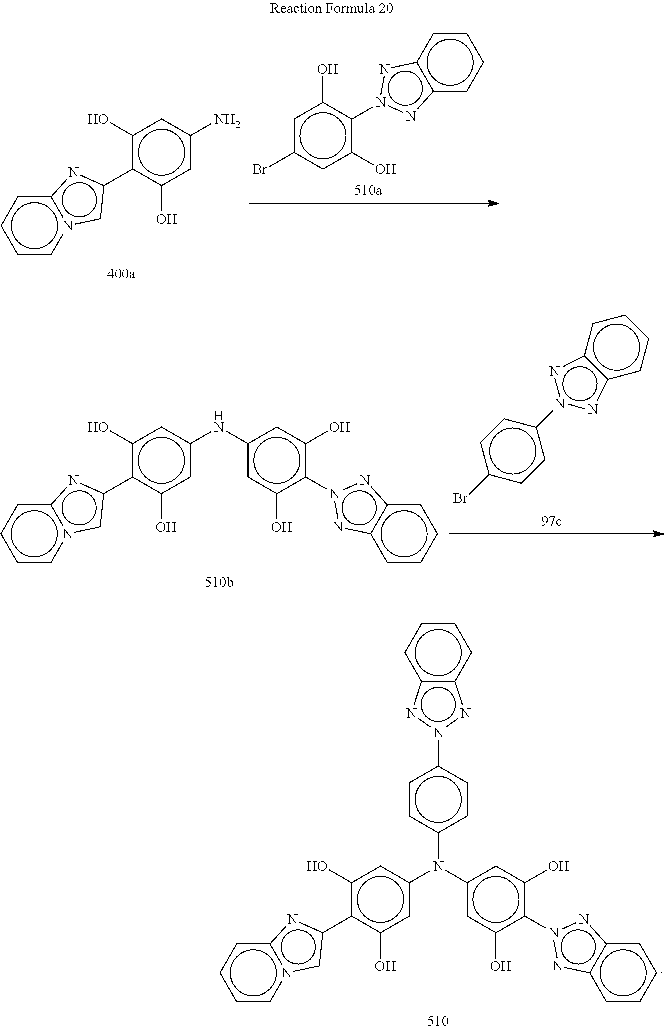

Compound 510b was obtained in substantially the same manner as in the synthesis of Compound 1, except that Intermediate Compound 400a was utilized instead of Intermediate Compound 1a and Intermediate Compound 510a was utilized instead of Intermediate Compound 1b. Compound 510 (4.6 g, yield: 70%) was then obtained in substantially the same manner as in the synthesis of Compound 1, utilizing Intermediate Compound 510b and Intermediate Compound 97c. The obtained compound was confirmed through HR mass measurement. (C₃₇H₂₅N₉O₄ M+1: 660.202)

### (21) Synthesis of Compound 532

Amine Compound 532 according to an embodiment may be synthesized by, for example, processes of Reaction Formula 21.

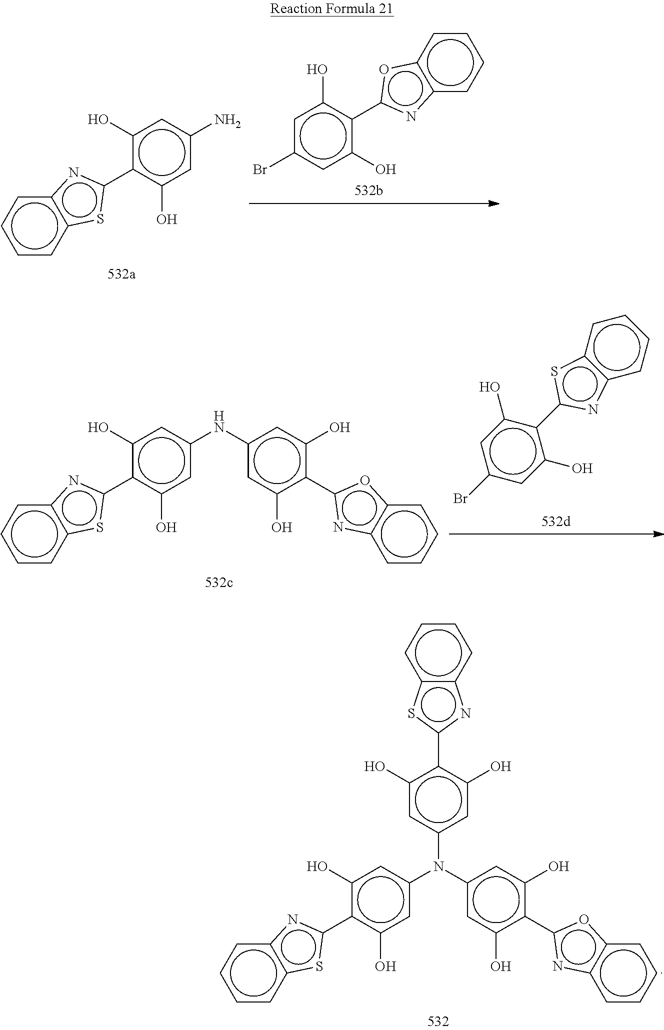

Compound 532c was obtained in substantially the same manner as in the synthesis of Compound 1, except that Intermediate Compound 532a was utilized instead of Intermediate Compound 1a and Intermediate Compound 532b was utilized instead of Intermediate Compound 1b. Compound 532 (5.1 g, yield: 70%) was then obtained in substantially the same manner as in the synthesis of Compound 1, utilizing Intermediate Compound 532c and Intermediate Compound 532d. The obtained compound was confirmed through HR mass measurement. (C₃₉H₂₄N₄O₆S₂ M+1: 725.108)

### (22) Synthesis of Compound 598

Amine Compound 598 according to an embodiment may be synthesized by, for example, processes of Reaction Formula 22.

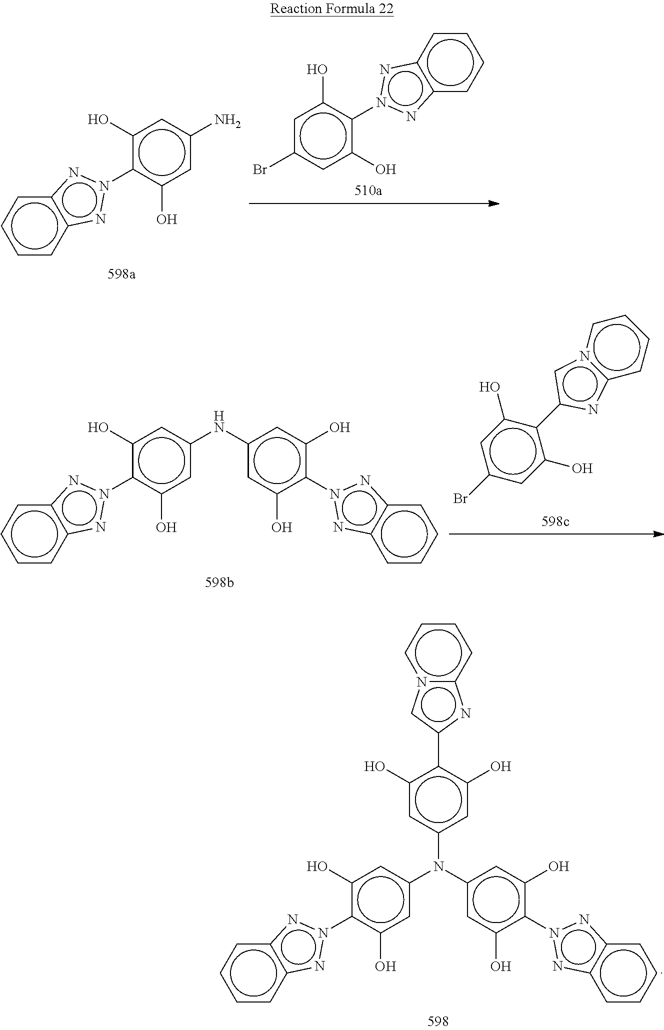

Compound 598b was obtained in substantially the same manner as in the synthesis of Compound 1, except that Intermediate Compound 598a was utilized instead of Intermediate Compound 1a and Intermediate Compound 510a was utilized instead of Intermediate Compound 1b. Compound 598 (4.5 g, yield: 65%) was then obtained in substantially the same manner as in the synthesis of Compound 1, utilizing Intermediate Compound 598b and Intermediate Compound 598c. The obtained compound was confirmed through HR mass measurement. (C₃₇H₂₅N₉O₆ M+1: 692.192)

### (23) Synthesis of Compound 715

Amine Compound 715 according to an embodiment may be synthesized by, for example, processes of Reaction Formula 23.

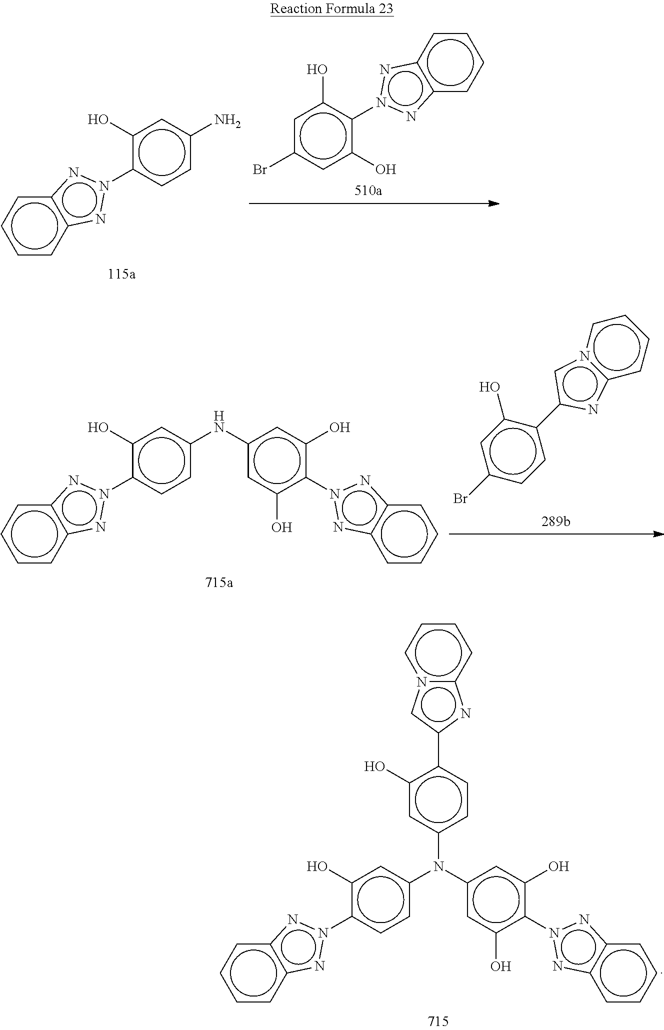

Compound 715a was obtained in substantially the same manner as in the synthesis of Compound 1, except that Intermediate Compound 115a was utilized instead of Intermediate Compound 1a and Intermediate Compound 510a was utilized instead of Intermediate Compound 1b. Compound 715 (4.6 g, yield: 70%) was then obtained in substantially the same manner as in the synthesis of Compound 1, utilizing Intermediate Compound 715a and Intermediate Compound 298b. The obtained compound was confirmed through HR mass measurement. (C₃₇H₂₅N₉O₄ M+1: 660.202)

### (24) Synthesis of Compound 772

Amine Compound 772 according to an embodiment may be synthesized by, for example, processes of Reaction Formula 24.

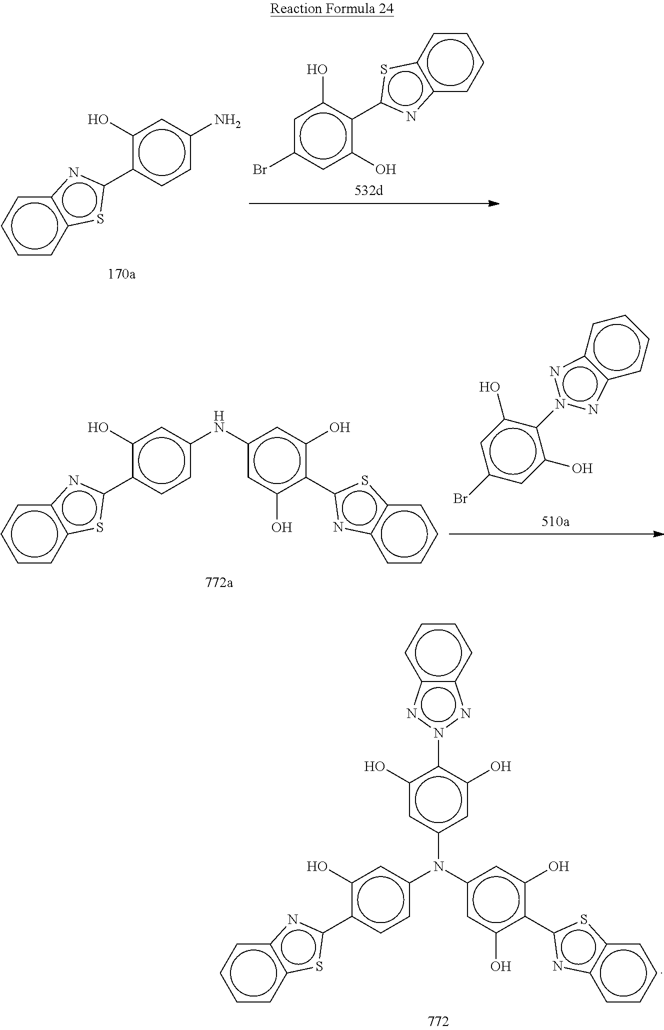

Compound 772a was obtained in substantially the same manner as in the synthesis of Compound 1, except that Intermediate Compound 170a was utilized instead of Intermediate Compound 1a and Intermediate Compound 532d was utilized instead of Intermediate Compound 1b. Compound 772 (4.6 g, yield: 65%) was then obtained in substantially the same manner as in the synthesis of Compound 1, utilizing Intermediate Compound 772a and Intermediate Compound 510a. The obtained compound was confirmed through HR mass measurement. (C₃₈H₂₄N₆O₅ S₂ M+1: 709.124)

### (25) Synthesis of Compound 882

Amine Compound 882 according to an embodiment may be synthesized by, for example, processes of Reaction Formula 25.

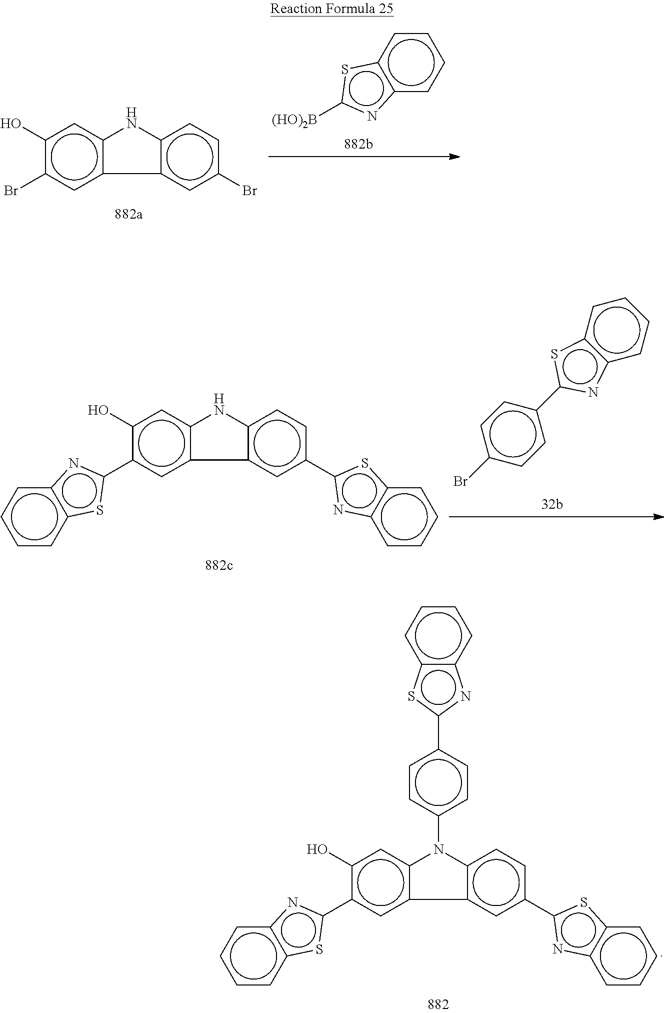

Compound 882c was obtained in substantially the same manner as in the synthesis of Compound 1, except that Intermediate Compound 882a was utilized instead of Intermediate Compound 1a and Intermediate Compound 882b was utilized instead of Intermediate Compound 1b. Compound 882 (4.6 g, yield: 71%) was then obtained in substantially the same manner as in the synthesis of Compound 1, utilizing Intermediate Compound 882c and Intermediate Compound 32b. The obtained compound was confirmed through HR mass measurement. (C₃₉H₂₂N₄OS₂ M+1: 659.095)

### (26) Synthesis of Compound 1081

Amine Compound 1081 according to an embodiment may be synthesized by, for example, processes of Reaction Formula 26.

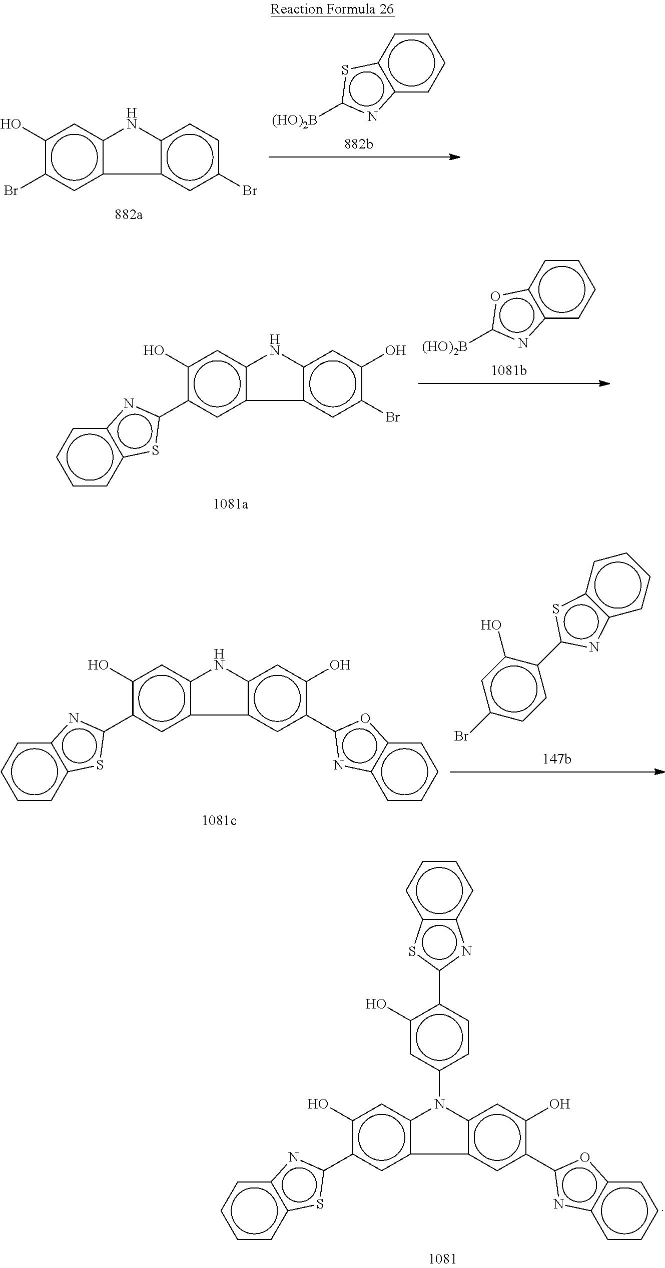

Compound 1081a was obtained in substantially the same manner as in the synthesis of Compound 1, except that Intermediate Compound 882a was utilized instead of Intermediate Compound 1a and Intermediate Compound 882b was utilized instead of Intermediate Compound 1b. Compound 1081c was then obtained in substantially the same manner as in the synthesis of Compound 1, utilizing Intermediate Compound 1081a and Intermediate Compound 1081b, and Compound 1081 (5.0 g, yield 70%) was obtained in substantially the same manner as in the synthesis of Compound 1 by adding Intermediate Compound 147b. The obtained compound was confirmed through HR mass measurement. (C₃₉H₂₂N₄O₄S₂ M+1: 675.108)

### (27) Synthesis of Compound 1343

Amine Compound 1343 according to an embodiment may be synthesized by, for example, processes of Reaction Formula 27.

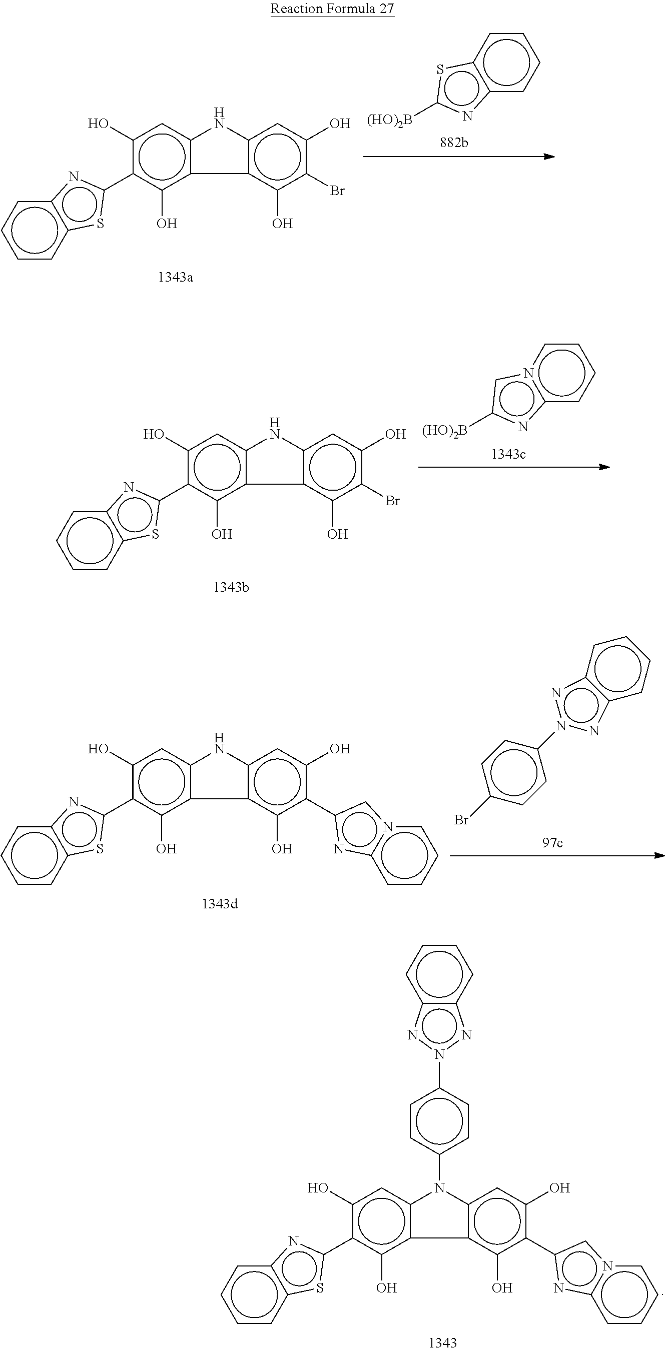

Compound 1343b was obtained in substantially the same manner as in the synthesis of Compound 1, except that Intermediate Compound 1343a was utilized instead of Intermediate Compound 1a and Intermediate Compound 882b was utilized instead of Intermediate Compound 1b. Compound 1343d was then obtained in substantially the same manner as in the synthesis of Compound 1, utilizing Intermediate Compound 1343b and Intermediate Compound 1343c, and Compound 1343 (5.0 g, yield: 74%) was obtained in substantially the same manner as in the synthesis of Compound 1 by adding Intermediate Compound 97c. The obtained compound was confirmed through HR mass measurement. (C₃₈H₂₄N₇O₄S M+1: 674.153)

### 2. Manufacture and Evaluation of Light Emitting Elements

Light emitting elements including compounds of Examples and Comparative Examples in a capping layer were evaluated utilizing a method described in more detail below. A process for manufacturing a light emitting element for evaluation is described in more detail below.

### (1) Manufacture of Light Emitting Elements

A glass substrate having a resistance of 150/cm² and patterned with ITO having a thickness of 120 nm was cut to a size of 50 mm×50 mm×0.7 mm, subjected to ultrasonic cleaning utilizing isopropyl alcohol for 5 minutes and pure water for 5 minutes, and ultraviolet irradiation for 30 minutes, and then exposed to ozone for cleaning. Thereafter, the cleaned ITO glass substrate was mounted on a vacuum deposition apparatus.

2-TNATA was vacuum deposited on an upper portion of the ITO glass substrate to form a hole injection layer having a thickness of 200 Å. 4,4′-bis[N-(1-naphthyl)-N-phenylamino]biphenyl (hereinafter, NPB) was then vacuum deposited to be 100 Å thick to form a hole transport layer.

9,10-di(naphthalen-2-yl)anthracene (hereinafter, ADN) as a blue fluorescent host and 4,4′-bis[2-(4-(N,N-diphenylamino)phenyl)vinyl]biphenyl (hereinafter, DPAVBi) as a blue fluorescent dopant were co-deposited at a weight ratio of 98:2 on the upper portion of the hole transport layer to form an emission layer having a thickness of 300 Å.

Alq₃ was deposited to be 200 Å thick on an upper portion of the emission layer to form an electron transport layer, and Yb, which is an alkali metal halide, was deposited to be 20 Å thick on an upper portion of the electron transport layer to form an electron injection layer. Mg:Ag=9:1 was vacuum deposited to be 100 Å thick on the electron injection layer to form a second electrode. Example compounds or Comparative Example compounds were vacuum deposited to be 800 Å thick on the second electrode to form a capping layer.

The compounds utilized for the hole injection layer, the hole transport layer, and the emission layer of light emitting elements are as follows.

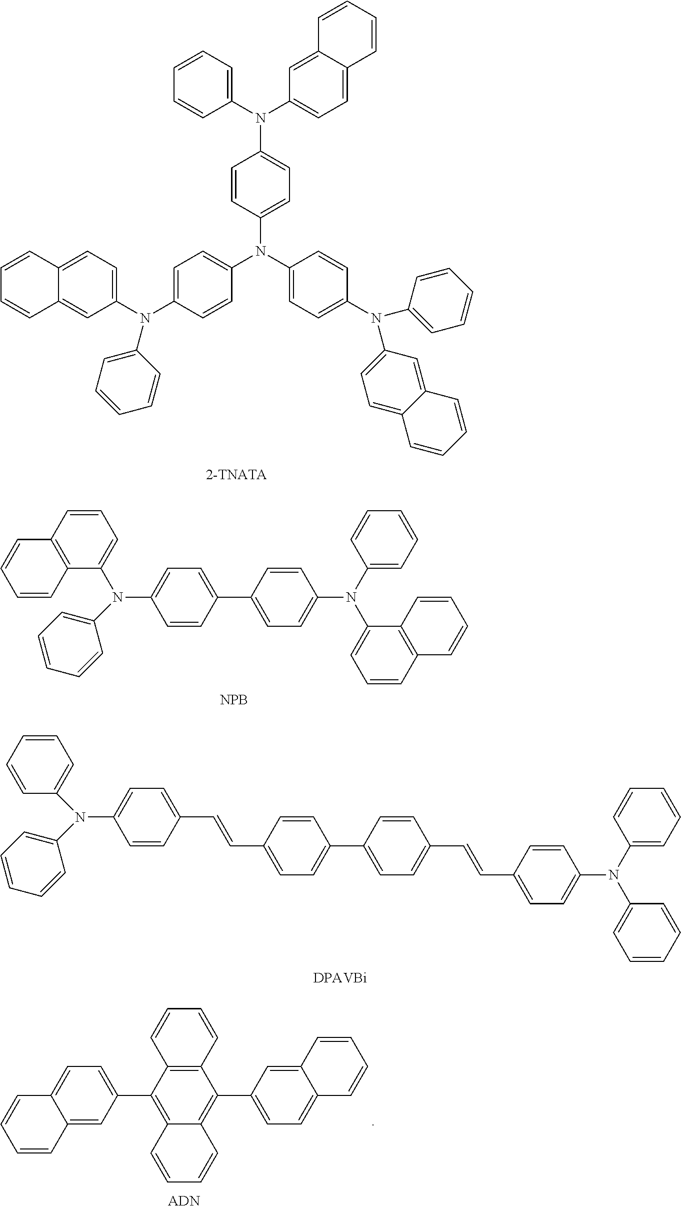

Comparative Example compounds utilized to manufacture light emitting elements of Comparative Examples are as follows.

### Comparative Example Compounds

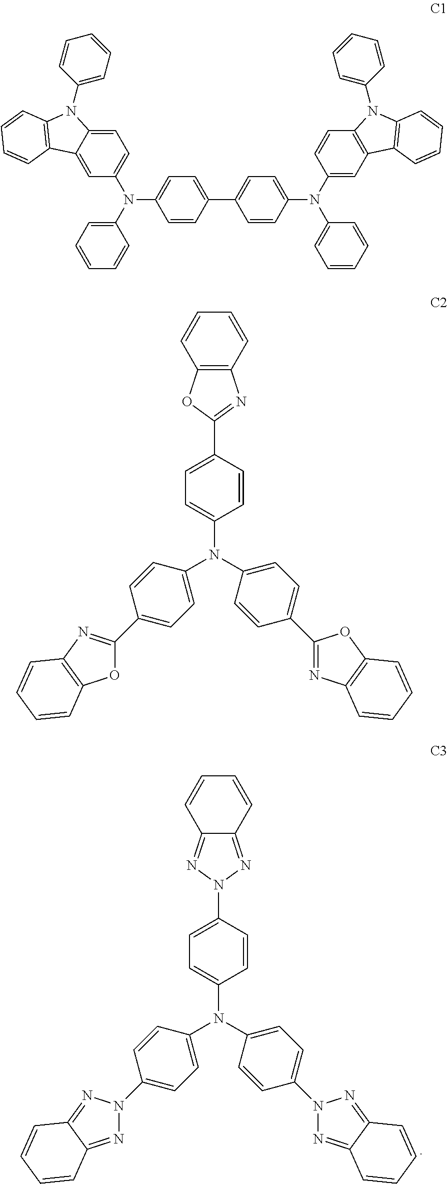 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc2oc(-c3ccc(N(c4ccc(-c5nc6ccccc6o5)cc4)c4ccc(-c5nc6ccccc6o5)cc4)cc3)nc2c1 ; c1ccc2nn(-c3ccc(N(c4ccc(-n5nc6ccccc6n5)cc4)c4ccc(-n5nc6ccccc6n5)cc4)cc3)nc2c1]

Capping layer materials utilized in the manufactured Examples and Comparative Examples are shown in Table 2.

TABLE 2

| Item | Capping layer compound |
|---|---|
| Example 1 | 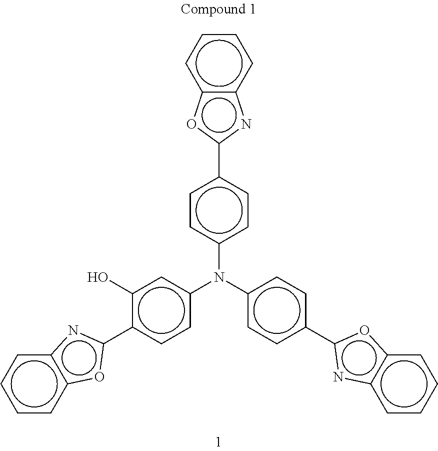 |
| Example 2 | 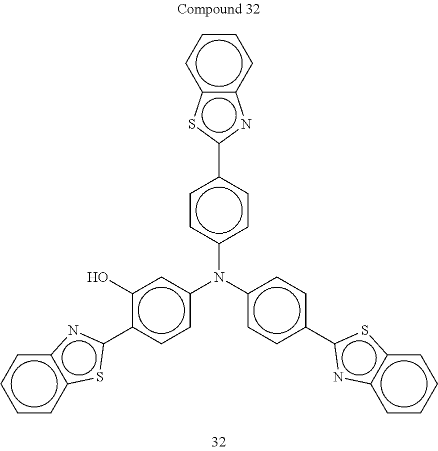 |
| Example 3 | 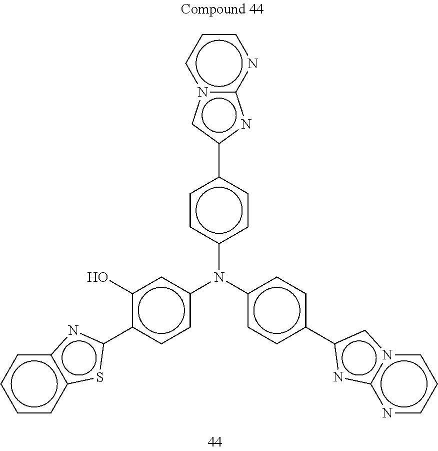 |
| Example 4 | 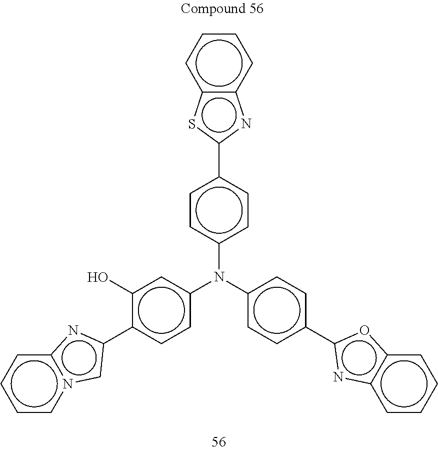 |
| Example 5 | 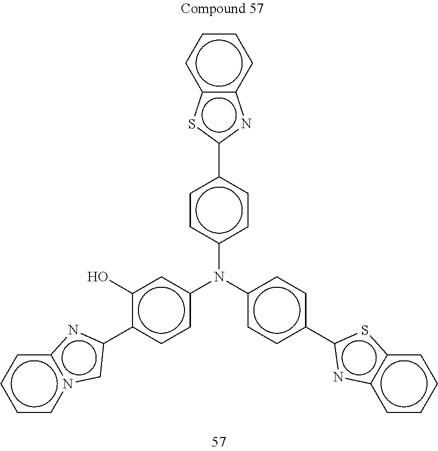 |
| Example 6 | 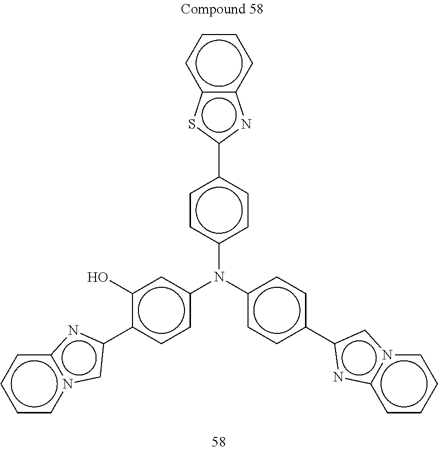 |
| Example 7 | 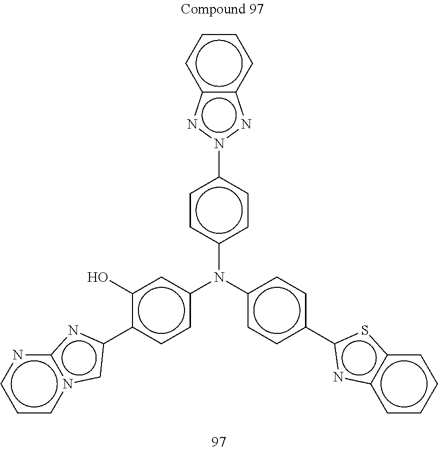 |
| Example 8 | 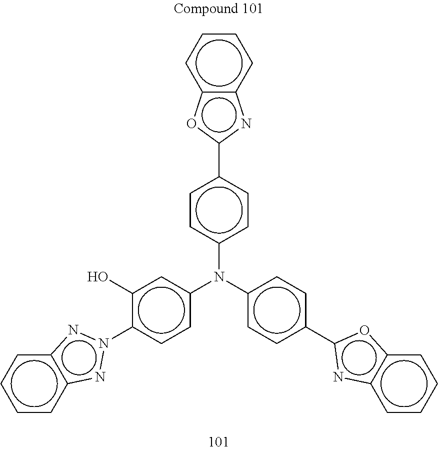 |
| Example 9 | 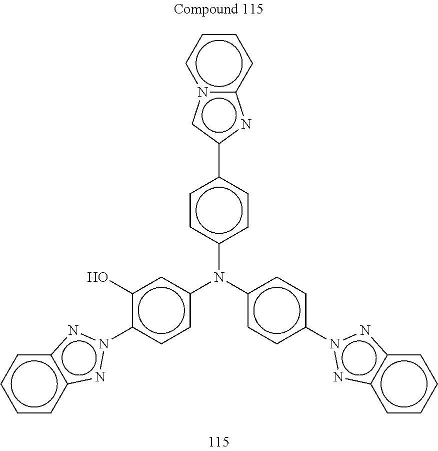 |
| Example 10 | 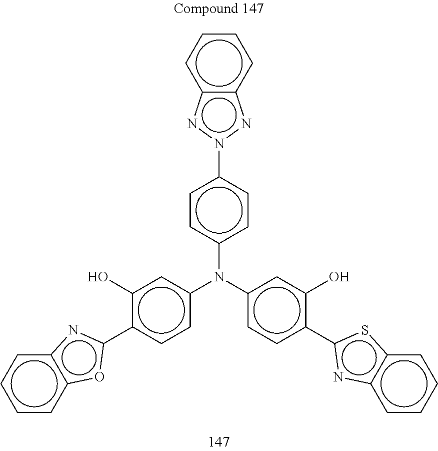 |
| Example 11 | 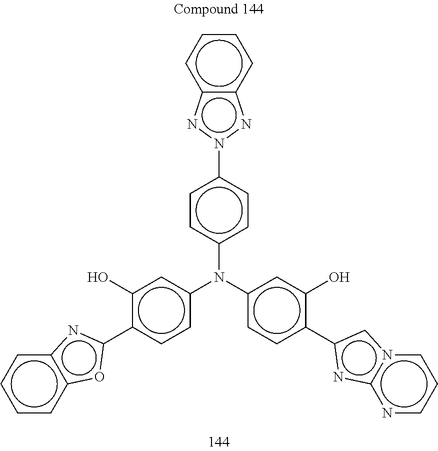 |
| Example 12 | 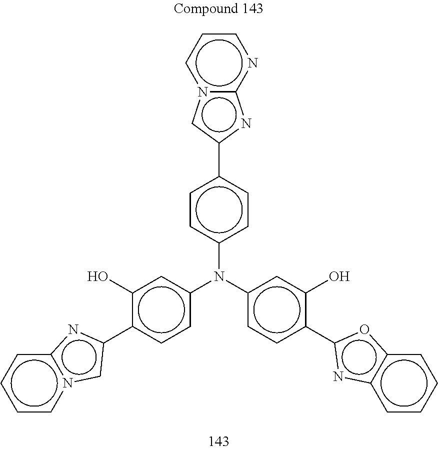 |
| Example 13 | 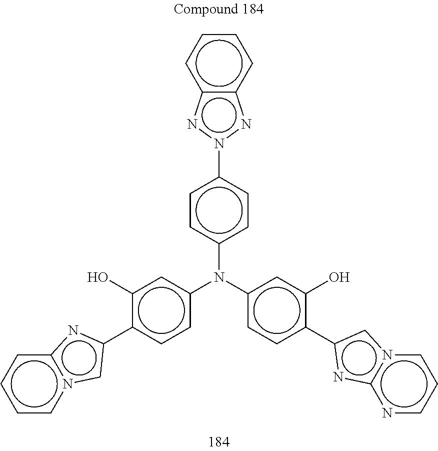 |
| Example 14 | 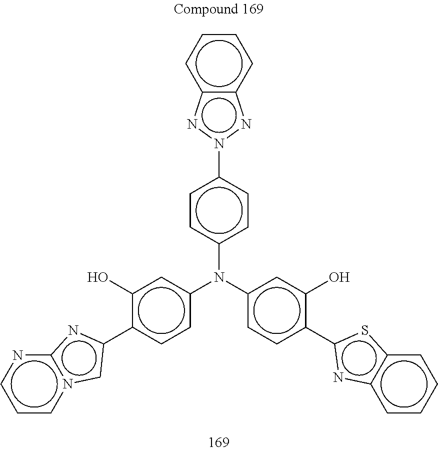 |
| Example 15 | 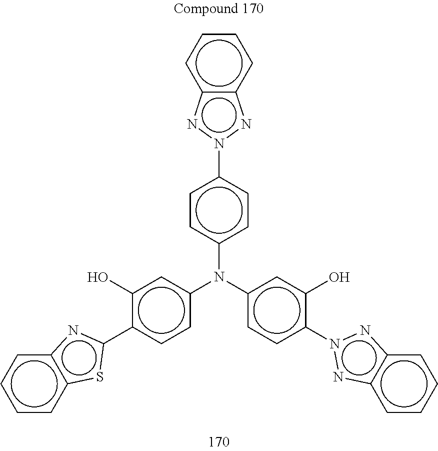 |
| Example 16 | 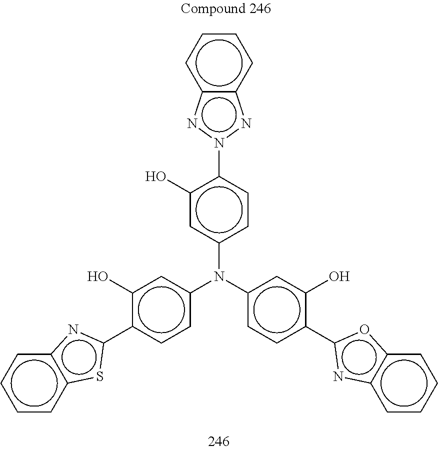 |
| Example 17 | 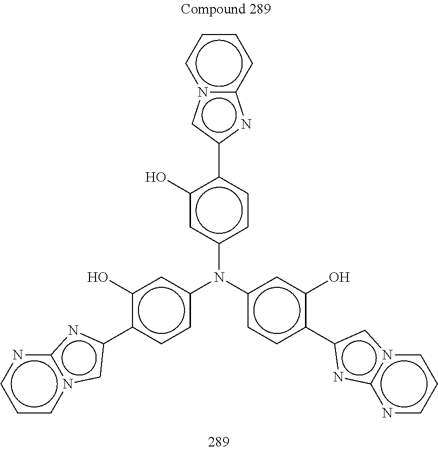 |
| Example 18 | 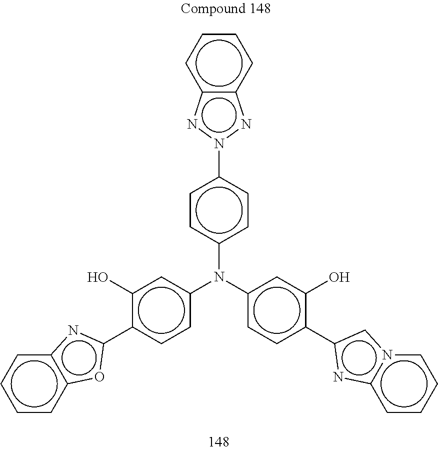 |
| Example 19 | 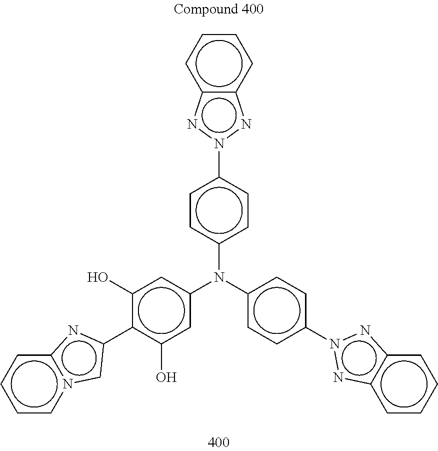 |
| Example 20 | 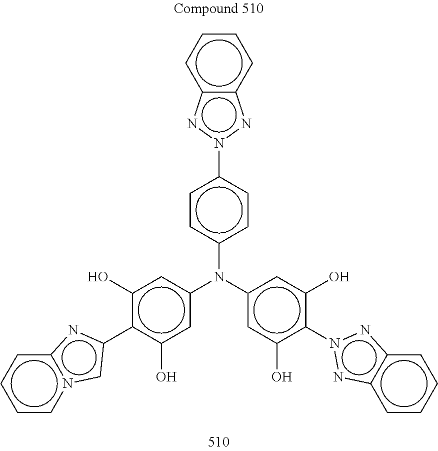 |
| Example 21 | 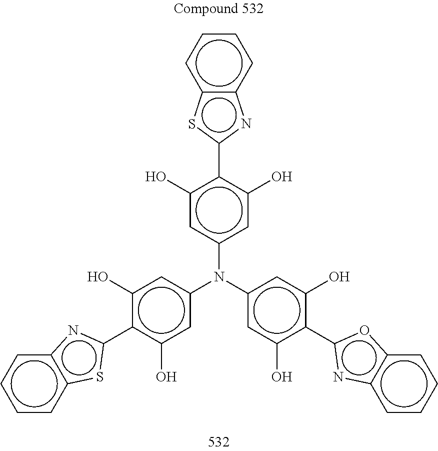 |
| Example 22 | 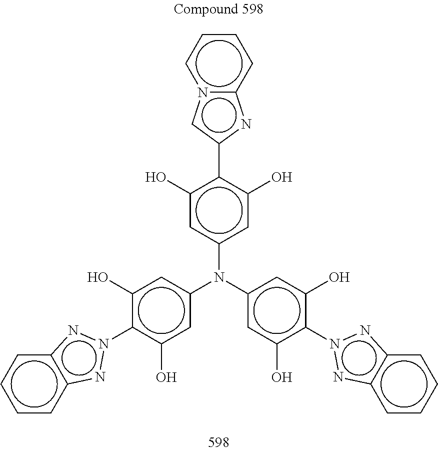 |
| Example 23 | 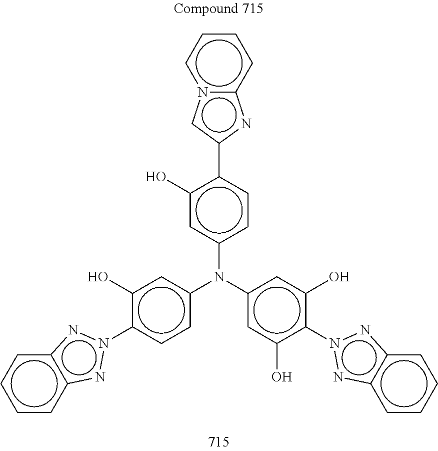 |
| Example 24 | 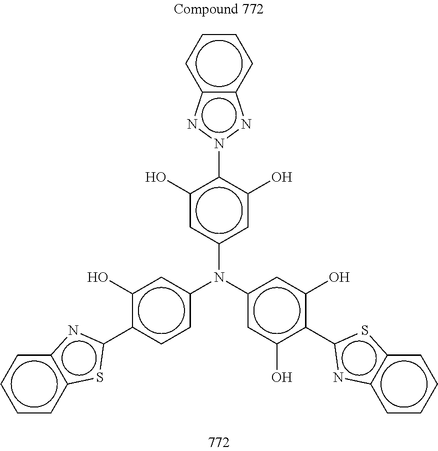 |
| Example 25 | 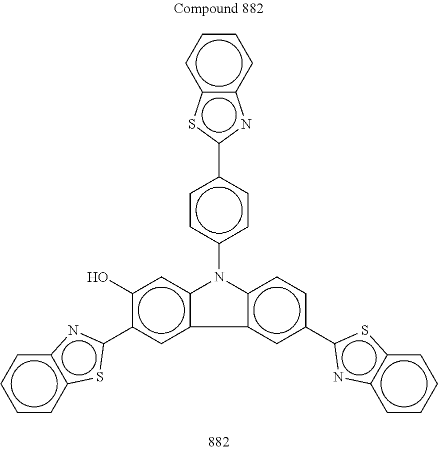 |
| Example 26 | 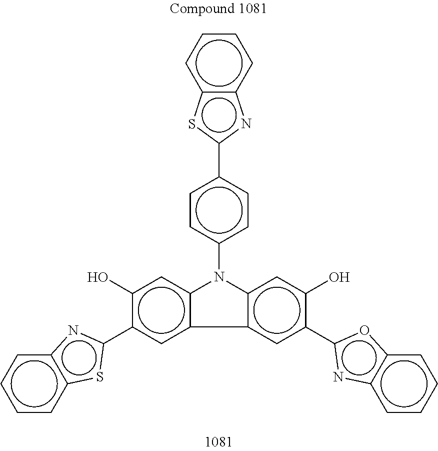 |
| Example 27 | 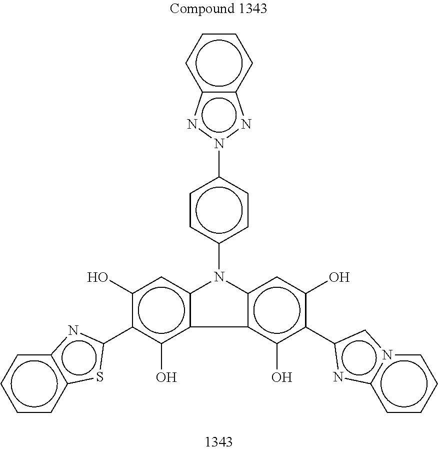 |
| Comparative | C1 |
| Example 1 | |
| Comparative | C2 |
| Example 2 | |
| Comparative | C3 |
| Example 3 | |

### (2) Evaluation of Light Emitting Elements

Table 3 shows results of evaluation on light emitting elements for Examples 1 to 27 and Comparative Examples 1 to 3. In Table 3, driving voltage, luminance at a 20 current density of 50 mA/cm², and luminous efficiency of each of the manufactured light emitting elements are compared and shown.

TABLE 3

| | Driving voltage | Luminance | Efficiency | Emitted |
|---|---|---|---|---|
| Item | (V) | (cd/m²) | (cd/A) | color |
| Comparative | 4.75 | 2645 | 5.29 | Blue |
| Example 1 | | | | |
| Comparative | 4.77 | 2670 | 5.34 | Blue |
| Example 2 | | | | |
| Comparative | 4.80 | 2705 | 5.41 | Blue |
| Example 3 | | | | |
| Example 1 | 4.75 | 2975 | 5.95 | Blue |
| Example 2 | 4.72 | 3055 | 6.11 | Blue |
| Example 3 | 4.75 | 3110 | 6.22 | Blue |
| Example 4 | 4.72 | 3070 | 6.14 | Blue |
| Example 5 | 4.72 | 3060 | 6.12 | Blue |
| Example 6 | 4.72 | 3060 | 6.12 | Blue |
| Example 7 | 4.72 | 3070 | 6.14 | Blue |
| Example 8 | 4.72 | 3105 | 6.21 | Blue |
| Example 9 | 4.72 | 3090 | 6.18 | Blue |
| Example 10 | 4.72 | 3045 | 6.09 | Blue |
| Example 11 | 4.72 | 3115 | 6.23 | Blue |
| Example 12 | 4.72 | 3120 | 6.24 | Blue |
| Example 13 | 4.72 | 3045 | 6.09 | Blue |
| Example 14 | 4.72 | 3115 | 6.23 | Blue |
| Example 15 | 4.72 | 3115 | 6.23 | Blue |
| Example 16 | 4.72 | 3045 | 6.09 | Blue |
| Example 17 | 4.72 | 3115 | 6.23 | Blue |
| Example 18 | 4.72 | 3110 | 6.22 | Blue |
| Example 19 | 4.72 | 3140 | 6.28 | Blue |
| Example 20 | 4.72 | 3005 | 6.01 | Blue |
| Example 21 | 4.72 | 3010 | 6.02 | Blue |
| Example 22 | 4.72 | 3020 | 6.04 | Blue |
| Example 23 | 4.72 | 3020 | 6.04 | Blue |
| Example 24 | 4.72 | 2985 | 5.97 | Blue |
| Example 25 | 4.72 | 2990 | 5.98 | Blue |
| Example 26 | 4.72 | 3115 | 6.23 | Blue |
| Example 27 | 4.72 | 3105 | 6.21 | Blue |

Referring to the results in Table 3, Examples 1 to 27 of the present disclosure exhibited high luminance and high efficiency element characteristics as compared with Comparative Examples 1 to 3. It is seen that the light emitting elements of Examples 1 to 27 and Comparative Examples 1 to 3 emit blue light, and in the blue light emitting region, the Examples exhibit excellent or suitable efficiency and increased luminance characteristics as compared with Comparative Examples. Without wanting to be bound by theory, it is believed that because Example compounds include at least one hydroxy to allow amine compound molecules to be arranged through a hydrogen bond, planarity of Example compounds in a capping layer is increased. For example, Example compounds are arranged to increase the planarity in the capping layer, thereby exhibiting excellent or suitable light extraction. Without wanting to be bound by theory, it is believed that the amine compound according to an embodiment that is included in the capping layer contributes to improvement of luminous efficiency and luminance of a light emitting element.

An amine compound according to an embodiment has a structure of a benzoazole derivative, and includes at least one hydroxy group, and may thus exhibit excellent or suitable light extraction efficiency. A light emitting element according to an embodiment includes the amine compound according to an embodiment in the capping layer, and may thus exhibit improved light emitting element characteristics such as a high luminance and a high efficiency.

A light emitting element according to an embodiment includes an amine compound according to an embodiment, and may thus exhibit high efficiency and high luminance characteristics.

An amine compound according to an embodiment may be utilized as a material for achieving improved light emitting element characteristics such as high efficiency and high luminance.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting device or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present disclosure as defined by the following claims and equivalents thereof.

What is claimed is:

1. A light emitting element comprising:
a first electrode;
a second electrode on the first electrode;
a functional layer between the first electrode and the second electrode; and
a capping layer on the second electrode and comprising an amine compound represented by Formula 1: 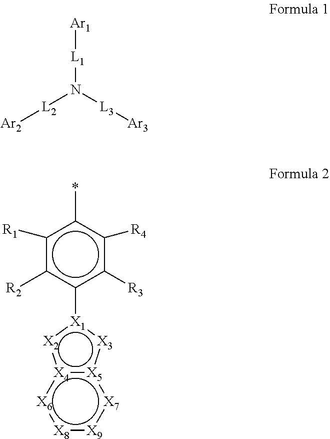
wherein in Formula 1,
*-L₁-Ar₁, *-L₂-Ar₂, and *-L₃-Ar₃ are each independently represented by Formula 2; and
in Formula 2,
R₁ to R₄ are each independently a hydroxy group or a hydrogen atom, or bonded to an adjacent group to form a single bond,
X₁ to X₉ are each independently CH, C, N, O, or S, and
at least one of *-L₁-Ar₁, *-L₂-Ar₂, or *-L₃-Ar₃ comprises the hydroxy group.

2. The light emitting element of claim 1, wherein Formula 2 is represented by any one selected from among I-1 to I-3, wherein in Formula 1, *-L₁-Ar₁, *-L₂-Ar₂, and *-L₃-Ar₃ are not all I-1: 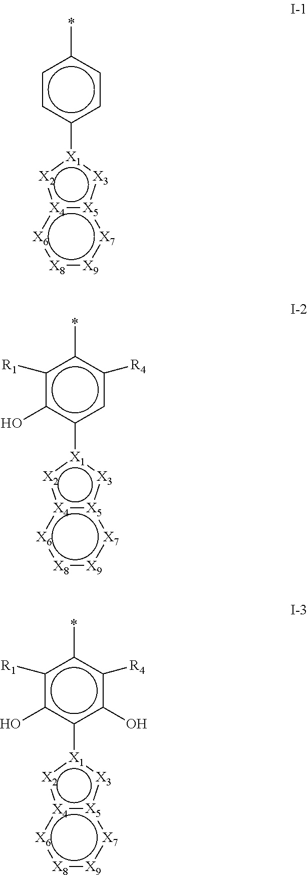
wherein in I-2 and I-3, R₁ and R₄ are each independently a hydrogen atom, or bonded to an adjacent group to form a single bond, and
in I-1 to I-3, X₁ to X₉ are the same as defined in Formula 2.

3. The light emitting element of claim 1, wherein Formula 2 is represented by any one selected from among I-4 to I-8: 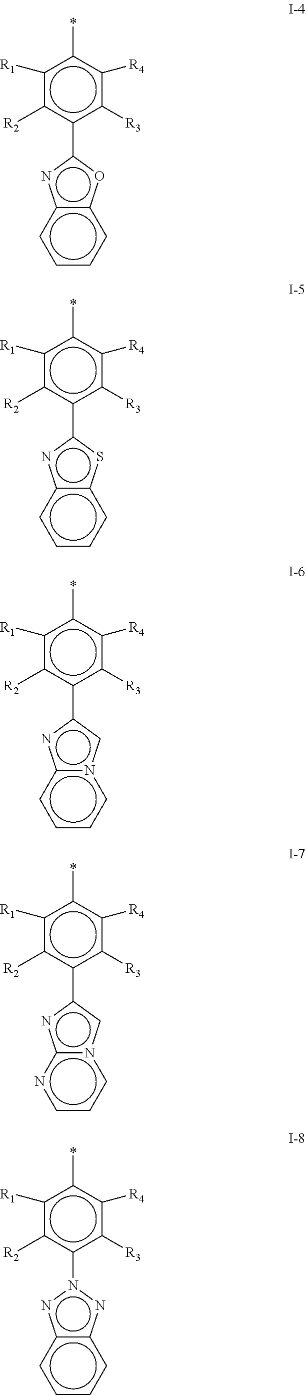
wherein in I-4 to I-8, R₁ to R₄ are the same as defined in Formula 2.

4. The light emitting element of claim 1, wherein in Formula 2, at least one selected from among X₁ to X₃ is N.

5. The light emitting element of claim 1, wherein Formula 1 is represented by Formula 1-1: 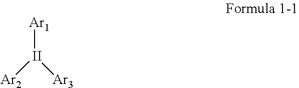
wherein in Formula 1-1, II is represented by II-A or II-B: 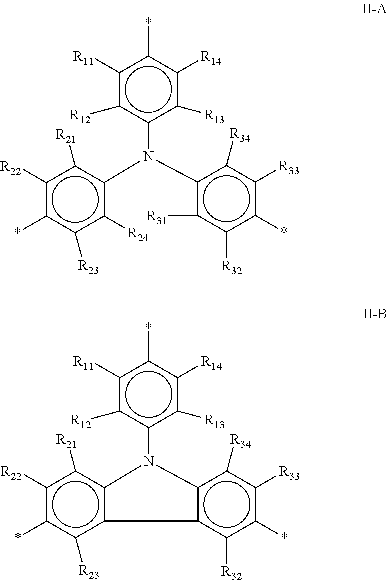
wherein in II-A, at least one selected from among R₁₁ to R₁₄, R₂₁ to R₂₄, and R₃₁ to R₃₄ is hydroxy and the R₁₁ to R₁₄, R₂₁ to R₂₄, and R₃₁ to R₃₄ that are not hydroxy are hydrogen atoms, and
in II-B, at least one selected from among R₁₁ to R₁₄, R₂₁ to R₂₃, and R₃₂ to R₃₄ is hydroxy and the R₁₁ to R₁₄, R₂₁ to R₂₃, and R₃₂ to R₃₄ that are not hydroxy are hydrogen atoms.

6. The light emitting element of claim 5, wherein in Formula 1-1, II is represented by any one selected from among II-1 to II-16: 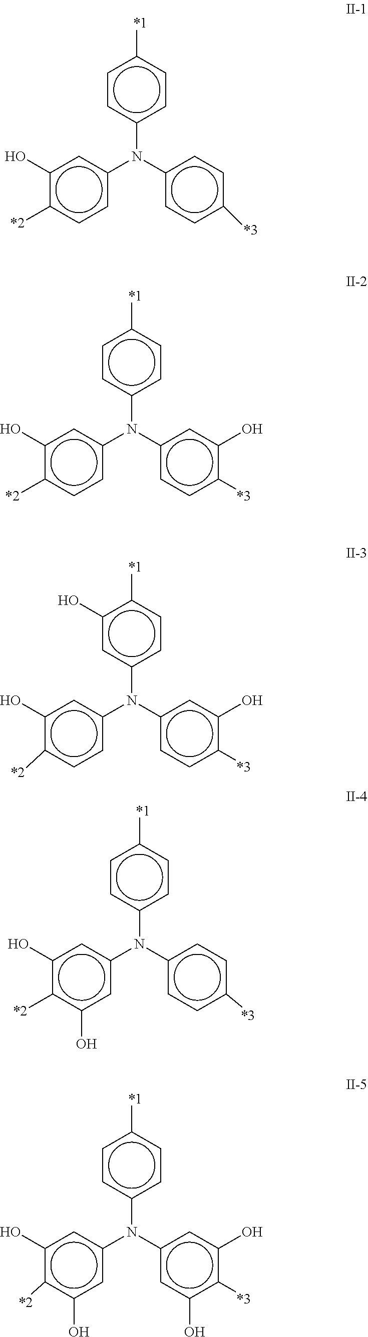 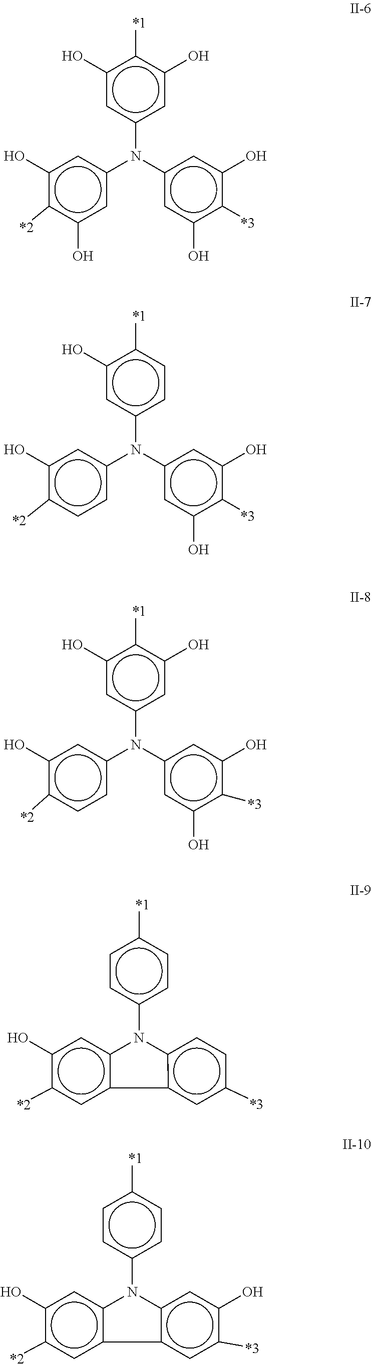 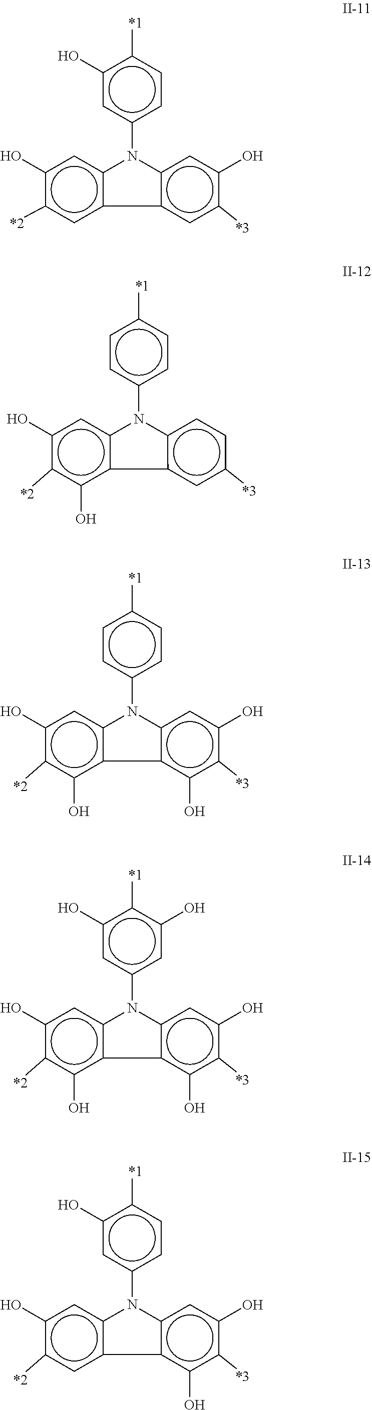 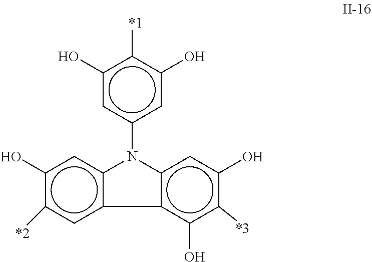
wherein in II-1 to II-16, *1 is a portion to which Ar₁ is bonded, *2 is a portion to which Ar₂ is bonded, and *3 is a portion to which Ar₃ is bonded.

7. The light emitting element of claim 5, wherein in Formula 1-1, Ar₁ to Ar₃ are each independently represented by any one selected from among III-1 to III-5:

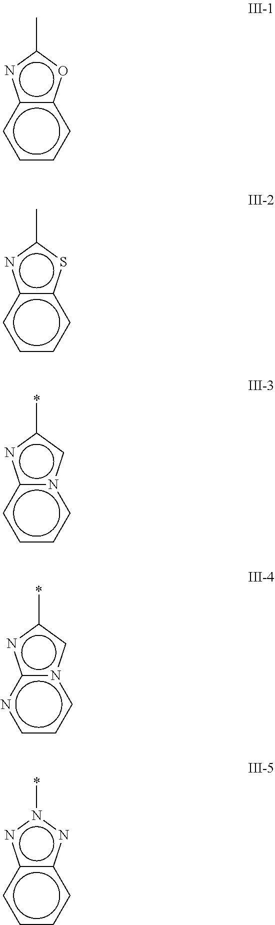

8. The light emitting element of claim 1, wherein the functional layer comprises an emission layer, a hole transport region between the first electrode and the emission layer, and an electron transport region between the emission layer and the second electrode.

9. The light emitting element of claim 8, wherein the emission layer comprises a compound represented by Formula E-1: 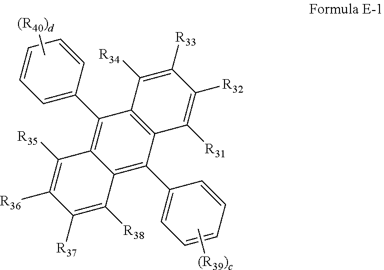
wherein in Formula E-1,
c and d are each independently an integer from 0 to 5, and
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

10. The light emitting element of claim 5, wherein the amine compound is represented by any one selected from among compounds below:

| Compound | | |
|---|---|---|
| classification | II | [Ar₂/Ar₁/Ar₃] |
| 1 | II-1 | [III-1/III-1/III-1] |
| 2 | II-1 | [III-1/III-1/III-2] |
| 3 | II-1 | [III-1/III-1/III-3] |
| 4 | II-1 | [III-1/III-1/III-4] |
| 5 | II-1 | [III-1/III-1/III-5] |
| 6 | II-1 | [III-1/III-2/III-1] |
| 7 | II-1 | [III-1/III-2/III-2] |
| 8 | II-1 | [III-1/III-2/III-3] |
| 9 | II-1 | [III-1/III-2/III-4] |
| 10 | II-1 | [III-1/III-2/III-5] |
| 11 | II-1 | [III-1/III-3/III-1] |
| 12 | II-1 | [III-1/III-3/III-2] |
| 13 | II-1 | [III-1/III-3/III-3] |
| 14 | II-1 | [III-1/III-3/III-4] |
| 15 | II-1 | [III-1/III-3/III-5] |
| 16 | II-1 | [III-1/III-4/III-1] |
| 17 | II-1 | [III-1/III-4/III-2] |
| 18 | II-1 | [III-1/III-4/III-3] |
| 19 | II-1 | [III-1/III-4/III-4] |
| 20 | II-1 | [III-1/III-4/III-5] |
| 21 | II-1 | [III-1/III-5/III-1] |
| 22 | II-1 | [III-1/III-5/III-2] |
| 23 | II-1 | [III-1/III-5/III-3] |
| 24 | II-1 | [III-1/III-5/III-4] |
| 25 | II-1 | [III-1/III-5/III-5] |
| 26 | II-1 | [III-2/III-1/III-1] |
| 27 | II-1 | [III-2/III-1/III-2] |
| 28 | II-1 | [III-2/III-1/III-3] |
| 29 | II-1 | [III-2/III-1/III-4] |
| 30 | II-1 | [III-2/III-1/III-5] |
| 31 | II-1 | [III-2/III-2/III-1] |
| 32 | II-1 | [III-2/III-2/III-2] |
| 33 | II-1 | [III-2/III-2/III-3] |
| 34 | II-1 | [III-2/III-2/III-4] |
| 35 | II-1 | [III-2/III-2/III-5] |
| 36 | II-1 | [III-2/III-3/III-1] |
| 37 | II-1 | [III-2/III-3/III-2] |
| 38 | II-1 | [III-2/III-3/III-3] |
| 39 | II-1 | [III-2/III-3/III-4] |
| 40 | II-1 | [III-2/III-3/III-5] |
| 41 | II-1 | [III-2/III-4/III-1] |
| 42 | II-1 | [III-2/III-4/III-2] |
| 43 | II-1 | [III-2/III-4/III-3] |
| 44 | II-1 | [III-2/III-4/III-4] |
| 45 | II-1 | [III-2/III-4/III-5] |
| 46 | II-1 | [III-2/III-5/III-1] |
| 47 | II-1 | [III-2/III-5/III-2] |
| 48 | II-1 | [III-2/III-5/III-3] |
| 49 | II-1 | [III-2/III-5/III-4] |
| 50 | II-1 | [III-2/III-5/III-5] |
| 51 | II-1 | [III-3/III-1/III-1] |
| 52 | II-1 | [III-3/III-1/III-2] |
| 53 | II-1 | [III-3/III-1/III-3] |
| 54 | II-1 | [III-3/III-1/III-4] |
| 55 | II-1 | [III-3/III-1/III-5] |
| 56 | II-1 | [III-3/III-2/III-1] |
| 57 | II-1 | [III-3/III-2/III-2] |
| 58 | II-1 | [III-3/III-2/III-3] |
| 59 | II-1 | [III-3/III-2/III-4] |
| 60 | II-1 | [III-3/III-2/III-5] |
| 61 | II-1 | [III-3/III-3/III-1] |
| 62 | II-1 | [III-3/III-3/III-2] |
| 63 | II-1 | [III-3/III-3/III-3] |
| 64 | II-1 | [III-3/III-3/III-4] |
| 65 | II-1 | [III-3/III-3/III-5] |
| 66 | II-1 | [III-3/III-4/III-1] |
| 67 | II-1 | [III-3/III-4/III-2] |
| 68 | II-1 | [III-3/III-4/III-3] |
| 69 | II-1 | [III-3/III-4/III-4] |
| 70 | II-1 | [III-3/III-4/III-5] |
| 71 | II-1 | [III-3/III-5/III-1] |
| 72 | II-1 | [III-3/III-5/III-2] |
| 73 | II-1 | [III-3/III-5/III-3] |
| 74 | II-1 | [III-3/III-5/III-4] |
| 75 | II-1 | [III-3/III-5/III-5] |
| 76 | II-1 | [III-4/III-1/III-1] |
| 77 | II-1 | [III-4/III-1/III-2] |
| 78 | II-1 | [III-4/III-1/III-3] |
| 79 | II-1 | [III-4/III-1/III-4] |
| 80 | II-1 | [III-4/III-1/III-5] |
| 81 | II-1 | [III-4/III-2/III-1] |
| 82 | II-1 | [III-4/III-2/III-2] |
| 83 | II-1 | [III-4/III-2/III-3] |
| 84 | II-1 | [III-4/III-2/III-4] |
| 85 | II-1 | [III-4/III-2/III-5] |
| 86 | II-1 | [III-4/III-3/III-1] |
| 87 | II-1 | [III-4/III-3/III-2] |
| 88 | II-1 | [III-4/III-3/III-3] |
| 89 | II-1 | [III-4/III-3/III-4] |
| 90 | II-1 | [III-4/III-3/III-5] |
| 91 | II-1 | [III-4/III-4/III-1] |
| 92 | II-1 | [III-4/III-4/III-2] |
| 93 | II-1 | [III-4/III-4/III-3] |
| 94 | II-1 | [III-4/III-4/III-4] |
| 95 | II-1 | [III-4/III-4/III-5] |
| 96 | II-1 | [III-4/III-5/III-1] |
| 97 | II-1 | [III-4/III-5/III-2] |
| 98 | II-1 | [III-4/III-5/III-3] |
| 99 | II-1 | [III-4/III-5/III-4] |
| 100 | II-1 | [III-4/III-5/III-5] |
| 101 | II-1 | [III-5/III-1/III-1] |
| 102 | II-1 | [III-5/III-1/III-2] |
| 103 | II-1 | [III-5/III-1/III-3] |
| 104 | II-1 | [III-5/III-1/III-4] |
| 105 | II-1 | [III-5/III-1/III-5] |
| 106 | II-1 | [III-5/III-2/III-1] |
| 107 | II-1 | [III-5/III-2/III-2] |
| 108 | II-1 | [III-5/III-2/III-3] |
| 109 | II-1 | [III-5/III-2/III-4] |
| 110 | II-1 | [III-5/III-2/III-5] |
| 111 | II-1 | [III-5/III-3/III-1] |
| 112 | II-1 | [III-5/III-3/III-2] |
| 113 | II-1 | [III-5/III-3/III-3] |
| 114 | II-1 | [III-5/III-3/III-4] |
| 115 | II-1 | [III-5/III-3/III-5] |
| 116 | II-1 | [III-5/III-4/III-1] |
| 117 | II-1 | [III-5/III-4/III-2] |
| 118 | II-1 | [III-5/III-4/III-3] |
| 119 | II-1 | [III-5/III-4/III-4] |
| 120 | II-1 | [III-5/III-4/III-5] |
| 121 | II-1 | [III-5/III-5/III-1] |
| 122 | II-1 | [III-5/III-5/III-2] |
| 123 | II-1 | [III-5/III-5/III-3] |
| 124 | II-1 | [III-5/III-5/III-4] |
| 125 | II-1 | [III-5/III-5/III-5] |
| 126 | II-2 | [III-1/III-1/III-1] |
| 127 | II-2 | [III-1/III-1/III-2] |
| 128 | II-2 | [III-1/III-1/III-3] |
| 129 | II-2 | [III-1/III-1/III-4] |
| 130 | II-2 | [III-1/III-1/III-5] |
| 131 | II-2 | [III-1/III-2/III-1] |
| 132 | II-2 | [III-1/III-2/III-2] |
| 133 | II-2 | [III-1/III-2/III-3] |
| 134 | II-2 | [III-1/III-2/III-4] |
| 135 | II-2 | [III-1/III-2/III-5] |
| 136 | II-2 | [III-1/III-3/III-1] |
| 137 | II-2 | [III-1/III-3/III-2] |
| 138 | II-2 | [III-1/III-3/III-3] |
| 139 | II-2 | [III-1/III-3/III-4] |
| 140 | II-2 | [III-1/III-3/III-5] |
| 141 | II-2 | [III-1/III-4/III-1] |
| 142 | II-2 | [III-1/III-4/III-2] |
| 143 | II-2 | [III-1/III-4/III-3] |
| 144 | II-2 | [III-1/III-4/III-4] |
| 145 | II-2 | [III-1/III-4/III-5] |
| 146 | II-2 | [III-1/III-5/III-1] |
| 147 | II-2 | [III-1/III-5/III-2] |
| 148 | II-2 | [III-1/III-5/III-3] |
| 149 | II-2 | [III-1/III-5/III-4] |
| 150 | II-2 | [III-1/III-5/III-5] |
| 151 | II-2 | [III-2/III-1/III-2] |
| 152 | II-2 | [III-2/III-1/III-3] |
| 153 | II-2 | [III-2/III-1/III-4] |
| 154 | II-2 | [III-2/III-1/III-5] |
| 155 | II-2 | [III-2/III-2/III-2] |
| 156 | II-2 | [III-2/III-2/III-3] |
| 157 | II-2 | [III-2/III-2/III-4] |
| 158 | II-2 | [III-2/III-2/III-5] |
| 159 | II-2 | [III-2/III-3/III-2] |
| 160 | II-2 | [III-2/III-3/III-3] |
| 161 | II-2 | [III-2/III-3/III-4] |
| 162 | II-2 | [III-2/III-3/III-5] |
| 163 | II-2 | [III-2/III-4/III-2] |
| 164 | II-2 | [III-2/III-4/III-3] |
| 165 | II-2 | [III-2/III-4/III-4] |
| 166 | II-2 | [III-2/III-4/III-5] |
| 167 | II-2 | [III-2/III-5/III-2] |
| 168 | II-2 | [III-2/III-5/III-3] |
| 169 | II-2 | [III-2/III-5/III-4] |
| 170 | II-2 | [III-2/III-5/III-5] |
| 171 | II-2 | [III-3/III-1/III-3] |
| 172 | II-2 | [III-3/III-1/III-4] |
| 173 | II-2 | [III-3/III-1/III-5] |
| 174 | II-2 | [III-3/III-2/III-3] |
| 175 | II-2 | [III-3/III-2/III-4] |
| 176 | II-2 | [III-3/III-2/III-5] |
| 177 | II-2 | [III-3/III-3/III-3] |
| 178 | II-2 | [III-3/III-3/III-4] |
| 179 | II-2 | [III-3/III-3/III-5] |
| 180 | II-2 | [III-3/III-4/III-3] |
| 181 | II-2 | [III-3/III-4/III-4] |
| 182 | II-2 | [III-3/III-4/III-5] |
| 183 | II-2 | [III-3/III-5/III-3] |
| 184 | II-2 | [III-3/III-5/III-4] |
| 185 | II-2 | [III-3/III-5/III-5] |
| 186 | II-2 | [III-4/III-1/III-4] |
| 187 | II-2 | [III-4/III-1/III-5] |
| 188 | II-2 | [III-4/III-2/III-4] |
| 189 | II-2 | [III-4/III-2/III-5] |
| 190 | II-2 | [III-4/III-3/III-4] |
| 191 | II-2 | [III-4/III-3/III-5] |
| 192 | II-2 | [III-4/III-4/III-4] |
| 193 | II-2 | [III-4/III-4/III-5] |
| 194 | II-2 | [III-4/III-5/III-4] |
| 195 | II-2 | [III-4/III-5/III-5] |
| 196 | II-2 | [III-5/III-1/III-5] |
| 197 | II-2 | [III-5/III-2/III-5] |
| 198 | II-2 | [III-5/III-1/III-1] |
| 199 | II-2 | [III-5/III-2/III-2] |
| 200 | II-2 | [III-5/III-3/III-2] |
| 201 | II-3 | [III-1/III-1/III-1] |
| 202 | II-3 | [III-1/III-1/III-2] |
| 203 | II-3 | [III-1/III-1/III-3] |
| 204 | II-3 | [III-1/III-1/III-4] |
| 205 | II-3 | [III-1/III-1/III-5] |
| 206 | II-3 | [III-1/III-2/III-1] |
| 207 | II-3 | [III-1/III-2/III-2] |
| 208 | II-3 | [III-1/III-2/III-3] |
| 209 | II-3 | [III-1/III-2/III-4] |
| 210 | II-3 | [III-1/III-2/III-5] |
| 211 | II-3 | [III-1/III-3/III-1] |
| 212 | II-3 | [III-1/III-3/III-2] |
| 213 | II-3 | [III-1/III-3/III-3] |
| 214 | II-3 | [III-1/III-3/III-4] |
| 215 | II-3 | [III-1/III-3/III-5] |
| 216 | II-3 | [III-1/III-4/III-1] |
| 217 | II-3 | [III-1/III-4/III-2] |
| 218 | II-3 | [III-1/III-4/III-3] |
| 219 | II-3 | [III-1/III-4/III-4] |
| 220 | II-3 | [III-1/III-4/III-5] |
| 221 | II-3 | [III-1/III-5/III-1] |
| 222 | II-3 | [III-1/III-5/III-2] |
| 223 | II-3 | [III-1/III-5/III-3] |
| 224 | II-3 | [III-1/III-5/III-4] |
| 225 | II-3 | [III-1/III-5/III-5] |
| 226 | II-3 | [III-2/III-1/III-1] |
| 227 | II-3 | [III-2/III-1/III-2] |
| 228 | II-3 | [III-2/III-1/III-3] |
| 229 | II-3 | [III-2/III-1/III-4] |
| 230 | II-3 | [III-2/III-1/III-5] |
| 231 | II-3 | [III-2/III-2/III-1] |
| 232 | II-3 | [III-2/III-2/III-2] |
| 233 | II-3 | [III-2/III-2/III-3] |
| 234 | II-3 | [III-2/III-2/III-4] |
| 235 | II-3 | [III-2/III-2/III-5] |
| 236 | II-3 | [III-2/III-3/III-1] |
| 237 | II-3 | [III-2/III-3/III-2] |
| 238 | II-3 | [III-2/III-3/III-3] |
| 239 | II-3 | [III-2/III-3/III-4] |
| 240 | II-3 | [III-2/III-3/III-5] |
| 241 | II-3 | [III-2/III-4/III-1] |
| 242 | II-3 | [III-2/III-4/III-2] |
| 243 | II-3 | [III-2/III-4/III-3] |
| 244 | II-3 | [III-2/III-4/III-4] |
| 245 | II-3 | [III-2/III-4/III-5] |
| 246 | II-3 | [III-2/III-5/III-1] |
| 247 | II-3 | [III-2/III-5/III-2] |
| 248 | II-3 | [III-2/III-5/III-3] |
| 249 | II-3 | [III-2/III-5/III-4] |
| 250 | II-3 | [III-2/III-5/III-5] |
| 251 | II-3 | [III-3/III-1/III-1] |
| 252 | II-3 | [III-3/III-1/III-2] |
| 253 | II-3 | [III-3/III-1/III-3] |
| 254 | II-3 | [III-3/III-1/III-4] |
| 255 | II-3 | [III-3/III-1/III-5] |
| 256 | II-3 | [III-3/III-2/III-1] |
| 257 | II-3 | [III-3/III-2/III-2] |
| 258 | II-3 | [III-3/III-2/III-3] |
| 259 | II-3 | [III-3/III-2/III-4] |
| 260 | II-3 | [III-3/III-2/III-5] |
| 261 | II-3 | [III-3/III-3/III-1] |
| 262 | II-3 | [III-3/III-3/III-2] |
| 263 | II-3 | [III-3/III-3/III-3] |
| 264 | II-3 | [III-3/III-3/III-4] |
| 265 | II-3 | [III-3/III-3/III-5] |
| 266 | II-3 | [III-3/III-4/III-1] |
| 267 | II-3 | [III-3/III-4/III-2] |
| 268 | II-3 | [III-3/III-4/III-3] |
| 269 | II-3 | [III-3/III-4/III-4] |
| 270 | II-3 | [III-3/III-4/III-5] |
| 271 | II-3 | [III-3/III-5/III-1] |
| 272 | II-3 | [III-3/III-5/III-2] |
| 273 | II-3 | [III-3/III-5/III-3] |
| 274 | II-3 | [III-3/III-5/III-4] |
| 275 | II-3 | [III-3/III-5/III-5] |
| 276 | II-3 | [III-4/III-1/III-1] |
| 277 | II-3 | [III-4/III-1/III-2] |
| 278 | II-3 | [III-4/III-1/III-3] |
| 279 | II-3 | [III-4/III-1/III-4] |
| 280 | II-3 | [III-4/III-1/III-5] |
| 281 | II-3 | [III-4/III-2/III-1] |
| 282 | II-3 | [III-4/III-2/III-2] |
| 283 | II-3 | [III-4/III-2/III-3] |
| 284 | II-3 | [III-4/III-2/III-4] |
| 285 | II-3 | [III-4/III-2/III-5] |
| 286 | II-3 | [III-4/III-3/III-1] |
| 287 | II-3 | [III-4/III-3/III-2] |
| 288 | II-3 | [III-4/III-3/III-3] |
| 289 | II-3 | [III-4/III-3/III-4] |
| 290 | II-3 | [III-4/III-3/III-5] |
| 291 | II-3 | [III-4/III-4/III-1] |
| 292 | II-3 | [III-4/III-4/III-2] |
| 293 | II-3 | [III-4/III-4/III-3] |
| 294 | II-3 | [III-4/III-4/III-4] |
| 295 | II-3 | [III-4/III-4/III-5] |
| 296 | II-3 | [III-4/III-5/III-1] |
| 297 | II-3 | [III-4/III-5/III-2] |
| 298 | II-3 | [III-4/III-5/III-3] |
| 299 | II-3 | [III-4/III-5/III-4] |
| 300 | II-3 | [III-4/III-5/III-5] |
| 301 | II-3 | [III-5/III-1/III-1] |
| 302 | II-3 | [III-5/III-1/III-2] |
| 303 | II-3 | [III-5/III-1/III-3] |
| 304 | II-3 | [III-5/III-1/III-4] |
| 305 | II-3 | [III-5/III-1/III-5] |
| 306 | II-3 | [III-5/III-2/III-1] |
| 307 | II-3 | [III-5/III-2/III-2] |
| 308 | II-3 | [III-5/III-2/III-3] |
| 309 | II-3 | [III-5/III-2/III-4] |
| 310 | II-3 | [III-5/III-2/III-5] |
| 311 | II-3 | [III-5/III-3/III-1] |
| 312 | II-3 | [III-5/III-3/III-2] |
| 313 | II-3 | [III-5/III-3/III-3] |
| 314 | II-3 | [III-5/III-3/III-4] |
| 315 | II-3 | [III-5/III-3/III-5] |
| 316 | II-3 | [III-5/III-4/III-1] |
| 317 | II-3 | [III-5/III-4/III-2] |
| 318 | II-3 | [III-5/III-4/III-3] |
| 319 | II-3 | [III-5/III-4/III-4] |
| 320 | II-3 | [III-5/III-4/III-5] |
| 321 | II-3 | [III-5/III-5/III-1] |
| 322 | II-3 | [III-5/III-5/III-2] |
| 323 | II-3 | [III-5/III-5/III-3] |
| 324 | II-3 | [III-5/III-5/III-4] |
| 325 | II-3 | [III-5/III-5/III-5] |
| 326 | II-4 | [III-1/III-1/III-1] |
| 327 | II-4 | [III-1/III-1/III-2] |
| 328 | II-4 | [III-1/III-1/III-3] |
| 329 | II-4 | [III-1/III-1/III-4] |
| 330 | II-4 | [III-1/III-1/III-5] |
| 331 | II-4 | [III-1/III-2/III-1] |
| 332 | II-4 | [III-1/III-2/III-2] |
| 333 | II-4 | [III-1/III-2/III-3] |
| 334 | II-4 | [III-1/III-2/III-4] |
| 335 | II-4 | [III-1/III-2/III-5] |
| 336 | II-4 | [III-1/III-3/III-1] |
| 337 | II-4 | [III-1/III-3/III-2] |
| 338 | II-4 | [III-1/III-3/III-3] |
| 339 | II-4 | [III-1/III-3/III-4] |
| 340 | II-4 | [III-1/III-3/III-5] |
| 341 | II-4 | [III-1/III-4/III-1] |
| 342 | II-4 | [III-1/III-4/III-2] |
| 343 | II-4 | [III-1/III-4/III-3] |
| 344 | II-4 | [III-1/III-4/III-4] |
| 345 | II-4 | [III-1/III-4/III-5] |
| 346 | II-4 | [III-1/III-5/III-1] |
| 347 | II-4 | [III-1/III-5/III-2] |
| 348 | II-4 | [III-1/III-5/III-3] |
| 349 | II-4 | [III-1/III-5/III-4] |
| 350 | II-4 | [III-1/III-5/III-5] |
| 351 | II-4 | [III-2/III-1/III-1] |
| 352 | II-4 | [III-2/III-1/III-2] |
| 353 | II-4 | [III-2/III-1/III-3] |
| 354 | II-4 | [III-2/III-1/III-4] |
| 355 | II-4 | [III-2/III-1/III-5] |
| 356 | II-4 | [III-2/III-2/III-1] |
| 357 | II-4 | [III-2/III-2/III-2] |
| 358 | II-4 | [III-2/III-2/III-3] |
| 359 | II-4 | [III-2/III-2/III-4] |
| 360 | II-4 | [III-2/III-2/III-5] |
| 361 | II-4 | [III-2/III-3/III-1] |
| 362 | II-4 | [III-2/III-3/III-2] |
| 363 | II-4 | [III-2/III-3/III-3] |
| 364 | II-4 | [III-2/III-3/III-4] |
| 365 | II-4 | [III-2/III-3/III-5] |
| 366 | II-4 | [III-2/III-4/III-1] |
| 367 | II-4 | [III-2/III-4/III-2] |
| 368 | II-4 | [III-2/III-4/III-3] |
| 369 | II-4 | [III-2/III-4/III-4] |
| 370 | II-4 | [III-2/III-4/III-5] |
| 371 | II-4 | [III-2/III-5/III-1] |
| 372 | II-4 | [III-2/III-5/III-2] |
| 373 | II-4 | [III-2/III-5/III-3] |
| 374 | II-4 | [III-2/III-5/III-4] |
| 375 | II-4 | [III-2/III-5/III-5] |
| 376 | II-4 | [III-3/III-1/III-1] |
| 377 | II-4 | [III-3/III-1/III-2] |
| 378 | II-4 | [III-3/III-1/III-3] |
| 379 | II-4 | [III-3/III-1/III-4] |
| 380 | II-4 | [III-3/III-1/III-5] |
| 381 | II-4 | [III-3/III-2/III-1] |
| 382 | II-4 | [III-3/III-2/III-2] |
| 383 | II-4 | [III-3/III-2/III-3] |
| 384 | II-4 | [III-3/III-2/III-4] |
| 385 | II-4 | [III-3/III-2/III-5] |
| 386 | II-4 | [III-3/III-3/III-1] |
| 387 | II-4 | [III-3/III-3/III-2] |
| 388 | II-4 | [III-3/III-3/III-3] |
| 389 | II-4 | [III-3/III-3/III-4] |
| 390 | II-4 | [III-3/III-3/III-5] |
| 391 | II-4 | [III-3/III-4/III-1] |
| 392 | II-4 | [III-3/III-4/III-2] |
| 393 | II-4 | [III-3/III-4/III-3] |
| 394 | II-4 | [III-3/III-4/III-4] |
| 395 | II-4 | [III-3/III-4/III-5] |
| 396 | II-4 | [III-3/III-5/III-1] |
| 397 | II-4 | [III-3/III-5/III-2] |
| 398 | II-4 | [III-3/III-5/III-3] |
| 399 | II-4 | [III-3/III-5/III-4] |
| 400 | II-4 | [III-3/III-5/III-5] |
| 401 | II-4 | [III-4/III-1/III-1] |
| 402 | II-4 | [III-4/III-1/III-2] |
| 403 | II-4 | [III-4/III-1/III-3] |
| 404 | II-4 | [III-4/III-1/III-4] |
| 405 | II-4 | [III-4/III-1/III-5] |
| 406 | II-4 | [III-4/III-2/III-1] |
| 407 | II-4 | [III-4/III-2/III-2] |
| 408 | II-4 | [III-4/III-2/III-3] |
| 409 | II-4 | [III-4/III-2/III-4] |
| 410 | II-4 | [III-4/III-2/III-5] |
| 411 | II-4 | [III-4/III-3/III-1] |
| 412 | II-4 | [III-4/III-3/III-2] |
| 413 | II-4 | [III-4/III-3/III-3] |
| 414 | II-4 | [III-4/III-3/III-4] |
| 415 | II-4 | [III-4/III-3/III-5] |
| 416 | II-4 | [III-4/III-4/III-1] |
| 417 | II-4 | [III-4/III-4/III-2] |
| 418 | II-4 | [III-4/III-4/III-3] |
| 419 | II-4 | [III-4/III-4/III-4] |
| 420 | II-4 | [III-4/III-4/III-5] |
| 421 | II-4 | [III-4/III-5/III-1] |
| 422 | II-4 | [III-4/III-5/III-2] |
| 423 | II-4 | [III-4/III-5/III-3] |
| 424 | II-4 | [III-4/III-5/III-4] |
| 425 | II-4 | [III-4/III-5/III-5] |
| 426 | II-4 | [III-5/III-1/III-1] |
| 427 | II-4 | [III-5/III-1/III-2] |
| 428 | II-4 | [III-5/III-1/III-3] |
| 429 | II-4 | [III-5/III-1/III-4] |
| 430 | II-4 | [III-5/III-1/III-5] |
| 431 | II-4 | [III-5/III-2/III-1] |
| 432 | II-4 | [III-5/III-2/III-2] |
| 433 | II-4 | [III-5/III-2/III-3] |
| 434 | II-4 | [III-5/III-2/III-4] |
| 435 | II-4 | [III-5/III-2/III-5] |
| 436 | II-4 | [III-5/III-3/III-1] |
| 437 | II-4 | [III-5/III-3/III-2] |
| 438 | II-4 | [III-5/III-3/III-3] |
| 439 | II-4 | [III-5/III-3/III-4] |
| 440 | II-4 | [III-5/III-3/III-5] |
| 441 | II-4 | [III-5/III-4/III-1] |
| 442 | II-4 | [III-5/III-4/III-2] |
| 443 | II-4 | [III-5/III-4/III-3] |
| 444 | II-4 | [III-5/III-4/III-4] |
| 445 | II-4 | [III-5/III-4/III-5] |
| 446 | II-4 | [III-5/III-5/III-1] |
| 447 | II-4 | [III-5/III-5/III-2] |
| 448 | II-4 | [III-5/III-5/III-3] |
| 449 | II-4 | [III-5/III-5/III-4] |
| 450 | II-4 | [III-5/III-5/III-5] |
| 451 | II-5 | [III-1/III-1/III-1] |
| 452 | II-5 | [III-1/III-1/III-2] |
| 453 | II-5 | [III-1/III-1/III-3] |
| 454 | II-5 | [III-1/III-1/III-4] |
| 455 | II-5 | [III-1/III-1/III-5] |
| 456 | II-5 | [III-1/III-2/III-1] |
| 457 | II-5 | [III-1/III-2/III-2] |
| 458 | II-5 | [III-1/III-2/III-3] |
| 459 | II-5 | [III-1/III-2/III-4] |
| 460 | II-5 | [III-1/III-2/III-5] |
| 461 | II-5 | [III-1/III-3/III-1] |
| 462 | II-5 | [III-1/III-3/III-2] |
| 463 | II-5 | [III-1/III-3/III-3] |
| 464 | II-5 | [III-1/III-3/III-4] |
| 465 | II-5 | [III-1/III-3/III-5] |
| 466 | II-5 | [III-1/III-4/III-1] |
| 467 | II-5 | [III-1/III-4/III-2] |
| 468 | II-5 | [III-1/III-4/III-3] |
| 469 | II-5 | [III-1/III-4/III-4] |
| 470 | II-5 | [III-1/III-4/III-5] |
| 471 | II-5 | [III-1/III-5/III-1] |
| 472 | II-5 | [III-1/III-5/III-2] |
| 473 | II-5 | [III-1/III-5/III-3] |
| 474 | II-5 | [III-1/III-5/III-4] |
| 475 | II-5 | [III-1/III-5/III-5] |
| 476 | II-5 | [III-2/III-1/III-2] |
| 477 | II-5 | [III-2/III-1/III-3] |
| 478 | II-5 | [III-2/III-1/III-4] |
| 479 | II-5 | [III-2/III-1/III-5] |
| 480 | II-5 | [III-2/III-2/III-2] |
| 481 | II-5 | [III-2/III-2/III-3] |
| 482 | II-5 | [III-2/III-2/III-4] |
| 483 | II-5 | [III-2/III-2/III-5] |
| 484 | II-5 | [III-2/III-3/III-2] |
| 485 | II-5 | [III-2/III-3/III-3] |
| 486 | II-5 | [III-2/III-3/III-4] |
| 487 | II-5 | [III-3/III-3/III-5] |
| 488 | II-5 | [III-2/III-4/III-2] |
| 489 | II-5 | [III-2/III-4/III-3] |
| 490 | II-5 | [III-2/III-4/III-4] |
| 491 | II-5 | [III-2/III-4/III-5] |
| 492 | II-5 | [III-2/III-5/III-2] |
| 493 | II-5 | [III-2/III-5/III-3] |
| 494 | II-5 | [III-2/III-5/III-4] |
| 495 | II-5 | [III-2/III-5/III-5] |
| 496 | II-5 | [III-3/III-1/III-3] |
| 497 | II-5 | [III-3/III-1/III-4] |
| 498 | II-5 | [III-3/III-1/III-5] |
| 499 | II-5 | [III-3/III-2/III-3] |
| 500 | II-5 | [III-3/III-2/III-4] |
| 501 | II-5 | [III-3/III-2/III-5] |
| 502 | II-5 | [III-3/III-3/III-3] |
| 503 | II-5 | [III-3/III-3/III-4] |
| 504 | II-5 | [III-3/III-3/III-5] |
| 505 | II-5 | [III-3/III-4/III-3] |
| 506 | II-5 | [III-3/III-4/III-4] |
| 507 | II-5 | [III-3/III-4/III-5] |
| 508 | II-5 | [III-3/III-5/III-3] |
| 509 | II-5 | [III-3/III-5/III-4] |
| 510 | II-5 | [III-3/III-5/III-5] |
| 511 | II-5 | [III-4/III-1/III-4] |
| 512 | II-5 | [III-4/III-1/III-5] |
| 513 | II-5 | [III-4/III-2/III-4] |
| 514 | II-5 | [III-4/III-2/III-5] |
| 515 | II-5 | [III-4/III-3/III-4] |
| 516 | II-5 | [III-4/III-3/III-5] |
| 517 | II-5 | [III-4/III-4/III-4] |
| 518 | II-5 | [III-4/III-4/III-5] |
| 519 | II-5 | [III-4/III-5/III-4] |
| 520 | II-5 | [III-4/III-5/III-5] |
| 521 | II-5 | [III-5/III-1/III-5] |
| 522 | II-5 | [III-5/III-2/III-5] |
| 523 | II-5 | [III-5/III-3/III-5] |
| 524 | II-5 | [III-5/III-4/III-5] |
| 525 | II-5 | [III-5/III-5/III-5] |
| 526 | II-6 | [III-1/III-1/III-1] |
| 527 | II-6 | [III-1/III-1/III-2] |
| 528 | II-6 | [III-1/III-1/III-3] |
| 529 | II-6 | [III-1/III-1/III-4] |
| 530 | II-6 | [III-1/III-1/III-5] |
| 531 | II-6 | [III-1/III-2/III-1] |
| 532 | II-6 | [III-1/III-2/III-2] |
| 533 | II-6 | [III-1/III-2/III-3] |
| 534 | II-6 | [III-1/III-2/III-4] |
| 535 | II-6 | [III-1/III-2/III-5] |
| 536 | II-6 | [III-1/III-3/III-1] |
| 537 | II-6 | [III-1/III-3/III-2] |
| 538 | II-6 | [III-1/III-3/III-3] |
| 539 | II-6 | [III-1/III-3/III-4] |
| 540 | II-6 | [III-1/III-3/III-5] |
| 541 | II-6 | [III-1/III-4/III-1] |
| 542 | II-6 | [III-1/III-4/III-2] |
| 543 | II-6 | [III-1/III-4/III-3] |
| 544 | II-6 | [III-1/III-4/III-4] |
| 545 | II-6 | [III-1/III-4/III-5] |
| 546 | II-6 | [III-1/III-5/III-1] |
| 547 | II-6 | [III-1/III-5/III-2] |
| 548 | II-6 | [III-1/III-5/III-3] |
| 549 | II-6 | [III-1/III-5/III-4] |
| 550 | II-6 | [III-1/III-5/III-5] |
| 551 | II-6 | [III-2/III-1/III-2] |
| 552 | II-6 | [III-2/III-1/III-3] |
| 553 | II-6 | [III-2/III-1/III-4] |
| 554 | II-6 | [III-2/III-1/III-5] |
| 555 | II-6 | [III-2/III-2/III-2] |
| 556 | II-6 | [III-2/III-2/III-3] |
| 557 | II-6 | [III-2/III-2/III-4] |
| 558 | II-6 | [III-2/III-2/III-5] |
| 559 | II-6 | [III-2/III-3/III-3] |
| 560 | II-6 | [III-2/III-3/III-4] |
| 561 | II-6 | [III-2/III-3/III-5] |
| 562 | II-6 | [III-2/III-4/III-2] |
| 563 | II-6 | [III-2/III-4/III-3] |
| 564 | II-6 | [III-2/III-4/III-4] |
| 565 | II-6 | [III-2/III-4/III-5] |
| 566 | II-6 | [III-2/III-5/III-1] |
| 567 | II-6 | [III-2/III-5/III-2] |
| 568 | II-6 | [III-2/III-5/III-3] |
| 569 | II-6 | [III-2/III-5/III-4] |
| 570 | II-6 | [III-2/III-5/III-5] |
| 571 | II-6 | [III-3/III-1/III-3] |
| 572 | II-6 | [III-3/III-1/III-4] |
| 573 | II-6 | [III-3/III-1/III-5] |
| 574 | II-6 | [III-3/III-2/III-3] |
| 575 | II-6 | [III-3/III-2/III-4] |
| 576 | II-6 | [III-3/III-2/III-5] |
| 577 | II-6 | [III-3/III-3/III-3] |
| 578 | II-6 | [III-3/III-3/III-4] |
| 579 | II-6 | [III-3/III-3/III-5] |
| 580 | II-6 | [III-3/III-4/III-3] |
| 581 | II-6 | [III-3/III-4/III-4] |
| 582 | II-6 | [III-3/III-4/III-5] |
| 583 | II-6 | [III-3/III-5/III-3] |
| 584 | II-6 | [III-3/III-5/III-4] |
| 585 | II-6 | [III-3/III-5/III-5] |
| 586 | II-6 | [III-4/III-1/III-4] |
| 587 | II-6 | [III-4/III-1/III-5] |
| 588 | II-6 | [III-4/III-2/III-4] |
| 589 | II-6 | [III-4/III-2/III-5] |
| 590 | II-6 | [III-4/III-3/III-4] |
| 591 | II-6 | [III-4/III-3/III-5] |
| 592 | II-6 | [III-4/III-4/III-4] |
| 593 | II-6 | [III-4/III-4/III-5] |
| 594 | II-6 | [III-4/III-5/III-4] |
| 595 | II-6 | [III-4/III-5/III-5] |
| 596 | II-6 | [III-5/III-1/III-5] |
| 597 | II-6 | [III-5/III-2/III-5] |
| 598 | II-6 | [III-5/III-3/III-5] |
| 599 | II-6 | [III-5/III-4/III-5] |
| 600 | II-6 | [III-5/III-5/III-5] |
| 601 | II-7 | [III-1/III-1/III-1] |
| 602 | II-7 | [III-1/III-1/III-2] |
| 603 | II-7 | [III-1/III-1/III-3] |
| 604 | II-7 | [III-1/III-1/III-4] |
| 605 | II-7 | [III-1/III-1/III-5] |
| 606 | II-7 | [III-1/III-2/III-1] |
| 607 | II-7 | [III-1/III-2/III-2] |
| 608 | II-7 | [III-1/III-2/III-3] |
| 609 | II-7 | [III-1/III-2/III-4] |
| 610 | II-7 | [III-1/III-2/III-5] |
| 611 | II-7 | [III-1/III-3/III-1] |
| 612 | II-7 | [III-1/III-3/III-2] |
| 613 | II-7 | [III-1/III-3/III-3] |
| 614 | II-7 | [III-1/III-3/III-4] |
| 615 | II-7 | [III-1/III-3/III-5] |
| 616 | II-7 | [III-1/III-4/III-1] |
| 617 | II-7 | [III-1/III-4/III-2] |
| 618 | II-7 | [III-1/III-4/III-3] |
| 619 | II-7 | [III-1/III-4/III-4] |
| 620 | II-7 | [III-1/III-4/III-5] |
| 621 | II-7 | [III-1/III-5/III-1] |
| 622 | II-7 | [III-1/III-5/III-2] |
| 623 | II-7 | [III-1/III-5/III-3] |
| 624 | II-7 | [III-4/III-5/III-4] |
| 625 | II-7 | [III-1/III-5/III-5] |
| 626 | II-7 | [III-2/III-1/III-1] |
| 627 | II-7 | [III-2/III-1/III-2] |
| 628 | II-7 | [III-2/III-1/III-3] |
| 629 | II-7 | [III-2/III-1/III-4] |
| 630 | II-7 | [III-2/III-1/III-5] |
| 631 | II-7 | [III-2/III-2/III-1] |
| 632 | II-7 | [III-2/III-2/III-2] |
| 633 | II-7 | [III-2/III-2/III-3] |
| 634 | II-7 | [III-2/III-2/III-4] |
| 635 | II-7 | [III-2/III-2/III-5] |
| 636 | II-7 | [III-2/III-3/III-1] |
| 637 | II-7 | [III-2/III-3/III-2] |
| 638 | II-7 | [III-2/III-3/III-3] |
| 639 | II-7 | [III-2/III-3/III-4] |
| 640 | II-7 | [III-2/III-3/III-5] |
| 641 | II-7 | [III-2/III-4/III-1] |
| 642 | II-7 | [III-2/III-4/III-2] |
| 643 | II-7 | [III-2/III-4/III-3] |
| 644 | II-7 | [III-2/III-4/III-4] |
| 645 | II-7 | [III-2/III-4/III-5] |
| 646 | II-7 | [III-2/III-5/III-1] |
| 647 | II-7 | [III-2/III-5/III-2] |
| 648 | II-7 | [III-2/III-5/III-3] |
| 649 | II-7 | [III-2/III-5/III-4] |
| 650 | II-7 | [III-2/III-5/III-5] |
| 651 | II-7 | [III-3/III-1/III-1] |
| 652 | II-7 | [III-3/III-1/III-2] |
| 653 | II-7 | [III-3/III-1/III-3] |
| 654 | II-7 | [III-3/III-1/III-4] |
| 655 | II-7 | [III-3/III-1/III-5] |
| 656 | II-7 | [III-3/III-2/III-1] |
| 657 | II-7 | [III-3/III-2/III-2] |
| 658 | II-7 | [III-3/III-2/III-3] |
| 659 | II-7 | [III-3/III-2/III-4] |
| 660 | II-7 | [III-3/III-2/III-5] |
| 661 | II-7 | [III-3/III-3/III-1] |
| 662 | II-7 | [III-3/III-3/III-2] |
| 663 | II-7 | [III-3/III-3/III-3] |
| 664 | II-7 | [III-3/III-3/III-4] |
| 665 | II-7 | [III-3/III-3/III-5] |
| 666 | II-7 | [III-3/III-4/III-1] |
| 667 | II-7 | [III-3/III-4/III-2] |
| 668 | II-7 | [III-3/III-4/III-3] |
| 669 | II-7 | [III-3/III-4/III-4] |
| 670 | II-7 | [III-3/III-4/III-5] |
| 671 | II-7 | [III-3/III-5/III-1] |
| 672 | II-7 | [III-3/III-5/III-2] |
| 673 | II-7 | [III-3/III-5/III-3] |
| 674 | II-7 | [III-3/III-5/III-4] |
| 675 | II-7 | [III-3/III-5/III-5] |
| 676 | II-7 | [III-4/III-1/III-1] |
| 677 | II-7 | [III-4/III-1/III-2] |
| 678 | II-7 | [III-4/III-1/III-3] |
| 679 | II-7 | [III-4/III-1/III-4] |
| 680 | II-7 | [III-4/III-1/III-5] |
| 681 | II-7 | [III-4/III-2/III-1] |
| 682 | II-7 | [III-4/III-2/III-2] |
| 683 | II-7 | [III-4/III-2/III-3] |
| 684 | II-7 | [III-4/III-2/III-4] |
| 685 | II-7 | [III-4/III-2/III-5] |
| 686 | II-7 | [III-4/III-3/III-1] |
| 687 | II-7 | [III-4/III-3/III-2] |
| 688 | II-7 | [III-4/III-3/III-3] |
| 689 | II-7 | [III-4/III-3/III-4] |
| 690 | II-7 | [III-4/III-3/III-5] |
| 691 | II-7 | [III-4/III-4/III-1] |
| 692 | II-7 | [III-4/III-4/III-2] |
| 693 | II-7 | [III-4/III-4/III-3] |
| 694 | II-7 | [III-4/III-4/III-4] |
| 695 | II-7 | [III-4/III-4/III-5] |
| 696 | II-7 | [III-4/III-5/III-1] |
| 697 | II-7 | [III-4/III-5/III-2] |
| 698 | II-7 | [III-4/III-5/III-3] |
| 699 | II-7 | [III-4/III-5/III-4] |
| 700 | II-7 | [III-4/III-5/III-5] |
| 701 | II-7 | [III-5/III-1/III-1] |
| 702 | II-7 | [III-5/III-1/III-2] |
| 703 | II-7 | [III-5/III-1/III-3] |
| 704 | II-7 | [III-5/III-1/III-4] |
| 705 | II-7 | [III-5/III-1/III-5] |
| 706 | II-7 | [III-5/III-2/III-1] |
| 707 | II-7 | [III-5/III-2/III-2] |
| 708 | II-7 | [III-5/III-2/III-3] |
| 709 | II-7 | [III-5/III-2/III-4] |
| 710 | II-7 | [III-5/III-2/III-5] |
| 711 | II-7 | [III-5/III-3/III-1] |
| 712 | II-7 | [III-5/III-3/III-2] |
| 713 | II-7 | [III-5/III-3/III-3] |
| 714 | II-7 | [III-5/III-3/III-4] |
| 715 | II-7 | [III-5/III-3/III-5] |
| 716 | II-7 | [III-5/III-4/III-1] |
| 717 | II-7 | [III-5/III-4/III-2] |
| 718 | II-7 | [III-5/III-4/III-3] |
| 719 | II-7 | [III-5/III-4/III-4] |
| 720 | II-7 | [III-5/III-4/III-5] |
| 721 | II-7 | [III-5/III-5/III-1] |
| 722 | II-7 | [III-5/III-5/III-2] |
| 723 | II-7 | [III-5/III-5/III-3] |
| 724 | II-7 | [III-5/III-5/III-4] |
| 725 | II-7 | [III-5/III-5/III-5] |
| 726 | II-8 | [III-1/III-1/III-1] |
| 727 | II-8 | [III-1/III-1/III-2] |
| 728 | II-8 | [III-1/III-1/III-3] |
| 729 | II-8 | [III-1/III-1/III-4] |
| 730 | II-8 | [III-1/III-1/III-5] |
| 731 | II-8 | [III-1/III-2/III-1] |
| 732 | II-8 | [III-1/III-2/III-2] |
| 733 | II-8 | [III-1/III-2/III-3] |
| 734 | II-8 | [III-1/III-2/III-4] |
| 735 | II-8 | [III-1/III-2/III-5] |
| 736 | II-8 | [III-1/III-3/III-1] |
| 737 | II-8 | [III-1/III-3/III-2] |
| 738 | II-8 | [III-1/III-3/III-3] |
| 739 | II-8 | [III-1/III-3/III-4] |
| 740 | II-8 | [III-1/III-3/III-5] |
| 741 | II-8 | [III-1/III-4/III-1] |
| 742 | II-8 | [III-1/III-4/III-2] |
| 743 | II-8 | [III-1/III-4/III-3] |
| 744 | II-8 | [III-1/III-4/III-4] |
| 745 | II-8 | [III-1/III-4/III-5] |
| 746 | II-8 | [III-1/III-5/III-1] |
| 747 | II-8 | [III-1/III-5/III-2] |
| 748 | II-8 | [III-1/III-5/III-3] |
| 749 | II-8 | [III-1/III-5/III-4] |
| 750 | II-8 | [III-1/III-5/III-5] |
| 751 | II-8 | [III-2/III-1/III-1] |
| 752 | II-8 | [III-2/III-1/III-2] |
| 753 | II-8 | [III-2/III-1/III-3] |
| 754 | II-8 | [III-2/III-1/III-4] |
| 755 | II-8 | [III-2/III-1/III-5] |
| 756 | II-8 | [III-2/III-2/III-1] |
| 757 | II-8 | [III-2/III-2/III-2] |
| 758 | II-8 | [III-2/III-2/III-3] |
| 759 | II-8 | [III-2/III-2/III-4] |
| 760 | II-8 | [III-2/III-2/III-5] |
| 761 | II-8 | [III-2/III-3/III-1] |
| 762 | II-8 | [III-2/III-3/III-2] |
| 763 | II-8 | [III-2/III-3/III-3] |
| 764 | II-8 | [III-2/III-3/III-4] |
| 765 | II-8 | [III-2/III-3/III-5] |
| 766 | II-8 | [III-2/III-4/III-1] |
| 767 | II-8 | [III-2/III-4/III-2] |
| 768 | II-8 | [III-2/III-4/III-3] |
| 769 | II-8 | [III-2/III-4/III-4] |
| 770 | II-8 | [III-2/III-4/III-5] |
| 771 | II-8 | [III-2/III-5/III-1] |
| 772 | II-8 | [III-2/III-5/III-2] |
| 773 | II-8 | [III-2/III-5/III-3] |
| 774 | II-8 | [III-2/III-5/III-4] |
| 775 | II-8 | [III-2/III-5/III-5] |
| 776 | II-8 | [III-3/III-1/III-1] |
| 777 | II-8 | [III-3/III-1/III-2] |
| 778 | II-8 | [III-3/III-1/III-3] |
| 779 | II-8 | [III-3/III-1/III-4] |
| 780 | II-8 | [III-3/III-1/III-5] |
| 781 | II-8 | [III-3/III-2/III-1] |
| 782 | II-8 | [III-3/III-2/III-2] |
| 783 | II-8 | [III-3/III-2/III-3] |
| 784 | II-8 | [III-3/III-2/III-4] |
| 785 | II-8 | [III-3/III-2/III-5] |
| 786 | II-8 | [III-3/III-3/III-1] |
| 787 | II-8 | [III-3/III-3/III-2] |
| 788 | II-8 | [III-3/III-3/III-3] |
| 789 | II-8 | [III-3/III-3/III-4] |
| 790 | II-8 | [III-3/III-3/III-5] |
| 791 | II-8 | [III-3/III-4/III-1] |
| 792 | II-8 | [III-3/III-4/III-2] |
| 793 | II-8 | [III-3/III-4/III-3] |
| 794 | II-8 | [III-3/III-4/III-4] |
| 795 | II-8 | [III-3/III-4/III-5] |
| 796 | II-8 | [III-3/III-5/III-1] |
| 797 | II-8 | [III-3/III-5/III-2] |
| 798 | II-8 | [III-3/III-5/III-3] |
| 799 | II-8 | [III-3/III-5/III-4] |
| 800 | II-8 | [III-3/III-5/III-5] |
| 801 | II-8 | [III-4/III-1/III-1] |
| 802 | II-8 | [III-4/III-1/III-2] |
| 803 | II-8 | [III-4/III-1/III-3] |
| 804 | II-8 | [III-4/III-1/III-4] |
| 805 | II-8 | [III-4/III-1/III-5] |
| 806 | II-8 | [III-4/III-2/III-1] |
| 807 | II-8 | [III-4/III-2/III-2] |
| 808 | II-8 | [III-4/III-2/III-3] |
| 809 | II-8 | [III-4/III-2/III-4] |
| 810 | II-8 | [III-4/III-2/III-5] |
| 811 | II-8 | [III-4/III-3/III-1] |
| 812 | II-8 | [III-4/III-3/III-2] |
| 813 | II-8 | [III-4/III-3/III-3] |
| 814 | II-8 | [III-4/III-3/III-4] |
| 815 | II-8 | [III-4/III-3/III-5] |
| 816 | II-8 | [III-4/III-4/III-1] |
| 817 | II-8 | [III-4/III-4/III-2] |
| 818 | II-8 | [III-4/III-4/III-3] |
| 819 | II-8 | [III-4/III-4/III-4] |
| 820 | II-8 | [III-4/III-4/III-5] |
| 821 | II-8 | [III-4/III-5/III-1] |
| 822 | II-8 | [III-4/III-5/III-2] |
| 823 | II-8 | [III-4/III-5/III-3] |
| 824 | II-8 | [III-4/III-5/III-4] |
| 825 | II-8 | [III-4/III-5/III-5] |
| 826 | II-8 | [III-5/III-1/III-1] |
| 827 | II-8 | [III-5/III-1/III-2] |
| 828 | II-8 | [III-5/III-1/III-3] |
| 829 | II-8 | [III-5/III-1/III-4] |
| 830 | II-8 | [III-5/III-1/III-5] |
| 831 | II-8 | [III-5/III-2/III-1] |
| 832 | II-8 | [III-5/III-2/III-2] |
| 833 | II-8 | [III-5/III-2/III-3] |
| 834 | II-8 | [III-5/III-2/III-4] |
| 835 | II-8 | [III-5/III-2/III-5] |
| 836 | II-8 | [III-5/III-3/III-1] |
| 837 | II-8 | [III-5/III-3/III-2] |
| 838 | II-8 | [III-5/III-3/III-3] |
| 839 | II-8 | [III-5/III-3/III-4] |
| 840 | II-8 | [III-5/III-3/III-5] |
| 841 | II-8 | [III-5/III-4/III-1] |
| 842 | II-8 | [III-5/III-4/III-2] |
| 843 | II-8 | [III-5/III-4/III-3] |
| 844 | II-8 | [III-5/III-4/III-4] |
| 845 | II-8 | [III-5/III-4/III-5] |
| 846 | II-8 | [III-5/III-5/III-1] |
| 847 | II-8 | [III-5/III-5/III-2] |
| 848 | II-8 | [III-5/III-5/III-3] |
| 849 | II-8 | [III-5/III-5/III-4] |
| 850 | II-8 | [III-5/III-5/III-5] |
| 851 | II-9 | [III-1/III-1/III-1] |
| 852 | II-9 | [III-1/III-1/III-2] |
| 853 | II-9 | [III-1/III-1/III-3] |
| 854 | II-9 | [III-1/III-1/III-4] |
| 855 | II-9 | [III-1/III-1/III-5] |
| 856 | II-9 | [III-1/III-2/III-1] |
| 857 | II-9 | [III-1/III-2/III-2] |
| 858 | II-9 | [III-1/III-2/III-3] |
| 859 | II-9 | [III-1/III-2/III-4] |
| 860 | II-9 | [III-1/III-2/III-5] |
| 861 | II-9 | [III-1/III-3/III-1] |
| 862 | II-9 | [III-1/III-3/III-2] |
| 863 | II-9 | [III-1/III-3/III-3] |
| 864 | II-9 | [III-1/III-3/III-4] |
| 865 | II-9 | [III-1/III-3/III-5] |
| 866 | II-9 | [III-1/III-4/III-1] |
| 867 | II-9 | [III-1/III-4/III-2] |
| 868 | II-9 | [III-1/III-4/III-3] |
| 869 | II-9 | [III-1/III-4/III-4] |
| 870 | II-9 | [III-1/III-4/III-5] |
| 871 | II-9 | [III-1/III-5/III-1] |
| 872 | II-9 | [III-1/III-5/III-2] |
| 873 | II-9 | [III-1/III-5/III-3] |
| 874 | II-9 | [III-1/III-5/III-4] |
| 875 | II-9 | [III-1/III-5/III-5] |
| 876 | II-9 | [III-2/III-1/III-1] |
| 877 | II-9 | [III-2/III-1/III-2] |
| 878 | II-9 | [III-2/III-1/III-3] |
| 879 | II-9 | [III-2/III-1/III-4] |
| 880 | II-9 | [III-2/III-1/III-5] |
| 881 | II-9 | [III-2/III-2/III-1] |
| 882 | II-9 | [III-2/III-2/III-2] |
| 883 | II-9 | [III-2/III-2/III-3] |
| 884 | II-9 | [III-2/III-2/III-4] |
| 885 | II-9 | [III-2/III-2/III-5] |
| 886 | II-9 | [III-2/III-3/III-1] |
| 887 | II-9 | [III-2/III-3/III-2] |
| 888 | II-9 | [III-2/III-3/III-3] |
| 889 | II-9 | [III-2/III-3/III-4] |
| 890 | II-9 | [III-2/III-3/III-5] |
| 891 | II-9 | [III-2/III-4/III-1] |
| 892 | II-9 | [III-2/III-4/III-2] |
| 893 | II-9 | [III-2/III-4/III-3] |
| 894 | II-9 | [III-2/III-4/III-4] |
| 895 | II-9 | [III-2/III-4/III-5] |
| 896 | II-9 | [III-2/III-5/III-1] |
| 897 | II-9 | [III-2/III-5/III-2] |
| 898 | II-9 | [III-2/III-5/III-3] |
| 899 | II-9 | [III-2/III-5/III-4] |
| 900 | II-9 | [III-2/III-5/III-5] |
| 901 | II-9 | [III-3/III-1/III-1] |
| 902 | II-9 | [III-3/III-1/III-2] |
| 903 | II-9 | [III-3/III-1/III-3] |
| 904 | II-9 | [III-3/III-1/III-4] |
| 905 | II-9 | [III-3/III-1/III-5] |
| 906 | II-9 | [III-3/III-2/III-1] |
| 907 | II-9 | [III-3/III-2/III-2] |
| 908 | II-9 | [III-3/III-2/III-3] |
| 909 | II-9 | [III-3/III-2/III-4] |
| 910 | II-9 | [III-3/III-2/III-5] |
| 911 | II-9 | [III-3/III-3/III-1] |
| 912 | II-9 | [III-3/III-3/III-2] |
| 913 | II-9 | [III-3/III-3/III-3] |
| 914 | II-9 | [III-3/III-3/III-4] |
| 915 | II-9 | [III-3/III-3/III-5] |
| 916 | II-9 | [III-3/III-4/III-1] |
| 917 | II-9 | [III-3/III-4/III-2] |
| 918 | II-9 | [III-3/III-4/III-3] |
| 919 | II-9 | [III-3/III-4/III-4] |
| 920 | II-9 | [III-3/III-4/III-5] |
| 921 | II-9 | [III-3/III-5/III-1] |
| 922 | II-9 | [III-3/III-5/III-2] |
| 923 | II-9 | [III-3/III-5/III-3] |
| 924 | II-9 | [III-3/III-5/III-4] |
| 925 | II-9 | [III-3/III-5/III-5] |
| 926 | II-9 | [III-4/III-1/III-1] |
| 927 | II-9 | [III-4/III-1/III-2] |
| 928 | II-9 | [III-4/III-1/III-3] |
| 929 | II-9 | [III-4/III-1/III-4] |
| 930 | II-9 | [III-4/III-1/III-5] |
| 931 | II-9 | [III-4/III-2/III-1] |
| 932 | II-9 | [III-4/III-2/III-2] |
| 933 | II-9 | [III-4/III-2/III-3] |
| 934 | II-9 | [III-4/III-2/III-4] |
| 935 | II-9 | [III-4/III-2/III-5] |
| 936 | II-9 | [III-4/III-3/III-1] |
| 937 | II-9 | [III-4/III-3/III-2] |
| 938 | II-9 | [III-4/III-3/III-3] |
| 939 | II-9 | [III-4/III-3/III-4] |
| 940 | II-9 | [III-4/III-3/III-5] |
| 941 | II-9 | [III-4/III-4/III-1] |
| 942 | II-9 | [III-4/III-4/III-2] |
| 943 | II-9 | [III-4/III-4/III-3] |
| 944 | II-9 | [III-4/III-4/III-4] |
| 945 | II-9 | [III-4/III-4/III-5] |
| 946 | II-9 | [III-4/III-5/III-1] |
| 947 | II-9 | [III-4/III-5/III-2] |
| 948 | II-9 | [III-4/III-5/III-3] |
| 949 | II-9 | [III-4/III-5/III-4] |
| 950 | II-9 | [III-4/III-5/III-5] |
| 951 | II-9 | [III-5/III-1/III-1] |
| 952 | II-9 | [III-5/III-1/III-2] |
| 953 | II-9 | [III-5/III-1/III-3] |
| 954 | II-9 | [III-5/III-1/III-4] |
| 955 | II-9 | [III-5/III-1/III-5] |
| 956 | II-9 | [III-5/III-2/III-1] |
| 957 | II-9 | [III-5/III-2/III-2] |
| 958 | II-9 | [III-5/III-2/III-3] |
| 959 | II-9 | [III-5/III-2/III-4] |
| 960 | II-9 | [III-5/III-2/III-5] |
| 961 | II-9 | [III-5/III-3/III-1] |
| 962 | II-9 | [III-5/III-3/III-2] |
| 963 | II-9 | [III-5/III-3/III-3] |
| 964 | II-9 | [III-5/III-3/III-4] |
| 965 | II-9 | [III-5/III-3/III-5] |
| 966 | II-9 | [III-5/III-4/III-1] |
| 967 | II-9 | [III-5/III-4/III-2] |
| 968 | II-9 | [III-5/III-4/III-3] |
| 969 | II-9 | [III-5/III-4/III-4] |
| 970 | II-9 | [III-5/III-4/III-5] |
| 971 | II-9 | [III-5/III-5/III-1] |
| 972 | II-9 | [III-5/III-5/III-2] |
| 973 | II-9 | [III-5/III-5/III-3] |
| 974 | II-9 | [III-5/III-5/III-4] |
| 975 | II-9 | [III-5/III-5/III-5] |
| 976 | II-10 | [III-1/III-1/III-1] |
| 977 | II-10 | [III-1/III-1/III-2] |
| 978 | II-10 | [III-1/III-1/III-3] |
| 979 | II-10 | [III-1/III-1/III-4] |
| 980 | II-10 | [III-1/III-1/III-5] |
| 981 | II-10 | [III-1/III-2/III-1] |
| 982 | II-10 | [III-1/III-2/III-2] |
| 983 | II-10 | [III-1/III-2/III-3] |
| 984 | II-10 | [III-1/III-2/III-4] |
| 985 | II-10 | [III-1/III-2/III-5] |
| 986 | II-10 | [III-1/III-3/III-1] |
| 987 | II-10 | [III-1/III-3/III-2] |
| 988 | II-10 | [III-1/III-3/III-3] |
| 989 | II-10 | [III-1/III-3/III-4] |
| 990 | II-10 | [III-1/III-3/III-5] |
| 991 | II-10 | [III-1/III-4/III-1] |
| 992 | II-10 | [III-1/III-4/III-2] |
| 993 | II-10 | [III-1/III-4/III-3] |
| 994 | II-10 | [III-1/III-4/III-4] |
| 995 | II-10 | [III-1/III-4/III-5] |
| 996 | II-10 | [III-1/III-5/III-1] |
| 997 | II-10 | [III-1/III-5/III-2] |
| 998 | II-10 | [III-1/III-5/III-3] |
| 999 | II-10 | [III-1/III-5/III-4] |
| 1000 | II-10 | [III-1/III-5/III-5] |
| 1001 | II-10 | [III-2/III-1/III-2] |
| 1002 | II-10 | [III-2/III-1/III-3] |
| 1003 | II-10 | [III-2/III-1/III-4] |
| 1004 | II-10 | [III-2/III-1/III-5] |
| 1005 | II-10 | [III-2/III-2/III-2] |
| 1006 | II-10 | [III-2/III-2/III-3] |
| 1007 | II-10 | [III-2/III-2/III-4] |
| 1008 | II-10 | [III-2/III-2/III-5] |
| 1009 | II-10 | [III-2/III-3/III-2] |
| 1010 | II-10 | [III-2/III-3/III-3] |
| 1011 | II-10 | [III-2/III-3/III-4] |
| 1012 | II-10 | [III-2/III-3/III-5] |
| 1013 | II-10 | [III-2/III-4/III-2] |
| 1014 | II-10 | [III-2/III-4/III-3] |
| 1015 | II-10 | [III-2/III-4/III-4] |
| 1016 | II-10 | [III-2/III-4/III-5] |
| 1017 | II-10 | [III-2/III-5/III-2] |
| 1018 | II-10 | [III-2/III-5/III-3] |
| 1019 | II-10 | [III-2/III-5/III-4] |
| 1020 | II-10 | [III-2/III-5/III-5] |
| 1021 | II-10 | [III-3/III-1/III-3] |
| 1022 | II-10 | [III-3/III-1/III-4] |
| 1023 | II-10 | [III-3/III-1/III-5] |
| 1024 | II-10 | [III-3/III-2/III-3] |
| 1025 | II-10 | [III-3/III-2/III-4] |
| 1026 | II-10 | [III-3/III-2/III-5] |
| 1027 | II-10 | [III-3/III-3/III-3] |
| 1028 | II-10 | [III-3/III-3/III-4] |
| 1029 | II-10 | [III-3/III-3/III-5] |
| 1030 | II-10 | [III-3/III-4/III-3] |
| 1031 | II-10 | [III-3/III-4/III-4] |
| 1032 | II-10 | [III-3/III-4/III-5] |
| 1033 | II-10 | [III-3/III-5/III-3] |
| 1034 | II-10 | [III-3/III-5/III-4] |
| 1035 | II-10 | [III-3/III-5/III-5] |
| 1036 | II-10 | [III-4/III-1/III-4] |
| 1037 | II-10 | [III-4/III-1/III-5] |
| 1038 | II-10 | [III-4/III-2/III-4] |
| 1039 | II-10 | [III-4/III-2/III-5] |
| 1040 | II-10 | [III-4/III-3/III-4] |
| 1041 | II-10 | [III-4/III-3/III-5] |
| 1042 | II-10 | [III-4/III-4/III-4] |
| 1043 | II-10 | [III-4/III-4/III-5] |
| 1044 | II-10 | [III-4/III-5/III-4] |
| 1045 | II-10 | [III-4/III-5/III-5] |
| 1046 | II-10 | [III-5/III-1/III-5] |
| 1047 | II-10 | [III-5/III-2/III-5] |
| 1048 | II-10 | [III-5/III-3/III-5] |
| 1049 | II-10 | [III-5/III-4/III-5] |
| 1050 | II-10 | [III-5/III-5/III-5] |
| 1051 | II-11 | [III-1/III-1/III-1] |
| 1052 | II-11 | [III-1/III-1/III-2] |
| 1053 | II-11 | [III-1/III-1/III-3] |
| 1054 | II-11 | [III-1/III-1/III-4] |
| 1055 | II-11 | [III-1/III-1/III-5] |
| 1056 | II-11 | [III-1/III-2/III-1] |
| 1057 | II-11 | [III-1/III-2/III-2] |
| 1058 | II-11 | [III-1/III-2/III-3] |
| 1059 | II-11 | [III-1/III-2/III-4] |
| 1060 | II-11 | [III-1/III-2/III-5] |
| 1061 | II-11 | [III-1/III-3/III-1] |
| 1062 | II-11 | [III-1/III-3/III-2] |
| 1063 | II-11 | [III-1/III-3/III-3] |
| 1064 | II-11 | [III-1/III-3/III-4] |
| 1065 | II-11 | [III-1/III-3/III-5] |
| 1066 | II-11 | [III-1/III-4/III-1] |
| 1067 | II-11 | [III-1/III-4/III-2] |
| 1068 | II-11 | [III-1/III-4/III-3] |
| 1069 | II-11 | [III-1/III-4/III-4] |
| 1070 | II-11 | [III-1/III-4/III-5] |
| 1071 | II-11 | [III-1/III-5/III-1] |
| 1072 | II-11 | [III-1/III-5/III-2] |
| 1073 | II-11 | [III-1/III-5/III-3] |
| 1074 | II-11 | [III-1/III-5/III-4] |
| 1075 | II-11 | [III-1/III-5/III-5] |
| 1076 | II-11 | [III-2/III-1/III-1] |
| 1077 | II-11 | [III-2/III-1/III-2] |
| 1078 | II-11 | [III-2/III-1/III-3] |
| 1079 | II-11 | [III-2/III-1/III-4] |
| 1080 | II-11 | [III-2/III-1/III-5] |
| 1081 | II-11 | [III-2/III-2/III-1] |
| 1082 | II-11 | [III-2/III-2/III-2] |
| 1083 | II-11 | [III-2/III-2/III-3] |
| 1084 | II-11 | [III-2/III-2/III-4] |
| 1085 | II-11 | [III-2/III-2/III-5] |
| 1086 | II-11 | [III-2/III-3/III-1] |
| 1087 | II-11 | [III-2/III-3/III-2] |
| 1088 | II-11 | [III-2/III-3/III-3] |
| 1089 | II-11 | [III-2/III-3/III-4] |
| 1090 | II-11 | [III-2/III-3/III-5] |
| 1091 | II-11 | [III-2/III-4/III-1] |
| 1092 | II-11 | [III-2/III-4/III-2] |
| 1093 | II-11 | [III-2/III-4/III-3] |
| 1094 | II-11 | [III-2/III-4/III-4] |
| 1095 | II-11 | [III-2/III-4/III-5] |
| 1096 | II-11 | [III-2/III-5/III-1] |
| 1097 | II-11 | [III-2/III-5/III-2] |
| 1098 | II-11 | [III-2/III-5/III-3] |
| 1099 | II-11 | [III-2/III-5/III-4] |
| 1100 | II-11 | [III-2/III-5/III-5] |
| 1101 | II-11 | [III-3/III-1/III-1] |
| 1102 | II-11 | [III-3/III-1/III-2] |
| 1103 | II-11 | [III-3/III-1/III-3] |
| 1104 | II-11 | [III-3/III-1/III-4] |
| 1105 | II-11 | [III-3/III-1/III-5] |
| 1106 | II-11 | [III-3/III-2/III-1] |
| 1107 | II-11 | [III-3/III-2/III-2] |
| 1108 | II-11 | [III-3/III-2/III-3] |
| 1109 | II-11 | [III-3/III-2/III-4] |
| 1110 | II-11 | [III-3/III-2/III-5] |
| 1111 | II-11 | [III-3/III-3/III-1] |
| 1112 | II-11 | [III-3/III-3/III-2] |
| 1113 | II-11 | [III-3/III-3/III-3] |
| 1114 | II-11 | [III-3/III-3/III-4] |
| 1115 | II-11 | [III-3/III-3/III-5] |
| 1116 | II-11 | [III-3/III-4/III-1] |
| 1117 | II-11 | [III-3/III-4/III-2] |
| 1118 | II-11 | [III-3/III-4/III-3] |
| 1119 | II-11 | [III-3/III-4/III-4] |
| 1120 | II-11 | [III-3/III-4/III-5] |
| 1121 | II-11 | [III-3/III-5/III-1] |
| 1122 | II-11 | [III-3/III-5/III-2] |
| 1123 | II-11 | [III-3/III-5/III-3] |
| 1124 | II-11 | [III-3/III-5/III-4] |
| 1125 | II-11 | [III-3/III-5/III-5] |
| 1126 | II-11 | [III-4/III-1/III-1] |
| 1127 | II-11 | [III-4/III-1/III-2] |
| 1128 | II-11 | [III-4/III-1/III-3] |
| 1129 | II-11 | [III-4/III-1/III-4] |
| 1130 | II-11 | [III-4/III-1/III-5] |
| 1131 | II-11 | [III-4/III-2/III-1] |
| 1132 | II-11 | [III-4/III-2/III-2] |
| 1133 | II-11 | [III-4/III-2/III-3] |
| 1134 | II-11 | [III-4/III-2/III-4] |
| 1135 | II-11 | [III-4/III-2/III-5] |
| 1136 | II-11 | [III-4/III-3/III-1] |
| 1137 | II-11 | [III-4/III-3/III-2] |
| 1138 | II-11 | [III-4/III-3/III-3] |
| 1139 | II-11 | [III-4/III-3/III-4] |
| 1140 | II-11 | [III-4/III-3/III-5] |
| 1141 | II-11 | [III-4/III-4/III-1] |
| 1142 | II-11 | [III-4/III-4/III-2] |
| 1143 | II-11 | [III-4/III-4/III-3] |
| 1144 | II-11 | [III-4/III-4/III-4] |
| 1145 | II-11 | [III-4/III-4/III-5] |
| 1146 | II-11 | [III-4/III-5/III-1] |
| 1147 | II-11 | [III-4/III-5/III-2] |
| 1148 | II-11 | [III-4/III-5/III-3] |
| 1149 | II-11 | [III-4/III-5/III-4] |
| 1150 | II-11 | [III-4/III-5/III-5] |
| 1151 | II-11 | [III-5/III-1/III-1] |
| 1152 | II-11 | [III-5/III-1/III-2] |
| 1153 | II-11 | [III-5/III-1/III-3] |
| 1154 | II-11 | [III-5/III-1/III-4] |
| 1155 | II-11 | [III-5/III-1/III-5] |
| 1156 | II-11 | [III-5/III-2/III-1] |
| 1157 | II-11 | [III-5/III-2/III-2] |
| 1158 | II-11 | [III-5/III-2/III-3] |
| 1159 | II-11 | [III-5/III-2/III-4] |
| 1160 | II-11 | [III-5/III-2/III-5] |
| 1161 | II-11 | [III-5/III-3/III-1] |
| 1162 | II-11 | [III-5/III-3/III-2] |
| 1163 | II-11 | [III-5/III-3/III-3] |
| 1164 | II-11 | [III-5/III-3/III-4] |
| 1165 | II-11 | [III-5/III-3/III-5] |
| 1166 | II-11 | [III-5/III-4/III-1] |
| 1167 | II-11 | [III-5/III-4/III-2] |
| 1168 | II-11 | [III-5/III-4/III-3] |
| 1169 | II-11 | [III-5/III-4/III-4] |
| 1170 | II-11 | [III-5/III-4/III-5] |
| 1171 | II-11 | [III-5/III-5/III-1] |
| 1172 | II-11 | [III-5/III-5/III-2] |
| 1173 | II-11 | [III-5/III-5/III-3] |
| 1174 | II-11 | [III-5/III-5/III-4] |
| 1175 | II-11 | [III-5/III-5/III-5] |
| 1176 | II-12 | [III-1/III-1/III-1] |
| 1177 | II-12 | [III-1/III-1/III-2] |
| 1178 | II-12 | [III-1/III-1/III-3] |
| 1179 | II-12 | [III-1/III-1/III-4] |
| 1180 | II-12 | [III-1/III-1/III-5] |
| 1181 | II-12 | [III-1/III-2/III-1] |
| 1182 | II-12 | [III-1/III-2/III-2] |
| 1183 | II-12 | [III-1/III-2/III-3] |
| 1184 | II-12 | [III-1/III-2/III-4] |
| 1185 | II-12 | [III-1/III-2/III-5] |
| 1186 | II-12 | [III-1/III-3/III-1] |
| 1187 | II-12 | [III-1/III-3/III-2] |
| 1188 | II-12 | [III-1/III-3/III-3] |
| 1189 | II-12 | [III-1/III-3/III-4] |
| 1190 | II-12 | [III-1/III-3/III-5] |
| 1191 | II-12 | [III-1/III-4/III-1] |
| 1192 | II-12 | [III-1/III-4/III-2] |
| 1193 | II-12 | [III-1/III-4/III-3] |
| 1194 | II-12 | [III-1/III-4/III-4] |
| 1195 | II-12 | [III-1/III-4/III-5] |
| 1196 | II-12 | [III-1/III-5/III-1] |
| 1197 | II-12 | [III-1/III-5/III-2] |
| 1198 | II-12 | [III-1/III-5/III-3] |
| 1199 | II-12 | [III-1/III-5/III-4] |
| 1200 | II-12 | [III-1/III-5/III-5] |
| 1201 | II-12 | [III-2/III-1/III-1] |
| 1202 | II-12 | [III-2/III-1/III-2] |
| 1203 | II-12 | [III-2/III-1/III-3] |
| 1204 | II-12 | [III-2/III-1/III-4] |
| 1205 | II-12 | [III-2/III-1/III-5] |
| 1206 | II-12 | [III-2/III-2/III-1] |
| 1207 | II-12 | [III-2/III-2/III-2] |
| 1208 | II-12 | [III-2/III-2/III-3] |
| 1209 | II-12 | [III-2/III-2/III-4] |
| 1210 | II-12 | [III-2/III-2/III-5] |
| 1211 | II-12 | [III-2/III-3/III-1] |
| 1212 | II-12 | [III-2/III-3/III-2] |
| 1213 | II-12 | [III-2/III-3/III-3] |
| 1214 | II-12 | [III-2/III-3/III-4] |
| 1215 | II-12 | [III-2/III-3/III-5] |
| 1216 | II-12 | [III-2/III-4/III-1] |
| 1217 | II-12 | [III-2/III-4/III-2] |
| 1218 | II-12 | [III-2/III-4/III-3] |
| 1219 | II-12 | [III-2/III-4/III-4] |
| 1220 | II-12 | [III-2/III-4/III-5] |
| 1221 | II-12 | [III-2/III-5/III-4] |
| 1222 | II-12 | [III-2/III-5/III-2] |
| 1223 | II-12 | [III-2/III-5/III-3] |
| 1224 | II-12 | [III-2/III-5/III-4] |
| 1225 | II-12 | [III-2/III-5/III-5] |
| 1226 | II-12 | [III-3/III-1/III-1] |
| 1227 | II-12 | [III-3/III-1/III-2] |
| 1228 | II-12 | [III-3/III-1/III-3] |
| 1229 | II-12 | [III-3/III-1/III-4] |
| 1230 | II-12 | [III-3/III-1/III-5] |
| 1231 | II-12 | [III-3/III-2/III-1] |
| 1232 | II-12 | [III-3/III-2/III-2] |
| 1233 | II-12 | [III-3/III-2/III-3] |
| 1234 | II-12 | [III-3/III-2/III-4] |
| 1235 | II-12 | [III-3/III-2/III-5] |
| 1236 | II-12 | [III-3/III-3/III-1] |
| 1237 | II-12 | [III-3/III-3/III-2] |
| 1238 | II-12 | [III-3/III-3/III-3] |
| 1239 | II-12 | [III-3/III-3/III-4] |
| 1240 | II-12 | [III-3/III-3/III-5] |
| 1241 | II-12 | [III-3/III-4/III-1] |
| 1242 | II-12 | [III-3/III-4/III-2] |
| 1243 | II-12 | [III-3/III-4/III-3] |
| 1244 | II-12 | [III-3/III-4/III-4] |
| 1245 | II-12 | [III-3/III-4/III-5] |
| 1246 | II-12 | [III-3/III-5/III-1] |
| 1247 | II-12 | [III-3/III-5/III-2] |
| 1248 | II-12 | [III-3/III-5/III-3] |
| 1249 | II-12 | [III-3/III-5/III-4] |
| 1250 | II-12 | [III-3/III-5/III-5] |
| 1251 | II-12 | [III-4/III-1/III-1] |
| 1252 | II-12 | [III-4/III-1/III-2] |
| 1253 | II-12 | [III-4/III-1/III-3] |
| 1254 | II-12 | [III-4/III-1/III-4] |
| 1255 | II-12 | [III-4/III-1/III-5] |
| 1256 | II-12 | [III-4/III-2/III-1] |
| 1257 | II-12 | [III-4/III-2/III-2] |
| 1258 | II-12 | [III-4/III-2/III-3] |
| 1259 | II-12 | [III-4/III-2/III-4] |
| 1260 | II-12 | [III-4/III-2/III-5] |
| 1261 | II-12 | [III-4/III-3/III-1] |
| 1262 | II-12 | [III-4/III-3/III-2] |
| 1263 | II-12 | [III-4/III-3/III-3] |
| 1264 | II-12 | [III-4/III-3/III-4] |
| 1265 | II-12 | [III-4/III-3/III-5] |
| 1266 | II-12 | [III-4/III-4/III-1] |
| 1267 | II-12 | [III-4/III-4/III-2] |
| 1268 | II-12 | [III-4/III-4/III-3] |
| 1269 | II-12 | [III-4/III-4/III-4] |
| 1270 | II-12 | [III-4/III-4/III-5] |
| 1271 | II-12 | [III-4/III-5/III-1] |
| 1272 | II-12 | [III-4/III-5/III-2] |
| 1273 | II-12 | [III-4/III-5/III-3] |
| 1274 | II-12 | [III-4/III-5/III-4] |
| 1275 | II-12 | [III-4/III-5/III-5] |
| 1276 | II-12 | [III-5/III-1/III-1] |
| 1277 | II-12 | [III-5/III-1/III-2] |
| 1278 | II-12 | [III-5/III-1/III-3] |
| 1279 | II-12 | [III-5/III-1/III-4] |
| 1280 | II-12 | [III-5/III-1/III-5] |
| 1281 | II-12 | [III-5/III-2/III-1] |
| 1282 | II-12 | [III-5/III-2/III-2] |
| 1283 | II-12 | [III-5/III-2/III-3] |
| 1284 | II-12 | [III-5/III-2/III-4] |
| 1285 | II-12 | [III-5/III-2/III-5] |
| 1286 | II-12 | [III-5/III-3/III-1] |
| 1287 | II-12 | [III-5/III-3/III-2] |
| 1288 | II-12 | [III-5/III-3/III-3] |
| 1289 | II-12 | [III-5/III-3/III-4] |
| 1290 | II-12 | [III-5/III-3/III-5] |
| 1291 | II-12 | [III-5/III-4/III-1] |
| 1292 | II-12 | [III-5/III-4/III-2] |
| 1293 | II-12 | [III-5/III-4/III-3] |
| 1294 | II-12 | [III-5/III-4/III-4] |
| 1295 | II-12 | [III-5/III-4/III-5] |
| 1296 | II-12 | [III-5/III-5/III-1] |
| 1297 | II-12 | [III-5/III-5/III-2] |
| 1298 | II-12 | [III-5/III-5/III-3] |
| 1299 | II-12 | [III-5/III-5/III-4] |
| 1300 | II-12 | [III-5/III-5/III-5] |
| 1301 | II-13 | [III-1/III-1/III-1] |
| 1302 | II-13 | [III-1/III-1/III-2] |
| 1303 | II-13 | [III-1/III-1/III-3] |
| 1304 | II-13 | [III-1/III-1/III-4] |
| 1305 | II-13 | [III-1/III-1/III-5] |
| 1306 | II-13 | [III-1/III-2/III-1] |
| 1307 | II-13 | [III-1/III-2/III-2] |
| 1308 | II-13 | [III-1/III-2/III-3] |
| 1309 | II-13 | [III-1/III-2/III-4] |
| 1310 | II-13 | [III-1/III-2/III-5] |
| 1311 | II-13 | [III-1/III-3/III-1] |
| 1312 | II-13 | [III-1/III-3/III-2] |
| 1313 | II-13 | [III-1/III-3/III-3] |
| 1314 | II-13 | [III-1/III-3/III-4] |
| 1315 | II-13 | [III-1/III-3/III-5] |
| 1316 | II-13 | [III-1/III-4/III-1] |
| 1317 | II-13 | [III-1/III-4/III-2] |
| 1318 | II-13 | [III-1/III-4/III-3] |
| 1319 | II-13 | [III-1/III-4/III-4] |
| 1320 | II-13 | [III-1/III-4/III-5] |
| 1321 | II-13 | [III-1/III-5/III-1] |
| 1322 | II-13 | [III-1/III-5/III-2] |
| 1323 | II-13 | [III-1/III-5/III-3] |
| 1324 | II-13 | [III-1/III-5/III-4] |
| 1325 | II-13 | [III-1/III-5/III-5] |
| 1326 | II-13 | [III-2/III-1/III-2] |
| 1327 | II-13 | [III-2/III-1/III-3] |
| 1328 | II-13 | [III-2/III-1/III-4] |
| 1329 | II-13 | [III-2/III-1/III-5] |
| 1330 | II-13 | [III-2/III-2/III-2] |
| 1331 | II-13 | [III-2/III-2/III-3] |
| 1332 | II-13 | [III-2/III-2/III-4] |
| 1333 | II-13 | [III-2/III-2/III-5] |
| 1334 | II-13 | [III-2/III-3/III-2] |
| 1335 | II-13 | [III-2/III-3/III-3] |
| 1336 | II-13 | [III-2/III-3/III-4] |
| 1337 | II-13 | [III-2/III-3/III-5] |
| 1338 | II-13 | [III-2/III-4/III-2] |
| 1339 | II-13 | [III-2/III-4/III-3] |
| 1340 | II-13 | [III-2/III-4/III-4] |
| 1341 | II-13 | [III-2/III-4/III-5] |
| 1342 | II-13 | [III-2/III-5/III-2] |
| 1343 | II-13 | [III-2/III-5/III-3] |
| 1344 | II-13 | [III-2/III-5/III-4] |
| 1345 | II-13 | [III-2/III-5/III-5] |
| 1346 | II-13 | [III-3/III-1/III-3] |
| 1347 | II-13 | [III-3/III-1/III-4] |
| 1348 | II-13 | [III-3/III-1/III-5] |
| 1349 | II-13 | [III-3/III-2/III-3] |
| 1350 | II-13 | [III-3/III-2/III-4] |
| 1351 | II-13 | [III-3/III-2/III-5] |
| 1352 | II-13 | [III-3/III-3/III-3] |
| 1353 | II-13 | [III-3/III-3/III-4] |
| 1354 | II-13 | [III-3/III-3/III-5] |
| 1355 | II-13 | [III-3/III-4/III-3] |
| 1356 | II-13 | [III-3/III-4/III-4] |
| 1357 | II-13 | [III-3/III-4/III-5] |
| 1358 | II-13 | [III-3/III-5/III-3] |
| 1359 | II-13 | [III-3/III-5/III-4] |
| 1360 | II-13 | [III-3/III-5/III-5] |
| 1361 | II-13 | [III-4/III-1/III-4] |
| 1362 | II-13 | [III-4/III-1/III-5] |
| 1363 | II-13 | [III-4/III-2/III-4] |
| 1364 | II-13 | [III-4/III-2/III-5] |
| 1365 | II-13 | [III-4/III-3/III-4] |
| 1366 | II-13 | [III-4/III-3/III-5] |
| 1367 | II-13 | [III-4/III-4/III-4] |
| 1368 | II-13 | [III-4/III-4/III-5] |
| 1369 | II-13 | [III-4/III-5/III-4] |
| 1370 | II-13 | [III-4/III-5/III-5] |
| 1371 | II-13 | [III-5/III-1/III-5] |
| 1372 | II-13 | [III-5/III-2/III-5] |
| 1373 | II-13 | [III-5/III-3/III-5] |
| 1374 | II-13 | [III-5/III-4/III-5] |
| 1375 | II-13 | [III-5/III-5/III-5] |
| 1376 | II-14 | [III-1/III-1/III-1] |
| 1377 | II-14 | [III-1/III-1/III-2] |
| 1378 | II-14 | [III-1/III-1/III-3] |
| 1379 | II-14 | [III-1/III-1/III-4] |
| 1380 | II-14 | [III-1/III-1/III-5] |
| 1381 | II-14 | [III-1/III-2/III-1] |
| 1382 | II-14 | [III-1/III-2/III-2] |
| 1383 | II-14 | [III-1/III-2/III-3] |
| 1384 | II-14 | [III-1/III-2/III-4] |
| 1385 | II-14 | [III-1/III-2/III-5] |
| 1386 | II-14 | [III-1/III-3/III-1] |
| 1387 | II-14 | [III-1/III-3/III-2] |
| 1388 | II-14 | [III-1/III-3/III-3] |
| 1389 | II-14 | [III-1/III-3/III-4] |
| 1390 | II-14 | [III-1/III-3/III-5] |
| 1391 | II-14 | [III-1/III-4/III-1] |
| 1392 | II-14 | [III-1/III-4/III-2] |
| 1393 | II-14 | [III-1/III-4/III-3] |
| 1394 | II-14 | [III-1/III-4/III-4] |
| 1395 | II-14 | [III-1/III-4/III-5] |
| 1396 | II-14 | [III-1/III-5/III-1] |
| 1397 | II-14 | [III-1/III-5/III-2] |
| 1398 | II-14 | [III-1/III-5/III-3] |
| 1399 | II-14 | [III-1/III-5/III-4] |
| 1400 | II-14 | [III-1/III-5/III-5] |
| 1401 | II-14 | [III-2/III-1/III-2] |
| 1402 | II-14 | [III-2/III-1/III-3] |
| 1403 | II-14 | [III-2/III-1/III-4] |
| 1404 | II-14 | [III-2/III-1/III-5] |
| 1405 | II-14 | [III-2/III-2/III-2] |
| 1406 | II-14 | [III-2/III-2/III-3] |
| 1407 | II-14 | [III-2/III-2/III-4] |
| 1408 | II-14 | [III-2/III-2/III-5] |
| 1409 | II-14 | [III-2/III-3/III-2] |
| 1410 | II-14 | [III-2/III-3/III-3] |
| 1411 | II-14 | [III-2/III-3/III-4] |
| 1412 | II-14 | [III-2/III-3/III-5] |
| 1413 | II-14 | [III-2/III-4/III-2] |
| 1414 | II-14 | [III-2/III-4/III-3] |
| 1415 | II-14 | [III-2/III-4/III-4] |
| 1416 | II-14 | [III-2/III-4/III-5] |
| 1417 | II-14 | [III-2/III-5/III-2] |
| 1418 | II-14 | [III-2/III-5/III-3] |
| 1419 | II-14 | [III-2/III-5/III-4] |
| 1420 | II-14 | [III-2/III-5/III-5] |
| 1421 | II-14 | [III-3/III-1/III-3] |
| 1422 | II-14 | [III-3/III-1/III-4] |
| 1423 | II-14 | [III-3/III-1/III-5] |
| 1424 | II-14 | [III-3/III-2/III-3] |
| 1425 | II-14 | [III-3/III-2/III-4] |
| 1426 | II-14 | [III-3/III-2/III-5] |
| 1427 | II-14 | [III-3/III-3/III-3] |
| 1428 | II-14 | [III-3/III-3/III-4] |
| 1429 | II-14 | [III-3/III-3/III-5] |
| 1430 | II-14 | [III-3/III-4/III-3] |
| 1431 | II-14 | [III-3/III-4/III-4] |
| 1432 | II-14 | [III-3/III-4/III-5] |
| 1433 | II-14 | [III-3/III-5/III-3] |
| 1434 | II-14 | [III-3/III-5/III-4] |
| 1435 | II-14 | [III-3/III-5/III-5] |
| 1436 | II-14 | [III-4/III-1/III-4] |
| 1437 | II-14 | [III-4/III-1/III-5] |
| 1438 | II-14 | [III-4/III-2/III-4] |
| 1439 | II-14 | [III-4/III-2/III-5] |
| 1440 | II-14 | [III-4/III-3/III-4] |
| 1441 | II-14 | [III-4/III-3/III-5] |
| 1442 | II-14 | [III-4/III-4/III-4] |
| 1443 | II-14 | [III-4/III-4/III-5] |
| 1444 | II-14 | [III-4/III-5/III-4] |
| 1445 | II-14 | [III-4/III-5/III-5] |
| 1446 | II-14 | [III-5/III-1/III-5] |
| 1447 | II-14 | [III-5/III-2/III-5] |
| 1448 | II-14 | [III-5/III-3/III-5] |
| 1449 | II-14 | [III-5/III-4/III-5] |
| 1450 | II-14 | [III-5/III-5/III-5] |
| 1451 | II-15 | [III-1/III-1/III-1] |
| 1452 | II-15 | [III-1/III-1/III-2] |
| 1453 | II-15 | [III-1/III-1/III-3] |
| 1454 | II-15 | [III-1/III-1/III-4] |
| 1455 | II-15 | [III-1/III-1/III-5] |
| 1456 | II-15 | [III-1/III-2/III-1] |
| 1457 | II-15 | [III-1/III-2/III-2] |
| 1458 | II-15 | [III-1/III-2/III-3] |
| 1459 | II-15 | [III-1/III-2/III-4] |
| 1460 | II-15 | [III-1/III-2/III-5] |
| 1461 | II-15 | [III-1/III-3/III-1] |
| 1462 | II-15 | [III-1/III-3/III-2] |
| 1463 | II-15 | [III-1/III-3/III-3] |
| 1464 | II-15 | [III-1/III-3/III-4] |
| 1465 | II-15 | [III-1/III-3/III-5] |
| 1466 | II-15 | [III-1/III-4/III-1] |
| 1467 | II-15 | [III-1/III-4/III-2] |
| 1468 | II-15 | [III-1/III-4/III-3] |
| 1469 | II-15 | [III-1/III-4/III-4] |
| 1470 | II-15 | [III-1/III-4/III-5] |
| 1471 | II-15 | [III-1/III-5/III-1] |
| 1472 | II-15 | [III-1/III-5/III-2] |
| 1473 | II-15 | [III-1/III-5/III-3] |
| 1474 | II-15 | [III-1/III-5/III-4] |
| 1475 | II-15 | [III-1/III-5/III-5] |
| 1476 | II-15 | [III-2/III-1/III-1] |
| 1477 | II-15 | [III-2/III-1/III-2] |
| 1478 | II-15 | [III-2/III-1/III-3] |
| 1479 | II-15 | [III-2/III-1/III-4] |
| 1480 | II-15 | [III-2/III-1/III-5] |
| 1481 | II-15 | [III-2/III-2/III-1] |
| 1482 | II-15 | [III-2/III-2/III-2] |
| 1483 | II-15 | [III-2/III-2/III-3] |
| 1484 | II-15 | [III-2/III-2/III-4] |
| 1485 | II-15 | [III-2/III-2/III-5] |
| 1486 | II-15 | [III-2/III-3/III-1] |
| 1487 | II-15 | [III-2/III-3/III-2] |
| 1488 | II-15 | [III-2/III-3/III-3] |
| 1489 | II-15 | [III-2/III-3/III-4] |
| 1490 | II-15 | [III-2/III-3/III-5] |
| 1491 | II-15 | [III-2/III-4/III-1] |
| 1492 | II-15 | [III-2/III-4/III-2] |
| 1493 | II-15 | [III-2/III-4/III-3] |
| 1494 | II-15 | [III-2/III-4/III-4] |
| 1495 | II-15 | [III-2/III-4/III-5] |
| 1496 | II-15 | [III-2/III-5/III-1] |
| 1497 | II-15 | [III-2/III-5/III-2] |
| 1498 | II-15 | [III-2/III-5/III-3] |
| 1499 | II-15 | [III-2/III-5/III-4] |
| 1500 | II-15 | [III-2/III-5/III-5] |
| 1501 | II-15 | [III-3/III-1/III-1] |
| 1502 | II-15 | [III-3/III-1/III-2] |
| 1503 | II-15 | [III-3/III-1/III-3] |
| 1504 | II-15 | [III-3/III-1/III-4] |
| 1505 | II-15 | [III-3/III-1/III-5] |
| 1506 | II-15 | [III-3/III-2/III-1] |
| 1507 | II-15 | [III-3/III-2/III-2] |
| 1508 | II-15 | [III-3/III-2/III-3] |
| 1509 | II-15 | [III-3/III-2/III-4] |
| 1510 | II-15 | [III-3/III-2/III-5] |
| 1511 | II-15 | [III-3/III-3/III-1] |
| 1512 | II-15 | [III-3/III-3/III-2] |
| 1513 | II-15 | [III-3/III-3/III-3] |
| 1514 | II-15 | [III-3/III-3/III-4] |
| 1515 | II-15 | [III-3/III-3/III-5] |
| 1516 | II-15 | [III-3/III-4/III-1] |
| 1517 | II-15 | [III-3/III-4/III-2] |
| 1518 | II-15 | [III-3/III-4/III-3] |
| 1519 | II-15 | [III-3/III-4/III-4] |
| 1520 | II-15 | [III-3/III-4/III-5] |
| 1521 | II-15 | [III-3/III-5/III-1] |
| 1522 | II-15 | [III-3/III-5/III-2] |
| 1523 | II-15 | [III-3/III-5/III-3] |
| 1524 | II-15 | [III-3/III-5/III-4] |
| 1525 | II-15 | [III-3/III-5/III-5] |
| 1526 | II-15 | [III-4/III-1/III-1] |
| 1527 | II-15 | [III-4/III-1/III-2] |
| 1528 | II-15 | [III-4/III-1/III-3] |
| 1529 | II-15 | [III-4/III-1/III-4] |
| 1530 | II-15 | [III-4/III-1/III-5] |
| 1531 | II-15 | [III-4/III-2/III-1] |
| 1532 | II-15 | [III-4/III-2/III-2] |
| 1533 | II-15 | [III-4/III-2/III-3] |
| 1534 | II-15 | [III-4/III-2/III-4] |
| 1535 | II-15 | [III-4/III-2/III-5] |
| 1536 | II-15 | [III-4/III-3/III-1] |
| 1537 | II-15 | [III-4/III-3/III-2] |
| 1538 | II-15 | [III-4/III-3/III-3] |
| 1539 | II-15 | [III-4/III-3/III-4] |
| 1540 | II-15 | [III-4/III-3/III-5] |
| 1541 | II-15 | [III-4/III-4/III-1] |
| 1542 | II-15 | [III-4/III-4/III-2] |
| 1543 | II-15 | [III-4/III-4/III-3] |
| 1544 | II-15 | [III-4/III-4/III-4] |
| 1545 | II-15 | [III-4/III 4/III-5] |
| 1546 | II-15 | [III-4/III-5/III-1] |
| 1547 | II-15 | [III-4/III-5/III-2] |
| 1548 | II-15 | [III-4/III-5/III-3] |
| 1549 | II-15 | [III-4/III-5/III-4] |
| 1550 | II-15 | [III-4/III-5/III-5] |
| 1551 | II-15 | [III-5/III-1/III-1] |
| 1552 | II-15 | [III-5/III-1/III-2] |
| 1553 | II-15 | [III-5/III-1/III-3] |
| 1554 | II-15 | [III-5/III-1/III-4] |
| 1555 | II-15 | [III-5/III-1/III-5] |
| 1556 | II-15 | [III-5/III-2/III-1] |
| 1557 | II-15 | [III-5/III-2/III-2] |
| 1558 | II-15 | [III-5/III-2/III-3] |
| 1559 | II-15 | [III-5/III-2/III-4] |
| 1560 | II-15 | [III-5/III-2/III-5] |
| 1561 | II-15 | [III-5/III-3/III-1] |
| 1562 | II-15 | [III-5/III-3/III-2] |
| 1563 | II-15 | [III-5/III-3/III-3] |
| 1564 | II-15 | [III-5/III-3/III-4] |
| 1565 | II-15 | [III-5/III-3/III-5] |
| 1566 | II-15 | [III-5/III-4/III-1] |
| 1567 | II-15 | [III-5/III-4/III-2] |
| 1568 | II-15 | [III-5/III-4/III-3] |
| 1569 | II-15 | [III-5/III-4/III-4] |
| 1570 | II-15 | [III-5/III-4/III-5] |
| 1571 | II-15 | [III-5/III-5/III-1] |
| 1572 | II-15 | [III-5/III-5/III-2] |
| 1573 | II-15 | [III-5/III-5/III-3] |
| 1574 | II-15 | [III-5/III-5/III-4] |
| 1575 | II-15 | [III-5/III-5/III-5] |
| 1576 | II-16 | [III-1/III-1/III-1] |
| 1577 | II-16 | [III-1/III-1/III-2] |
| 1578 | II-16 | [III-1/III-1/III-3] |
| 1579 | II-16 | [III-1/III-1/III-4] |
| 1580 | II-16 | [III-1/III-1/III-5] |
| 1581 | II-16 | [III-1/III-2/III-1] |
| 1582 | II-16 | [III-1/III-2/III-2] |
| 1583 | II-16 | [III-1/III-2/III-3] |
| 1584 | II-16 | [III-1/III-2/III-4] |
| 1585 | II-16 | [III-1/III-2/III-5] |
| 1586 | II-16 | [III-1/III-3/III-1] |
| 1587 | II-16 | [III-1/III-3/III-2] |
| 1588 | II-16 | [III-1/III-3/III-3] |
| 1589 | II-16 | [III-1/III-3/III-4] |
| 1590 | II-16 | [III-1/III-3/III-5] |
| 1591 | II-16 | [III-1/III-4/III-1] |
| 1592 | II-16 | [III-1/III-4/III-2] |
| 1593 | II-16 | [III-1/III-4/III-3] |
| 1594 | II-16 | [III-1/III-4/III-4] |
| 1595 | II-16 | [III-1/III-4/III-5] |
| 1596 | II-16 | [III-1/III-5/III-1] |
| 1597 | II-16 | [III-1/III-5/III-2] |
| 1598 | II-16 | [III-1/III-5/III-3] |
| 1599 | II-16 | [III-1/III-5/III-4] |
| 1600 | II-16 | [III-1/III-5/III-5] |
| 1601 | II-16 | [III-2/III-1/III-1] |
| 1602 | II-16 | [III-2/III-1/III-2] |
| 1603 | II-16 | [III-2/III-1/III-3] |
| 1604 | II-16 | [III-2/III-1/III-4] |
| 1605 | II-16 | [III-2/III-1/III-5] |
| 1606 | II-16 | [III-2/III-2/III-1] |
| 1607 | II-16 | [III-2/III-2/III-2] |
| 1608 | II-16 | [III-2/III-2/III-3] |
| 1609 | II-16 | [III-2/III-2/III-4] |
| 1610 | II-16 | [III-2/III-2/III-5] |
| 1611 | II-16 | [III-2/III-3/III-1] |
| 1612 | II-16 | [III-2/III-3/III-2] |
| 1613 | II-16 | [III-2/III-3/III-3] |
| 1614 | II-16 | [III-2/III-3/III-4] |
| 1615 | II-16 | [III-2/III-3/III-5] |
| 1616 | II-16 | [III-2/III-4/III-1] |
| 1617 | II-16 | [III-2/III-4/III-2] |
| 1618 | II-16 | [III-2/III-4/III-3] |
| 1619 | II-16 | [III-2/III-4/III-4] |
| 1620 | II-16 | [III-2/III-4/III-5] |
| 1621 | II-16 | [III-2/III-5/III-1] |
| 1622 | II-16 | [III-2/III-5/III-2] |
| 1623 | II-16 | [III-2/III-5/III-3] |
| 1624 | II-16 | [III-2/III-5/III-4] |
| 1625 | II-16 | [III-2/III-5/III-5] |
| 1626 | II-16 | [III-3/III-1/III-1] |
| 1627 | II-16 | [III-3/III-1/III-2] |
| 1628 | II-16 | [III-3/III-1/III-3] |
| 1629 | II-16 | [III-3/III-1/III-4] |
| 1630 | II-16 | [III-3/III-1/III-5] |
| 1631 | II-16 | [III-3/III-2/III-1] |
| 1632 | II-16 | [III-3/III-2/III-2] |
| 1633 | II-16 | [III-3/III-2/III-3] |
| 1634 | II-16 | [III-3/III-2/III-4] |
| 1635 | II-16 | [III-3/III-2/III-5] |
| 1636 | II-16 | [III-3/III-3/III-1] |
| 1637 | II-16 | [III-3/III-3/III-2] |
| 1638 | II-16 | [III-3/III-3/III-3] |
| 1639 | II-16 | [III-3/III-3/III-4] |
| 1640 | II-16 | [III-3/III-3/III-5] |
| 1641 | II-16 | [III-3/III-4/III-1] |
| 1642 | II-16 | [III-3/III-4/III-2] |
| 1643 | II-16 | [III-3/III-4/III-3] |
| 1644 | II-16 | [III-3/III-4/III-4] |
| 1645 | II-16 | [III-3/III-4/III-5] |
| 1646 | II-16 | [III-3/III-5/III-1] |
| 1647 | II-16 | [III-3/III-5/III-2] |
| 1648 | II-16 | [III-3/III-5/III-3] |
| 1649 | II-16 | [III-3/III-5/III-4] |
| 1650 | II-16 | [III-3/III-5/III-5] |
| 1651 | II-16 | [III-4/III-1/III-1] |
| 1652 | II-16 | [III-4/III-1/III-2] |
| 1653 | II-16 | [III-4/III-1/III-3] |
| 1654 | II-16 | [III-4/III-1/III-4] |
| 1655 | II-16 | [III-4/III-1/III-5] |
| 1656 | II-16 | [III-4/III-2/III-1] |
| 1657 | II-16 | [III-4/III-2/III-2] |
| 1658 | II-16 | [III-4/III-2/III-3] |
| 1659 | II-16 | [III-4/III-2/III-4] |
| 1660 | II-16 | [III-4/III-2/III-5] |
| 1661 | II-16 | [III-4/III-3/III-1] |
| 1662 | II-16 | [III-4/III-3/III-2] |
| 1663 | II-16 | [III-4/III-3/III-3] |
| 1664 | II-16 | [III-4/III-3/III-4] |
| 1665 | II-16 | [III-4/III-3/III-5] |
| 1666 | II-16 | [III-4/III-4/III-1] |
| 1667 | II-16 | [III-4/III-4/III-2] |
| 1668 | II-16 | [III-4/III-4/III-3] |
| 1669 | II-16 | [III-4/III-4/III-4] |
| 1670 | II-16 | [III-4/III-4/III-5] |
| 1671 | II-16 | [III-4/III-5/III-1] |
| 1672 | II-16 | [III-4/III-5/III-2] |
| 1673 | II-16 | [III-4/III-5/III-3] |
| 1674 | II-16 | [III-4/III-5/III-4] |
| 1675 | II-16 | [III-4/III-5/III-5] |
| 1676 | II-16 | [III-5/III-1/III-1] |
| 1677 | II-16 | [III-5/III-1/III-2] |
| 1678 | II-16 | [III-5/III-1/III-3] |
| 1679 | II-16 | [III-5/III-1/III-4] |
| 1680 | II-16 | [III-5/III-1/III-5] |
| 1681 | II-16 | [III-5/III-2/III-1] |
| 1682 | II-16 | [III-5/III-2/III-2] |
| 1683 | II-16 | [III-5/III-2/III-3] |
| 1684 | II-16 | [III-5/III-2/III-4] |
| 1685 | II-16 | [III-5/III-2/III-5] |
| 1686 | II-16 | [III-5/III-3/III-1] |
| 1687 | II-16 | [III-5/III-3/III-2] |
| 1688 | II-16 | [III-5/III-3/III-3] |
| 1689 | II-16 | [III-5/III-3/III-4] |
| 1690 | II-16 | [III-5/III-3/III-5] |
| 1691 | II-16 | [III-5/III-4/III-1] |
| 1692 | II-16 | [III-5/III-4/III-2] |
| 1693 | II-16 | [III-5/III-4/III-3] |
| 1694 | II-16 | [III-5/III-4/III-4] |
| 1695 | II-16 | [III-5/III-4/III-5] |
| 1696 | II-16 | [III-5/III-5/III-1] |
| 1697 | II-16 | [III-5/III-5/III-2] |
| 1698 | II-16 | [III-5/III-5/III-3] |
| 1699 | II-16 | [III-5/III-5/III-4] |
| 1700 | II-16 | [III-5/III-5/III-5] |

11. An amine compound represented by Formula 1: 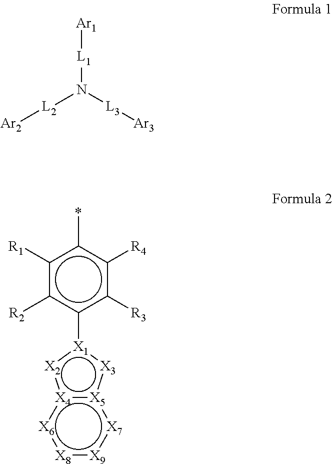
wherein in Formula 1,
*-L₁-Ar₁, *-L₂-Ar₂, and *-L₃-Ar₃ are each independently represented by Formula 2, and
in Formula 2,
R₁ to R₄ are each independently a hydroxy group or a hydrogen atom, or bonded to an adjacent group to form a single bond,
X₁ to X₉ are each independently CH, C, N, O, or S, and
at least one of *-L₁-Ar₁, *-L₂-Ar₂, or *-L₃-Ar₃ comprises the hydroxy group.

12. The amine compound of claim 11, wherein Formula 2 is represented by any one selected from among I-1 to I-3, wherein in Formula 1, *-L₁-Ar₁, *-L₂-Ar₂, and *-L₃-Ar₃ are not all I-1: 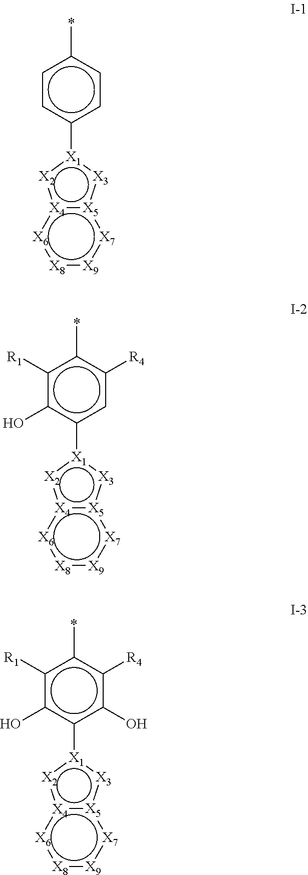
wherein in I-2 and I-3, R₁ and R₄ are each independently a hydrogen atom, or bonded to an adjacent group to form a single bond; and
in I-1 to I-3, X₁ to X₉ are the same as defined in Formula 2.

13. The amine compound of claim 11, wherein Formula 2 is represented by any one selected from among I-4 to I-8: 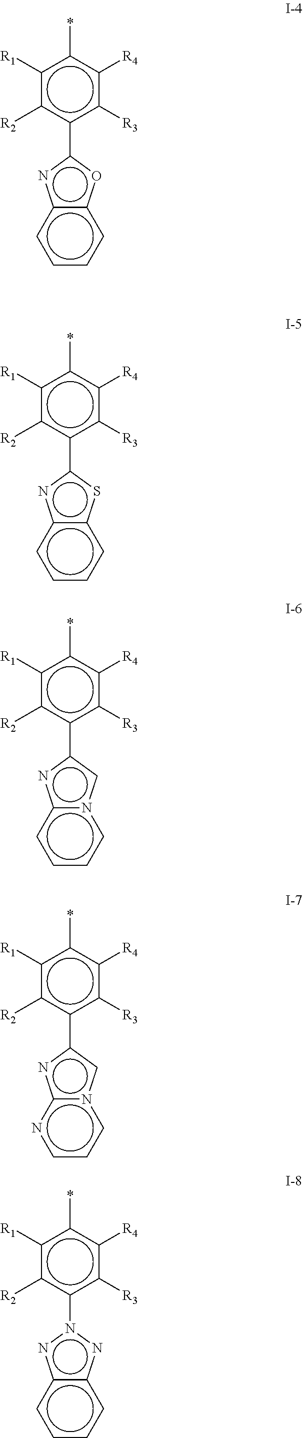
wherein in I-4 to I-8, R₁ to R₄ are the same as defined in Formula 2.

14. The amine compound of claim 11, wherein in Formula 2, at least one selected from among X₁ to X₃ is N.

15. The amine compound of claim 11, wherein Formula 1 is represented by Formula 1-1: 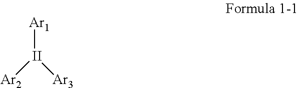
wherein in Formula 1-1, II is represented by II-A or II-B: 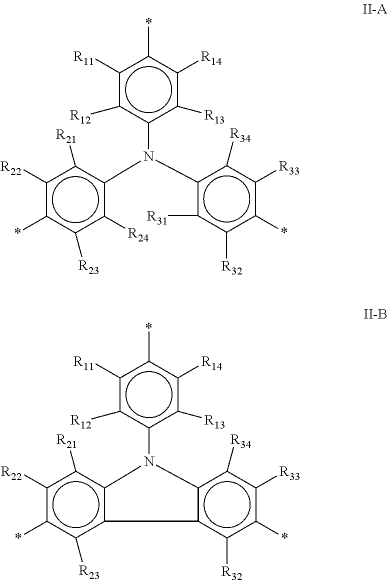
wherein in II-A, at least one selected from among R₁₁ to R₁₄, R₂₁ to R₂₄, and R₃₁ to R₃₄ is hydroxy and the R₁₁ to R₁₄, R₂₁ to R₂₄, and R₃₁ to R₃₄ that are not hydroxy are hydrogen atoms; and
in II-B, at least one selected from among R₁₁ to R₁₄, R₂₁ to R₂₃, and R₃₂ to R₃₄ is hydroxy and the R₁₁ to R₁₄, R₂₁ to R₂₃, and R₃₂ to R₃₄ that are not hydroxy are hydrogen atoms.

16. The amine compound of claim 15, wherein in Formula 1-1, II is represented by any one selected from among II-1 to II-16: 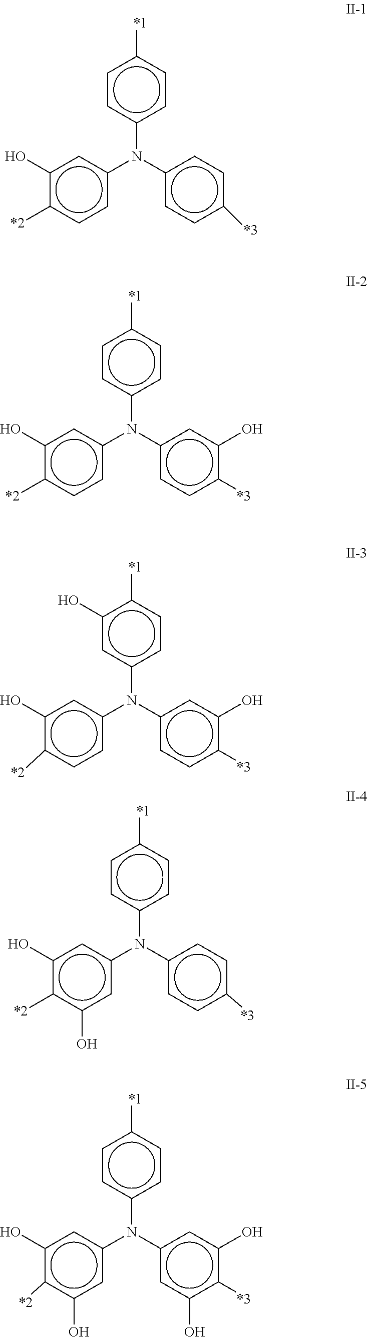 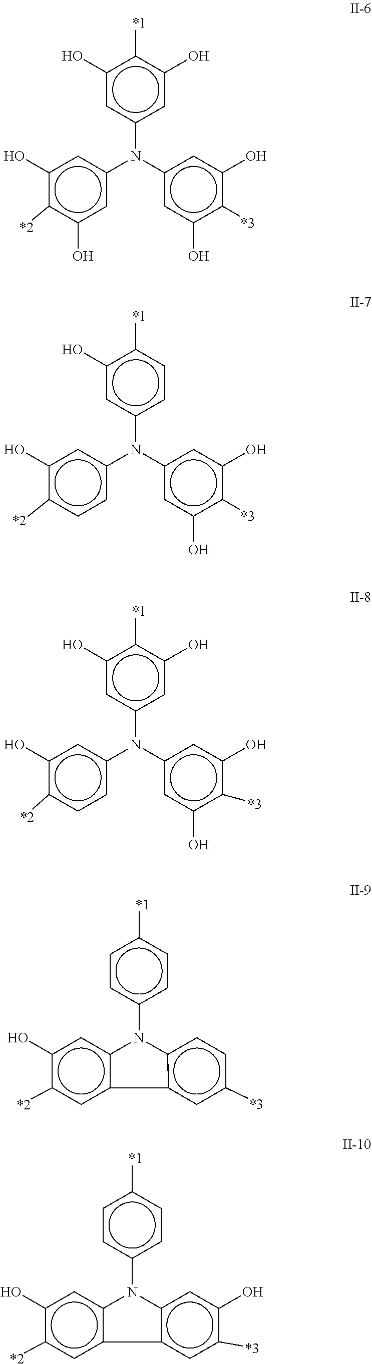 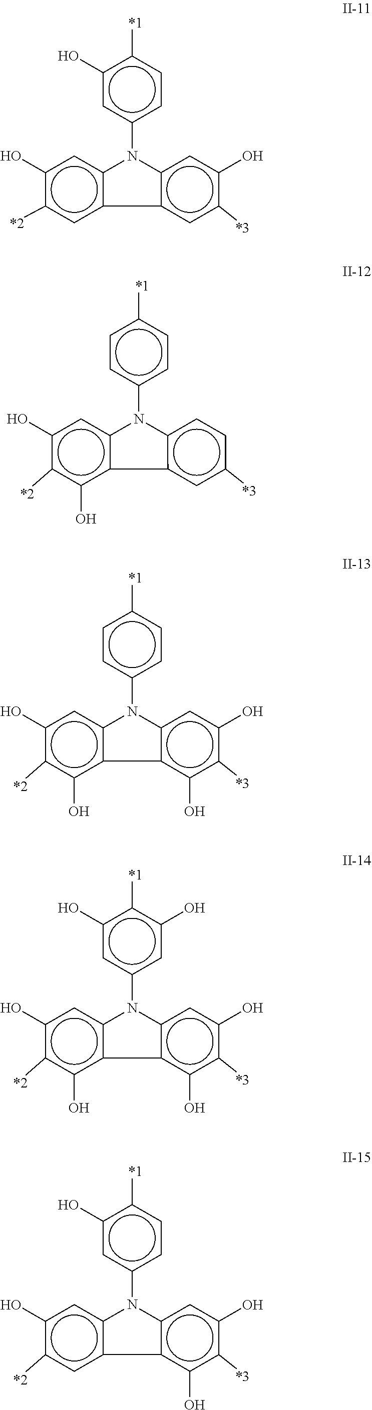 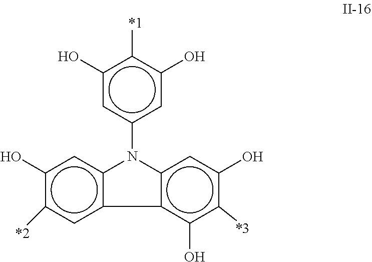
wherein in II-1 to II-16, *1 is a portion to which Ar₁ is bonded, *2 is a portion to which Ar₂ is bonded, and *3 is a portion to which Ar₃ is bonded.

17. The amine compound of claim 15, wherein in Formula 1-1, Ar₁ to Ar₃ are each independently represented by any one selected from among III-1 to III-5:

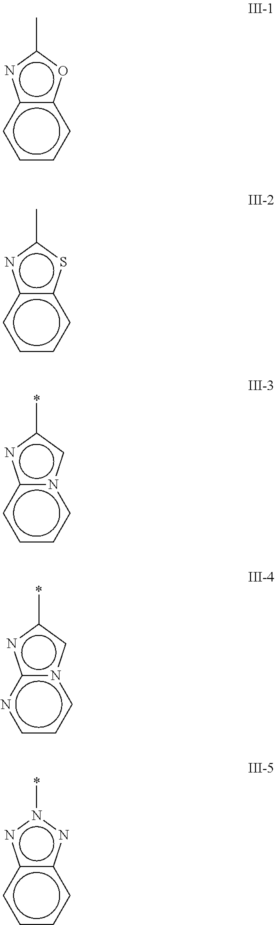

18. The amine compound of claim 15, wherein the amine compound is represented by any one selected from among compounds below:

| Compound | | |
|---|---|---|
| classification | II | [Ar₂/Ar₁/Ar₃] |
| 1 | II-1 | [III-1/III-1/III-1] |
| 2 | II-1 | [III-1/III-1/III-2] |
| 3 | II-1 | [III-1/III-1/III-3] |
| 4 | II-1 | [III-1/III-1/III-4] |
| 5 | II-1 | [III-1/III-1/III-5] |
| 6 | II-1 | [III-1/III-2/III-1] |
| 7 | II-1 | [III-1/III-2/III-2] |
| 8 | II-1 | [III-1/III-2/III-3] |
| 9 | II-1 | [III-1/III-2/III-4] |
| 10 | II-1 | [III-1/III-2/III-5] |
| 11 | II-1 | [III-1/III-3/III-1] |
| 12 | II-1 | [III-1/III-3/III-2] |
| 13 | II-1 | [III-1/III-3/III-3] |
| 14 | II-1 | [III-1/III-3/III-4] |
| 15 | II-1 | [III-1/III-3/III-5] |
| 16 | II-1 | [III-1/III-4/III-1] |
| 17 | II-1 | [III-1/III-4/III-2] |
| 18 | II-1 | [III-1/III-4/III-3] |
| 19 | II-1 | [III-1/III-4/III-4] |
| 20 | II-1 | [III-1/III-4/III-5] |
| 21 | II-1 | [III-1/III-5/III-1] |
| 22 | II-1 | [III-1/III-5/III-2] |
| 23 | II-1 | [III-1/III-5/III-3] |
| 24 | II-1 | [III-1/III-5/III-4] |
| 25 | II-1 | [III-1/III-5/III-5] |
| 26 | II-1 | [III-2/III-1/III-1] |
| 27 | II-1 | [III-2/III-1/III-2] |
| 28 | II-1 | [III-2/III-1/III-3] |
| 29 | II-1 | [III-2/III-1/III-4] |
| 30 | II-1 | [III-2/III-1/III-5] |
| 31 | II-1 | [III-2/III-2/III-1] |
| 32 | II-1 | [III-2/III-2/III-2] |
| 33 | II-1 | [III-2/III-2/III-3] |
| 34 | II-1 | [III-2/III-2/III-4] |
| 35 | II-1 | [III-2/III-2/III-5] |
| 36 | II-1 | [III-2/III-3/III-1] |
| 37 | II-1 | [III-2/III-3/III-2] |
| 38 | II-1 | [III-2/III-3/III-3] |
| 39 | II-1 | [III-2/III-3/III-4] |
| 40 | II-1 | [III-2/III-3/III-5] |
| 41 | II-1 | [III-2/III-4/III-1] |
| 42 | II-1 | [III-2/III-4/III-2] |
| 43 | II-1 | [III-2/III-4/III-3] |
| 44 | II-1 | [III-2/III-4/III-4] |
| 45 | II-1 | [III-2/III-4/III-5] |
| 46 | II-1 | [III-2/III-5/III-1] |
| 47 | II-1 | [III-2/III-5/III-2] |
| 48 | II-1 | [III-2/III-5/III-3] |
| 49 | II-1 | [III-2/III-5/III-4] |
| 50 | II-1 | [III-2/III-5/III-5] |
| 51 | II-1 | [III-3/III-1/III-1] |
| 52 | II-1 | [III-3/III-1/III-2] |
| 53 | II-1 | [III-3/III-1/III-3] |
| 54 | II-1 | [III-3/III-1/III-4] |
| 55 | II-1 | [III-3/III-1/III-5] |
| 56 | II-1 | [III-3/III-2/III-1] |
| 57 | II-1 | [III-3/III-2/III-2] |
| 58 | II-1 | [III-3/III-2/III-3] |
| 59 | II-1 | [III-3/III-2/III-4] |
| 60 | II-1 | [III-3/III-2/III-5] |
| 61 | II-1 | [III-3/III-3/III-1] |
| 62 | II-1 | [III-3/III-3/III-2] |
| 63 | II-1 | [III-3/III-3/III-3] |
| 64 | II-1 | [III-3/III-3/III-4] |
| 65 | II-1 | [III-3/III-3/III-5] |
| 66 | II-1 | [III-3/III-4/III-1] |
| 67 | II-1 | [III-3/III-4/III-2] |
| 68 | II-1 | [III-3/III-4/III-3] |
| 69 | II-1 | [III-3/III-4/III-4] |
| 70 | II-1 | [III-3/III-4/III-5] |
| 71 | II-1 | [III-3/III-5/III-1] |
| 72 | II-1 | [III-3/III-5/III-2] |
| 73 | II-1 | [III-3/III-5/III-3] |
| 74 | II-1 | [III-3/III-5/III-4] |
| 75 | II-1 | [III-3/III-5/III-5] |
| 76 | II-1 | [III-4/III-1/III-1] |
| 77 | II-1 | [III-4/III-1/III-2] |
| 78 | II-1 | [III-4/III-1/III-3] |
| 79 | II-1 | [III-4/III-1/III-4] |
| 80 | II-1 | [III-4/III-1/III-5] |
| 81 | II-1 | [III-4/III-2/III-1] |
| 82 | II-1 | [III-4/III-2/III-2] |
| 83 | II-1 | [III-4/III-2/III-3] |
| 84 | II-1 | [III-4/III-2/III-4] |
| 85 | II-1 | [III-4/III-2/III-5] |
| 86 | II-1 | [III-4/III-3/III-1] |
| 87 | II-1 | [III-4/III-3/III-2] |
| 88 | II-1 | [III-4/III-3/III-3] |
| 89 | II-1 | [III-4/III-3/III-4] |
| 90 | II-1 | [III-4/III-3/III-5] |
| 91 | II-1 | [III-4/III-4/III-1] |
| 92 | II-1 | [III-4/III-4/III-2] |
| 93 | II-1 | [III-4/III-4/III-3] |
| 94 | II-1 | [III-4/III-4/III-4] |
| 95 | II-1 | [III-4/III-4/III-5] |
| 96 | II-1 | [III-4/III-5/III-1] |
| 97 | II-1 | [III-4/III-5/III-2] |
| 98 | II-1 | [III-4/III-5/III-3] |
| 99 | II-1 | [III-4/III-5/III-4] |
| 100 | II-1 | [III-4/III-5/III-5] |
| 101 | II-1 | [III-5/III-1/III-1] |
| 102 | II-1 | [III-5/III-1/III-2] |
| 103 | II-1 | [III-5/III-1/III-3] |
| 104 | II-1 | [III-5/III-1/III-4] |
| 105 | II-1 | [III-5/III-1/III-5] |
| 106 | II-1 | [III-5/III-2/III-1] |
| 107 | II-1 | [III-5/III-2/III-2] |
| 108 | II-1 | [III-5/III-2/III-3] |
| 109 | II-1 | [III-5/III-2/III-4] |
| 110 | II-1 | [III-5/III-2/III-5] |
| 111 | II-1 | [III-5/III-3/III-1] |
| 112 | II-1 | [III-5/III-3/III-2] |
| 113 | II-1 | [III-5/III-3/III-3] |
| 114 | II-1 | [III-5/III-3/III-4] |
| 115 | II-1 | [III-5/III-3/III-5] |
| 116 | II-1 | [III-5/III-4/III-1] |
| 117 | II-1 | [III-5/III-4/III-2] |
| 118 | II-1 | [III-5/III-4/III-3] |
| 119 | II-1 | [III-5/III-4/III-4] |
| 120 | II-1 | [III-5/III-4/III-5] |
| 121 | II-1 | [III-5/III-5/III-1] |
| 122 | II-1 | [III-5/III-5/III-1] |
| 123 | II-1 | [III-5/III-5/III-3] |
| 124 | II-1 | [III-5/III-5/III-4] |
| 125 | II-1 | [III-5/III-5/III-5] |
| 126 | II-2 | [III-1/III-1/III-1] |
| 127 | II-2 | [III-1/III-1/III-2] |
| 128 | II-2 | [III-1/III-1/III-3] |
| 129 | II-2 | [III-1/III-1/III-4] |
| 130 | II-2 | [III-1/III-1/III-5] |
| 131 | II-2 | [III-1/III-2/III-1] |
| 132 | II-2 | [III-1/III-2/III-2] |
| 133 | II-2 | [III-1/III-2/III-3] |
| 134 | II-2 | [III-1/III-2/III-4] |
| 135 | II-2 | [III-1/III-2/III-5] |
| 136 | II-2 | [III-1/III-3/III-1] |
| 137 | II-2 | [III-1/III-3/III-2] |
| 138 | II-2 | [III-1/III-3/III-3] |
| 139 | II-2 | [III-1/III-3/III-4] |
| 140 | II-2 | [III-1/III-3/III-5] |
| 141 | II-2 | [III-1/III-4/III-1] |
| 142 | II-2 | [III-1/III-4/III-2] |
| 143 | II-2 | [III-1/III-4/III-3] |
| 144 | II-2 | [III-1/III-4/III-4] |
| 145 | II-2 | [III-1/III-4/III-5] |
| 146 | II-2 | [III-1/III-5/III-1] |
| 147 | II-2 | [III-1/III-5/III-2] |
| 148 | II-2 | [III-1/III-5/III-3] |
| 149 | II-2 | [III-1/III-5/III-4] |
| 150 | II-2 | [III-1/III-5/III-5] |
| 151 | II-2 | [III-2/III-1/III-2] |
| 152 | II-2 | [III-2/III-1/III-3] |
| 153 | II-2 | [III-2/III-1/III-4] |
| 154 | II-2 | [III-2/III-1/III-5] |
| 155 | II-2 | [III-2/III-2/III-2] |
| 156 | II-2 | [III-2/III-2/III-3] |
| 157 | II-2 | [III-2/III-2/III-4] |
| 158 | II-2 | [III-2/III-2/III-5] |
| 159 | II-2 | [III-2/III-3/III-2] |
| 160 | II-2 | [III-2/III-3/III-3] |
| 161 | II-2 | [III-2/III-3/III-4] |
| 162 | II-2 | [III-2/III-3/III-5] |
| 163 | II-2 | [III-2/III-4/III-2] |
| 164 | II-2 | [III-2/III-4/III-3] |
| 165 | II-2 | [III-2/III-4/III-4] |
| 166 | II-2 | [III-2/III-4/III-5] |
| 167 | II-2 | [III-2/III-5/III-2] |
| 168 | II-2 | [III-2/III-5/III-3] |
| 169 | II-2 | [III-2/III-5/III-4] |
| 170 | II-2 | [III-2/III-5/III-5] |
| 171 | II-2 | [III-3/III-1/III-3] |
| 172 | II-2 | [III-3/III-1/III-4] |
| 173 | II-2 | [III-3/III-1/III-5] |
| 174 | II-2 | [III-3/III-2/III-3] |
| 175 | II-2 | [III-3/III-2/III-4] |
| 176 | II-2 | [III-3/III-2/III-5] |
| 177 | II-2 | [III-3/III-3/III-3] |
| 178 | II-2 | [III-3/III-3/III-4] |
| 179 | II-2 | [III-3/III-3/III-5] |
| 180 | II-2 | [III-3/III-4/III-3] |
| 181 | II-2 | [III-3/III-4/III-4] |
| 182 | II-2 | [III-3/III-4/III-5] |
| 183 | II-2 | [III-3/III-5/III-3] |
| 184 | II-2 | [III-3/III-5/III-4] |
| 185 | II-2 | [III-3/III-5/III-5] |
| 186 | II-2 | [III-4/III-1/III-4] |
| 187 | II-2 | [III-4/III-1/III-5] |
| 188 | II-2 | [III-4/III-2/III-4] |
| 189 | II-2 | [III-4/III-2/III-5] |
| 190 | II-2 | [III-4/III-3/III-4] |
| 191 | II-2 | [III-4/III-3/III-5] |
| 192 | II-2 | [III-4/III-4/III-4] |
| 193 | II-2 | [III-4/III-4/III-5] |
| 194 | II-2 | [III-4/III-5/III-4] |
| 195 | II-2 | [III-4/III-5/III-5] |
| 196 | II-2 | [III-5/III-1/III-5] |
| 197 | II-2 | [III-5/III-2/III-5] |
| 198 | II-2 | [III-5/III-1/III-1] |
| 199 | II-2 | [III-5/III-2/III-2] |
| 200 | II-2 | [III-5/III-3/III-2] |
| 201 | II-3 | [III-1/III-1/III-1] |
| 202 | II-3 | [III-1/III-1/III-2] |
| 203 | II-3 | [III-1/III-1/III-3] |
| 204 | II-3 | [III-1/III-1/III-4] |
| 205 | II-3 | [III-1/III-1/III-5] |
| 206 | II-3 | [III-1/III-2/III-1] |
| 207 | II-3 | [III-1/III-2/III-2] |
| 208 | II-3 | [III-1/III-2/III-3] |
| 209 | II-3 | [III-1/III-2/III-4] |
| 210 | II-3 | [III-1/III-2/III-5] |
| 211 | II-3 | [III-1/III-3/III-1] |
| 212 | II-3 | [III-1/III-3/III-2] |
| 213 | II-3 | [III-1/III-3/III-3] |
| 214 | II-3 | [III-1/III-3/III-4] |
| 215 | II-3 | [III-1/III-3/III-5] |
| 216 | II-3 | [III-1/III-4/III-1] |
| 217 | II-3 | [III-1/III-4/III-2] |
| 218 | II-3 | [III-1/III-4/III-3] |
| 219 | II-3 | [III-1/III-4/III-4] |
| 220 | II-3 | [III-1/III-4/III-5] |
| 221 | II-3 | [III-1/III-5/III-1] |
| 222 | II-3 | [III-1/III-5/III-2] |
| 223 | II-3 | [III-1/III-5/III-3] |
| 224 | II-3 | [III-1/III-5/III-4] |
| 225 | II-3 | [III-1/III-5/III-5] |
| 226 | II-3 | [III-3/III-1/III-1] |
| 227 | II-3 | [III-2/III-1/III-2] |
| 228 | II-3 | [III-2/III-1/III-3] |
| 229 | II-3 | [III-2/III-1/III-4] |
| 230 | II-3 | [III-2/III-1/III-5] |
| 231 | II-3 | [III-2/III-2/III-1] |
| 232 | II-3 | [III-2/III-2/III-2] |
| 233 | II-3 | [III-2/III-2/III-3] |
| 234 | II-3 | [III-2/III-2/III-4] |
| 235 | II-3 | [III-2/III-2/III-5] |
| 236 | II-3 | [III-2/III-3/III-1] |
| 237 | II-3 | [III-2/III-3/III-2] |
| 238 | II-3 | [III-2/III-3/III-3] |
| 239 | II-3 | [III-2/III-3/III-4] |
| 240 | II-3 | [III-2/III-3/III-5] |
| 241 | II-3 | [III-2/III-4/III-1] |
| 242 | II-3 | [III-2/III-4/III-2] |
| 243 | II-3 | [III-2/III-4/III-3] |
| 244 | II-3 | [III-2/III-4/III-4] |
| 245 | II-3 | [III-2/III-4/III-5] |
| 246 | II-3 | [III-2/III-5/III-1] |
| 247 | II-3 | [III-2/III-5/III-2] |
| 248 | II-3 | [III-2/III-5/III-3] |
| 249 | II-3 | [III-2/III-5/III-4] |
| 250 | II-3 | [III-2/III-5/III-5] |
| 251 | II-3 | [III-3/III-1/III-1] |
| 252 | II-3 | [III-3/III-1/III-2] |
| 253 | II-3 | [III-3/III-1/III-3] |
| 254 | II-3 | [III-3/III-1/III-4] |
| 255 | II-3 | [III-3/III-1/III-5] |
| 256 | II-3 | [III-3/III-2/III-1] |
| 257 | II-3 | [III-3/III-2/III-2] |
| 258 | II-3 | [III-3/III-2/III-3] |
| 259 | II-3 | [III-3/III-2/III-4] |
| 260 | II-3 | [III-3/III-2/III-5] |
| 261 | II-3 | [III-3/III-3/III-1] |
| 262 | II-3 | [III-3/III-3/III-2] |
| 263 | II-3 | [III-3/III-3/III-3] |
| 264 | II-3 | [III-3/III-3/III-4] |
| 265 | II-3 | [III-3/III-3/III-5] |
| 266 | II-3 | [III-3/III-4/III-1] |
| 267 | II-3 | [III-3/III-4/III-2] |
| 268 | II-3 | [III-3/III-4/III-3] |
| 269 | II-3 | [III-3/III-4/III-4] |
| 270 | II-3 | [III-3/III-4/III-5] |
| 271 | II-3 | [III-3/III-5/III-1] |
| 272 | II-3 | [III-3/III-5/III-2] |
| 273 | II-3 | [III-3/III-5/III-3] |
| 274 | II-3 | [III-3/III-5/III-4] |
| 275 | II-3 | [III-3/III-5/III-5] |
| 276 | II-3 | [III-4/III-1/III-1] |
| 277 | II-3 | [III-4/III-1/III-2] |
| 278 | II-3 | [III-4/III-1/III-3] |
| 279 | II-3 | [III-4/III-1/III-4] |
| 280 | II-3 | [III-4/III-1/III-5] |
| 281 | II-3 | [III-4/III-2/III-1] |
| 282 | II-3 | [III-4/III-2/III-2] |
| 283 | II-3 | [III-4/III-2/III-3] |
| 284 | II-3 | [III-4/III-2/III-4] |
| 285 | II-3 | [III-4/III-2/III-5] |
| 286 | II-3 | [III-4/III-3/III-1] |
| 287 | II-3 | [III-4/III-3/III-2] |
| 288 | II-3 | [III-4/III-3/III-3] |
| 289 | II-3 | [III-4/III-3/III-4] |
| 290 | II-3 | [III-4/III-3/III-5] |
| 291 | II-3 | [III-4/III-4/III-1] |
| 292 | II-3 | [III-4/III-4/III-2] |
| 293 | II-3 | [III-4/III-4/III-3] |
| 294 | II-3 | [III-4/III-4/III-4] |
| 295 | II-3 | [III-4/III-4/III-5] |
| 296 | II-3 | [III-4/III-5/III-1] |
| 297 | II-3 | [III-4/III-5/III-2] |
| 293 | II-3 | [III-4/III-5/III-3] |
| 299 | II-3 | [III-4/III-5/III-4] |
| 300 | II-3 | [III-4/III-5/III-5] |
| 301 | II-3 | [III-5/III-1/III-1] |
| 302 | II-3 | [III-5/III-1/III-2] |
| 303 | II-3 | [III-5/III-1/III-3] |
| 304 | II-3 | [III-5/III-1/III-4] |
| 305 | II-3 | [III-5/III-1/III-5] |
| 306 | II-3 | [III-5/III-2/III-1] |
| 307 | II-3 | [III-5/III-2/III-2] |
| 308 | II-3 | [III-5/III-2/III-3] |
| 309 | II-3 | [III-5/III-2/III-4] |
| 310 | II-3 | [III-5/III-2/III-5] |
| 311 | II-3 | [III-5/III-3/III-1] |
| 312 | II-3 | [III-5/III-3/III-2] |
| 313 | II-3 | [III-5/III-3/III-3] |
| 314 | II-3 | [III-5/III-3/III-4] |
| 315 | II-3 | [III-5/III-3/III-5] |
| 316 | II-3 | [III-5/III-4/III-1] |
| 317 | II-3 | [III-5/III-4/III-2] |
| 318 | II-3 | [III-5/III-4/III-3] |
| 319 | II-3 | [III-5/III-4/III-4] |
| 320 | II-3 | [III-5/III-4/III-5] |
| 321 | II-3 | [III-5/III-5/III-1] |
| 322 | II-3 | [III-5/III-5/III-2] |
| 323 | II-3 | [III-5/III-5/III-3] |
| 324 | II-3 | [III-5/III-5/III-4] |
| 325 | II-3 | [III-5/III-5/III-5] |
| 326 | II-4 | [III-1/III-1/III-1] |
| 327 | II-4 | [III-1/III-1/III-2] |
| 328 | II-4 | [III-1/III-1/III-3] |
| 329 | II-4 | [III-1/III-1/III-4] |
| 330 | II-4 | [III-1/III-1/III-5] |
| 331 | II-4 | [III-1/III-2/III-1] |
| 332 | II-4 | [III-1/III-2/III-2] |
| 333 | II-4 | [III-1/III-2/III-3] |
| 334 | II-4 | [III-1/III-2/III-4] |
| 335 | II-4 | [III-1/III-2/III-5] |
| 336 | II-4 | [III-1/III-3/III-1] |
| 337 | II-4 | [III-1/III-3/III-2] |
| 338 | II-4 | [III-1/III-3/III-3] |
| 339 | II-4 | [III-1/III-3/III-4] |
| 340 | II-4 | [III-1/III-3/III-5] |
| 341 | II-4 | [III-1/III-4/III-1] |
| 342 | II-4 | [III-1/III-4/III-2] |
| 343 | II-4 | [III-1/III-4/III-3] |
| 344 | II-4 | [III-1/III-4/III-4] |
| 345 | II-4 | [III-1/III-4/III-5] |
| 346 | II-4 | [III-1/III-5/III-1] |
| 347 | II-4 | [III-1/III-5/III-2] |
| 348 | II-4 | [III-1/III-5/III-3] |
| 349 | II-4 | [III-1/III-5/III-4] |
| 350 | II-4 | [III-1/III-5/III-5] |
| 351 | II-4 | [III-2/III-1/III-1] |
| 352 | II-4 | [III-2/III-1/III-2] |
| 353 | II-4 | [III-2/III-1/III-3] |
| 354 | II-4 | [III-2/III-1/III-4] |
| 355 | II-4 | [III-2/III-1/III-5] |
| 356 | II-4 | [III-2/III-2/III-1] |
| 357 | II-4 | [III-2/III-2/III-2] |
| 358 | II-4 | [III-2/III-2/III-3] |
| 359 | II-4 | [III-2/III-2/III-4] |
| 360 | II-4 | [III-2/III-2/III-5] |
| 361 | II-4 | [III-2/III-3/III-1] |
| 362 | II-4 | [III-2/III-3/III-2] |
| 363 | II-4 | [III-2/III-3/III-3] |
| 364 | II-4 | [III-2/III-3/III-4] |
| 365 | II-4 | [III-2/III-3/III-5] |
| 366 | II-4 | [III-2/III-4/III-1] |
| 367 | II-4 | [III-2/III-4/III-2] |
| 368 | II-4 | [III-2/III-4/III-3] |
| 369 | II-4 | [III-2/III-4/III-4] |
| 370 | II-4 | [III-2/III-4/III-5] |
| 371 | II-4 | [III-2/III-5/III-1] |
| 372 | II-4 | [III-2/III-5/III-2] |
| 373 | II-4 | [III-2/III-5/III-3] |
| 374 | II-4 | [III-2/III-5/III-4] |
| 375 | II-4 | [III-2/III-5/III-5] |
| 376 | II-4 | [III-3/III-1/III-1] |
| 377 | II-4 | [III-3/III-1/III-2] |
| 378 | II-4 | [III-3/III-1/III-3] |
| 379 | II-4 | [III-3/III-1/III-4] |
| 380 | II-4 | [III-3/III-1/III-5] |
| 381 | II-4 | [III-3/III-2/III-1] |
| 382 | II-4 | [III-3/III-2/III-2] |
| 383 | II-4 | [III-3/III-2/III-3] |
| 384 | II-4 | [III-3/III-2/III-4] |
| 385 | II-4 | [III-3/III-2/III-5] |
| 386 | II-4 | [III-3/III-3/III-1] |
| 387 | II-4 | [III-3/III-3/III-2] |
| 388 | II-4 | [III-3/III-3/III-3] |
| 389 | II-4 | [III-3/III-3/III-4] |
| 390 | II-4 | [III-3/III-3/III-5] |
| 391 | II-4 | [III-3/III-4/III-1] |
| 392 | II-4 | [III-3/III-4/III-2] |
| 393 | II-4 | [III-3/III-4/III-3] |
| 394 | II-4 | [III-3/III-4/III-4] |
| 395 | II-4 | [III-3/III-4/III-5] |
| 396 | II-4 | [III-3/III-5/III-1] |
| 397 | II-4 | [III-3/III-5/III-2] |
| 398 | II-4 | [III-3/III-5/III-3] |
| 399 | II-4 | [III-3/III-5/III-4] |
| 400 | II-4 | [III-3/III-5/III-5] |
| 401 | II-4 | [III-4/III-1/III-1] |
| 402 | II-4 | [III-4/III-1/III-2] |
| 403 | II-4 | [III-4/III-1/III-3] |
| 404 | II-4 | [III-4/III-1/III-4] |
| 405 | II-4 | [III-4/III-1/III-5] |
| 406 | II-4 | [III-4/III-2/III-1] |
| 407 | II-4 | [III-4/III-2/III-2] |
| 408 | II-4 | [III-4/III-2/III-3] |
| 409 | II-4 | [III-4/III-2/III-4] |
| 410 | II-4 | [III-4/III-2/III-5] |
| 411 | II-4 | [III-4/III-3/III-1] |
| 412 | II-4 | [III-4/III-3/III-2] |
| 413 | II-4 | [III-4/III-3/III-3] |
| 414 | II-4 | [III-4/III-3/III-4] |
| 415 | II-4 | [III-4/III-3/III-5] |
| 416 | II-4 | [III-4/III-4/III-1] |
| 417 | II-4 | [III-4/III-4/III-2] |
| 418 | II-4 | [III-4/III-4/III-3] |
| 419 | II-4 | [III-4/III-4/III-4] |
| 420 | II-4 | [III-4/III-4/III-5] |
| 421 | II-4 | [III-4/III-5/III-1] |
| 422 | II-4 | [III-4/III-5/III-2] |
| 423 | II-4 | [III-4/III-5/III-3] |
| 424 | II-4 | [III-4/III-5/III-4] |
| 425 | II-4 | [III-4/III-5/III-5] |
| 426 | II-4 | [III-5/III-1/III-1] |
| 427 | II-4 | [III-5/III-1/III-2] |
| 428 | II-4 | [III-5/III-1/III-3] |
| 429 | II-4 | [III-5/III-1/III-4] |
| 430 | II-4 | [III-5/III-1/III-5] |
| 431 | II-4 | [III-5/III-2/III-1] |
| 432 | II-4 | [III-5/III-2/III-2] |
| 433 | II-4 | [III-5/III-2/III-3] |
| 434 | II-4 | [III-5/III-2/III-4] |
| 435 | II-4 | [III-5/III-2/III-5] |
| 436 | II-4 | [III-5/III-3/III-1] |
| 437 | II-4 | [III-5/III-3/III-2] |
| 438 | II-4 | [III-5/III-3/III-3] |
| 439 | II-4 | [III-5/III-3/III-4] |
| 440 | II-4 | [III-5/III-3/III-5] |
| 441 | II-4 | [III-5/III-4/III-1] |
| 442 | II-4 | [III-5/III-4/III-2] |
| 443 | II-4 | [III-5/III-4/III-3] |
| 444 | II-4 | [III-5/III-4/III-4] |
| 445 | II-4 | [III-5/III-4/III-5] |
| 446 | II-4 | [III-5/III-5/III-1] |
| 447 | II-4 | [III-5/III-5/III-2] |
| 448 | II-4 | [III-5/III-5/III-3] |
| 449 | II-4 | [III-5/III-5/III-4] |
| 450 | II-4 | [III-5/III-5/III-5] |
| 451 | II-5 | [III-1/III-1/III-1] |
| 452 | II-5 | [III-1/III-1/III-2] |
| 453 | II-5 | [III-1/III-1/III-3] |
| 454 | II-5 | [III-1/III-1/III-4] |
| 455 | II-5 | [III-1/III-1/III-5] |
| 456 | II-5 | [III-1/III-2/III-1] |
| 457 | II-5 | [III-1/III-2/III-2] |
| 458 | II-5 | [III-1/III-2/III-3] |
| 459 | II-5 | [III-1/III-2/III-4] |
| 460 | II-5 | [III-1/III-2/III-5] |
| 461 | II-5 | [III-1/III-3/III-1] |
| 462 | II-5 | [III-1/III-3/III-2] |
| 463 | II-5 | [III-1/III-3/III-3] |
| 464 | II-5 | [III-1/III-3/III-4] |
| 465 | II-5 | [III-1/III-3/III-5] |
| 466 | II-5 | [III-1/III-4/III-1] |
| 467 | II-5 | [III-1/III-4/III-2] |
| 468 | II-5 | [III-1/III-4/III-3] |
| 469 | II-5 | [III-1/III-4/III-4] |
| 470 | II-5 | [III-1/III-4/III-5] |
| 471 | II-5 | [III-1/III-5/III-1] |
| 472 | II-5 | [III-1/III-5/III-2] |
| 473 | II-5 | [III-1/III-5/III-3] |
| 474 | II-5 | [III-1/III-5/III-4] |
| 475 | II-5 | [III-1/III-5/III-5] |
| 476 | II-5 | [III-2/III-1/III-2] |
| 477 | II-5 | [III-2/III-1/III-3] |
| 478 | II-5 | [III-2/III-1/III-4] |
| 479 | II-5 | [III-2/III-1/III-5] |
| 480 | II-5 | [III-2/III-2/III-2] |
| 481 | II-5 | [III-2/III-2/III-3] |
| 482 | II-5 | [III-2/III-2/III-4] |
| 483 | II-5 | [III-2/III-2/III-5] |
| 484 | II-5 | [III-2/III-3/III-2] |
| 485 | II-5 | [III-2/III-3/III-3] |
| 486 | II-5 | [III-2/III-3/III-4] |
| 487 | II-5 | [III-2/III-3/III-5] |
| 488 | II-5 | [III-2/III-4/III-2] |
| 489 | II-5 | [III-2/III-4/III-3] |
| 490 | II-5 | [III-2/III-4/III-4] |
| 491 | II-5 | [III-2/III-4/III-5] |
| 492 | II-5 | [III-2/III-5/III-2] |
| 493 | II-5 | [III-2/III-5/III-3] |
| 494 | II-5 | [III-2/III-5/III-4] |
| 495 | II-5 | [III-2/III-5/III-5] |
| 496 | II-5 | [III-3/III-1/III-3] |
| 497 | II-5 | [III-3/III-1/III-4] |
| 498 | II-5 | [III-3/III-1/III-5] |
| 499 | II-5 | [III-3/III-2/III-3] |
| 500 | II-5 | [III-3/III-2/III-4] |
| 501 | II-5 | [III-3/III-2/III-5] |
| 502 | II-5 | [III-3/III-3/III-3] |
| 503 | II-5 | [III-3/III-3/III-4] |
| 504 | II-5 | [III-3/III-3/III-5] |
| 505 | II-5 | [III-3/III-4/III-3] |
| 506 | II-5 | [III-3/III-4/III-4] |
| 507 | II-5 | [III-3/III-4/III-5] |
| 508 | II-5 | [III-3/III-5/III-3] |
| 509 | II-5 | [III-3/III-5/III-4] |
| 510 | II-5 | [III-3/III-5/III-5] |
| 511 | II-5 | [III-4/III-1/III-4] |
| 512 | II-5 | [III-4/III-1/III-5] |
| 513 | II-5 | [III-4/III-2/III-4] |
| 514 | II-5 | [III-4/III-2/III-5] |
| 515 | II-5 | [III-4/III-3/III-4] |
| 516 | II-5 | [III-4/III-3/III-5] |
| 517 | II-5 | [III-4/III-4/III-4] |
| 518 | II-5 | [III-4/III-4/III-5] |
| 519 | II-5 | [III-4/III-5/III-4] |
| 520 | II-5 | [III-4/III-5/III-5] |
| 521 | II-5 | [III-5/III-1/III-5] |
| 522 | II-5 | [III-5/III-2/III-5] |
| 523 | II-5 | [III-5/III-3/III-5] |
| 524 | II-5 | [III-5/III-4/III-5] |
| 525 | II-5 | [III-5/III-5/III-5] |
| 526 | II-6 | [III-1/III-1/III-1] |
| 527 | II-6 | [III-1/III-1/III-2] |
| 528 | II-6 | [III-1/III-1/III-3] |
| 529 | II-6 | [III-1/III-1/III-4] |
| 530 | II-6 | [III-1/III-1/III-5] |
| 531 | II-6 | [III-1/III-2/III-1] |
| 532 | II-6 | [III-1/III-2/III-2] |
| 533 | II-6 | [III-1/III-2/III-3] |
| 534 | II-6 | [III-1/III-2/III-4] |
| 535 | II-6 | [III-1/III-2/III-5] |
| 536 | II-6 | [III-1/III-3/III-1] |
| 537 | II-6 | [III-1/III-3/III-2] |
| 538 | II-6 | [III-1/III-3/III-3] |
| 539 | II-6 | [III-1/III-3/III-4] |
| 540 | II-6 | [III-3/III-3/III-5] |
| 541 | II-6 | [III-1/III-4/III-1] |
| 542 | II-6 | [III-1/III-4/III-2] |
| 543 | II-6 | [III-1/III-4/III-3] |
| 544 | II-6 | [III-1/III-4/III-4] |
| 545 | II-6 | [III-1/III-4/III-5] |
| 546 | II-6 | [III-1/III-5/III-1] |
| 547 | II-6 | [III-1/III-5/III-2] |
| 548 | II-6 | [III-1/III-5/III-3] |
| 549 | II-6 | [III-1/III-5/III-4] |
| 550 | II-6 | [III-1/III-5/III-5] |
| 551 | II-6 | [III-2/III-1/III-2] |
| 552 | II-6 | [III-2/III-1/III-3] |
| 553 | II-6 | [III-2/III-1/III-4] |
| 554 | II-6 | [III-2/III-1/III-5] |
| 555 | II-6 | [III-2/III-2/III-2] |
| 556 | II-6 | [III-2/III-2/III-3] |
| 557 | II-6 | [III-2/III-2/III-4] |
| 558 | II-6 | [III-2/III-2/III-5] |
| 559 | II-6 | [III-2/III-3/III-3] |
| 560 | II-6 | [III-2/III-3/III-4] |
| 561 | II-6 | [III-2/III-3/III-5] |
| 562 | II-6 | [III-2/III-4/III-2] |
| 563 | II-6 | [III-2/III-4/III-3] |
| 564 | II-6 | [III-2/III-4/III-4] |
| 565 | II-6 | [III-2/III-4/III-5] |
| 566 | II-6 | [III-2/III-5/III-1] |
| 567 | II-6 | [III-2/III-5/III-2] |
| 568 | II-6 | [III-2/III-5/III-3] |
| 569 | II-6 | [III-2/III-5/III-4] |
| 570 | II-6 | [III-2/III-5/III-5] |
| 571 | II-6 | [III-3/III-1/III-3] |
| 572 | II-6 | [III-3/III-1/III-4] |
| 573 | II-6 | [III-3/III-1/III-5] |
| 574 | II-6 | [III-3/III-2/III-3] |
| 575 | II-6 | [III-3/III-2/III-4] |
| 576 | II-6 | [III-3/III-2/III-5] |
| 577 | II-6 | [III-3/III-3/III-3] |
| 578 | II-6 | [III-3/III-3/III-4] |
| 579 | II-6 | [III-3/III-3/III-5] |
| 580 | II-6 | [III-3/III-4/III-3] |
| 581 | II-6 | [III-3/III-4/III-4] |
| 582 | II-6 | [III-3/III-4/III-5] |
| 583 | II-6 | [III-3/III-5/III-3] |
| 584 | II-6 | [III-3/III-5/III-4] |
| 585 | II-6 | [III-3/III-5/III-5] |
| 586 | II-6 | [III-4/III-1/III-4] |
| 587 | II-6 | [III-4/III-1/III-5] |
| 588 | II-6 | [III-4/III-2/III-4] |
| 589 | II-6 | [III-4/III-2/III-5] |
| 590 | II-6 | [III-4/III-3/III-4] |
| 591 | II-6 | [III-4/III-3/III-5] |
| 592 | II-6 | [III-4/III-4/III-4] |
| 593 | II-6 | [III-4/III-4/III-5] |
| 594 | II-6 | [III-4/III-5/III-4] |
| 595 | II-6 | [III-4/III-5/III-5] |
| 596 | II-6 | [III-5/III-1/III-5] |
| 597 | II-6 | [III-5/III-2/III-5] |
| 598 | II-6 | [III-5/III-3/III-5] |
| 599 | II-6 | [III-5/III-4/III-5] |
| 600 | II-6 | [III-5/III-5/III-5] |
| 601 | II-7 | [III-1/III-1/III-1] |
| 602 | II-7 | [III-1/III-1/III-2] |
| 603 | II-7 | [III-1/III-1/III-3] |
| 604 | II-7 | [III-1/III-1/III-4] |
| 605 | II-7 | [III-1/III-1/III-5] |
| 606 | II-7 | [III-1/III-2/III-1] |
| 607 | II-7 | [III-1/III-2/III-2] |
| 608 | II-7 | [III-1/III-2/III-3] |
| 609 | II-7 | [III-1/III-2/III-4] |
| 610 | II-7 | [III-1/III-2/III-5] |
| 611 | II-7 | [III-1/III-3/III-1] |
| 612 | II-7 | [III-1/III-3/III-2] |
| 613 | II-7 | [III-1/III-3/III-3] |
| 614 | II-7 | [III-1/III-3/III-4] |
| 615 | II-7 | [III-1/III-3/III-5] |
| 616 | II-7 | [III-1/III-4/III-1] |
| 617 | II-7 | [III-1/III-4/III-2] |
| 618 | II-7 | [III-1/III-4/III-3] |
| 619 | II-7 | [III-1/III-4/III-4] |
| 620 | II-7 | [III-1/III-4/III-5] |
| 621 | II-7 | [III-1/III-5/III-1] |
| 622 | II-7 | [III-1/III-5/III-2] |
| 623 | II-7 | [III-1/III-5/III-3] |
| 624 | II-7 | [III-1/III-5/III-4] |
| 625 | II-7 | [III-1/III-5/III-5] |
| 626 | II-7 | [III-2/III-1/III-1] |
| 627 | II-7 | [III-2/III-1/III-2] |
| 628 | II-7 | [III-2/III-1/III-3] |
| 629 | II-7 | [III-2/III-1/III-4] |
| 630 | II-7 | [III-2/III-1/III-5] |
| 631 | II-7 | [III-2/III-2/III-1] |
| 632 | II-7 | [III-2/III-2/III-2] |
| 633 | II-7 | [III-2/III-2/III-3] |
| 634 | II-7 | [III-2/III-2/III-4] |
| 635 | II-7 | [III-2/III-2/III-5] |
| 636 | II-7 | [III-2/III-3/III-1] |
| 637 | II-7 | [III-2/III-3/III-2] |
| 638 | II-7 | [III-2/III-3/III-3] |
| 639 | II-7 | [III-2/III-3/III-4] |
| 640 | II-7 | [III-2/III-3/III-5] |
| 641 | II-7 | [III-2/III-4/III-1] |
| 642 | II-7 | [III-2/III-4/III-2] |
| 643 | II-7 | [III-2/III-4/III-3] |
| 644 | II-7 | [III-2/III-4/III-4] |
| 645 | II-7 | [III-2/III-4/III-5] |
| 646 | II-7 | [III-2/III-5/III-1] |
| 647 | II-7 | [III-2/III-5/III-2] |
| 648 | II-7 | [III-2/III-5/III-3] |
| 649 | II-7 | [III-2/III-5/III-4] |
| 650 | II-7 | [III-2/III-5/III-5] |
| 651 | II-7 | [III-3/III-1/III-1] |
| 652 | II-7 | [III-3/III-1/III-2] |
| 653 | II-7 | [III-3/III-1/III-3] |
| 654 | II-7 | [III-3/III-1/III-4] |
| 655 | II-7 | [III-3/III-1/III-5] |
| 656 | II-7 | [III-3/III-2/III-1] |
| 657 | II-7 | [III-3/III-2/III-2] |
| 658 | II-7 | [III-3/III-2/III-3] |
| 659 | II-7 | [III-3/III-2/III-4] |
| 660 | II-7 | [III-3/III-2/III-5] |
| 661 | II-7 | [III-3/III-3/III-1] |
| 662 | II-7 | [III-3/III-3/III-2] |
| 663 | II-7 | [III-3/III-3/III-3] |
| 664 | II-7 | [III-3/III-3/III-4] |
| 665 | II-7 | [III-3/III-3/III-5] |
| 666 | II-7 | [III-3/III-4/III-1] |
| 667 | II-7 | [III-3/III-4/III-2] |
| 668 | II-7 | [III-3/III-4/III-3] |
| 669 | II-7 | [III-3/III-4/III-4] |
| 670 | II-7 | [III-3/III-4/III-5] |
| 671 | II-7 | [III-3/III-5/III-1] |
| 672 | II-7 | [III-3/III-5/III-2] |
| 673 | II-7 | [III-3/III-5/III-3] |
| 674 | II-7 | [III-3/III-5/III-4] |
| 675 | II-7 | [III-3/III-5/III-5] |
| 676 | II-7 | [III-4/III-1/III-1] |
| 677 | II-7 | [III-4/III-1/III-2] |
| 678 | II-7 | [III-4/III-1/III-3] |
| 679 | II-7 | [III-4/III-1/III-4] |
| 680 | II-7 | [III-4/III-1/III-5] |
| 681 | II-7 | [III-4/III-2/III-1] |
| 682 | II-7 | [III-4/III-2/III-2] |
| 683 | II-7 | [III-4/III-2/III-3] |
| 684 | II-7 | [III-4/III-2/III-4] |
| 685 | II-7 | [III-4/III-2/III-5] |
| 686 | II-7 | [III-4/III-3/III-1] |
| 687 | II-7 | [III-4/III-3/III-2] |
| 688 | II-7 | [III-4/III-3/III-3] |
| 689 | II-7 | [III-4/III-3/III-4] |
| 690 | II-7 | [III-4/III-3/III-5] |
| 691 | II-7 | [III-4/III-4/III-1] |
| 692 | II-7 | [III-4/III-4/III-2] |
| 693 | II-7 | [III-4/III-4/III-3] |
| 694 | II-7 | [III-4/III-4/III-4] |
| 695 | II-7 | [III-4/III-4/III-5] |
| 696 | II-7 | [III-4/III-5/III-1] |
| 697 | II-7 | [III-4/III-5/III-2] |
| 698 | II-7 | [III-4/III-5/III-3] |
| 699 | II-7 | [III-4/III-5/III-4] |
| 700 | II-7 | [III-4/III-5/III-5] |
| 701 | II-7 | [III-5/III-1/III-1] |
| 702 | II-7 | [III-5/III-1/III-2] |
| 703 | II-7 | [III-5/III-1/III-3] |
| 704 | II-7 | [III-5/III-1/III-4] |
| 705 | II-7 | [III-5/III-1/III-5] |
| 706 | II-7 | [III-5/III-2/III-1] |
| 707 | II-7 | [III-5/III-2/III-2] |
| 708 | II-7 | [III-5/III-2/III-3] |
| 709 | II-7 | [III-5/III-2/III-4] |
| 710 | II-7 | [III-5/III-2/III-5] |
| 711 | II-7 | [III-5/III-3/III-1] |
| 712 | II-7 | [III-5/III-3/III-2] |
| 713 | II-7 | [III-5/III-3/III-3] |
| 714 | II-7 | [III-5/III-3/III-4] |
| 715 | II-7 | [III-5/III-3/III-5] |
| 716 | II-7 | [III-5/III-4/III-1] |
| 717 | II-7 | [III-5/III-4/III-2] |
| 718 | II-7 | [III-5/III-4/III-3] |
| 719 | II-7 | [III-5/III-4/III-4] |
| 720 | II-7 | [III-5/III-4/III-5] |
| 721 | II-7 | [III-5/III-5/III-1] |
| 722 | II-7 | [III-5/III-5/III-2] |
| 723 | II-7 | [III-5/III-5/III-3] |
| 724 | II-7 | [III-5/III-5/III-4] |
| 725 | II-7 | [III-5/III-5/III-5] |
| 726 | II-8 | [III-1/III-1/III-1] |
| 727 | II-8 | [III-1/III-1/III-2] |
| 728 | II-8 | [III-1/III-1/III-3] |
| 729 | II-8 | [III-1/III-1/III-4] |
| 730 | II-8 | [III-1/III-1/III-5] |
| 731 | II-8 | [III-1/III-2/III-1] |
| 732 | II-8 | [III-1/III-2/III-2] |
| 733 | II-8 | [III-1/III-2/III-3] |
| 734 | II-8 | [III-1/III-2/III-4] |
| 735 | II-8 | [III-1/III-2/III-5] |
| 736 | II-8 | [III-1/III-3/III-1] |
| 737 | II-8 | [III-1/III-3/III-2] |
| 738 | II-8 | [III-1/III-3/III-3] |
| 739 | II-8 | [III-1/III-3/III-4] |
| 740 | II-8 | [III-1/III-3/III-5] |
| 741 | II-8 | [III-1/III-4/III-1] |
| 742 | II-8 | [III-1/III-4/III-2] |
| 743 | II-8 | [III-1/III-4/III-3] |
| 744 | II-8 | [III-1/III-4/III-4] |
| 745 | II-8 | [III-1/III-4/III-5] |
| 746 | II-8 | [III-1/III-5/III-1] |
| 747 | II-8 | [III-1/III-5/III-2] |
| 748 | II-8 | [III-1/III-5/III-3] |
| 749 | II-8 | [III-1/III-5/III-4] |
| 750 | II-8 | [III-1/III-5/III-5] |
| 751 | II-8 | [III-2/III-1/III-1] |
| 752 | II-8 | [III-2/III-1/III-2] |
| 753 | II-8 | [III-2/III-1/III-3] |
| 754 | II-8 | [III-2/III-1/III-4] |
| 755 | II-8 | [III-2/III-1/III-5] |
| 756 | II-8 | [III-2/III-2/III-1] |
| 757 | II-8 | [III-2/III-2/III-2] |
| 758 | II-8 | [III-2/III-2/III-3] |
| 759 | II-8 | [III-2/III-2/III-4] |
| 760 | II-8 | [III-2/III-2/III-5] |
| 761 | II-8 | [III-2/III-3/III-1] |
| 762 | II-8 | [III-2/III-3/III-2] |
| 763 | II-8 | [III-2/III-3/III-3] |
| 764 | II-8 | [III-2/III-3/III-4] |
| 765 | II-8 | [III-2/III-3/III-5] |
| 766 | II-8 | [III-2/III-4/III-1] |
| 767 | II-8 | [III-2/III-4/III-2] |
| 768 | II-8 | [III-2/III-4/III-3] |
| 769 | II-8 | [III-2/III-4/III-4] |
| 770 | II-8 | [III-2/III-4/III-5] |
| 771 | II-8 | [III-2/III-5/III-1] |
| 772 | II-8 | [III-2/III-5/III-2] |
| 773 | II-8 | [III-2/III-5/III-3] |
| 774 | II-8 | [III-2/III-5/III-4] |
| 775 | II-8 | [III-2/III-5/III-5] |
| 776 | II-8 | [III-3/III-1/III-1] |
| 777 | II-8 | [III-3/III-1/III-2] |
| 778 | II-8 | [III-3/III-1/III-3] |
| 779 | II-8 | [III-3/III-1/III-4] |
| 780 | II-8 | [III-3/III-1/III-5] |
| 781 | II-8 | [III-3/III-2/III-1] |
| 782 | II-8 | [III-3/III-2/III-2] |
| 783 | II-8 | [III-3/III-2/III-3] |
| 784 | II-8 | [III-3/III-2/III-4] |
| 785 | II-8 | [III-3/III-2/III-5] |
| 786 | II-8 | [III-3/III-3/III-1] |
| 787 | II-8 | [III-3/III-3/III-2] |
| 788 | II-8 | [III-3/III-3/III-3] |
| 789 | II-8 | [III-3/III-3/III-4] |
| 790 | II-8 | [III-3/III-3/III-5] |
| 791 | II-8 | [III-3/III-4/III-1] |
| 792 | II-8 | [III-3/III-4/III-2] |
| 793 | II-8 | [III-3/III-4/III-3] |
| 794 | II-8 | [III-3/III-4/III-4] |
| 795 | II-8 | [III-3/III-4/III-5] |
| 796 | II-8 | [III-3/III-5/III-1] |
| 797 | II-8 | [III-3/III-5/III-2] |
| 798 | II-8 | [III-3/III-5/III-3] |
| 799 | II-8 | [III-3/III-5/III-4] |
| 800 | II-8 | [III-3/III-5/III-5] |
| 801 | II-8 | [III-4/III-1/III-1] |
| 802 | II-8 | [III-4/III-1/III-2] |
| 803 | II-8 | [III-4/III-1/III-3] |
| 804 | II-8 | [III-4/III-1/III-4] |
| 805 | II-8 | [III-4/III-1/III-5] |
| 806 | II-8 | [III-4/III-2/III-1] |
| 807 | II-8 | [III-4/III-2/III-2] |
| 808 | II-8 | [III-4/III-2/III-3] |
| 809 | II-8 | [III-4/III-2/III-4] |
| 810 | II-8 | [III-4/III-2/III-5] |
| 811 | II-8 | [III-4/III-3/III-1] |
| 812 | II-8 | [III-4/III-3/III-2] |
| 813 | II-8 | [III-4/III-3/III-3] |
| 814 | II-8 | [III-4/III-3/III-4] |
| 815 | II-8 | [III-4/III-3/III-5] |
| 816 | II-8 | [III-4/III-4/III-1] |
| 817 | II-8 | [III-4/III-4/III-2] |
| 818 | II-8 | [III-4/III-4/III-3] |
| 819 | II-8 | [III-4/III-4/III-4] |
| 820 | II-8 | [III-4/III-4/III-5] |
| 821 | II-8 | [III-4/III-5/III-1] |
| 822 | II-8 | [III-4/III-5/III-2] |
| 823 | II-8 | [III-4/III-5/III-3] |
| 824 | II-8 | [III-4/III-5/III-4] |
| 825 | II-8 | [III-4/III-5/III-5] |
| 826 | II-8 | [III-5/III-1/III-1] |
| 827 | II-8 | [III-5/III-1/III-2] |
| 828 | II-8 | [III-5/III-1/III-3] |
| 829 | II-8 | [III-5/III-1/III-4] |
| 830 | II-8 | [III-5/III-1/III-5] |
| 831 | II-8 | [III-5/III-2/III-1] |
| 832 | II-8 | [III-5/III-2/III-2] |
| 833 | II-8 | [III-5/III-2/III-3] |
| 834 | II-8 | [III-5/III-2/III-4] |
| 835 | II-8 | [III-5/III-2/III-5] |
| 836 | II-8 | [III-5/III-3/III-1] |
| 837 | II-8 | [III-5/III-3/III-2] |
| 838 | II-8 | [III-5/III-3/III-3] |
| 839 | II-8 | [III-5/III-3/III-4] |
| 840 | II-8 | [III-5/III-3/III-5] |
| 841 | II-8 | [III-5/III-4/III-1] |
| 842 | II-8 | [III-5/III-4/III-2] |
| 843 | II-8 | [III-5/III-4/III-3] |
| 844 | II-8 | [III-5/III-4/III-4] |
| 845 | II-8 | [III-5/III-4/III-5] |
| 846 | II-8 | [III-5/III-5/III-1] |
| 847 | II-8 | [III-5/III-5/III-2] |
| 848 | II-8 | [III-5/III-5/III-3] |
| 849 | II-8 | [III-5/III-5/III-4] |
| 850 | II-8 | [III-5/III-5/III-5] |
| 851 | II-9 | [III-1/III-1/III-1] |
| 852 | II-9 | [III-1/III-1/III-2] |
| 853 | II-9 | [III-1/III-1/III-3] |
| 854 | II-9 | [III-1/III-1/III-4] |
| 855 | II-9 | [III-1/III-1/III-5] |
| 856 | II-9 | [III-1/III-2/III-1] |
| 857 | II-9 | [III-1/III-2/III-2] |
| 858 | II-9 | [III-1/III-2/III-3] |
| 859 | II-9 | [III-1/III-2/III-4] |
| 860 | II-9 | [III-1/III-2/III-5] |
| 861 | II-9 | [III-1/III-3/III-1] |
| 862 | II-9 | [III-1/III-3/III-2] |
| 863 | II-9 | [III-1/III-3/III-3] |
| 864 | II-9 | [III-1/III-3/III-4] |
| 865 | II-9 | [III-1/III-3/III-5] |
| 866 | II-9 | [III-1/III-4/III-1] |
| 867 | II-9 | [III-1/III-4/III-2] |
| 868 | II-9 | [III-1/III-4/III-3] |
| 869 | II-9 | [III-1/III-4/III-4] |
| 870 | II-9 | [III-1/III-4/III-5] |
| 871 | II-9 | [III-1/III-5/III-1] |
| 872 | II-9 | [III-1/III-5/III-2] |
| 873 | II-9 | [III-1/III-5/III-3] |
| 874 | II-9 | [III-1/III-5/III-4] |
| 875 | II-9 | [III-1/III-5/III-5] |
| 876 | II-9 | [III-2/III-1/III-1] |
| 877 | II-9 | [III-2/III-1/III-2] |
| 878 | II-9 | [III-2/III-1/III-3] |
| 879 | II-9 | [III-2/III-1/III-4] |
| 880 | II-9 | [III-2/III-1/III-5] |
| 881 | II-9 | [III-2/III-2/III-1] |
| 882 | II-9 | [III-2/III-2/III-2] |
| 883 | II-9 | [III-2/III-2/III-3] |
| 884 | II-9 | [III-2/III-2/III-4] |
| 885 | II-9 | [III-2/III-2/III-5] |
| 886 | II-9 | [III-2/III-3/III-1] |
| 887 | II-9 | [III-2/III-3/III-2] |
| 888 | II-9 | [III-2/III-3/III-3] |
| 889 | II-9 | [III-2/III-3/III-4] |
| 890 | II-9 | [III-2/III-3/III-5] |
| 891 | II-9 | [III-2/III-4/III-1] |
| 892 | II-9 | [III-2/III-4/III-2] |
| 893 | II-9 | [III-2/III-4/III-3] |
| 894 | II-9 | [III-2/III-4/III-4] |
| 895 | II-9 | [III-2/III-4/III-5] |
| 896 | II-9 | [III-2/III-5/III-1] |
| 897 | II-9 | [III-2/III-5/III-2] |
| 898 | II-9 | [III-2/III-5/III-3] |
| 899 | II-9 | [III-2/III-5/III-4] |
| 900 | II-9 | [III-2/III-5/III-5] |
| 901 | II-9 | [III-3/III-1/III-1] |
| 902 | II-9 | [III-3/III-1/III-2] |
| 903 | II-9 | [III-3/III-1/III-3] |
| 904 | II-9 | [III-3/III-1/III-4] |
| 905 | II-9 | [III-3/III-1/III-5] |
| 906 | II-9 | [III-3/III-2/III-1] |
| 907 | II-9 | [III-3/III-2/III-2] |
| 908 | II-9 | [III-3/III-2/III-3] |
| 909 | II-9 | [III-3/III-2/III-4] |
| 910 | II-9 | [III-3/III-2/III-5] |
| 911 | II-9 | [III-3/III-3/III-1] |
| 912 | II-9 | [III-3/III-3/III-2] |
| 913 | II-9 | [III-3/III-3/III-3] |
| 914 | II-9 | [III-3/III-3/III-4] |
| 915 | II-9 | [III-3/III-3/III-5] |
| 916 | II-9 | [III-3/III-4/III-1] |
| 917 | II-9 | [III-3/III-4/III-2] |
| 918 | II-9 | [III-3/III-4/III-3] |
| 919 | II-9 | [III-3/III-4/III-4] |
| 920 | II-9 | [III-3/III-4/III-5] |
| 921 | II-9 | [III-3/III-5/III-1] |
| 922 | II-9 | [III-3/III-5/III-2] |
| 923 | II-9 | [III-3/III-5/III-3] |
| 924 | II-9 | [III-3/III-5/III-4] |
| 925 | II-9 | [III-3/III-5/III-5] |
| 926 | II-9 | [III-4/III-1/III-1] |
| 927 | II-9 | [III-4/III-1/III-2] |
| 928 | II-9 | [III-4/III-1/III-3] |
| 929 | II-9 | [III-4/III-1/III-4] |
| 930 | II-9 | [III-4/III-1/III-5] |
| 931 | II-9 | [III-4/III-2/III-1] |
| 932 | II-9 | [III-4/III-2/III-2] |
| 933 | II-9 | [III-4/III-2/III-3] |
| 934 | II-9 | [III-4/III-2/III-4] |
| 935 | II-9 | [III-4/III-2/III-5] |
| 936 | II-9 | [III-4/III-3/III-1] |
| 937 | II-9 | [III-4/III-3/III-2] |
| 938 | II-9 | [III-4/III-3/III-3] |
| 939 | II-9 | [III-4/III-3/III-4] |
| 940 | II-9 | [III-4/III-3/III-5] |
| 941 | II-9 | [III-4/III-4/III-1] |
| 942 | II-9 | [III-4/III-4/III-2] |
| 943 | II-9 | [III-4/III-4/III-3] |
| 944 | II-9 | [III-4/III-4/III-4] |
| 945 | II-9 | [III-4/III-4/III-5] |
| 946 | II-9 | [III-4/III-5/III-1] |
| 947 | II-9 | [III-4/III-5/III-2] |
| 948 | II-9 | [III-4/III-5/III-3] |
| 949 | II-9 | [III-4/III-5/III-4] |
| 950 | II-9 | [III-4/III-5/III-5] |
| 951 | II-9 | [III-5/III-1/III-1] |
| 952 | II-9 | [III-5/III-1/III-2] |
| 953 | II-9 | [III-5/III-1/III-3] |
| 954 | II-9 | [III-5/III-1/III-4] |
| 955 | II-9 | [III-5/III-1/III-5] |
| 956 | II-9 | [III-5/III-2/III-1] |
| 957 | II-9 | [III-5/III-2/III-2] |
| 958 | II-9 | [III-5/III-2/III-3] |
| 959 | II-9 | [III-5/III-2/III-4] |
| 960 | II-9 | [III-5/III-2/III-5] |
| 961 | II-9 | [III-5/III-3/III-1] |
| 962 | II-9 | [III-5/III-3/III-2] |
| 963 | II-9 | [III-5/III-3/III-3] |
| 964 | II-9 | [III-5/III-3/III-4] |
| 965 | II-9 | [III-5/III-3/III-5] |
| 966 | II-9 | [III-5/III-4/III-1] |
| 967 | II-9 | [III-5/III-4/III-2] |
| 968 | II-9 | [III-5/III-4/III-3] |
| 969 | II-9 | [III-5/III-4/III-4] |
| 970 | II-9 | [III-5/III-4/III-5] |
| 971 | II-9 | [III-5/III-5/III-1] |
| 972 | II-9 | [III-5/III-5/III-2] |
| 973 | II-9 | [III-5/III-5/III-3] |
| 974 | II-9 | [III-5/III-5/III-4] |
| 975 | II-9 | [III-5/III-5/III-5] |
| 976 | II-10 | [III-1/III-1/III-1] |
| 977 | II-10 | [III-1/III-1/III-2] |
| 978 | II-10 | [III-1/III-1/III-3] |
| 979 | II-10 | [III-1/III-1/III-4] |
| 980 | II-10 | [III-1/III-1/III-5] |
| 981 | II-10 | [III-1/III-2/III-1] |
| 982 | II-10 | [III-1/III-2/III-2] |
| 983 | II-10 | [III-1/III-2/III-3] |
| 984 | II-10 | [III-1/III-2/III-4] |
| 985 | II-10 | [III-1/III-2/III-5] |
| 986 | II-10 | [III-1/III-3/III-1] |
| 987 | II-10 | [III-1/III-3/III-2] |
| 988 | II-10 | [III-1/III-3/III-3] |
| 989 | II-10 | [III-1/III-3/III-4] |
| 990 | II-10 | [III-1/III-3/III-5] |
| 991 | II-10 | [III-1/III-4/III-1] |
| 992 | II-10 | [III-1/III-4/III-2] |
| 993 | II-10 | [III-1/III-4/III-3] |
| 994 | II-10 | [III-1/III-4/III-4] |
| 995 | II-10 | [III-1/III-4/III-5] |
| 996 | II-10 | [III-1/III-5/III-1] |
| 997 | II-10 | [III-1/III-5/III-2] |
| 998 | II-10 | [III-1/III-5/III-3] |
| 999 | II-10 | [III-1/III-5/III-4] |
| 1000 | II-10 | [III-1/III-5/III-5] |
| 1001 | II-10 | [III-2/III-1/III-2] |
| 1002 | II-10 | [III-2/III-1/III-3] |
| 1003 | II-10 | [III-2/III-1/III-4] |
| 1004 | II-10 | [III-2/III-1/III-5] |
| 1005 | II-10 | [III-2/III-2/III-2] |
| 1006 | II-10 | [III-2/III-2/III-3] |
| 1007 | II-10 | [III-2/III-2/III-4] |
| 1008 | II-10 | [III-2/III-2/III-5] |
| 1009 | II-10 | [III-2/III-3/III-2] |
| 1010 | II-10 | [III-2/III-3/III-3] |
| 1011 | II-10 | [III-2/III-3/III-4] |
| 1012 | II-10 | [III-2/III-3/III-5] |
| 1013 | II-10 | [III-2/III-4/III-2] |
| 1014 | II-10 | [III-2/III-4/III-3] |
| 1015 | II-10 | [III-2/III-4/III-4] |
| 1016 | II-10 | [III-2/III-4/III-5] |
| 1017 | II-10 | [III-2/III-5/III-2] |
| 1018 | II-10 | [III-2/III-5/III-3] |
| 1019 | II-10 | [III-2/III-5/III-4] |
| 1020 | II-10 | [III-2/III-5/III-5] |
| 1021 | II-10 | [III-3/III-1/III-3] |
| 1022 | II-10 | [III-3/III-1/III-4] |
| 1023 | II-10 | [III-3/III-1/III-5] |
| 1024 | II-10 | [III-3/III-2/III-3] |
| 1025 | II-10 | [III-3/III-2/III-4] |
| 1026 | II-10 | [III-3/III-2/III-5] |
| 1027 | II-10 | [III-3/III-3/III-3] |
| 1028 | II-10 | [III-3/III-3/III-4] |
| 1029 | II-10 | [III-3/III-3/III-5] |
| 1030 | II-10 | [III-3/III-4/III-3] |
| 1031 | II-10 | [III-3/III-4/III-4] |
| 1032 | II-10 | [III-3/III-4/III-5] |
| 1033 | II-10 | [III-3/III-5/III-3] |
| 1034 | II-10 | [III-3/III-5/III-4] |
| 1035 | II-10 | [III-3/III-5/III-5] |
| 1036 | II-10 | [III-4/III-1/III-4] |
| 1037 | II-10 | [III-4/III-1/III-5] |
| 1038 | II-10 | [III-4/III-2/III-4] |
| 1039 | II-10 | [III-4/III-2/III-5] |
| 1040 | II-10 | [III-4/III-3/III-4] |
| 1041 | II-10 | [III-4/III-3/III-5] |
| 1042 | II-10 | [III-4/III-4/III-4] |
| 1043 | II-10 | [III-4/III-4/III-5] |
| 1044 | II-10 | [III-4/III-5/III-4] |
| 1045 | II-10 | [III-4/III-5/III-5] |
| 1046 | II-10 | [III-5/III-1/III-5] |
| 1047 | II-10 | [III-5/III-2/III-5] |
| 1048 | II-10 | [III-5/III-3/III-5] |
| 1049 | II-10 | [III-5/III-4/III-5] |
| 1050 | II-10 | [III-5/III-5/III-5] |
| 1051 | II-11 | [III-1/III-1/III-1] |
| 1052 | II-11 | [III-1/III-1/III-2] |
| 1053 | II-11 | [III-1/III-1/III-3] |
| 1054 | II-11 | [III-1/III-1/III-4] |
| 1055 | II-11 | [III-1/III-1/III-5] |
| 1056 | II-11 | [III-1/III-2/III-1] |
| 1057 | II-11 | [III-1/III-2/III-2] |
| 1058 | II-11 | [III-1/III-2/III-3] |
| 1059 | II-11 | [III-1/III-2/III-4] |
| 1060 | II-11 | [III-1/III-2/III-5] |
| 1061 | II-11 | [III-1/III-3/III-1] |
| 1062 | II-11 | [III-1/III-3/III-2] |
| 1063 | II-11 | [III-1/III-3/III-3] |
| 1064 | II-11 | [III-1/III-3/III-4] |
| 1065 | II-11 | [III-1/III-3/III-5] |
| 1066 | II-11 | [III-1/III-4/III-1] |
| 1067 | II-11 | [III-1/III-4/III-2] |
| 1068 | II-11 | [III-1/III-4/III-3] |
| 1069 | II-11 | [III-1/III-4/III-4] |
| 1070 | II-11 | [III-1/III-4/III-5] |
| 1071 | II-11 | [III-1/III-5/III-1] |
| 1072 | II-11 | [III-1/III-5/III-2] |
| 1073 | II-11 | [III-1/III-5/III-3] |
| 1074 | II-11 | [III-1/III-5/III-4] |
| 1075 | II-11 | [III-1/III-5/III-5] |
| 1076 | II-11 | [III-2/III-1/III-1] |
| 1077 | II-11 | [III-2/III-1/III-2] |
| 1078 | II-11 | [III-2/III-1/III-3] |
| 1079 | II-11 | [III-2/III-1/III-4] |
| 1080 | II-11 | [III-2/III-1/III-5] |
| 1081 | II-11 | [III-2/III-2/III-1] |
| 1082 | II-11 | [III-2/III-2/III-2] |
| 1083 | II-11 | [III-2/III-2/III-3] |
| 1084 | II-11 | [III-2/III-2/III-4] |
| 1085 | II-11 | [III-2/III-2/III-5] |
| 1086 | II-11 | [III-2/III-3/III-1] |
| 1087 | II-11 | [III-2/III-3/III-2] |
| 1088 | II-11 | [III-2/III-3/III-3] |
| 1089 | II-11 | [III-2/III-3/III-4] |
| 1090 | II-11 | [III-2/III-3/III-5] |
| 1091 | II-11 | [III-2/III-4/III-1] |
| 1092 | II-11 | [III-2/III-4/III-2] |
| 1093 | II-11 | [III-2/III-4/III-3] |
| 1094 | II-11 | [III-2/III-4/III-4] |
| 1095 | II-11 | [III-2/III-4/III-5] |
| 1096 | II-11 | [III-2/III-5/III-1] |
| 1097 | II-11 | [III-2/III-5/III-2] |
| 1098 | II-11 | [III-2/III-5/III-3] |
| 1099 | II-11 | [III-2/III-5/III-4] |
| 1100 | II-11 | [III-2/III-5/III-5] |
| 1101 | II-11 | [III-3/III-1/III-1] |
| 1102 | II-11 | [III-3/III-1/III-2] |
| 1103 | II-11 | [III-3/III-1/III-3] |
| 1104 | II-11 | [III-3/III-1/III-4] |
| 1105 | II-11 | [III-3/III-1/III-5] |
| 1106 | II-11 | [III-3/III-2/III-1] |
| 1107 | II-11 | [III-3/III-2/III-2] |
| 1108 | II-11 | [III-3/III-2/III-3] |
| 1109 | II-11 | [III-3/III-2/III-4] |
| 1110 | II-11 | [III-3/III-2/III-5] |
| 1111 | II-11 | [III-3/III-3/III-1] |
| 1112 | II-11 | [III-3/III-3/III-2] |
| 1113 | II-11 | [III-3/III-3/III-3] |
| 1114 | II-11 | [III-3/III-3/III-4] |
| 1115 | II-11 | [III-3/III-3/III-5] |
| 1116 | II-11 | [III-3/III-4/III-1] |
| 1117 | II-11 | [III-3/III-4/III-2] |
| 1118 | II-11 | [III-3/III-4/III-3] |
| 1119 | II-11 | [III-3/III-4/III-4] |
| 1120 | II-11 | [III-3/III-4/III-5] |
| 1121 | II-11 | [III-3/III-5/III-1] |
| 1122 | II-11 | [III-3/III-5/III-2] |
| 1123 | II-11 | [III-3/III-5/III-3] |
| 1124 | II-11 | [III-3/III-5/III-4] |
| 1125 | II-11 | [III-3/III-5/III-5] |
| 1126 | II-11 | [III-4/III-1/III-1] |
| 1127 | II-11 | [III-4/III-1/III-2] |
| 1128 | II-11 | [III-4/III-1/III-3] |
| 1129 | II-11 | [III-4/III-1/III-4] |
| 1130 | II-11 | [III-4/III-1/III-5] |
| 1131 | II-11 | [III-4/III-2/III-1] |
| 1132 | II-11 | [III-4/III-2/III-2] |
| 1133 | II-11 | [III-4/III-2/III-3] |
| 1134 | II-11 | [III-4/III-2/III-4] |
| 1135 | II-11 | [III-4/III-2/III-5] |
| 1136 | II-11 | [III-4/III-3/III-1] |
| 1137 | II-11 | [III-4/III-3/III-2] |
| 1138 | II-11 | [III-4/III-3/III-3] |
| 1139 | II-11 | [III-4/III-3/III-4] |
| 1140 | II-11 | [III-4/III-3/III-5] |
| 1141 | II-11 | [III-4/III-4/III-1] |
| 1142 | II-11 | [III-4/III-4/III-2] |
| 1143 | II-11 | [III-4/III-4/III-3] |
| 1144 | II-11 | [III-4/III-4/III-4] |
| 1145 | II-11 | [III-4/III-4/III-5] |
| 1146 | II-11 | [III-4/III-5/III-1] |
| 1147 | II-11 | [III-4/III-5/III-2] |
| 1148 | II-11 | [III-4/III-5/III-3] |
| 1149 | II-11 | [III-4/III-5/III-4] |
| 1150 | II-11 | [III-4/III-5/III-5] |
| 1151 | II-11 | [III-5/III-1/III-1] |
| 1152 | II-11 | [III-5/III-1/III-2] |
| 1153 | II-11 | [III-5/III-1/III-3] |
| 1154 | II-11 | [III-5/III-1/III-4] |
| 1155 | II-11 | [III-5/III-1/III-5] |
| 1156 | II-11 | [III-5/III-2/III-1] |
| 1157 | II-11 | [III-5/III-2/III-2] |
| 1158 | II-11 | [III-5/III-2/III-3] |
| 1159 | II-11 | [III-5/III-2/III-4] |
| 1160 | II-11 | [III-5/III-2/III-5] |
| 1161 | II-11 | [III-5/III-3/III-1] |
| 1162 | II-11 | [III-5/III-3/III-2] |
| 1163 | II-11 | [III-5/III-3/III-3] |
| 1164 | II-11 | [III-5/III-3/III-4] |
| 1165 | II-11 | [III-5/III-3/III-5] |
| 1166 | II-11 | [III-5/III-4/III-1] |
| 1167 | II-11 | [III-5/III-4/III-2] |
| 1168 | II-11 | [III-5/III-4/III-3] |
| 1169 | II-11 | [III-5/III-4/III-4] |
| 1170 | II-11 | [III-5/III-4/III-5] |
| 1171 | II-11 | [III-5/III-5/III-1] |
| 1172 | II-11 | [III-5/III-5/III-2] |
| 1173 | II-11 | [III-5/III-5/III-3] |
| 1174 | II-11 | [III-5/III-5/III-4] |
| 1175 | II-11 | [III-5/III-5/III-5] |
| 1176 | II-12 | [III-1/III-1/III-1] |
| 1177 | II-12 | [III-1/III-1/III-2] |
| 1178 | II-12 | [III-1/III-1/III-3] |
| 1179 | II-12 | [III-1/III-1/III-4] |
| 1180 | II-12 | [III-1/III-1/III-5] |
| 1181 | II-12 | [III-1/III-2/III-1] |
| 1182 | II-12 | [III-1/III-2/III-2] |
| 1183 | II-12 | [III-1/III-2/III-3] |
| 1184 | II-12 | [III-1/III-2/III-4] |
| 1185 | II-12 | [III-1/III-2/III-5] |
| 1186 | II-12 | [III-1/III-3/III-1] |
| 1187 | II-12 | [III-1/III-3/III-2] |
| 1188 | II-12 | [III-1/III-3/III-3] |
| 1189 | II-12 | [III-1/III-3/III-4] |
| 1190 | II-12 | [III-1/III-3/III-5] |
| 1191 | II-12 | [III-1/III-4/III-1] |
| 1192 | II-12 | [III-1/III-4/III-2] |
| 1193 | II-12 | [III-1/III-4/III-3] |
| 1194 | II-12 | [III-1/III-4/III-4] |
| 1195 | II-12 | [III-1/III-4/III-5] |
| 1196 | II-12 | [III-1/III-5/III-1] |
| 1197 | II-12 | [III-1/III-5/III-2] |
| 1198 | II-12 | [III-1/III-5/III-3] |
| 1199 | II-12 | [III-1/III-5/III-4] |
| 1200 | II-12 | [III-1/III-5/III-5] |
| 1201 | II-12 | [III-2/III-1/III-1] |
| 1202 | II-12 | [III-2/III-1/III-2] |
| 1203 | II-12 | [III-2/III-1/III-3] |
| 1204 | II-12 | [III-2/III-1/III-4] |
| 1205 | II-12 | [III-2/III-1/III-5] |
| 1206 | II-12 | [III-2/III-2/III-1] |
| 1207 | II-12 | [III-2/III-2/III-2] |
| 1208 | II-12 | [III-2/III-2/III-3] |
| 1209 | II-12 | [III-2/III-2/III-4] |
| 1210 | II-12 | [III-2/III-2/III-5] |
| 1211 | II-12 | [III-2/III-3/III-1] |
| 1212 | II-12 | [III-2/III-3/III-2] |
| 1213 | II-12 | [III-2/III-3/III-3] |
| 1214 | II-12 | [III-2/III-3/III-4] |
| 1215 | II-12 | [III-2/III-3/III-5] |
| 1216 | II-12 | [III-2/III-4/III-1] |
| 1217 | II-12 | [III-2/III-4/III-2] |
| 1218 | II-12 | [III-2/III-4/III-3] |
| 1219 | II-12 | [III-2/III-4/III-4] |
| 1220 | II-12 | [III-2/III-4/III-5] |
| 1221 | II-12 | [III-2/III-5/III-1] |
| 1222 | II-12 | [III-2/III-5/III-2] |
| 1223 | II-12 | [III-2/III-5/III-3] |
| 1224 | II-12 | [III-2/III-5/III-4] |
| 1225 | II-12 | [III-2/III-5/III-5] |
| 1226 | II-12 | [III-3/III-1/III-1] |
| 1227 | II-12 | [III-3/III-1/III-2] |
| 1228 | II-12 | [III-3/III-1/III-3] |
| 1229 | II-12 | [III-3/III-1/III-4] |
| 1230 | II-12 | [III-3/III-1/III-5] |
| 1231 | II-12 | [III-3/III-2/III-1] |
| 1232 | II-12 | [III-3/III-2/III-2] |
| 1233 | II-12 | [III-3/III-2/III-3] |
| 1234 | II-12 | [III-3/III-2/III-4] |
| 1235 | II-12 | [III-3/III-2/III-5] |
| 1236 | II-12 | [III-3/III-3/III-1] |
| 1237 | II-12 | [III-3/III-3/III-2] |
| 1238 | II-12 | [III-3/III-3/III-3] |
| 1239 | II-12 | [III-3/III-3/III-4] |
| 1240 | II-12 | [III-3/III-3/III-5] |
| 1241 | II-12 | [III-3/III-4/III-1] |
| 1242 | II-12 | [III-3/III-4/III-2] |
| 1243 | II-12 | [III-3/III-4/III-3] |
| 1244 | II-12 | [III-3/III-4/III-4] |
| 1245 | II-12 | [III-3/III-4/III-5] |
| 1246 | II-12 | [III-3/III-5/III-1] |
| 1247 | II-12 | [III-3/III-5/III-2] |
| 1248 | II-12 | [III-3/III-5/III-3] |
| 1249 | II-12 | [III-3/III-5/III-4] |
| 1250 | II-12 | [III-3/III-5/III-5] |
| 1251 | II-12 | [III-4/III-1/III-1] |
| 1252 | II-12 | [III-4/III-1/III-2] |
| 1253 | II-12 | [III-4/III-1/III-3] |
| 1254 | II-12 | [III-4/III-1/III-4] |
| 1255 | II-12 | [III-4/III-1/III-5] |
| 1256 | II-12 | [III-4/III-2/III-1] |
| 1257 | II-12 | [III-4/III-2/III-2] |
| 1258 | II-12 | [III-4/III-2/III-3] |
| 1259 | II-12 | [III-4/III-2/III-4] |
| 1260 | II-12 | [III-4/III-2/III-5] |
| 1261 | II-12 | [III-4/III-3/III-1] |
| 1262 | II-12 | [III-4/III-3/III-2] |
| 1263 | II-12 | [III-4/III-3/III-3] |
| 1264 | II-12 | [III-4/III-3/III-4] |
| 1265 | II-12 | [III-4/III-3/III-5] |
| 1266 | II-12 | [III-4/III-4/III-1] |
| 1267 | II-12 | [III-4/III-4/III-2] |
| 1268 | II-12 | [III-4/III-4/III-3] |
| 1269 | II-12 | [III-4/III-4/III-4] |
| 1270 | II-12 | [III-4/III-4/III-5] |
| 1271 | II-12 | [III-4/III-5/III-1] |
| 1272 | II-12 | [III-4/III-5/III-2] |
| 1273 | II-12 | [III-4/III-5/III-3] |
| 1274 | II-12 | [III-4/III-5/III-4] |
| 1275 | II-12 | [III-4/III-5/III-5] |
| 1276 | II-12 | [III-5/III-1/III-1] |
| 1277 | II-12 | [III-5/III-1/III-2] |
| 1278 | II-12 | [III-5/III-1/III-3] |
| 1279 | II-12 | [III-5/III-1/III-4] |
| 1280 | II-12 | [III-5/III-1/III-5] |
| 1281 | II-12 | [III-5/III-2/III-1] |
| 1282 | II-12 | [III-5/III-2/III-2] |
| 1283 | II-12 | [III-5/III-2/III-3] |
| 1284 | II-12 | [III-5/III-2/III-4] |
| 1285 | II-12 | [III-5/III-2/III-5] |
| 1286 | II-12 | [III-5/III-3/III-1] |
| 1287 | II-12 | [III-5/III-3/III-2] |
| 1288 | II-12 | [III-5/III-3/III-3] |
| 1289 | II-12 | [III-5/III-3/III-4] |
| 1290 | II-12 | [III-5/III-3/III-5] |
| 1291 | II-12 | [III-5/III-4/III-1] |
| 1292 | II-12 | [III-5/III-4/III-2] |
| 1293 | II-12 | [III-5/III-4/III-3] |
| 1294 | II-12 | [III-5/III-4/III-4] |
| 1295 | II-12 | [III-5/III-4/III-5] |
| 1296 | II-12 | [III-5/III-5/III-1] |
| 1297 | II-12 | [III-5/III-5/III-2] |
| 1298 | II-12 | [III-5/III-5/III-3] |
| 1299 | II-12 | [III-5/III-5/III-4] |
| 1300 | II-12 | [III-5/III-5/III-5] |
| 1301 | II-13 | [III-1/III-1/III-1] |
| 1302 | II-13 | [III-1/III-1/III-2] |
| 1303 | II-13 | [III-1/III-1/III-3] |
| 1304 | II-13 | [III-1/III-1/III-4] |
| 1305 | II-13 | [III-1/III-1/III-5] |
| 1306 | II-13 | [III-1/III-2/III-1] |
| 1307 | II-13 | [III-1/III-2/III-2] |
| 1308 | II-13 | [III-1/III-2/III-3] |
| 1309 | II-13 | [III-1/III-2/III-4] |
| 1310 | II-13 | [III-1/III-2/III-5] |
| 1311 | II-13 | [III-1/III-3/III-1] |
| 1312 | II-13 | [III-1/III-3/III-2] |
| 1313 | II-13 | [III-1/III-3/III-3] |
| 1314 | II-13 | [III-1/III-3/III-4] |
| 1315 | II-13 | [III-1/III-3/III-5] |
| 1316 | II-13 | [III-1/III-4/III-1] |
| 1317 | II-13 | [III-1/III-4/III-2] |
| 1318 | II-13 | [III-1/III-4/III-3] |
| 1319 | II-13 | [III-1/III-4/III-4] |
| 1320 | II-13 | [III-1/III-4/III-5] |
| 1321 | II-13 | [III-1/III-5/III-1] |
| 1322 | II-13 | [III-1/III-5/III-2] |
| 1323 | II-13 | [III-1/III-5/III-3] |
| 1324 | II-13 | [III-1/III-5/III-4] |
| 1325 | II-13 | [III-1/III-5/III-5] |
| 1326 | II-13 | [III-2/III-1/III-2] |
| 1327 | II-13 | [III-2/III-1/III-3] |
| 1328 | II-13 | [III-2/III-1/III-4] |
| 1329 | II-13 | [III-2/III-1/III-5] |
| 1330 | II-13 | [III-2/III-2/III-2] |
| 1331 | II-13 | [III-2/III-2/III-3] |
| 1332 | II-13 | [III-2/III-2/III-4] |
| 1333 | II-13 | [III-2/III-2/III-5] |
| 1334 | II-13 | [III-2/III-3/III-2] |
| 1335 | II-13 | [III-2/III-3/III-3] |
| 1336 | II-13 | [III-2/III-3/III-4] |
| 1337 | II-13 | [III-2/III-3/III-5] |
| 1338 | II-13 | [III-2/III-4/III-2] |
| 1339 | II-13 | [III-2/III-4/III-3] |
| 1340 | II-13 | [III-2/III-4/III-4] |
| 1341 | II-13 | [III-2/III-4/III-5] |
| 1342 | II-13 | [III-2/III-5/III-2] |
| 1343 | II-13 | [III-2/III-5/III-3] |
| 1344 | II-13 | [III-2/III-5/III-4] |
| 1345 | II-13 | [III-2/III-5/III-5] |
| 1346 | II-13 | [III-3/III-1/III-3] |
| 1347 | II-13 | [III-3/III-1/III-4] |
| 1348 | II-13 | [III-3/III-1/III-5] |
| 1349 | II-13 | [III-3/III-2/III-3] |
| 1350 | II-13 | [III-3/III-2/III-4] |
| 1351 | II-13 | [III-3/III-2/III-5] |
| 1352 | II-13 | [III-3/III-3/III-3] |
| 1353 | II-13 | [III-3/III-3/III-4] |
| 1354 | II-13 | [III-3/III-3/III-5] |
| 1355 | II-13 | [III-3/III-4/III-3] |
| 1356 | II-13 | [III-3/III-4/III-4] |
| 1357 | II-13 | [III-3/III-4/III-5] |
| 1358 | II-13 | [III-3/III-5/III-3] |
| 1359 | II-13 | [III-3/III-5/III-4] |
| 1360 | II-13 | [III-3/III-5/III-5] |
| 1361 | II-13 | [III-4/III-1/III-4] |
| 1362 | II-13 | [III-4/III-1/III-5] |
| 1363 | II-13 | [III-4/III-2/III-4] |
| 1364 | II-13 | [III-4/III-2/III-5] |
| 1365 | II-13 | [III-4/III-3/III-4] |
| 1366 | II-13 | [III-4/III-3/III-5] |
| 1367 | II-13 | [III-4/III-4/III-4] |
| 1368 | II-13 | [III-4/III-4/III-5] |
| 1369 | II-13 | [III-4/III-5/III-4] |
| 1370 | II-13 | [III-4/III-5/III-5] |
| 1371 | II-13 | [III-5/III-1/III-5] |
| 1372 | II-13 | [III-5/III-2/III-5] |
| 1373 | II-13 | [III-5/III-3/III-5] |
| 1374 | II-13 | [III-5/III-4/III-5] |
| 1375 | II-13 | [III-5/III-5/III-5] |
| 1376 | II-14 | [III-1/III-1/III-1] |
| 1377 | II-14 | [III-1/III-1/III-2] |
| 1378 | II-14 | [III-1/III-1/III-3] |
| 1379 | II-14 | [III-1/III-1/III-4] |
| 1380 | II-14 | [III-1/III-1/III-5] |
| 1381 | II-14 | [III-1/III-2/III-1] |
| 1382 | II-14 | [III-1/III-2/III-2] |
| 1383 | II-14 | [III-1/III-2/III-3] |
| 1384 | II-14 | [III-1/III-2/III-4] |
| 1385 | II-14 | [III-1/III-2/III-5] |
| 1386 | II-14 | [III-1/III-3/III-1] |
| 1387 | II-14 | [III-1/III-3/III-2] |
| 1388 | II-14 | [III-1/III-3/III-3] |
| 1389 | II-14 | [III-1/III-3/III-4] |
| 1390 | II-14 | [III-1/III-3/III-5] |
| 1391 | II-14 | [III-1/III-4/III-1] |
| 1392 | II-14 | [III-1/III-4/III-2] |
| 1393 | II-14 | [III-1/III-4/III-3] |
| 1394 | II-14 | [III-1/III-4/III-4] |
| 1395 | II-14 | [III-1/III-4/III-5] |
| 1396 | II-14 | [III-1/III-5/III-1] |
| 1397 | II-14 | [III-1/III-5/III-2] |
| 1398 | II-14 | [III-1/III-5/III-3] |
| 1399 | II-14 | [III-1/III-5/III-4] |
| 1400 | II-14 | [III-1/III-5/III-5] |
| 1401 | II-14 | [III-2/III-1/III-2] |
| 1402 | II-14 | [III-2/III-1/III-3] |
| 1403 | II-14 | [III-2/III-1/III-4] |
| 1404 | II-14 | [III-2/III-1/III-5] |
| 1405 | II-14 | [III-2/III-2/III-2] |
| 1406 | II-14 | [III-2/III-2/III-3] |
| 1407 | II-14 | [III-2/III-2/III-4] |
| 1408 | II-14 | [III-2/III-2/III-5] |
| 1409 | II-14 | [III-2/III-3/III-2] |
| 1410 | II-14 | [III-2/III-3/III-3] |
| 1411 | II-14 | [III-2/III-3/III-4] |
| 1412 | II-14 | [III-2/III-3/III-5] |
| 1413 | II-14 | [III-2/III-4/III-2] |
| 1414 | II-14 | [III-2/III-4/III-3] |
| 1415 | II-14 | [III-2/III-4/III-4] |
| 1416 | II-14 | [III-2/III-4/III-5] |
| 1417 | II-14 | [III-2/III-5/III-2] |
| 1418 | II-14 | [III-2/III-5/III-3] |
| 1419 | II-14 | [III-2/III-5/III-4] |
| 1420 | II-14 | [III-2/III-5/III-5] |
| 1421 | II-14 | [III-3/III-1/III-3] |
| 1422 | II-14 | [III-3/III-1/III-4] |
| 1423 | II-14 | [III-3/III-1/III-5] |
| 1424 | II-14 | [III-3/III-2/III-3] |
| 1425 | II-14 | [III-3/III-2/III-4] |
| 1426 | II-14 | [III-3/III-2/III-5] |
| 1427 | II-14 | [III-3/III-3/III-3] |
| 1428 | II-14 | [III-3/III-3/III-4] |
| 1429 | II-14 | [III-3/III-3/III-5] |
| 1430 | II-14 | [III-3/III-4/III-3] |
| 1431 | II-14 | [III-3/III-4/III-4] |
| 1432 | II-14 | [III-3/III-4/III-5] |
| 1433 | II-14 | [III-3/III-5/III-3] |
| 1434 | II-14 | [III-3/III-5/III-4] |
| 1435 | II-14 | [III-3/III-5/III-5] |
| 1436 | II-14 | [III-4/III-1/III-4] |
| 1437 | II-14 | [III-4/III-1/III-5] |
| 1438 | II-14 | [III-4/III-2/III-4] |
| 1439 | II-14 | [III-4/III-2/III-5] |
| 1440 | II-14 | [III-4/III-3/III-4] |
| 1441 | II-14 | [III-4/III-3/III-5] |
| 1442 | II-14 | [III-4/III-4/III-4] |
| 1443 | II-14 | [III-4/III-4/III-5] |
| 1444 | II-14 | [III-4/III-5/III-4] |
| 1445 | II-14 | [III-4/III-5/III-5] |
| 1446 | II-14 | [III-5/III-1/III-5] |
| 1447 | II-14 | [III-5/III-2/III-5] |
| 1448 | II-14 | [III-5/III-3/III-5] |
| 1449 | II-14 | [III-5/III-4/III-5] |
| 1450 | II-14 | [III-5/III-5/III-5] |
| 1451 | II-15 | [III-1/III-1/III-1] |
| 1452 | II-15 | [III-1/III-1/III-2] |
| 1453 | II-15 | [III-1/III-1/III-3] |
| 1454 | II-15 | [III-1/III-1/III-4] |
| 1455 | II-15 | [III-1/III-1/III-5] |
| 1456 | II-15 | [III-1/III-2/III-1] |
| 1457 | II-15 | [III-1/III-2/III-2] |
| 1458 | II-15 | [III-1/III-2/III-3] |
| 1459 | II-15 | [III-1/III-2/III-4] |
| 1460 | II-15 | [III-1/III-2/III-5] |
| 1461 | II-15 | [III-1/III-3/III-1] |
| 1462 | II-15 | [III-1/III-3/III-2] |
| 1463 | II-15 | [III-1/III-3/III-3] |
| 1464 | II-15 | [III-1/III-3/III-4] |
| 1465 | II-15 | [III-1/III-3/III-5] |
| 1466 | II-15 | [III-1/III-4/III-1] |
| 1467 | II-15 | [III-1/III-4/III-2] |
| 1468 | II-15 | [III-1/III-4/III-3] |
| 1469 | II-15 | [III-1/III-4/III-4] |
| 1470 | II-15 | [III-1/III-4/III-5] |
| 1471 | II-15 | [III-1/III-5/III-1] |
| 1472 | II-15 | [III-1/III-5/III-2] |
| 1473 | II-15 | [III-1/III-5/III-3] |
| 1474 | II-15 | [III-1/III-5/III-4] |
| 1475 | II-15 | [III-1/III-5/III-5] |
| 1476 | II-15 | [III-2/III-1/III-1] |
| 1477 | II-15 | [III-2/III-1/III-2] |
| 1478 | II-15 | [III-2/III-1/III-3] |
| 1479 | II-15 | [III-2/III-1/III-4] |
| 1480 | II-15 | [III-2/III-1/III-5] |
| 1481 | II-15 | [III-2/III-2/III-1] |
| 1482 | II-15 | [III-2/III-2/III-2] |
| 1483 | II-15 | [III-2/III-2/III-3] |
| 1484 | II-15 | [III-2/III-2/III-4] |
| 1485 | II-15 | [III-2/III-2/III-5] |
| 1486 | II-15 | [III-2/III-3/III-1] |
| 1487 | II-15 | [III-2/III-3/III-2] |
| 1488 | II-15 | [III-2/III-3/III-3] |
| 1489 | II-15 | [III-2/III-3/III-4] |
| 1490 | II-15 | [III-2/III-3/III-5] |
| 1491 | II-15 | [III-2/III-4/III-1] |
| 1492 | II-15 | [III-2/III-4/III-2] |
| 1493 | II-15 | [III-2/III-4/III-3] |
| 1494 | II-15 | [III-2/III-4/III-4] |
| 1495 | II-15 | [III-2/III-4/III-5] |
| 1496 | II-15 | [III-2/III-5/III-1] |
| 1497 | II-15 | [III-2/III-5/III-2] |
| 1498 | II-15 | [III-2/III-5/III-3] |
| 1499 | II-15 | [III-2/III-5/III-4] |
| 1500 | II-15 | [III-2/III-5/III-5] |
| 1501 | II-15 | [III-3/III-1/III-1] |
| 1502 | II-15 | [III-3/III-1/III-2] |
| 1503 | II-15 | [III-3/III-1/III-3] |
| 1504 | II-15 | [III-3/III-1/III-4] |
| 1505 | II-15 | [III-3/III-1/III-5] |
| 1506 | II-15 | [III-3/III-2/III-1] |
| 1507 | II-15 | [III-3/III-2/III-2] |
| 1508 | II-15 | [III-3/III-2/III-3] |
| 1509 | II-15 | [III-3/III-2/III-4] |
| 1510 | II-15 | [III-3/III-2/III-5] |
| 1511 | II-15 | [III-3/III-3/III-1] |
| 1512 | II-15 | [III-3/III-3/III-2] |
| 1513 | II-15 | [III-3/III-3/III-3] |
| 1514 | II-15 | [III-3/III-3/III-4] |
| 1515 | II-15 | [III-3/III-3/III-5] |
| 1516 | II-15 | [III-3/III-4/III-1] |
| 1517 | II-15 | [III-3/III-4/III-2] |
| 1518 | II-15 | [III-3/III-4/III-3] |
| 1519 | II-15 | [III-3/III-4/III-4] |
| 1520 | II-15 | [III-3/III-4/III-5] |
| 1521 | II-15 | [III-3/III-5/III-1] |
| 1522 | II-15 | [III-3/III-5/III-2] |
| 1523 | II-15 | [III-3/III-5/III-3] |
| 1524 | II-15 | [III-3/III-5/III-4] |
| 1525 | II-15 | [III-3/III-5/III-5] |
| 1526 | II-15 | [III-4/III-1/III-1] |
| 1527 | II-15 | [III-4/III-1/III-2] |
| 1528 | II-15 | [III-4/III-1/III-3] |
| 1529 | II-15 | [III-4/III-1/III-4] |
| 1530 | II-15 | [III-4/III-1/III-5] |
| 1531 | II-15 | [III-4/III-2/III-1] |
| 1532 | II-15 | [III-4/III-2/III-2] |
| 1533 | II-15 | [III-4/III-2/III-3] |
| 1534 | II-15 | [III-4/III-2/III-4] |
| 1535 | II-15 | [III-4/III-2/III-5] |
| 1536 | II-15 | [III-4/III-3/III-1] |
| 1537 | II-15 | [III-4/III-3/III-2] |
| 1538 | II-15 | [III-4/III-3/III-3] |
| 1539 | II-15 | [III-4/III-3/III-4] |
| 1540 | II-15 | [III-4/III-3/III-5] |
| 1541 | II-15 | [III-4/III-4/III-1] |
| 1542 | II-15 | [III-4/III-4/III-2] |
| 1543 | II-15 | [III-4/III-4/III-3] |
| 1544 | II-15 | [III-4/III-4/III-4] |
| 1545 | II-15 | [III-4/III-4/III-5] |
| 1546 | II-15 | [III-4/III-5/III-1] |
| 1547 | II-15 | [III-4/III-5/III-2] |
| 1548 | II-15 | [III-4/III-5/III-3] |
| 1549 | II-15 | [III-4/III-5/III-4] |
| 1550 | II-15 | [III-4/III-5/III-5] |
| 1551 | II-15 | [III-5/III-1/III-1] |
| 1552 | II-15 | [III-5/III-1/III-2] |
| 1553 | II-15 | [III-5/III-1/III-3] |
| 1554 | II-15 | [III-5/III-1/III-4] |
| 1555 | II-15 | [III-5/III-1/III-5] |
| 1556 | II-15 | [III-5/III-2/III-1] |
| 1557 | II-15 | [III-5/III-2/III-2] |
| 1558 | II-15 | [III-5/III-2/III-3] |
| 1559 | II-15 | [III-5/III-2/III-4] |
| 1560 | II-15 | [III-5/III-2/III-5] |
| 1561 | II-15 | [III-5/III-3/III-1] |
| 1562 | II-15 | [III-5/III-3/III-2] |
| 1563 | II-15 | [III-5/III-3/III-3] |
| 1564 | II-15 | [III-5/III-3/III-4] |
| 1565 | II-15 | [III-5/III-3/III-5] |
| 1566 | II-15 | [III-5/III-4/III-1] |
| 1567 | II-15 | [III-5/III-4/III-2] |
| 1568 | II-15 | [III-5/III-4/III-3] |
| 1569 | II-15 | [III-5/III-4/III-4] |
| 1570 | II-15 | [III-5/III-4/III-5] |
| 1571 | II-15 | [III-5/III-5/III-1] |
| 1572 | II-15 | [III-5/III-5/III-2] |
| 1573 | II-15 | [III-5/III-5/III-3] |
| 1574 | II-15 | [III-5/III-5/III-4] |
| 1575 | II-15 | [III-5/III-5/III-5] |
| 1576 | II-16 | [III-1/III-1/III-1] |
| 1577 | II-16 | [III-1/III-1/III-2] |
| 1578 | II-16 | [III-1/III-1/III-3] |
| 1579 | II-16 | [III-1/III-1/III-4] |
| 1580 | II-16 | [III-1/III-1/III-5] |
| 1581 | II-16 | [III-1/III-2/III-1] |
| 1582 | II-16 | [III-1/III-2/III-2] |
| 1583 | II-16 | [III-1/III-2/III-3] |
| 1584 | II-16 | [III-1/III-2/III-4] |
| 1585 | II-16 | [III-1/III-2/III-5] |
| 1586 | II-16 | [III-1/III-3/III-1] |
| 1587 | II-16 | [III-1/III-3/III-2] |
| 1588 | II-16 | [III-1/III-3/III-3] |
| 1589 | II-16 | [III-1/III-3/III-4] |
| 1590 | II-16 | [III-1/III-3/III-5] |
| 1591 | II-16 | [III-1/III-4/III-1] |
| 1592 | II-16 | [III-1/III-4/III-2] |
| 1593 | II-16 | [III-1/III-4/III-3] |
| 1594 | II-16 | [III-1/III-4/III-4] |
| 1595 | II-16 | [III-1/III-4/III-5] |
| 1596 | II-16 | [III-1/III-5/III-1] |
| 1597 | II-16 | [III-1/III-5/III-2] |
| 1598 | II-16 | [III-1/III-5/III-3] |
| 1599 | II-16 | [III-1/III-5/III-4] |
| 1600 | II-16 | [III-1/III-5/III-5] |
| 1601 | II-16 | [III-2/III-1/III-1] |
| 1602 | II-16 | [III-2/III-1/III-2] |
| 1603 | II-16 | [III-2/III-1/III-3] |
| 1604 | II-16 | [III-2/III-1/III-4] |
| 1605 | II-16 | [III-2/III-1/III-5] |
| 1606 | II-16 | [III-2/III-2/III-1] |
| 1607 | II-16 | [III-2/III-2/III-2] |
| 1608 | II-16 | [III-2/III-2/III-3] |
| 1609 | II-16 | [III-2/III-2/III-4] |
| 1610 | II-16 | [III-2/III-2/III-5] |
| 1611 | II-16 | [III-2/III-3/III-1] |
| 1612 | II-16 | [III-2/III-3/III-2] |
| 1613 | II-16 | [III-2/III-3/III-3] |
| 1614 | II-16 | [III-2/III-3/III-4] |
| 1615 | II-16 | [III-2/III-3/III-5] |
| 1616 | II-16 | [III-2/III-4/III-1] |
| 1617 | II-16 | [III-2/III-4/III-2] |
| 1618 | II-16 | [III-2/III-4/III-3] |
| 1619 | II-16 | [III-2/III-4/III-4] |
| 1620 | II-16 | [III-2/III-4/III-5] |
| 1621 | II-16 | [III-2/III-5/III-1] |
| 1622 | II-16 | [III-2/III-5/III-2] |
| 1623 | II-16 | [III-2/III-5/III-3] |
| 1624 | II-16 | [III-2/III-5/III-4] |
| 1625 | II-16 | [III-2/III-5/III-5] |
| 1626 | II-16 | [III-3/III-1/III-1] |
| 1627 | II-16 | [III-3/III-1/III-2] |
| 1628 | II-16 | [III-3/III-1/III-3] |
| 1629 | II-16 | [III-3/III-1/III-4] |
| 1630 | II-16 | [III-3/III-1/III-5] |
| 1631 | II-16 | [III-3/III-2/III-1] |
| 1632 | II-16 | [III-3/III-2/III-2] |
| 1633 | II-16 | [III-3/III-2/III-3] |
| 1634 | II-16 | [III-3/III-2/III-4] |
| 1635 | II-16 | [III-3/III-2/III-5] |
| 1636 | II-16 | [III-3/III-3/III-1] |
| 1637 | II-16 | [III-3/III-3/III-2] |
| 1638 | II-16 | [III-3/III-3/III-3] |
| 1639 | II-16 | [III-3/III-3/III-4] |
| 1640 | II-16 | [III-3/III-3/III-5] |
| 1641 | II-16 | [III-3/III-4/III-1] |
| 1642 | II-16 | [III-3/III-4/III-2] |
| 1643 | II-16 | [III-3/III-4/III-3] |
| 1644 | II-16 | [III-3/III-4/III-4] |
| 1645 | II-16 | [III-3/III-4/III-5] |
| 1646 | II-16 | [III-3/III-5/III-1] |
| 1647 | II-16 | [III-3/III-5/III-2] |
| 1648 | II-16 | [III-3/III-5/III-3] |
| 1649 | II-16 | [III-3/III-5/III-4] |
| 1650 | II-16 | [III-3/III-5/III-5] |
| 1651 | II-16 | [III-4/III-1/III-1] |
| 1652 | II-16 | [III-4/III-1/III-2] |
| 1653 | II-16 | [III-4/III-1/III-3] |
| 1654 | II-16 | [III-4/III-1/III-4] |
| 1653 | II-16 | [III-4/III-1/III-5] |
| 1656 | II-16 | [III-4/III-2/III-1] |
| 1657 | II-16 | [III-4/III-2/III-2] |
| 1658 | II-16 | [III-4/III-2/III-3] |
| 1659 | II-16 | [III-4/III-2/III-4] |
| 1660 | II-16 | [III-4/III-2/III-5] |
| 1661 | II-16 | [III-4/III-3/III-1] |
| 1662 | II-16 | [III-4/III-3/III-2] |
| 1663 | II-16 | [III-4/III-3/III-3] |
| 1664 | II-16 | [III-4/III-3/III-4] |
| 1665 | II-16 | [III-4/III-3/III-5] |
| 1666 | II-16 | [III-4/III-4/III-1] |
| 1667 | II-16 | [III-4/III-4/III-2] |
| 1668 | II-16 | [III-4/III-4/III-3] |
| 1669 | II-16 | [III-4/III-4/III-4] |
| 1670 | II-16 | [III-4/III-4/III-5] |
| 1671 | II-16 | [III-4/III-5/III-1] |
| 1672 | II-16 | [III-4/III-5/III-2] |
| 1673 | II-16 | [III-4/III-5/III-3] |
| 1674 | II-16 | [III-4/III-5/III-4] |
| 1675 | II-16 | [III-4/III-5/III-5] |
| 1676 | II-16 | [III-5/III-1/III-1] |
| 1677 | II-16 | [III-5/III-1/III-2] |
| 1678 | II-16 | [III-5/III-1/III-3] |
| 1679 | II-16 | [III-5/III-1/III-4] |
| 1680 | II-16 | [III-5/III-1/III-5] |
| 1681 | II-16 | [III-5/III-2/III-1] |
| 1682 | II-16 | [III-5/III-2/III-2] |
| 1683 | II-16 | [III-5/III-2/III-3] |
| 1684 | II-16 | [III-5/III-2/III-4] |
| 1685 | II-16 | [III-5/III-2/III-5] |
| 1686 | II-16 | [III-5/III-3/III-1] |
| 1687 | II-16 | [III-5/III-3/III-2] |
| 1688 | II-16 | [III-5/III-3/III-3] |
| 1689 | II-16 | [III-5/III-3/III-4] |
| 1690 | II-16 | [III-5/III-3/III-5] |
| 1691 | II-16 | [III-5/III-4/III-1] |
| 1692 | II-16 | [III-5/III-4/III-2] |
| 1693 | II-16 | [III-5/III-4/III-3] |
| 1694 | II-16 | [III-5/III-4/III-4] |
| 1695 | II-16 | [III-5/III-4/III-5] |
| 1696 | II-16 | [III-5/III-5/III-1] |
| 1697 | II-16 | [III-5/III-5/III-2] |
| 1698 | II-16 | [III-5/III-5/III-3] |
| 1699 | II-16 | [III-5/III-5/III-4] |
| 1700 | II-16 | [III-5/III-5/III-5] |

19. An amine compound represented by Formula 1-1: 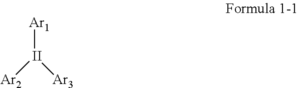
wherein in Formula 1-1, II is represented by II-A or II-B, and
Ar₁ to Ar₃ are each independently a substituted or unsubstituted benzoazole derivative: 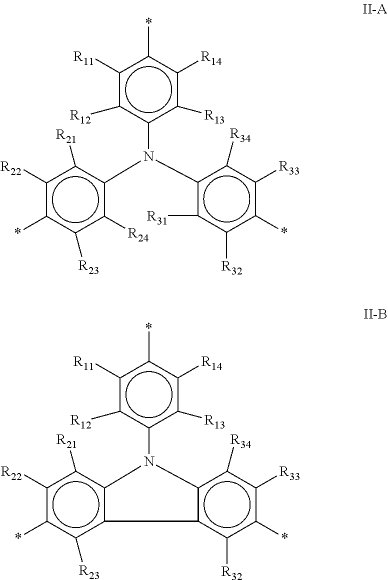
wherein in II-A, at least one selected from among R₁₁ to R₁₄, R₂₁ to R₂₄, and R₃₁ to R₃₄ is hydroxy and the R₁₁ to R₁₄, R₂₁ to R₂₄, and R₃₁ to R₃₄ that are not hydroxy are hydrogen atoms, and
in II-B, at least one selected from among R₁₁ to R₁₄, R₂₁ to R₂₃, and R₃₂ to R₃₄ is hydroxy and the R₁₁ to R₁₄, R₂₁ to R₂₃, and R₃₂ to R₃₄ that are not hydroxy are hydrogen atoms.

20. The amine compound of claim 19, wherein in Formula 1-1, II is represented by any one selected from among II-1 to II-16, and Ar₁ to Ar₃ are each independently represented by any one selected from among III-1 to III-5: 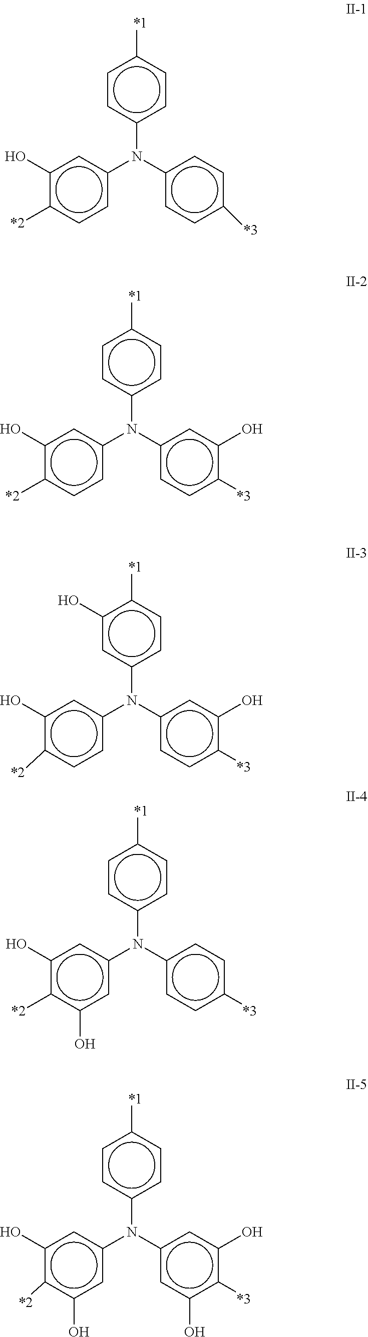 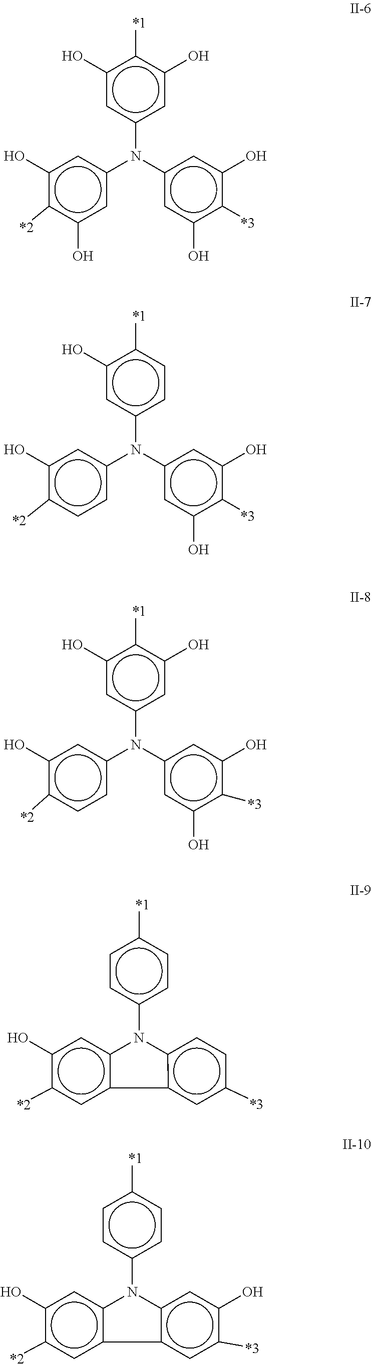 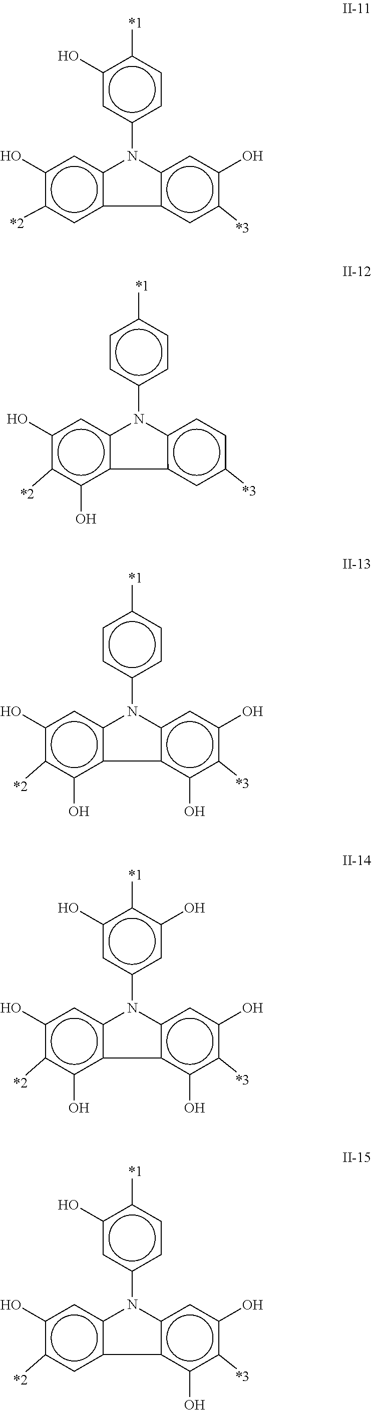 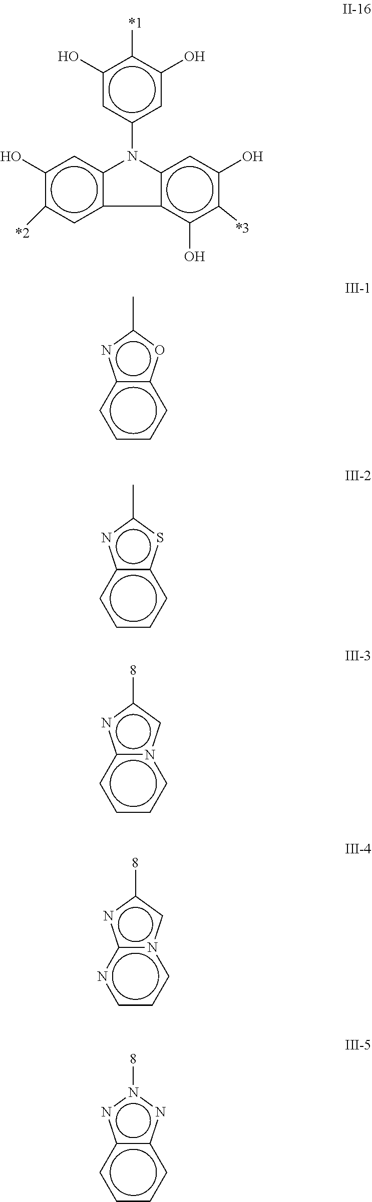
wherein in II-1 to II-16, *1 is a portion to which Ar₁ is bonded, *2 is a portion to which Ar₂ is bonded, and *3 is a portion to which Ar₃ is bonded.
